(19) United States
(12) Patent Application Publication
(10) Pub. No.: US 2022/0280608 A1
(43) Pub. Date: Sep. 8, 2022

(54) TREATMENT OF DIABETIC RETINOPATHY WITH FULLY-HUMAN POST-TRANSLATIONALLY MODIFIED ANTI-VEGF FAB

(71) Applicant: REGENXBIO INC., Rockville, MD (US)

(72) Inventors: Stephen Joseph Pakola, Irvington, NY (US); Sherri Van Everen, Menlo Park, CA (US); Jesse I. Yoo, Atlanta, GA (US); Samir Maganbhai Patel, Columbus, NJ (US); Avanti Arvind Ghanekar, St. Louis, MO (US); Anthony Ray O'Berry, Clarksburg, MD (US); Kim Rees Irwin-Pack, Milcreek, UT (US); Darin Thomas Curtiss, Potomac, MD (US)

(21) Appl. No.: 17/638,517

(22) PCT Filed: Aug. 25, 2020
(86) PCT No.: PCT/US2020/047733

§ 371 Date: Feb. 25, 2022

Related U.S. Application Data
(60) Provisional application No. 62/891,799, filed on Aug. 26, 2019.
(60) Provisional application No. 62/902,352, filed on Sep. 18, 2019.
(60) Provisional application No. 63/004,258, filed on Apr. 2, 2020.

(51) Int. Cl. A61K 38/18; A61P 27/02; A61B 5/01; C07K 16/22

(52) U.S. Cl. CPC A61K 38/1866; A61P 27/02; A61B 5/01; C07K 16/22; C07K 2317/41; C07K 2317/55

(57) ABSTRACT

Compositions and methods are described for the delivery of a fully human post-translaionally modified (HuPTM) monoclonal antibody (“mAh”) or the antigen-binding fragment of a mAh against human vascular endothelial growth factor (“hVEGF”)—such as, e.g., a fully human-glycosylated (HuGly) anti-hVEGF antigen-binding fragment—to the retina/vitreal humour in the eye(s) of human subjects diagnosed with diabetic retinopathy.

### CROSS-REFERNECE TO RELATED APPLICATIONS

This application claims the benefit of U.S. Provisional Application No. 62/891,799 filed Aug. 26, 2019, U.S. Provisional Application No. 62/902,352 filed Sep. 18, 2019 and U.S. Provisional Application No. 63/004,258 filed Apr. 2, 2020, the content of each of which is incorporated herein by reference in its entirety.

### REFERENCE TO SEQUENCE LISTING SUBMITTED ELECTRONICALLY

This application incorporates by reference a Sequence Listing submitted with this application as text file entitled “12656-127-228_Sequence_Listing.TXT” created on Aug. 12, 2020 and having a size of 97,447bytes.

### 1. INTRODUCTION

Compositions and methods are described for the delivery of a fully human post-translationally modified (HuPTM) monoclonal antibody (“mAb”) or the antigen-binding fragment of a mAb against vascular endothelial growth factor (“VEGF”)—such as, e.g., a fully human-glycosylated (HuGly) anti-VEGF antigen-binding fragment—to the retina/vitreal humour in the eye(s) of human subjects diagnosed with ocular diseases, in particular an ocular disease caused by increased neovascularization, for example, diabetic retinopathy (DR).

### 2. BACKGROUND OF THE INVENTION

Diabetic eye disease is a leading cause of visual impairment in working-age adults in the United States; the prevalence rate in adults with diabetes aged 40 and older is approximately 28.4% (4.2 million adults) (AAO PPP Retina/Vitreous Panel, Hoskins Center for Quality Eye Care, “Diabetic retinopathy PPP—Updated 2017”). Given the increasing rates of diabetes across the United States and other developed countries, the societal impact of diabetic retinopathy (DR) and the impact on blindness is expected to rise. Retina specialists recognize that they play a critical role in the prevention, diagnosis, and management of diabetic eye disease, which can often precede other systemic complications of diabetes mellitus. The potential to limit sight-threatening diabetic complications in the working-age population could have significant impact on public health.

Diabetic retinopathy is an ocular complication of diabetes, characterized by microaneurysms, hard exudates, hemorrhages, and venous abnormalities in the non-proliferative form and neovascularization, preretinal or vitreous hemorrhages, and fibrovascular proliferation in the proliferative form. Hyperglycemia induces microvascular retinal changes, leading to blurred vision, dark spots or flashing lights, and sudden loss of vision (Cai & McGinnis, 2016, Journal of Diabetes Research, Vol. 2016, Article ID 3789217).

Diabetic retinopathy ranges from mild nonproliferative disease to severe proliferative disease. The most common early clinically visible manifestations of nonproliferative diabetic retinopathy (NPDR) include microaneurysm formation and intraretinal hemorrhages. Microvascular damage leads to retinal capillary nonperfusion, cotton wool spots, increased numbers of hemorrhages, venous abnormalities, and intraretinal microvascular abnormalities. At any stage in the course of the disease, increased vasopermeability can result in retinal thickening (edema) and/or exudates that may lead to a loss in central visual acuity (VA). The proliferative diabetic retinopathy (PDR) stage results from closure of arterioles and venules with secondary proliferation of new vessels on the retina, optic disc, or anterior segment. Common complications of DR that risk a patient's vision and require either urgent medical or surgical intervention include center involved-diabetic macular edema (CI-DME), tractional retinal detachments, epiretinal membranes, and vitreous hemorrhage. The risk of these complications usually increases as the severity of DR increases, although DME can be present at any stage of DR (Aiello et al., 1994, N Engl J Med. 331(22):1480-1487). The link between diabetic ischemia and subsequent proliferation of angiogenic factors including vascular endothelial growth factor (VEGF) has been established.

In the landmark Early Treatment Diabetic Retinopathy Study (ETDRS) study from the 1990s, patients with baseline severe NPDR had an approximate 50% risk of progression to PDR and a 15% risk of developing high-risk PDR. Furthermore, for patients with very severe NPDR, their risk of worsening to high-risk PDR increases to 75% within 1 year. Given that the average age of patients in diabetic eye studies is around 50 years, avoiding conversion to PDR and its associated sight-threatening complications can improve patient quality of life for several decades. As a result, the decision about prophylactic treatment of NPDR and non high-risk PDR (mild to moderate PDR) is an ongoing discussion within the retina community.

### 3. SUMMARY OF THE INVENTION

Compositions and methods are described for the delivery of a fully human post-translationally modified (HuPTM) antibody against VEGF to the retina/vitreal humour in the eye(s) of patients (human subjects) diagnosed with an ocular disease, in particular an ocular disease caused by increased neovascularization, for example, diabetic retinopathy (DR). In certain aspects, described herein are compositions and methods for the subretinal administration of a fully human post-translationally modified (HuPTM) antibody against VEGF to the subretinal space in the eye(s) of patients (human subjects) diagnosed with diabetic retinopathy (DR). Antibodies include, but are not limited to, monoclonal antibodies, polyclonal antibodies, recombinantly produced antibodies, human antibodies, humanized antibodies, chimeric antibodies, synthetic antibodies, tetrameric antibodies comprising two heavy chain and two light chain molecules, antibody light chain monomers, antibody heavy chain monomers, antibody light chain dimers, antibody heavy chain dimers, antibody light chain-heavy chain pairs, intrabodies, heteroconjugate antibodies, monovalent antibodies, antigen-binding fragments of full-length antibodies, and fusion proteins of the above. Such antigen-binding fragments include, but are not limited to, single-domain antibodies (variable domain of heavy chain antibodies (VHHs) or nanobodies), Fabs, F(ab′)₂s, and scFvs (single-chain variable fragments) of full-length anti-VEGF antibodies (preferably, full-length anti-VEGF monoclonal antibodies (mAbs) (collectively referred to herein as “antigen-binding fragments”). In a preferred embodiment, the fully human post-translationally modified antibody against VEGF is a fully human post-translationally modified antigen-binding fragment of a monoclonal antibody (mAb) against VEGF (“HuPTMFabVEGFi”). In a further preferred embodiment, the HuPTMFabVEGFi is a fully human glycosylated antigen-binding fragment of an anti-VEGF mAb (“HuGlyFabVEGFi”). In an alternative embodiment, full-length mAbs can be used. In a preferred embodiment, delivery is accomplished via gene therapy—e.g., by administering a viral vector or other DNA expression construct encoding an anti-VEGF antigen-binding fragment or mAb (or a hyperglycosylated derivative (see, e.g., FIG. 3)) to the suprachoroidal space, subretinal space (from a transvitreal approach or with a catheter through the suprachoroidal space), intraretinal space, vitreous cavity, and/or outer surface of the sclera (i.e., juxtascleral administration) in the eye(s) of patients (human subjects) diagnosed with diabetic retinopathy (DR), to create a permanent depot in the eye that continuously supplies the human PTM, e.g., human-glycosylated, transgene product. In a preferred embodiment, the viral vector used for delivering the transgene should have a tropism for human retinal cells or photoreceptor cells. Such vectors can include non-replicating recombinant adeno-associated virus vectors (“rAAV”), particularly those bearing an AAV8 capsid are preferred. In a specific embodiment, the viral vector or other DNA expression construct described herein is Construct I, wherein the Construct I comprises the following components: (1) AAV8 inverted terminal repeats that flank the expression cassette; (2) control elements, which include a) the CB7 promoter, comprising the CMV enhancer/chicken β-actin promoter, b) a chicken β-actin intron and c) a rabbit β-globin poly A signal; and (3) nucleic acid sequences coding for the heavy and light chains of anti-VEGF antigen-binding fragment, separated by a self-cleaving furin (F)/F2A linker, ensuring expression of equal amounts of the heavy and the light chain polypeptides. In another specific embodiment, the viral vector or other DNA expression construct described herein is Construct II, wherein the Construct II comprise the following components: (1) AAV2 inverted terminal repeats that flank the expression cassette; (2) control elements, which include a) the CB7 promoter, comprising the CMV enhancer/chicken β-actin promoter, b) a chicken β-actin intron and c) a rabbit β-globin poly A signal; and (3) nucleic acid sequences coding for the heavy and light chains of anti-VEGF antigen-binding fragment, separated by a self-cleaving furin (F)/F2A linker, ensuring expression of equal amounts of the heavy and the light chain polypeptides.

In certain aspects, described herein are methods of treating a human subject diagnosed with diabetic retinopathy (DR), comprising delivering to the retina of said human subject a therapeutically effective amount of anti-hVEGF antigen-binding fragment produced by human retinal cells. In a specific aspect, described herein are methods of treating a human subject diagnosed with diabetic retinopathy (DR) comprising delivering to the retina of said human subject a therapeutically effective amount of anti-hVEGF antigen-binding fragment produced by human retinal cells, by administering to the suprachoroidal space, subretinal space(with vitrectomy, or without vitrectomy), intraretinal space, vitreous cavity, or outer surface of the sclera in the eye of said human subject (e.g., by suprachoroidal injection (for example, via a suprachoroidal drug delivery device such as a microinjector with a microneedle), subretinal injection via transvitreal approach (a surgical procedure), subretinal administration via the suprachoroidal space (for example, a surgical procedure via a subretinal drug delivery device comprising a catheter that can be inserted and tunneled through the suprachoroidal space toward the posterior pole, where a small needle injects into the subretinal space), or a posterior juxtascleral depot procedure (for example, via a juxtascleral drug delivery device comprising a cannula whose tip can be inserted and kept in direct apposition to the scleral surface)) an expression vector encoding the anti-hVEGF antigen-binding fragment. In a specific aspect, described herein are methods of treating diabetic retinopathy (DR), comprising delivering to the retina of said human subject a therapeutically effective amount of anti-hVEGF antigen-binding fragment produced by human retinal cells, by the use of a suprachoroidal drug delivery device such as a microinjector.

In certain aspects, described herein are methods of treating a human subject diagnosed with diabetic retinopathy (DR), comprising delivering to the retina of said human subject a therapeutically effective amount of anti-hVEGF antigen-binding fragment produced by human photoreceptor cells (e.g., cone cells and/or rod cells), horizontal cells, bipolar cells, amacrine cells, retina ganglion cells (e.g., midget cells, parasol cells, bistratified cells, giant retina ganglion cells, photosensitive ganglion cells, and/or Múller glia), and/or retinal pigment epithelial cells in the external limiting membrane. In a specific aspect, described herein are methods of treating a human subject diagnosed with diabetic retinopathy (DR), comprising delivering to the retina of said human subject a therapeutically effective amount of anti-hVEGF antigen-binding fragment produced by human photoreceptor cells (e.g., cone cells and/or rod cells), horizontal cells, bipolar cells, amacrine cells, retina ganglion cells (e.g., midget cells, parasol cells, bistratified cells, giant retina ganglion cells, photosensitive ganglion cells, and/or Múller glia), and/or retinal pigment epithelial cells in the external limiting membrane, by administering to the suprachoroidal space, subretinal space, intraretinal space, vitreous cavity, or outer surface of the sclera in the eye of said human subject (e.g., by suprachoroidal injection (for example, via a suprachoroidal drug delivery device such as a microinjector with a microneedle), subretinal injection via the transvitreal approach (a surgical procedure), subretinal administration via the suprachoroidal space (for example, a surgical procedure via a subretinal drug delivery device comprising a catheter that can be inserted and tunneled through the suprachoroidal space toward the posterior pole, where a small needle injects into the subretinal space), or a posterior juxtascleral depot procedure (for example, via a juxtascleral drug delivery device comprising a cannula whose tip can be inserted and kept in direct apposition to the scleral surface)) an expression vector encoding the anti-hVEGF antigen-binding fragment. In a specific aspect, described herein are methods of treating a human subject diagnosed with diabetic retinopathy (DR), comprising delivering to the retina of said human subject a therapeutically effective amount of anti-hVEGF antigen-binding fragment produced by human photoreceptor cells (e.g., cone cells and/or rod cells), horizontal cells, bipolar cells, amacrine cells, retina ganglion cells (e.g., midget cells, parasol cells, bistratified cells, giant retina ganglion cells, photosensitive ganglion cells, and/or Múller glia), and/or retinal pigment epithelial cells in the external limiting membrane, by the use of a suprachoroidal drug delivery device such as a microinjector.

In certain aspects, described herein are methods of treating a human subject diagnosed with diabetic retinopathy (DR), comprising delivering to the eye of said human subject a therapeutically effective amount of anti-hVEGF antigen-binding fragment produced by human retinal cells. In a specific aspect, described herein are methods of treating a human subject diagnosed with diabetic retinopathy (DR), comprising delivering to the eye of said human subject a therapeutically effective amount of anti-hVEGF antigen-binding fragment produced by human retinal cells, by administering to the suprachoroidal space, subretinal space, intraretinal space, vitreous cavity or outer surface of the sclera in the eye of said human subject (e.g., by suprachoroidal injection (for example, via a suprachoroidal drug delivery device such as a microinjector with a microneedle), subretinal injection via the transvitreal approach (a surgical procedure), subretinal administration via the suprachoroidal space (for example, a surgical procedure via a subretinal drug delivery device comprising a catheter that can be inserted and tunneled through the suprachoroidal space toward the posterior pole, where a small needle injects into the subretinal space), or a posterior juxtascleral depot procedure (for example, via a juxtascleral drug delivery device comprising a cannula whose tip can be inserted and kept in direct apposition to the scleral surface)) an expression vector encoding the anti-hVEGF antigen-binding fragment. In a specific aspect, described herein are methods of treating a human subject diagnosed with diabetic retinopathy (DR), comprising delivering to the eye of said human subject a therapeutically effective amount of anti-hVEGF antigen-binding fragment produced by human retinal cells, by the use of a suprachoroidal drug delivery device such as a microinjector.

In certain aspects, described herein are methods of treating a human subject diagnosed with diabetic retinopathy (DR), comprising delivering to the eye of said human subject a therapeutically effective amount of anti-hVEGF antigen-binding fragment produced by human photoreceptor cells (e.g., cone cells and/or rod cells), horizontal cells, bipolar cells, amacrine cells, retina ganglion cells (e.g., midget cells, parasol cells, bistratified cells, giant retina ganglion cells, photosensitive ganglion cells, and/or Múller glia), and/or retinal pigment epithelial cells in the external limiting membrane. In a specific aspect, described herein are methods of treating a human subject diagnosed with diabetic retinopathy (DR) , comprising delivering to the eye of said human subject a therapeutically effective amount of anti-hVEGF antigen-binding fragment produced by human photoreceptor cells (e.g., cone cells and/or rod cells), horizontal cells, bipolar cells, amacrine cells, retina ganglion cells (e.g., midget cells, parasol cells, bistratified cells, giant retina ganglion cells, photosensitive ganglion cells, and/or Múller glia), and/or retinal pigment epithelial cells in the external limiting membrane, by administering to the suprachoroidal space, subretinal space, intraretinal space, vitreous cavity or outer surface of the sclera in the eye of said human subject (e.g., by suprachoroidal injection (for example, via a suprachoroidal drug delivery device such as a microinjector with a microneedle), subretinal injection via the transvitreal approach (a surgical procedure), subretinal administration via the suprachoroidal space (for example, a surgical procedure via a subretinal drug delivery device comprising a catheter that can be inserted and tunneled through the suprachoroidal space toward the posterior pole, where a small needle injects into the subretinal space), or a posterior juxtascleral depot procedure (for example, via a juxtascleral drug delivery device comprising a cannula whose tip can be inserted and kept in direct apposition to the scleral surface)) an expression vector encoding the anti-hVEGF antigen-binding fragment. In a specific aspect, described herein are methods of treating a human subject diagnosed with diabetic retinopathy (DR), comprising delivering to the eye of said human subject a therapeutically effective amount of anti-hVEGF antigen-binding fragment produced by human photoreceptor cells (e.g., cone cells and/or rod cells), horizontal cells, bipolar cells, amacrine cells, retina ganglion cells (e.g., midget cells, parasol cells, bistratified cells, giant retina ganglion cells, photosensitive ganglion cells, and/or Múller glia), and/or retinal pigment epithelial cells in the external limiting membrane, by the use of a suprachoroidal drug delivery device such as a microinjector.

In certain aspects, described herein are methods of treating a human subject diagnosed with diabetic retinopathy (DR), comprising delivering to the eye of said human subject a therapeutically effective amount of anti-hVEGF antibody produced by human retinal cells. In a specific aspect, described herein are methods of treating a human subject diagnosed with diabetic retinopathy (DR), comprising delivering to the eye of said human subject a therapeutically effective amount of anti-hVEGF antibody produced by human retinal cells, by administering to the suprachoroidal space, subretinal space, intraretinal space, vitreous cavity or outer surface of the sclera in the eye of said human subject (e.g., by suprachoroidal injection (for example, via a suprachoroidal drug delivery device such as a microinjector with a microneedle), subretinal injection via the transvitreal approach (a surgical procedure), subretinal administration via the suprachoroidal space (for example, a surgical procedure via a subretinal drug delivery device comprising a catheter that can be inserted and tunneled through the suprachoroidal space toward the posterior pole, where a small needle injects into the subretinal space), or a posterior juxtascleral depot procedure (for example, via a juxtascleral drug delivery device comprising a cannula whose tip can be inserted and kept in direct apposition to the scleral surface)) an expression vector encoding the anti-hVEGF antibody.

In certain aspects, described herein are methods of treating a human subject diagnosed with retinopathy (DR), comprising delivering to the eye of said human subject a therapeutically effective amount of anti-hVEGF antibody produced by human photoreceptor cells (e.g., cone cells and/or rod cells), horizontal cells, bipolar cells, amacrine cells, retina ganglion cells (e.g., midget cells, parasol cells, bistratified cells, giant retina ganglion cells, photosensitive ganglion cells, and/or Múller glia), and/or retinal pigment epithelial cells in the external limiting membrane. In a specific aspect, described herein are methods of treating a human subject diagnosed with diabetic retinopathy (DR), comprising delivering to the eye of said human subject a therapeutically effective amount of anti-hVEGF antibody produced by human photoreceptor cells (e.g., cone cells and/or rod cells), horizontal cells, bipolar cells, amacrine cells, retina ganglion cells (e.g., midget cells, parasol cells, bistratified cells, giant retina ganglion cells, photosensitive ganglion cells, and/or Múller glia), and/or retinal pigment epithelial cells in the external limiting membrane, by administering to the suprachoroidal space, subretinal space, intraretinal space, vitreous cavity or outer surface of the sclera in the eye of said human subject (e.g., by suprachoroidal injection (for example, via a suprachoroidal drug delivery device such as a microinjector with a microneedle), subretinal injection via the transvitreal approach (a surgical procedure), subretinal administration via the suprachoroidal space (for example, a surgical procedure via a subretinal drug delivery device comprising a catheter that can be inserted and tunneled through the suprachoroidal space toward the posterior pole, where a small needle injects into the subretinal space), or a posterior juxtascleral depot procedure (for example, via a juxtascleral drug delivery device comprising a cannula whose tip can be inserted and kept in direct apposition to the scleral surface) an expression vector encoding the anti-hVEGF antibody.

In certain aspects, described herein are methods of treating a human subject diagnosed with diabetic retinopathy (DR), comprising delivering to the retina of said human subject a therapeutically effective amount of anti-hVEGF antibody produced by human retinal cells. In a specific aspect, described herein are methods of treating a human subject diagnosed with diabetic retinopathy (DR), comprising delivering to the retina of said human subject a therapeutically effective amount of anti-hVEGF antibody produced by human retinal cells, by administering to the suprachoroidal space, subretinal space, intraretinal space, vitreous cavity or outer surface of the sclera in the eye of said human subject (e.g., by suprachoroidal injection (for example, via a suprachoroidal drug delivery device such as a microinjector with a microneedle), subretinal injection via the transvitreal approach (a surgical procedure), subretinal administration via the suprachoroidal space (for example, a surgical procedure via a subretinal drug delivery device comprising a catheter that can be inserted and tunneled through the suprachoroidal space toward the posterior pole, where a small needle injects into the subretinal space), or a posterior juxtascleral depot procedure (for example, via a juxtascleral drug delivery device comprising a cannula whose tip can be inserted and kept in direct apposition to the scleral surface)) an expression vector encoding the anti-hVEGF antibody.

In certain aspects, described herein are methods of treating a human subject diagnosed with diabetic retinopathy (DR), comprising delivering to the retina of said human subject a therapeutically effective amount of anti-hVEGF antibody produced by human photoreceptor cells (e.g., cone cells and/or rod cells), horizontal cells, bipolar cells, amacrine cells, retina ganglion cells (e.g., midget cells, parasol cells, bistratified cells, giant retina ganglion cells, photosensitive ganglion cells, and/or Múller glia), and/or retinal pigment epithelial cells in the external limiting membrane. In a specific aspect, described herein are methods of treating a human subject diagnosed with diabetic retinopathy (DR), comprising delivering to the retina of said human subject a therapeutically effective amount of anti-hVEGF antibody produced by human photoreceptor cells (e.g., cone cells and/or rod cells), horizontal cells, bipolar cells, amacrine cells, retina ganglion cells (e.g., midget cells, parasol cells, bistratified cells, giant retina ganglion cells, photosensitive ganglion cells, and/or Múller glia), and/or retinal pigment epithelial cells in the external limiting membrane, by administering to the suprachoroidal space, subretinal space, intraretinal space, vitreous cavity or outer surface of the sclera in the eye of said human subject (e.g., by suprachoroidal injection (for example, via a suprachoroidal drug delivery device such as a microinjector with a microneedle), subretinal injection via the transvitreal approach (a surgical procedure), subretinal administration via the suprachoroidal space (for example, a surgical procedure via a subretinal drug delivery device comprising a catheter that can be inserted and tunneled through the suprachoroidal space toward the posterior pole, where a small needle injects into the subretinal space), or a posterior juxtascleral depot procedure (for example, via a juxtascleral drug delivery device comprising a cannula whose tip can be inserted and kept in direct apposition to the scleral surface)) an expression vector encoding the anti-hVEGF antibody.

In a specific aspect, the method comprises performing a vitrectomy on the eye of said human patient. In a specific aspect, the vitrectomy is a partial vitrectomy.

In a specific aspect, the administering step is by injecting the recombinant viral vector into the vitreous cavity using an intravitreal drug delivery device. In a specific aspect, the intravitreal drug delivery device is a microinjector.

Described herein are anti-human vascular endothelial growth factor (hVEGF) antibodies, for example, anti-hVEGF antigen-binding fragments, produced by human retinal cells. Human VEGF (hVEGF) is a human protein encoded by the VEGF (VEGFA, VEGFB, VEGFC, or VEGFD) gene. An exemplary amino acid sequence of hVEGF may be found at GenBank Accession No. AAA35789.1. An exemplary nucleic acid sequence of hVEGF may be found at GenBank Accession No. M32977.1.

In certain aspects of the methods described herein, the antigen-binding fragment comprises a heavy chain comprising the amino acid sequence of SEQ ID NO. 2 or SEQ ID NO. 4, and a light chain comprising the amino acid sequence of SEQ ID NO. 1, or SEQ ID NO. 3.

In certain aspects of the methods described herein, the antigen-binding fragment comprises light chain CDRs 1-3 of SEQ ID NOs: 14-16 and heavy chain CDRs 1-3 of SEQ ID NOs:17-19 or SEQ ID NOs: 20, 18, and 21.

In a specific embodiment of the methods described herein, the antigen-binding fragment comprises light chain CDRs 1-3 of SEQ ID NOs: 14-16 and heavy chain CDRs 1-3 of SEQ ID NOs: 20, 18, and 21, wherein the second amino acid residue of the light chain CDR3 (i.e., the second Q in QQYSTVPWTF (SEQ ID NO. 16)) does not carry one or more of the following chemical modifications: oxidation, acetylation, deamidation, and pyroglutamation (pyro Glu). In a specific embodiment, the antigen-binding fragment comprises light chain CDRs 1-3 of SEQ ID NOs: 14-16 and heavy chain CDRs 1-3 of SEQ ID NOs: 20, 18, and 21, wherein the eighth and eleventh amino acid residues of the light chain CDR1 (i.e., the two Ns in SASQDISNYLN (SEQ ID NO. 14) each carries one or more of the following chemical modifications: oxidation, acetylation, deamidation, and pyroglutamation (pyro Glu), and the second amino acid residue of the light chain CDR3 (i.e., the second Q in QQYSTVPWTF (SEQ ID NO. 16)) does not carry one or more of the following chemical modifications: oxidation, acetylation, deamidation, and pyroglutamation (pyro Glu). In a specific embodiment, the antigen-binding fragment comprises light chain CDRs 1-3 of SEQ ID NOs: 14-16 and heavy chain CDRs 1-3 of SEQ ID NOs: 20, 18, and 21, wherein the second amino acid residue of the light chain CDR3 (i.e., the second Q in QQYSTVPWTF (SEQ ID NO. 16)) is not acetylated. In a specific embodiment, the antigen-binding fragment comprises light chain CDRs 1-3 of SEQ ID NOs: 14-16 and heavy chain CDRs 1-3 of SEQ ID NOs: 20, 18, and 21, wherein the eighth and eleventh amino acid residues of the light chain CDR1 (i.e., the two Ns in SASQDISNYLN (SEQ ID NO. 14) each carries one or more of the following chemical modifications: oxidation, acetylation, deamidation, and pyroglutamation (pyro Glu), and the second amino acid residue of the light chain CDR3 (i.e., the second Q in QQYSTVPWTF (SEQ ID NO. 16)) is not acetylated. In a preferred embodiment, the chemical modification(s) or lack of chemical modification(s) (as the case may be) described herein is determined by mass spectrometry.

In a specific embodiment of the methods described herein, the antigen-binding fragment comprises light chain CDRs 1-3 of SEQ ID NOs: 14-16 and heavy chain CDRs 1-3 of SEQ ID NOs: 20, 18, and 21, wherein the last amino acid residue of the heavy chain CDR1 (i.e., the N in GYDFTHYGMN (SEQ ID NO. 20)) does not carry one or more of the following chemical modifications: oxidation, acetylation, deamidation, and pyroglutamation (pyro Glu). In a specific embodiment, the antigen-binding fragment comprises light chain CDRs 1-3 of SEQ ID NOs: 14-16 and heavy chain CDRs 1-3 of SEQ ID NOs: 20, 18, and 21, wherein the ninth amino acid residue of the heavy chain CDR1 (i.e., the M in GYDFTHYGMN (SEQ ID NO. 20)) carries one or more of the following chemical modifications: acetylation, deamidation, and pyroglutamation (pyro Glu), the third amino acid residue of the heavy chain CDR2 (i.e., the N in WINTYTGEPTYAADFKR (SEQ ID NO. 18) carries one or more of the following chemical modifications: acetylation, deamidation, and pyroglutamation (pyro Glu), and the last amino acid residue of the heavy chain CDR1 (i.e., the N in GYDFTHYGMN (SEQ ID NO. 20)) does not carry one or more of the following chemical modifications: oxidation, acetylation, deamidation, and pyroglutamation (pyro Glu). In a specific embodiment, the antigen-binding fragment comprises light chain CDRs 1-3 of SEQ ID NOs: 14-16 and heavy chain CDRs 1-3 of SEQ ID NOs: 20, 18, and 21, wherein the last amino acid residue of the heavy chain CDR1 (i.e., the N in GYDFTHYGMN (SEQ ID NO. 20)) is not acetylated. In a specific embodiment, the antigen-binding fragment comprises light chain CDRs 1-3 of SEQ ID NOs: 14-16 and heavy chain CDRs 1-3 of SEQ ID NOs: 20, 18, and 21, wherein the ninth amino acid residue of the heavy chain CDR1 (i.e., the M in GYDFTHYGMN (SEQ ID NO. 20)) carries one or more of the following chemical modifications: acetylation, deamidation, and pyroglutamation (pyro Glu), the third amino acid residue of the heavy chain CDR2 (i.e., the N in WINTYTGEPTYAADFKR (SEQ ID NO. 18) carries one or more of the following chemical modifications: acetylation, deamidation, and pyroglutamation (pyro Glu), and the last amino acid residue of the heavy chain CDR1 (i.e., the N in GYDFTHYGMN (SEQ ID NO. 20)) is not acetylated. In a preferred embodiment, the chemical modification(s) or lack of chemical modification(s) (as the case may be) described herein is determined by mass spectrometry.

In a specific embodiment of the methods described herein, the antigen-binding fragment comprises light chain CDRs 1-3 of SEQ ID NOs: 14-16 and heavy chain CDRs 1-3 of SEQ ID NOs: 20, 18, and 21, wherein the last amino acid residue of the heavy chain CDR1 (i.e., the N in GYDFTHYGMN (SEQ ID NO. 20)) does not carry one or more of the following chemical modifications: oxidation, acetylation, deamidation, and pyroglutamation (pyro Glu), and the second amino acid residue of the light chain CDR3 (i.e., the second Q in QQYSTVPWTF (SEQ ID NO. 16)) does not carry one or more of the following chemical modifications: oxidation, acetylation, deamidation, and pyroglutamation (pyro Glu). In a specific embodiment, the antigen-binding fragment comprises light chain CDRs 1-3 of SEQ ID NOs: 14-16 and heavy chain CDRs 1-3 of SEQ ID NOs: 20, 18, and 21, wherein: (1) the ninth amino acid residue of the heavy chain CDR1 (i.e., the M in GYDFTHYGMN (SEQ ID NO. 20)) carries one or more of the following chemical modifications: acetylation, deamidation, and pyroglutamation (pyro Glu), the third amino acid residue of the heavy chain CDR2 (i.e., the N in WINTYTGEPTYAADFKR (SEQ ID NO. 18) carries one or more of the following chemical modifications: acetylation, deamidation, and pyroglutamation (pyro Glu), and the last amino acid residue of the heavy chain CDR1 (i.e., the N in GYDFTHYGMN (SEQ ID NO. 20)) does not carry one or more of the following chemical modifications: oxidation, acetylation, deamidation, and pyroglutamation (pyro Glu); and (2) the eighth and eleventh amino acid residues of the light chain CDR1 (i.e., the two Ns in SASQDISNYLN (SEQ ID NO. 14) each carries one or more of the following chemical modifications: oxidation, acetylation, deamidation, and pyroglutamation (pyro Glu), and the second amino acid residue of the light chain CDR3 (i.e., the second Q in QQYSTVPWTF (SEQ ID NO. 16)) does not carry one or more of the following chemical modifications: oxidation, acetylation, deamidation, and pyroglutamation (pyro Glu). In a specific embodiment, the antigen-binding fragment comprises light chain CDRs 1-3 of SEQ ID NOs: 14-16 and heavy chain CDRs 1-3 of SEQ ID NOs: 20, 18, and 21, wherein the last amino acid residue of the heavy chain CDR1 (i.e., the N in GYDFTHYGMN (SEQ ID NO. 20)) is not acetylated, and the second amino acid residue of the light chain CDR3 (i.e., the second Q in QQYSTVPWTF (SEQ ID NO. 16)) is not acetylated. In a specific embodiment, the antigen-binding fragment comprises light chain CDRs 1-3 of SEQ ID NOs: 14-16 and heavy chain CDRs 1-3 of SEQ ID NOs: 20, 18, and 21, wherein: (1) the ninth amino acid residue of the heavy chain CDR1 (i.e., the M in GYDFTHYGMN (SEQ ID NO. 20)) carries one or more of the following chemical modifications: acetylation, deamidation, and pyroglutamation (pyro Glu), the third amino acid residue of the heavy chain CDR2 (i.e., the N in WINTYTGEPTYAADFKR (SEQ ID NO. 18) carries one or more of the following chemical modifications: acetylation, deamidation, and pyroglutamation (pyro Glu), and the last amino acid residue of the heavy chain CDR1 (i.e., the N in GYDFTHYGMN (SEQ ID NO. 20)) is not acetylated; and (2) the eighth and eleventh amino acid residues of the light chain CDR1 (i.e., the two Ns in SASQDISNYLN (SEQ ID NO. 14) each carries one or more of the following chemical modifications: oxidation, acetylation, deamidation, and pyroglutamation (pyro Glu), and the second amino acid residue of the light chain CDR3 (i.e., the second Q in QQYSTVPWTF (SEQ ID NO. 16)) is not acetylated. In a preferred embodiment, the chemical modification(s) or lack of chemical modification(s) (as the case may be) described herein is determined by mass spectrometry.

In certain aspects, described herein are methods of treating a human subject diagnosed with diabetic retinopathy (DR), comprising: delivering to the eye of said human subject, a therapeutically effective amount of an antigen-binding fragment of a mAb against hVEGF, said antigen-binding fragment containing a α2,6-sialylated glycan. In a specific aspect, described herein are methods of treating a human subject diagnosed with diabetic retinopathy (DR), comprising: delivering to the eye of said human subject, a therapeutically effective amount of an antigen-binding fragment of a mAb against hVEGF, said antigen-binding fragment containing a α2,6-sialylated glycan, by administering to the suprachoroidal space, subretinal space, intraretinal space, vitreous cavity or outer surface of the sclera in the eye of said human subject (e.g., by suprachoroidal injection (for example, via a suprachoroidal drug delivery device such as a microinjector with a microneedle), subretinal injection via the transvitreal approach (a surgical procedure), subretinal administration via the suprachoroidal space (for example, a surgical procedure via a subretinal drug delivery device comprising a catheter that can be inserted and tunneled through the suprachoroidal space toward the posterior pole, where a small needle injects into the subretinal space), or a posterior juxtascleral depot procedure (for example, via a juxtascleral drug delivery device comprising a cannula whose tip can be inserted and kept in direct apposition to the scleral surface)) an expression vector encoding the antigen-binding fragment of a mAb against hVEGF.

In certain aspects, described herein are methods of treating a human subject diagnosed with diabetic retinopathy (DR), comprising: delivering to the eye of said human subject, a therapeutically effective amount of a glycosylated antigen-binding fragment of a mAb against hVEGF, wherein said antigen-binding fragment does not contain detectable NeuGc and/or α-Gal antigen (i.e., as used herein, “detectable” means levels detectable by standard assays described infra). In a specific embodiment, described herein are methods of treating a human subject diagnosed with diabetic retinopathy (DR), comprising: delivering to the eye of said human subject, a therapeutically effective amount of a glycosylated antigen-binding fragment of a mAb against hVEGF, by administering to the suprachoroidal space, subretinal space, intraretinal space, vitreous cavity, or outer surface of the sclera in the eye of said human subject (e.g., by suprachoroidal injection (for example, via a suprachoroidal drug delivery device such as a microinjector with a microneedle), subretinal injection via the transvitreal approach (a surgical procedure), subretinal administration via the suprachoroidal space (for example, a surgical procedure via a subretinal drug delivery device comprising a catheter that can be inserted and tunneled through the suprachoroidal space toward the posterior pole, where a small needle injects into the subretinal space), or a posterior juxtascleral depot procedure (for example, via a juxtascleral drug delivery device comprising a cannula whose tip can be inserted and kept in direct apposition to the scleral surface)) an expression vector encoding the glycosylated antigen-binding fragment of a mAb against hVEGF, wherein said antigen-binding fragment does not contain detectable NeuGc and/or α-Gal antigen.

In certain aspects, described herein are methods of treating a human subject diagnosed with diabetic retinopathy (DR), wherein the method comprises: administering to the suprachoroidal space, subretinal space, intraretinal space, vitreous cavity, or outer surface of the sclera in the eye of said human subject an expression vector encoding an antigen-binding fragment of a mAb against hVEGF (e.g., by suprachoroidal injection, subretinal injection via the transvitreal approach (a surgical procedure), subretinal administration via the suprachoroidal space, or a posterior juxtascleral depot procedure), wherein expression of said antigen-binding fragment is α2,6-sialylated upon expression from said expression vector in a human, immortalized retina-derived cell.

In certain aspects, described herein are methods of treating a human subject diagnosed with diabetic retinopathy (DR), wherein the method comprises: administering or delivering to the retina of said human subject via the suprachoroidal space in the eye of said human subject (e.g., via a suprachoroidal drug delivery device such as a microinjector with a microneedle) an expression vector encoding an antigen-binding fragment of a mAb against hVEGF, wherein expression of said antigen-binding fragment is α2,6-sialylated upon expression from said expression vector in a human, immortalized retina-derived cell.

In certain aspects, described herein are methods of treating a human subject diagnosed with retinopathy (DR), wherein the method comprises: administering to the subretinal and/or intraretinal space of said human subject via the suprachoroidal space in the eye of said human subject (e.g., via a subretinal drug delivery device comprising a catheter that can be inserted and tunneled through the suprachoroidal space) an expression vector encoding an antigen-binding fragment of a mAb against hVEGF, wherein expression of said antigen-binding fragment is α2,6-sialylated upon expression from said expression vector in a human, immortalized retina-derived cell. In certain aspects, described herein are methods of treating a human subject diagnosed with diabetic retinopathy (DR), wherein the method comprises: administering to the suprachoroidal space, subretinal space, intraretinal space, vitreous cavity, or outer surface of the sclera in the eye of said human subject an expression vector encoding an antigen-binding fragment against hVEGF (e.g., by suprachoroidal injection, subretinal injection via the transvitreal approach (a surgical procedure), subretinal administration via the suprachoroidal space, or a posterior juxtascleral depot procedure), wherein expression of said antigen-binding fragment is α2,6-sialylated upon expression from said expression vector in a human, immortalized retina-derived cell, wherein said antigen-binding fragment does not contain detectable NeuGc and/or α-Gal antigen.

In certain aspects, described herein are methods of treating a human subject diagnosed with diabetic retinopathy (DR), wherein the method comprises: administering or delivering to the retina of said human subject via the suprachoroidal space in the eye of said human subject (e.g., via a suprachoroidal drug delivery device such as a microinjector with a microneedle) an expression vector encoding an antigen-binding fragment against hVEGF, wherein expression of said antigen-binding fragment is α2,6-sialylated upon expression from said expression vector in a human, immortalized retina-derived cell, wherein said antigen-binding fragment does not contain detectable NeuGc and/or α-Gal antigen.

In certain aspects, described herein are methods of treating a human subject diagnosed with diabetic retinopathy (DR), wherein the method comprises: administering to the subretinal space and/or intraretinal of said human subject via the suprachoroidal space in the eye of said human subject (e.g., via a subretinal drug delivery device comprising a catheter that can be inserted and tunneled through the suprachoroidal space toward the posterior pole, where a small needle injects into the subretinal space) an expression vector encoding an antigen-binding fragment against hVEGF, wherein expression of said antigen-binding fragment is α2,6-sialylated upon expression from said expression vector in a human, immortalized retina-derived cell, wherein said antigen-binding fragment does not contain detectable NeuGc and/or α-Gal antigen.

In certain aspects, described herein are methods of treating a human subject diagnosed with diabetic retinopathy (DR), comprising: administering to the suprachoroidal space, subretinal space, intraretinal space, vitreous cavity, or outer surface of the sclera in the eye of said human subject, a therapeutically effective amount of a recombinant nucleotide expression vector encoding an antigen-binding fragment of a mAb against hVEGF (e.g. by suprachoroidal injection, subretinal injection via the transvitreal approach (a surgical procedure), subretinal administration via the suprachoroidal space, or a posterior juxtascleral depot procedure), so that a depot is formed that releases said antigen-binding fragment containing a α2,6-sialylated glycan.

In certain aspects, described herein are methods of treating a human subject diagnosed with diabetic retinopathy (DR), comprising: administering or delivering to the retina of said human subject via the suprachoroidal space in the eye of said human subject (e.g., via a suprachoroidal drug delivery device such as a microinjector with a microneedle), a therapeutically effective amount of a recombinant nucleotide expression vector encoding an antigen-binding fragment of a mAb against hVEGF, so that a depot is formed that releases said antigen-binding fragment containing a α2,6-sialylated glycan.

In certain aspects, described herein are methods of treating a human subject diagnosed with diabetic retinopathy (DR), comprising: administering to the subretinal and/or intraretinal space of said human subject via the suprachoroidal space in the eye of said human subject (e.g., via a subretinal drug delivery device comprising a catheter that can be inserted and tunneled through the suprachoroidal space toward the posterior pole, where a small needle injects into the subretinal space), a therapeutically effective amount of a recombinant nucleotide expression vector encoding an antigen-binding fragment of a mAb against hVEGF, so that a depot is formed that releases said antigen-binding fragment containing a α2,6-sialylated glycan.

In certain aspects, described herein are methods of treating a human subject diagnosed with diabetic retinopathy (DR), comprising: administering to the suprachoroidal space, subretinal space, intraretinal space, vitreous cavity, or outer surface of the sclera in the eye of said human subject, a therapeutically effective amount of a recombinant nucleotide expression vector encoding an antigen-binding fragment of a mAb against hVEGF (e.g., by suprachoroidal injection, subretinal injection via the transvitreal approach (a surgical procedure), subretinal administration via the suprachoroidal space, or a posterior juxtascleral depot procedure), so that a depot is formed that releases said antigen-binding fragment wherein said antigen-binding fragment is glycosylated but does not contain detectable NeuGc and/or α-Gal antigen.

In certain aspects, described herein are methods of treating a human subject diagnosed with diabetic retinopathy (DR), comprising: administering or delivering to the retina of said human subject via the suprachoroidal space in the eye of said human subject (e.g., via a suprachoroidal drug delivery device such as a microinjector with a microneedle), a therapeutically effective amount of a recombinant nucleotide expression vector encoding an antigen-binding fragment of a mAb against hVEGF, so that a depot is formed that releases said antigen-binding fragment wherein said antigen-binding fragment is glycosylated but does not contain detectable NeuGc and/or α-Gal antigen.

In certain aspects, described herein are methods of treating a human subject diagnosed with diabetic retinopathy (DR), comprising: administering to the subretinal and/or intraretinal space of said human subject via the suprachoroidal space in the eye of said human subject (e.g., via a subretinal drug delivery device comprising a catheter that can be inserted and tunneled through the suprachoroidal space toward the posterior pole, where a small needle injects into the subretinal space), a therapeutically effective amount of a recombinant nucleotide expression vector encoding an antigen-binding fragment of a mAb against hVEGF, so that a depot is formed that releases said antigen-binding fragment wherein said antigen-binding fragment is glycosylated but does not contain detectable NeuGc and/or α-Gal antigen. In certain aspects, described herein are methods of treating a human subject diagnosed with diabetic retinopathy (DR), comprising administering to the subretinal space and/or intraretinal space of said human subject via the suprachoroidal space in the eye of said human subject an expression vector encoding an anti-human vascular endothelial growth factor (hVEGF) antibody. In a specific aspect, the expression vector is administered via subretinal delivery in a single dose about 1.6×10¹¹ GC/eye at a concentration of 6.4×10¹¹ GC/mL or about 2.5×10¹¹ GC/eye at a concentration of 1.0×10¹² GC/mL.

In certain aspects, described herein are methods of treating a human subject diagnosed with diabetic retinopathy (DR), comprising: administering to the subretinal and/or intraretinal space of said human subject via the suprachoroidal space in the eye of said human subject (e.g., via a subretinal drug delivery device comprising a catheter that can be inserted and tunneled through the suprachoroidal space toward the posterior pole, where a small needle injects into the subretinal space), a therapeutically effective amount of a recombinant nucleotide expression vector encoding an antigen-binding fragment of a mAb against hVEGF, so that a depot is formed that releases said antigen-binding fragment wherein said antigen-binding fragment is glycosylated but does not contain detectable NeuGc and/or α-Gal antigen. In certain aspects, described herein are methods of treating a human subject diagnosed with diabetic retinopathy (DR), comprising administering to the subretinal and/or intraretinal space of said human subject via the suprachoroidal space in the eye of said human subject an expression vector encoding an anti-human vascular endothelial growth factor (hVEGF) antibody. In a specific aspect, the expression vector is administered via subretinal delivery in a single dose about 1.6×10¹¹ GC/eye at a concentration of 6.2×10¹¹ GC/mL or about 2.5×10¹¹ GC/eye at a concentration of 1.0×10¹² GC/mL. In a specific aspect, the expression vector is administered via subretinal delivery in a single dose about 1.55×10¹¹ GC/eye at a concentration of 6.2×10¹¹ GC/mL or about 2.5×10¹¹ GC/eye at a concentration of 1.0×10¹² GC/mL.

In a specific aspect, the anti-hVEGF antibody comprises a heavy chain comprising the amino acid sequence of SEQ ID NO. 2 or SEQ ID NO. 4, and a light chain comprising the amino acid sequence of SEQ ID NO. 1, or SEQ ID NO. 3. In a specific aspect, the expression vector is an AAV8 vector.

In certain aspects of the methods described herein, the antigen-binding fragment transgene encodes a leader peptide. A leader peptide may also be referred to as a signal peptide or leader sequence herein.

In certain aspects, described herein are methods of treating a human subject diagnosed with diabetic retinopathy (DR), comprising: administering to the suprachoroidal space, subretinal space, intraretinal space, vitreous cavity, or outer surface of the sclera in the eye of said human subject, a therapeutically effective amount of a recombinant nucleotide expression vector encoding an antigen-binding fragment of a mAb against hVEGF (e.g., by suprachoroidal injection, subretinal injection via the transvitreal approach (a surgical procedure), subretinal administration via the suprachoroidal space, or a posterior juxtascleral depot procedure)), so that a depot is formed that releases said antigen-binding fragment containing a α2,6-sialylated glycan; wherein said recombinant vector, when used to transduce PER.C6 or RPE cells in culture results in production of said antigen-binding fragment containing a α2,6-sialylated glycan in said cell culture.

In certain aspects, described herein are methods of treating a human subject diagnosed with diabetic retinopathy (DR) (in particular, wet AMD), comprising: administering or delivering to the retina of said human subject via the suprachoroidal space in the eye of said human subject (e.g., via a suprachoroidal drug delivery device such as a microinjector with a microneedle), a therapeutically effective amount of a recombinant nucleotide expression vector encoding an antigen-binding fragment of a mAb against hVEGF, so that a depot is formed that releases said antigen-binding fragment containing a α2,6-sialylated glycan; wherein said recombinant vector, when used to transduce PER.C6 or RPE cells in culture results in production of said antigen-binding fragment containing a α2,6-sialylated glycan in said cell culture.

In certain aspects, described herein are methods of treating a human subject diagnosed with diabetic retinopathy (DR), comprising: administering to the subretinal and/or intraretinal space of said human subject via the suprachoroidal space in the eye of said human subject (e.g., via a subretinal drug delivery device comprising a catheter that can be inserted and tunneled through the suprachoroidal space toward the posterior pole, where a small needle injects into the subretinal space), a therapeutically effective amount of a recombinant nucleotide expression vector encoding an antigen-binding fragment of a mAb against hVEGF, so that a depot is formed that releases said antigen-binding fragment containing a α2,6-sialylated glycan; wherein said recombinant vector, when used to transduce PER.C6 or RPE cells in culture results in production of said antigen-binding fragment containing a α2,6-sialylated glycan in said cell culture.

In certain aspects, described herein are methods of treating a human subject diagnosed with diabetic retinopathy (DR), comprising: administering to the suprachoroidal space, subretinal space, intraretinal space, vitreous cavity, or outer surface of the sclera in the eye of said human subject, a therapeutically effective amount of a recombinant nucleotide expression vector encoding an antigen-binding fragment of a mAb against hVEGF (e.g., by suprachoroidal injection, subretinal injection via the transvitreal approach (a surgical procedure), subretinal administration via the suprachoroidal space, or a posterior juxtascleral depot procedure), so that a depot is formed that releases said antigen-binding fragment wherein said antigen-binding fragment is glycosylated but does not contain detectable NeuGc and/or α-Gal antigen; wherein said recombinant vector, when used to transduce PER.C6 or RPE cells in culture results in production of said antigen-binding fragment that is glycosylated but does not contain detectable NeuGc and/or α-Gal antigen in said cell culture.

In certain aspects, described herein are methods of treating a human subject diagnosed with diabetic retinopathy (DR), comprising: administering to the subretinal and/or intraretinal space of said human subject via the suprachoroidal space in the eye of said human subject (e.g., via a subretinal drug delivery device comprising a catheter that can be inserted and tunneled through the suprachoroidal space toward the posterior pole, where a small needle injects into the subretinal space), a therapeutically effective amount of a recombinant nucleotide expression vector encoding an antigen-binding fragment of a mAb against hVEGF, so that a depot is formed that releases said antigen-binding fragment wherein said antigen-binding fragment is glycosylated but does not contain detectable NeuGc and/or α-Gal antigen; wherein said recombinant vector, when used to transduce PER.C6 or RPE cells in culture results in production of said antigen-binding fragment that is glycosylated but does not contain detectable NeuGc and/or α-Gal antigen in said cell culture.

In certain aspects, described herein are methods of treating a human subject diagnosed with diabetic retinopathy (DR), comprising: administering to the subretinal and/or intraretinal space of said human subject via the suprachoroidal space in the eye of said human subject (e.g., via a subretinal drug delivery device comprising a catheter that can be inserted and tunneled through the suprachoroidal space toward the posterior pole, where a small needle injects into the subretinal space), a therapeutically effective amount of a recombinant nucleotide expression vector encoding an antigen-binding fragment of a mAb against hVEGF, so that a depot is formed that releases said antigen-binding fragment wherein said antigen-binding fragment is glycosylated but does not contain detectable NeuGc and/or α-Gal antigen; wherein said recombinant vector, when used to transduce PER.C6 or RPE cells in culture results in production of said antigen-binding fragment that is glycosylated but does not contain detectable NeuGc and/or α-Gal antigen in said cell culture.

In certain aspects of the methods described herein, the human subject has a Best-corrected visual acuity (BCVA) of >69 ETDRS letters (approximate Snellen equivalent 20/40 or better).

In certain aspects of the methods described herein, the BCVA is the BCVA in the eye to be treated in the human subject.

In certain aspects of the methods described herein, delivering to the eye comprises delivering to the retina, choroid, and/or vitreous humor of the eye. In certain aspects of the methods described herein, the antigen-binding fragment comprises a heavy chain that comprises one, two, three, or four additional amino acids at the C-terminus.

Subjects to whom such gene therapy is administered should be those responsive to anti-VEGF therapy. In particular embodiments, the methods encompass treating patients who have been diagnosed with retinopathy (DR) and identified as responsive to treatment with an anti-VEGF antibody. In more specific embodiments, the patients are responsive to treatment with an anti-VEGF antigen-binding fragment. In certain embodiments, the patients have been shown to be responsive to treatment with an anti-VEGF antigen-binding fragment injected intravitreally prior to treatment with gene therapy. In specific embodiments, the patients have previously been treated with LUCENTIS® (ranibizumab), EYLEA® (aflibercept), and/or AVASTIN® (bevacizumab), and have been found to be responsive to one or more of said LUCENTIS (ranibizumab), EYLEA® (aflibercept), and/or AVASTIN® (bevacizumab).

Subjects to whom such viral vector or other DNA expression construct is delivered should be responsive to the anti-hVEGF antigen-binding fragment encoded by the transgene in the viral vector or expression construct. To determine responsiveness, the anti-VEGF antigen-binding fragment transgene product (e.g., produced in cell culture, bioreactors, etc.) may be administered directly to the subject, such as by intravitreal injection.

In certain aspects of the methods described herein, the antigen-binding fragment comprises a heavy chain that does not comprise an additional amino acid at the C-terminus.

In certain aspects of the methods described herein produces a population of antigen-binding fragment molecules, wherein the antigen-binding fragment molecules comprise a heavy chain, and wherein 0.5%, 1%, 2%, 3%, 4%, 5%, 10%, or 20%, or less of the population of antigen-binding fragment molecules comprises one, two, three, or four additional amino acids at the C-terminus of the heavy chain. In certain aspects of the methods described herein produces a population of antigen-binding fragment molecules, wherein the antigen-binding fragment molecules comprise a heavy chain, and wherein 0.5%, 1%, 2%, 3%, 4%, 5%, 10%, or 20%, or less but more than 0% of the population of antigen-binding fragment molecules comprises one, two, three, or four additional amino acids at the C-terminus of the heavy chain.

In certain aspects of the methods described herein produces a population of antigen-binding fragment molecules, wherein the antigen-binding fragment molecules comprise a heavy chain, and wherein 0.5-1%, 0.5%-2%, 0.5%-3%, 0.5%-4%, 0.5%-5%, 0.5%-10%, 0.5%-20%, 1%-2%, 1%-3%, 1%-4%, 1%-5%, 1%-10%, 1%-20%, 2%-3%, 2%-4%, 2%-5%, 2%-10%, 2%-20%, 3%-4%, 3%-5%, 3%-10%, 3%-20%, 4%-5%, 4%-10%, 4%-20%, 5%-10%, 5%-20%, or 10%-20% of the population of antigen-binding fragment molecules comprises one, two, three, or four additional amino acids at the C-terminus of the heavy chain.

The HuPTMFabVEGFi, e.g., HuGlyFabVEGFi, encoded by the transgene can include, but is not limited to an antigen-binding fragment of an antibody that binds to hVEGF, such as bevacizumab; an anti-hVEGF Fab moiety such as ranibizumab; or such bevacizumab or ranibizumab Fab moieties engineered to contain additional glycosylation sites on the Fab domain (e.g., see Courtois et al., 2016, mAbs 8: 99-112 which is incorporated by reference herein in its entirety for it description of derivatives of bevacizumab that are hyperglycosylated on the Fab domain of the full length antibody).

The recombinant vector used for delivering the transgene should have a tropism for human retinal cells or photoreceptor cells. Such vectors can include non-replicating recombinant adeno-associated virus vectors (“rAAV”), particularly those bearing an AAV8 capsid are preferred. However, other viral vectors may be used, including but not limited to lentiviral vectors, vaccinia viral vectors, or non-viral expression vectors referred to as “naked DNA” constructs. Preferably, the HuPTMFabVEGFi, e.g., HuGlyFabVEGFi, transgene should be controlled by appropriate expression control elements, for example, the CB7 promoter (a chicken β-actin promoter and CMV enhancer), the RPE65 promoter, or opsin promoter to name a few, and can include other expression control elements that enhance expression of the transgene driven by the vector (e.g., introns such as the chicken β-actin intron, minute virus of mice (MVM) intron, human factor IX intron (e.g., FIX truncated intron 1), β-globin splice donor/immunoglobulin heavy chain spice acceptor intron, adenovirus splice donor/immunoglobulin splice acceptor intron, SV40 late splice donor/splice acceptor (19S/16S) intron, and hybrid adenovirus splice donor/IgG splice acceptor intron and polyA signals such as the rabbit β-globin polyA signal, human growth hormone (hGH) polyA signal, SV40 late polyA signal, synthetic polyA (SPA) signal, and bovine growth hormone (bGH) polyA signal). See, e.g., Powell and Rivera-Soto, 2015, Discov. Med., 19(102):49-57.

Gene therapy constructs are designed such that both the heavy and light chains are expressed. More specifically, the heavy and light chains should be expressed at about equal amounts, in other words, the heavy and light chains are expressed at approximately a 1:1 ratio of heavy chains to light chains. The coding sequences for the heavy and light chains can be engineered in a single construct in which the heavy and light chains are separated by a cleavable linker or IRES so that separate heavy and light chain polypeptides are expressed. See, e.g., Section 5.2.4 for specific leader sequences and Section 5.2.5 for specific IRES, 2A, and other linker sequences that can be used with the methods and compositions provided herein.

In certain embodiments, gene therapy constructs are supplied as a frozen sterile, single use solution of the AAV vector active ingredient in a formulation buffer. In a specific embodiment, the pharmaceutical compositions suitable for subretinal administration comprise a suspension of the recombinant (e.g., rHuGlyFabVEGFi) vector in a formulation buffer comprising a physiologically compatible aqueous buffer, a surfactant and optional excipients. In a specific embodiment, the construct is formulated in Dulbecco's phosphate buffered saline and 0.001% Pluronic F68, pH=7.4.

In certain embodiments, gene therapy constructs are supplied as a frozen sterile, single use solution of the AAV vector active ingredient in a formulation buffer. In a specific embodiment, the pharmaceutical compositions suitable for suprachoroidal, subretinal, juxtascleral, intravitreal, subconjunctival, and/or intraretinal administration comprise a suspension of the recombinant (e.g., rHuGlyFabVEGFi) vector in a formulation buffer comprising a physiologically compatible aqueous buffer, a surfactant and optional excipients.

Therapeutically effective doses of the recombinant vector should be administered subretinally and/or intraretinally (e.g., by subretinal injection via the transvitreal approach (a surgical procedure), or subretinal administration via the suprachoroidal space) in a volume ranging from ≥0.1 mL to ≤0.5 mL, preferably in 0.1 to 0.30 mL (100-300 μl), and most preferably, in a volume of 0.25 mL (250 μl). Therapeutically effective doses of the recombinant vector should be administered suprachoroidally (e.g., by suprachoroidal injection) in a volume of 100 μl or less, for example, in a volume of 50-100 μl. Therapeutically effective doses of the recombinant vector should be administered to the outer surface of the sclera (e.g., by a posterior juxtascleral depot procedure) in a volume of 500 μl or less, for example, in a volume of 10-20 μl, 20-50 μl, 50-100 μl, 100-200 μl, 200-300 μl, 300-400 μl, or 400-500 μl. Subretinal injection is a surgical procedure performed by trained retinal surgeons that involves a vitrectomy with the subject under local anesthesia, and subretinal injection of the gene therapy into the retina (see, e.g., Campochiaro et al., 2017, Hum Gen Ther 28(1):99-111, which is incorporated by reference herein in its entirety). In a specific embodiment, the subretinal administration is performed via the suprachoroidal space using a suprachoroidal catheter which injects drug into the subretinal space, such as a subretinal drug delivery device that comprises a catheter which can be inserted and tunneled through the suprachoroidal space to the posterior pole, where a small needle injects into the subretinal space (see, e.g., Baldassarre et al., 2017, Subretinal Delivery of Cells via the Suprachoroidal Space: Janssen Trial. In: Schwartz et al. (eds) Cellular Therapies for Retinal Disease, Springer, Cham; International Patent Application Publication No. WO 2016/040635 A1; each of which is incorporated by reference herein in its entirety). Suprachoroidal administration procedures involve administration of a drug to the suprachoroidal space of the eye, and are normally performed using a suprachoroidal drug delivery device such as a microinjector with a microneedle (see, e.g., Hariprasad, 2016, Retinal Physician 13: 20-23; Goldstein, 2014, Retina Today 9(5): 82-87; each of which is incorporated by reference herein in its entirety). The suprachoroidal drug delivery devices that can be used to deposit the expression vector in the suprachoroidal space according to the invention described herein include, but are not limited to, suprachoroidal drug delivery devices manufactured by Clearside® Biomedical, Inc. (see, for example, Hariprasad, 2016, Retinal Physician 13: 20-23) and MedOne suprachoroidal catheters. The subretinal drug delivery devices that can be used to deposit the expression vector in the subretinal space via the suprachoroidal space according to the invention described herein include, but are not limited to, subretinal drug delivery devices manufactured by Janssen Pharmaceuticals, Inc. (see, for example, International Patent Application Publication No. WO 2016/040635 A1). In a specific embodiment, administration to the outer surface of the sclera is performed by a juxtascleral drug delivery device comprising a cannula whose tip can be inserted and kept in direct apposition to the scleral surface. See Section 5.3.2 for more details of the different modes of administration. Suprachoroidal, subretinal, juxtascleral, intravitreal, subconjunctival, and/or intraretinal administration should result in delivery of the soluble transgene product to the retina, the vitreous humor, and/or the aqueous humor. The expression of the transgene product (e.g., the encoded anti-VEGF antibody) by retinal cells, e.g., rod, cone, retinal pigment epithelial, horizontal, bipolar, amacrine, ganglion, and/or Múller cells, results in delivery and maintenance of the transgene product in the retina, the vitreous humor, and/or the aqueous humor. In a specific embodiment, doses that maintain a concentration of the transgene product at a Cmin of at least 0.330 pg/mL in the Vitreous humour, or 0.110 pg/mL in the Aqueous humour (the anterior chamber of the eye) for three months are desired; thereafter, Vitreous Cmin concentrations of the transgene product ranging from 1.70 to 6.60 pg/mL, and/or Aqueous Cmin concentrations ranging from 0.567 to 2.20 pg/mL should be maintained. However, because the transgene product is continuously produced, maintenance of lower concentrations can be effective. The concentration of the transgene product can be measured in patient samples of the vitreous humour and/or aqueous from the anterior chamber of the treated eye. Alternatively, vitreous humour concentrations can be estimated and/or monitored by measuring the patient's serum concentrations of the transgene product—the ratio of systemic to vitreal exposure to the transgene product is about 1:90,000. (E.g., see, vitreous humor and serum concentrations of ranibizumab reported in Xu L, et al., 2013, Invest. Opthal. Vis. Sci. 54: 1616-1624, at p. 1621 and Table 5 at p. 1623, which is incorporated by reference herein in its entirety).

In a specific embodiment, the subretinal administration is performed with a subretinal drug delivery device that comprises the micro volume injector delivery system, which is manufactured by Altaviz (see FIGS. 9A and 9B) (see, e.g. International Patent Application Publication No. WO 2013/177215, United States Patent Application Publication No. 2019/0175825, and United States Patent Application Publication No. 2019/0167906) that can be used for any administration route described herein for eye administration. The micro volume injector delivery system may include a gas-powered module providing high force delivery and improved precision, as described in United States Patent Application Publication No. 2019/0175825 and United States Patent Application Publication No. 2019/0167906. In addition, the micro volume injector delivery system may include a hydraulic drive for providing a consistent dose rate, and a low-force activation lever for controlling the gas-powered module and, in turn, the fluid delivery. In certain embodiment, the micro volume injector delivery system can be used for micro volume injector is a micro volume injector with dose guidance and can be used with, for example, a suprachoroidal needle (for example, the Clearside® needle), a subretinal needle, an intravitreal needle, a juxtascleral needle, a subconjunctival needle, and/or intraretinal needle. The benefits of using micro volume injector include: (a) more controlled delivery (for example, due to having precision injection flow rate control and dose guidance), (b) single surgeon, single hand, one finger operation; (c) pneumatic drive with 10 μL increment dosage; (d) divorced from the vitrectomy machine; (e) 400 μL syringe dose; (f) digitally guided delivery; (g) digitally recorded delivery; and (h) agnostic tip (for example, the MedOne 38 g needle and the Dorc 41 g needle can be used for subretinal delivery, while the Clearside® needle and the Visionisti OY adaptor can be used for subretinal delivery).

In certain embodiments of the methods described herein, the recombinant vector is administered suprachoroidally (e.g., by suprachoroidal injection). In a specific embodiment, suprachoroidal administration (e.g., an injection into the suprachoroidal space) is performed using a suprachoroidal drug delivery device. Suprachoroidal drug delivery devices are often used in suprachoroidal administration procedures, which involve administration of a drug to the suprachoroidal space of the eye (see, e.g., Hariprasad, 2016, Retinal Physician 13: 20-23; Goldstein, 2014, Retina Today 9(5): 82-87; Baldassarre et al., 2017; each of which is incorporated by reference herein in its entirety). The suprachoroidal drug delivery devices that can be used to deposit the recombinant vector in the suprachoroidal space according to the invention described herein include, but are not limited to, suprachoroidal drug delivery devices manufactured by Clearside® Biomedical, Inc. (see, for example, Hariprasad, 2016, Retinal Physician 13: 20-23) and MedOne suprachoroidal catheters. In another embodiment, the suprachoroidal drug delivery device that can be used in accordance with the methods described herein comprises the micro volume injector delivery system, which is manufactured by Altaviz (see FIGS. 9A and 9B) (see, e.g. International Patent Application Publication No. WO 2013/177215, United States Patent Application Publication No. 2019/0175825, and United States Patent Application Publication No. 2019/0167906) that can be used for any administration route described herein for eye administration. The micro volume injector delivery system may include a gas-powered module providing high force delivery and improved precision, as described in United States Patent Application Publication No. 2019/0175825 and United States Patent Application Publication No. 2019/0167906. In addition, the micro volume injector delivery system may include a hydraulic drive for providing a consistent dose rate, and a low-force activation lever for controlling the gas-powered module and, in turn, the fluid delivery. The micro volume injector is a micro volume injector with dose guidance and can be used with, for example, a suprachoroidal needle (for example, the Clearside® needle) or a subretinal needle. The benefits of using micro volume injector include: (a) more controlled delivery (for example, due to having precision injection flow rate control and dose guidance), (b) single surgeon, single hand, one finger operation; (c) pneumatic drive with 10 μL increment dosage; (d) divorced from the vitrectomy machine; (e) 400 μL syringe dose; (f) digitally guided delivery; (g) digitally recorded delivery; and (h) agnostic tip (for example, the MedOne 38g needle and the Dorc 41 g needle can be used for subretinal delivery, while the Clearside® needle and the Visionisti OY adaptor can be used for suprachoroidal delivery). In another embodiment, the suprachoroidal drug delivery device that can be used in accordance with the methods described herein is a tool that comprises a normal length hypodermic needle with an adaptor (and preferably also a needle guide) manufactured by Visionisti OY, which adaptor turns the normal length hypodermic needle into a suprachoroidal needle by controlling the length of the needle tip exposing from the adapter (see FIG. 8) (see, for example, U.S. Design Pat. No. D878,575; and International Patent Application. Publication No. WO/2016/083669) In a specific embodiment, the suprachoroidal drug delivery device is a syringe with a 1 millimeter 30 gauge needle (see FIG. 5). During an injection using this device, the needle pierces to the base of the sclera and fluid containing drug enters the suprachoroidal space, leading to expansion of the suprachoroidal space. As a result, there is tactile and visual feedback during the injection. Following the injection, the fluid flows posteriorly and absorbs dominantly in the choroid and retina. This results in the production of therapeutic product from all retinal cell layers and choroidal cells. Using this type of device and procedure allows for a quick and easy in-office procedure with low risk of complications. A max volume of 100 μl can be injected into the suprachoroidal space.

In a specific embodiment, the intravitreal administration is performed with a intravitreal drug delivery device that comprises the micro volume injector delivery system, which is manufactured by Altaviz. (see FIGS. 9A and 9B) (see, e.g. International Patent Application Publication No. WO 2013/177215), United States Patent Application Publication No. 2019/0175825, and United States Patent Application Publication No. 2019/0167906) that can be used for any administration route described herein for eye administration. The micro volume injector delivery system may include a gas-powered module providing high force delivery and improved precision, as described in United States Patent Application Publication No. 2019/0175825 and United States Patent Application Publication No. 2019/0167906. In addition, the micro volume injector delivery system may include a hydraulic drive for providing a consistent dose rate, and a low-force activation lever for controlling the gas-powered module and, in turn, the fluid delivery. The micro volume injector is a micro volume injector with dose guidance and can be used with, for example, a intravitreal needle. The benefits of using micro volume injector include: (a) more controlled delivery (for example, due to having precision injection flow rate control and dose guidance), (b) single surgeon, single hand, one finger operation; (c) pneumatic drive with 10 μL increment dosage; (d) divorced from the vitrectomy machine; (e) 400 μL syringe dose; (f) digitally guided delivery; (g) digitally recorded delivery; and (h) agnostic tip.

In a specific embodiment, the juxtascleral administration is performed with a juxtascleral drug delivery device that comprises the micro volume injector delivery system, which is manufactured by Altaviz. (see FIGS. 9A and 9B) (see, e.g. International Patent Application Publication No. WO 2013/177215) , United States Patent Application Publication No. 2019/0175825, and United States Patent Application Publication No. 2019/0167906) that can be used for any administration route described herein for eye administration. The micro volume injector delivery system may include a gas-powered module providing high force delivery and improved precision, as described in United States Patent Application Publication No. 2019/0175825 and United States Patent Application Publication No. 2019/0167906. In addition, the micro volume injector delivery system may include a hydraulic drive for providing a consistent dose rate, and a low-force activation lever for controlling the gas-powered module and, in turn, the fluid delivery. Micro volume injector is a micro volume injector with dose guidance and can be used with, for example, a subretinal needle. The benefits of using micro volume injector include: (a) more controlled delivery (for example, due to having precision injection flow rate control and dose guidance), (b) single surgeon, single hand, one finger operation; (c) pneumatic drive with 10 μL increment dosage; (d) divorced from the vitrectomy machine; (e) 400 μL syringe dose; (f) digitally guided delivery; (g) digitally recorded delivery; and (h) agnostic tip .

In certain embodiments, dosages are measured by genome copies per ml or the number of genome copies administered to the eye of the patient (e.g., by suprachoroidal injection (for example, via a suprachoroidal drug delivery device such as a microinjector with a microneedle), subretinal injection via the transvitreal approach (a surgical procedure), or subretinal administration via the suprachoroidal space). In certain embodiments, 2.4×10¹¹ genome copies per ml to 1×10¹³ genome copies per ml are administered. In a specific embodiment, 2.4×10¹¹ genome copies per ml to 5×10¹¹ genome copies per ml are administered. In another specific embodiment, 5×10¹¹ genome copies per ml to 1×10¹² genome copies per ml are administered. In another specific embodiment, 1×10¹² genome copies per ml to 5×10¹² genome copies per ml are administered. In another specific embodiment, 5×10¹² genome copies per ml to 1×10¹³ genome copies per ml are administered. In another specific embodiment, about 2.4×10¹¹ genome copies per ml are administered. In another specific embodiment, about 5×10¹¹ genome copies per ml are administered. In another specific embodiment, about 1×10¹² genome copies per ml are administered. In another specific embodiment, about 5×10¹² genome copies per ml are administered. In another specific embodiment, about 1×10¹³ genome copies per ml are administered. In certain embodiments, 1×10⁹ to 1×10¹² genome copies are administered. In specific embodiments, 3×10⁹ to 2.5×10¹¹ genome copies are administered. In specific embodiments, 1×10⁹ to 2.5×10¹¹ genome copies are administered. In specific embodiments, 1×10⁹ to 1×10¹¹ genome copies are administered. In specific embodiments, 1×10⁹ to 5×10⁹ genome copies are administered. In specific embodiments, 6×10⁹ to 3×10¹⁰ genome copies are administered. In specific embodiments, 4 x 10¹⁰ to 1×10¹¹ genome copies are administered. In specific embodiments, 2×10¹¹ to 1×10¹² genome copies are administered. In a specific embodiment, about 3×10⁹ genome copies are administered (which corresponds to about 1.2×10¹⁰ genome copies per ml in a volume of 250 μl). In another specific embodiment, about 1×10¹⁰ genome copies are administered (which corresponds to about 4×10¹⁰ genome copies per ml in a volume of 250 μl). In another specific embodiment, about 6×10¹⁰ genome copies are administered (which corresponds to about 2.4×10¹¹ genome copies per ml in a volume of 250 μl). In another specific embodiment, about 1.6×10¹¹ genome copies are administered (which corresponds to about 6.2×10¹¹ genome copies per ml in a volume of 250 μl). In another specific embodiment, about 1.6×10¹¹ genome copies are administered (which corresponds to about 6.4×10¹¹ genome copies per ml in a volume of 250 μl). In another specific embodiment, about 1.55×10¹¹ genome copies are administered (which corresponds to about 6.2×10¹¹ genome copies per ml in a volume of 250 μl). In another specific embodiment, about 2.5×10¹¹ genome copies (which corresponds to about 1.0×10¹² in a volume of 250 μl) are administered.

In certain embodiments, about 3.0×10¹³ genome copies per eye are administered. In certain embodiments, up to 3.0×10¹³ genome copies per eye are administered.

In certain embodiments, about 6.0×10¹⁰ genome copies per eye are administered. In certain embodiments, about 1.6×10¹¹ genome copies per eye are administered. In certain embodiments, about 2.5×10¹¹ genome copies per eye are administered. In certain embodiments, about 5.0×10¹¹ genome copies per eye are administered. In certain embodiments, about 3×10¹² genome copies per eye are administered. In certain embodiments, about 1.0×10¹² genome copies per ml per eye are administered. In certain embodiments, about 2.5×10¹² genome copies per ml per eye are administered.

In certain embodiments, about 6.0×10¹⁰ genome copies per eye are administered by subretinal injection. In certain embodiments, about 1.6×10¹¹ genome copies per eye are administered by subretinal injection. In certain embodiments, about 2.5×10¹¹ genome copies per eye are administered by subretinal injection. In certain embodiments, about 3.0×10¹³ genome copies per eye are administered by subretinal injection. In certain embodiments, up to 3.0×10¹³ genome copies per eye are administered by subretinal injection.

In certain embodiments, about 2.5×10¹¹ genome copies per eye are administered by suprachoroidal injection. In certain embodiments, about 5.0×10¹¹ genome copies per eye are administered by suprachoroidal injection. In certain embodiments, about 3×10¹² genome copies per eye are administered by suprachoroidal injection. In certain embodiments, about 2.5×10¹¹ genome copies per eye are administered by a single suprachoroidal injection. In certain embodiments, about 5.0×10¹¹ genome copies per eye are administered by double suprachoroidal injections. In certain embodiments, about 3.0×10¹³ genome copies per eye are administered by suprachoroidal injection. In certain embodiments, up to 3.0×10¹³ genome copies per eye are administered by suprachoroidal injection. In certain embodiments, about 2.5×10¹² genome copies per ml per eye are administered by a single suprachoroidal injection in a volume of 100 μl. In certain embodiments, about 2.5×10¹² genome copies per ml per eye are administered by double suprachoroidal injections, wherein each injection is in a volume of 100

As used herein and unless otherwise specified, the term “about” means within plus or minus 10% of a given value or range. In certain embodiments, the term “about” encompasses the exact number recited.

The invention has several advantages over standard of care treatments that involve repeated ocular injections of high dose boluses of the VEGF inhibitor that dissipate over time resulting in peak and trough levels. Sustained expression of the transgene product antibody, as opposed to injecting an antibody repeatedly, allows for a more consistent levels of antibody to be present at the site of action, and is less risky and more convenient for patients, since fewer injections need to be made, resulting in fewer doctor visits. Consistent protein production may leads to better clinical outcomes as edema rebound in the retina is less likely to occur. Furthermore, antibodies expressed from transgenes are post-translationally modified in a different manner than those that are directly injected because of the different microenvironment present during and after translation. Without being bound by any particular theory, this results in antibodies that have different diffusion, bioactivity, distribution, affinity, pharmacokinetic, and immunogenicity characteristics, such that the antibodies delivered to the site of action are “biobetters” in comparison with directly injected antibodies.

In addition, antibodies expressed from transgenes in vivo are not likely to contain degradation products associated with antibodies produced by recombinant technologies, such as protein aggregation and protein oxidation. Aggregation is an issue associated with protein production and storage due to high protein concentration, surface interaction with manufacturing equipment and containers, and purification with certain buffer systems. These conditions, which promote aggregation, do not exist in transgene expression in gene therapy. Oxidation, such as methionine, tryptophan, and histidine oxidation, is also associated with protein production and storage, and is caused by stressed cell culture conditions, metal and air contact, and impurities in buffers and excipients. The proteins expressed from transgenes in vivo may also oxidize in a stressed condition. However, humans, and many other organisms, are equipped with an antioxidation defense system, which not only reduces the oxidation stress, but sometimes also repairs and/or reverses the oxidation. Thus, proteins produced in vivo are not likely to be in an oxidized form. Both aggregation and oxidation could affect the potency, pharmacokinetics (clearance), and immunogenicity.

Without being bound by theory, the methods and compositions provided herein are based, in part, on the following principles:
(i) Human retinal cells are secretory cells that possess the cellular machinery for post-translational processing of secreted proteins—including glycosylation and tyrosine-O-sulfation, a robust process in retinal cells. (See, e.g., Wang et al., 2013, Analytical Biochem. 427: 20-28 and Adamis et al., 1993, BBRC 193: 631-638 reporting the production of glycoproteins by retinal cells; and Kanan et al., 2009, Exp. Eye Res. 89: 559-567 and Kanan & Al-Ubaidi, 2015, Exp. Eye Res. 133: 126-131 reporting the production of tyrosine-sulfated glycoproteins secreted by retinal cells, each of which is incorporated by reference in its entirety for post-translational modifications made by human retinal cells).
(ii) Contrary to the state of the art understanding, anti-VEGF antigen-binding fragments, such as ranibizumab (and the Fab domain of full length anti-VEGF mAbs such as bevacizumab) do indeed possess N-linked glycosylation sites. For example, see FIG. 1 which identifies non-consensus asparaginal (“N”) glycosylation sites in the C_{H} domain (TVSWN¹⁶⁵SGAL) and in the C_{L} domain (QSGN¹⁵⁸SQE), as well as glutamine (“Q”) residues that are glycosylation sites in the V_{H} domain (Q¹¹⁵GT) and V_{L} domain (TFQ¹⁰⁰GT) of ranibizumab (and corresponding sites in the Fab of bevacizumab). (See, e.g., Valliere-Douglass et al., 2009, J. Biol. Chem. 284: 32493-32506, and Valliere-Douglass et al., 2010, J. Biol. Chem. 285: 16012-16022, each of which is incorporated by reference in its entirety for the identification of N-linked glycosylation sites in antibodies).
(iii) While such non-canonical sites usually result in low level glycosylation (e.g., about 1-5%) of the antibody population, the functional benefits may be significant in immunoprivileged organs, such as the eye (See, e.g., van de Bovenkamp et al., 2016, J. Immunol. 196:1435-1441). For example, Fab glycosylation may affect the stability, half-life, and binding characteristics of an antibody. To determine the effects of Fab glycosylation on the affinity of the antibody for its target, any technique known to one of skill in the art may be used, for example, enzyme linked immunosorbent assay (ELISA), or surface plasmon resonance (SPR). To determine the effects of Fab glycosylation on the half-life of the antibody, any technique known to one of skill in the art may be used, for example, by measurement of the levels of radioactivity in the blood or organs (e.g., the eye) in a subject to whom a radiolabelled antibody has been administered. To determine the effects of Fab glycosylation on the stability, for example, levels of aggregation or protein unfolding, of the antibody, any technique known to one of skill in the art may be used, for example, differential scanning calorimetry (DSC), high performance liquid chromatography (HPLC), e.g., size exclusion high performance liquid chromatography (SEC-HPLC), capillary electrophoresis, mass spectrometry, or turbidity measurement. Provided herein, the HuPTMFabVEGFi, e.g., HuGlyFabVEGFi, transgene results in production of a Fab which is 0.5%, 1%, 2%, 3%, 4%, 5%, 6%, 7%, 8%, 9%, or 10% or more glycosylated at non-canonical sites. In certain embodiments, 0.5%, 1%, 2%, 3%, 4%, 5%, 6%, 7%, 8%, 9%, or 10% or more Fabs from a population of Fabs are glycosylated at non-canonical sites. In certain embodiments, 0.5%, 1%, 2%, 3%, 4%, 5%, 6%, 7%, 8%, 9%, or 10% or more non-canonical sites are glycosylated. In certain embodiments, the glycosylation of the Fab at these non-canonical sites is 25%, 50%, 100%, 200%, 300%, 400%, 500%, or more greater than the amount of glycosylation of these non-canonical sites in a Fab produced in HEK293 cells.
(iv) In addition to the glycosylation sites, anti-VEGF Fabs such as ranibizumab (and the Fab of bevacizumab) contain tyrosine (“Y”) sulfation sites in or near the CDRs; see FIG. 1 which identifies tyrosine-O-sulfation sites in the VH (EDTAVY⁹⁴Y⁹⁵) and V_{L} (EDFATY⁸⁶) domains of ranibizumab (and corresponding sites in the Fab of bevacizumab). (See, e.g., Yang et al., 2015, Molecules 20:2138-2164, esp. at p. 2154 which is incorporated by reference in its entirety for the analysis of amino acids surrounding tyrosine residues subjected to protein tyrosine sulfation. The “rules” can be summarized as follows: Y residues with E or D within +5 to −5 position of Y, and where position −1 of Y is a neutral or acidic charged amino acid—but not a basic amino acid, e.g., R, K, or H that abolishes sulfation). Human IgG antibodies can manifest a number of other post-translational modifications, such as N-terminal modifications, C-terminal modifications, degradation or oxidation of amino acid residues, cysteine related variants, and glycation (See, e.g., Liu etal., 2014, mAbs 6(5):1145-1154).
(v) Glycosylation of anti-VEGF Fabs, such as ranibizumab or the Fab fragment of bevacizumab by human retinal cells will result in the addition of glycans that can improve stability, half-life and reduce unwanted aggregation and/or immunogenicity of the transgene product. (See, e.g., Bovenkamp etal., 2016, J. Immunol. 196: 1435-1441 for a review of the emerging importance of Fab glycosylation). Significantly, glycans that can be added to HuPTMFabVEGFi, e.g., HuGlyFabVEGFi, provided herein, are highly processed complex-type biantennary N-glycans that contain 2,6-sialic acid (e.g., see FIG. 2 depicting the glycans that may be incorporated into HuPTMFabVEGFi, e.g., HuGlyFabVEGFi) and bisecting GlcNAc, but not NGNA (N-Glycolylneuraminic acid, Neu5Gc). Such glycans are not present in ranibizumab (which is made in E. coli and is not glycosylated at all) or in bevacizumab (which is made in CHO cells that do not have the 2,6-sialyltransferase required to make this post-translational modification, nor do CHO cells product bisecting GlcNAc, although they do add Neu5Gc (NGNA) as sialic acid not typical (and potentially immunogenic) to humans instead of Neu5Ac (NANA)). See, e.g., Dumont et al., 2015, Crit. Rev. Biotechnol. (Early Online, published online September 18, 2015, pp. 1-13 at p. 5). Moreover, CHO cells can also produce an immunogenic glycan, the α-Gal antigen, which reacts with anti-α-Gal antibodies present in most individuals, and at high concentrations can trigger anaphylaxis. See, e.g., Bosques, 2010, Nat Biotech 28: 1153-1156. The human glycosylation pattern of the HuPTMFabVEGFi, e.g., HuGlyFabVEGFi, provided herein, should reduce immunogenicity of the transgene product and improve efficacy.
(vi) Tyrosine-sulfation of anti-VEGF Fabs, such as ranibizumab or the Fab fragment of bevacizumab—a robust post-translational process in human retinal cells—could result in transgene products with increased avidity for VEGF. Indeed, tyrosine-sulfation of the Fab of therapeutic antibodies against other targets has been shown to dramatically increase avidity for antigen and activity. (See, e.g., Loos et al., 2015, PNAS 112: 12675-12680, and Choe et al., 2003, Cell 114: 161-170). Such post-translational modifications are not present on ranibizumab (which is made in E. coli a host that does not possess the enzymes required for tyrosine-sulfation), and at best is under-represented in bevacizumab—a CHO cell product. Unlike human retinal cells, CHO cells are not secretory cells and have a limited capacity for post-translational tyrosine-sulfation. (See, e.g., Mikkelsen & Ezban, 1991, Biochemistry 30: 1533-1537, esp. discussion at p. 1537).

For the foregoing reasons, the production of HuPTMFabVEGFi, e.g., HuGlyFabVEGFi, should result in a “biobetter” molecule for the treatment of diabetic retinopathy (DR) accomplished via gene therapy—e.g., by administering a viral vector or other DNA expression construct encoding HuPTMFabVEGFi, e.g., HuGlyFabVEGFi, to the suprachoroidal space, subretinal space, intraretinal space, vitreous cavity, or the outer surface of the sclera in the eye(s) of patients (human subjects) diagnosed with diabetic retinopathy (DR) (e.g., by suprachoroidal injection (for example, via a suprachoroidal drug delivery device such as a microinjector with a microneedle), subretinal injection via the transvitreal approach (a surgical procedure), subretinal administration via the suprachoroidal space, or a posterior juxtascleral depot procedure), to create a permanent depot in the eye that continuously supplies the fully-human post-translationally modified, e.g., human-glycosylated, sulfated transgene product produced by transduced retinal cells. The cDNA construct for the FabVEGFi should include a signal peptide that ensures proper co- and post-translational processing (glycosylation and protein sulfation) by the transduced retinal cells. Such signal sequences used by retinal cells may include but are not limited to:

(VEGF-A signal peptide)
(SEQ ID NO: 5)
MNFLLSWVHW SLALLLYLHH AKWSQA
(Fibulin-1 signal peptide)
(SEQ ID NO: 6)
MERAAPSRRV PLPLLLLGGL ALLAAGVDA
(Vitronectin signal peptide)
(SEQ ID NO: 7)
MAPLRPLLIL ALLAWVALA
(Complement Factor H signal peptide)
(SEQ ID NO: 8)
MRLLAKIICLMLWAICVA
(Opticin signal peptide)
(SEQ ID NO: 9)
MRLLAFLSLL ALVLQETGT
(Albumin signal peptide)
(SEQ ID NO: 22)
MKWVTFISLLFLFSSAYS
(Chymotrypsinogen signal peptide)
(SEQ ID NO: 23)
MAFLWLLSCWALLGTTFG
(Interleukin-2 signal peptide)
(SEQ ID NO: 24)
MYRMQLLSCIALILALVTNS
(Trypsinogen-2 signal peptide)
(SEQ ID NO: 25)
MNLLLILTFVAAAVA.

See, e.g., Stern et al., 2007, Trends Cell. Mol. Biol., 2:1-17 and Dalton & Barton, 2014, Protein Sci, 23: 517-525, each of which is incorporated by reference herein in its entirety for the signal peptides that can be used.

As an alternative, or an additional treatment to gene therapy, the HuPTMFabVEGFi product, e.g., HuGlyFabVEGFi glycoprotein, can be produced in human cell lines by recombinant DNA technology, and administered to patients diagnosed with diabetic retinopathy (DR) by intravitreal or subretinal injection. The HuPTMFabVEGFi product, e.g., glycoprotein, may also be administered to patients with diabetic retinopathy (DR). Human cell lines that can be used for such recombinant glycoprotein production include but are not limited to human embryonic kidney 293 cells (HEK293), fibrosarcoma HT-1080, HKB-11, CAP, HuH-7, and retinal cell lines, PER.C6, or RPE to name a few (e.g., see Dumont et al., 2015, Crit. Rev. Biotechnol. (Early Online, published online Sep. 18, 2015, pp. 1-13) “Human cell lines for biopharmaceutical manufacturing: history, status, and future perspectives” which is incorporated by reference in its entirety for a review of the human cell lines that could be used for the recombinant production of the HuPTMFabVEGFi product, e.g., HuGlyFabVEGFi glycoprotein). To ensure complete glycosylation, especially sialylation, and tyrosine-sulfation, the cell line used for production can be enhanced by engineering the host cells to co-express α-2,6-sialyltransferase (or both α-2,3- and α-2,6-sialyltransferases) and/or TPST-1 and TPST-2 enzymes responsible for tyrosine-O-sulfation in retinal cells.

Combinations of delivery of the HuPTMFabVEGFi, e.g., HuGlyFabVEGFi, to the eye/retina accompanied by delivery of other available treatments are encompassed by the methods provided herein. The additional treatments may be administered before, concurrently or subsequent to the gene therapy treatment. Available treatments for diabetic retinopathy (DR) that could be combined with the gene therapy provided herein include but are not limited to laser photocoagulation, photodynamic therapy with verteporfin, and intravitreal (IVT) injections with anti-VEGF agents, including but not limited to pegaptanib, ranibizumab, aflibercept, or bevacizumab. Additional treatments with anti-VEGF agents, such as biologics, may be referred to as “rescue” therapy.

Unlike small molecule drugs, biologics usually comprise a mixture of many variants with different modifications or forms that have a different potency, pharmacokinetics, and safety profile. It is not essential that every molecule produced either in the gene therapy or protein therapy approach be fully glycosylated and sulfated. Rather, the population of glycoproteins produced should have sufficient glycosylation (from about 1% to about 10% of the population), including 2,6-sialylation, and sulfation to demonstrate efficacy. The goal of gene therapy treatment provided herein is to slow or arrest the progression of retinal degeneration, and to slow or prevent loss of vision with minimal intervention/invasive procedures. Efficacy may be monitored by measuring BCVA (Best-Corrected Visual Acuity), intraocular pressure, slit lamp biomicroscopy, indirect ophthalmoscopy, SD-OCT (SD-Optical Coherence Tomography), electroretinography (ERG). Signs of vision loss, infection, inflammation and other safety events, including retinal detachment may also be monitored. Retinal thickness may be monitored to determine efficacy of the treatments provided herein. Without being bound by any particular theory, thickness of the retina may be used as a clinical readout, wherein the greater reduction in retinal thickness or the longer period of time before thickening of the retina, the more efficacious the treatment. Retinal thickness may be determined, for example, by SD-OCT. SD-OCT is a three-dimensional imaging technology which uses low-coherence interferometry to determine the echo time delay and magnitude of backscattered light reflected off an object of interest. OCT can be used to scan the layers of a tissue sample (e.g., the retina) with 3 to 15 μm axial resolution, and SD-OCT improves axial resolution and scan speed over previous forms of the technology (Schuman, 2008, Trans. Am. Opthamol. Soc. 106:426-458). Retinal function may be determined, for example, by ERG. ERG is a non-invasive electrophysiologic test of retinal function, approved by the FDA for use in humans, which examines the light sensitive cells of the eye (the rods and cones), and their connecting ganglion cells, in particular, their response to a flash stimulation.

In preferred embodiments, the antigen-binding fragments do not contain detectable NeuGc and/or α-Gal. The phrase “detectable NeuGc and/or α-Gal” used herein means NeuGc and/or α-Gal moieties detectable by standard assay methods known in the art. For example, NeuGc may be detected by HPLC according to Hara et al., 1989, “Highly Sensitive Determination of N-Acetyl-and N-Glycolylneuraminic Acids in Human Serum and Urine and Rat Serum by Reversed-Phase Liquid Chromatography with Fluorescence Detection.” J. Chromatogr., B: Biomed. 377: 111-119, which is hereby incorporated by reference for the method of detecting NeuGc. Alternatively, NeuGc may be detected by mass spectrometry. The α-Gal may be detected using an ELISA, see, for example, Galili et al., 1998, “A sensitive assay for measuring alpha-Gal epitope expression on cells by a monoclonal anti-Gal antibody.” Transplantation. 65(8):1129-32, or by mass spectrometry, see, for example, Ayoub et al., 2013, “Correct primary structure assessment and extensive glyco-profiling of cetuximab by a combination of intact, middle-up, middle-down and bottom-up ESI and MALDI mass spectrometry techniques.” Landes Bioscience. 5(5): 699-710. See also the references cited in Platts-Mills et al., 2015, “Anaphylaxis to the Carbohydrate Side-Chain Alpha-gal” Immunol Allergy Clin North Am. 35(2): 247-260.

In certain aspects, also provided herein are anti-VEGF antigen-binding fragments (i.e., antigen-binding fragments that immunospecifically binds to VEGF) comprising light chain CDRs 1-3 of SEQ ID NOs: 14-16 and heavy chain CDRs 1-3 of SEQ ID NOs: 20,18, and 21, wherein the second amino acid residue of the light chain CDR3 (i.e., the second Q in QQYSTVPWTF (SEQ ID NO. 16)) does not carry one or more of the following chemical modifications: oxidation, acetylation, deamidation, and pyroglutamation (pyro Glu). In a specific embodiment, the antigen-binding fragment comprises light chain CDRs 1-3 of SEQ ID NOs: 14-16 and heavy chain CDRs 1-3 of SEQ ID NOs: 20,18, and 21, wherein the eighth and eleventh amino acid residues of the light chain CDR1 (i.e., the two Ns in SASQDISNYLN (SEQ ID NO. 14) each carries one or more of the following chemical modifications: oxidation, acetylation, deamidation, and pyroglutamation (pyro Glu), and the second amino acid residue of the light chain CDR3 (i.e., the second Q in QQYSTVPWTF (SEQ ID NO. 16)) does not carry one or more of the following chemical modifications: oxidation, acetylation, deamidation, and pyroglutamation (pyro Glu). In a specific embodiment, the antigen-binding fragment comprises light chain CDRs 1-3 of SEQ ID NOs: 14-16 and heavy chain CDRs 1-3 of SEQ ID NOs: 20, 18, and 21, wherein the second amino acid residue of the light chain CDR3 (i.e., the second Q in QQYSTVPWTF (SEQ ID NO. 16)) is not acetylated. In a specific embodiment, the antigen-binding fragment comprises light chain CDRs 1-3 of SEQ ID NOs: 14-16 and heavy chain CDRs 1-3 of SEQ ID NOs: 20, 18, and 21, wherein the eighth and eleventh amino acid residues of the light chain CDR1 (i.e., the two Ns in SASQDISNYLN (SEQ ID NO. 14) each carries one or more of the following chemical modifications: oxidation, acetylation, deamidation, and pyroglutamation (pyro Glu), and the second amino acid residue of the light chain CDR3 (i.e., the second Q in QQYSTVPWTF (SEQ ID NO. 16)) is not acetylated. The anti-VEGF antigen-binding fragments provided herein can be used in any method according to the invention described herein. In a preferred embodiment, the chemical modification(s) or lack of chemical modification(s) (as the case may be) described herein is determined by mass spectrometry.

In certain aspects, also provided herein are anti-VEGF antigen-binding fragments comprising light chain CDRs 1-3 of SEQ ID NOs: 14-16 and heavy chain CDRs 1-3 of SEQ ID NOs: 20, 18, and 21, wherein the last amino acid residue of the heavy chain CDR1 (i.e., the N in GYDFTHYGMN (SEQ ID NO. 20)) does not carry one or more of the following chemical modifications: oxidation, acetylation, deamidation, and pyroglutamation (pyro Glu). In a specific embodiment, the antigen-binding fragment comprises light chain CDRs 1-3 of SEQ ID NOs: 14-16 and heavy chain CDRs 1-3 of SEQ ID NOs: 20, 18, and 21, wherein the ninth amino acid residue of the heavy chain CDR1 (i.e., the M in GYDFTHYGMN (SEQ ID NO. 20)) carries one or more of the following chemical modifications: acetylation, deamidation, and pyroglutamation (pyro Glu), the third amino acid residue of the heavy chain CDR2 (i.e., the N in WINTYTGEPTYAADFKR (SEQ ID NO. 18) carries one or more of the following chemical modifications: acetylation, deamidation, and pyroglutamation (pyro Glu), and the last amino acid residue of the heavy chain CDR1 (i.e., the N in GYDFTHYGMN (SEQ ID NO. 20)) does not carry one or more of the following chemical modifications: oxidation, acetylation, deamidation, and pyroglutamation (pyro Glu). In a specific embodiment, the antigen-binding fragment comprises light chain CDRs 1-3 of SEQ ID NOs: 14-16 and heavy chain CDRs 1-3 of SEQ ID NOs: 20, 18, and 21, wherein the last amino acid residue of the heavy chain CDR1 (i.e., the N in GYDFTHYGMN (SEQ ID NO. 20)) is not acetylated. In a specific embodiment, the antigen-binding fragment comprises light chain CDRs 1-3 of SEQ ID NOs: 14-16 and heavy chain CDRs 1-3 of SEQ ID NOs: 20, 18, and 21, wherein the ninth amino acid residue of the heavy chain CDR1 (i.e., the M in GYDFTHYGMN (SEQ ID NO. 20)) carries one or more of the following chemical modifications: acetylation, deamidation, and pyroglutamation (pyro Glu), the third amino acid residue of the heavy chain CDR2 (i.e., the N in WINTYTGEPTYAADFKR (SEQ ID NO. 18) carries one or more of the following chemical modifications: acetylation, deamidation, and pyroglutamation (pyro Glu), and the last amino acid residue of the heavy chain CDR1 (i.e., the N in GYDFTHYGMN (SEQ ID NO. 20)) is not acetylated. The anti-VEGF antigen-binding fragments provided herein can be used in any method according to the invention described herein. In a preferred embodiment, the chemical modification(s) or lack of chemical modification(s) (as the case may be) described herein is determined by mass spectrometry.

In certain aspects, also provided herein are anti-VEGF antigen-binding fragments comprising light chain CDRs 1-3 of SEQ ID NOs: 14-16 and heavy chain CDRs 1-3 of SEQ ID NOs: 20, 18, and 21, wherein the last amino acid residue of the heavy chain CDR1 (i.e., the N in GYDFTHYGMN (SEQ ID NO. 20)) does not carry one or more of the following chemical modifications: oxidation, acetylation, deamidation, and pyroglutamation (pyro Glu), and the second amino acid residue of the light chain CDR3 (i.e., the second Q in QQYSTVPWTF (SEQ ID NO. 16)) does not carry one or more of the following chemical modifications: oxidation, acetylation, deamidation, and pyroglutamation (pyro Glu). In a specific embodiment, the antigen-binding fragment comprises light chain CDRs 1-3 of SEQ ID NOs: 14-16 and heavy chain CDRs 1-3 of SEQ ID NOs: 20, 18, and 21, wherein: (1) the ninth amino acid residue of the heavy chain CDR1 (i.e., the M in GYDFTHYGMN (SEQ ID NO. 20)) carries one or more of the following chemical modifications: acetylation, deamidation, and pyroglutamation (pyro Glu), the third amino acid residue of the heavy chain CDR2 (i.e., the N in WINTYTGEPTYAADFKR (SEQ ID NO. 18) carries one or more of the following chemical modifications: acetylation, deamidation, and pyroglutamation (pyro Glu), and the last amino acid residue of the heavy chain CDR1 (i.e., the N in GYDFTHYGMN (SEQ ID NO. 20)) does not carry one or more of the following chemical modifications: oxidation, acetylation, deamidation, and pyroglutamation (pyro Glu); and (2) the eighth and eleventh amino acid residues of the light chain CDR1 (i.e., the two Ns in SASQDISNYLN (SEQ ID NO. 14) each carries one or more of the following chemical modifications: oxidation, acetylation, deamidation, and pyroglutamation (pyro Glu), and the second amino acid residue of the light chain CDR3 (i.e., the second Q in QQYSTVPWTF (SEQ ID NO. 16)) does not carry one or more of the following chemical modifications: oxidation, acetylation, deamidation, and pyroglutamation (pyro Glu). In a specific embodiment, the antigen-binding fragment comprises light chain CDRs 1-3 of SEQ ID NOs: 14-16 and heavy chain CDRs 1-3 of SEQ ID NOs: 20, 18, and 21, wherein the last amino acid residue of the heavy chain CDR1 (i.e., the N in GYDFTHYGMN (SEQ ID NO. 20)) is not acetylated, and the second amino acid residue of the light chain CDR3 (i.e., the second Q in QQYSTVPWTF (SEQ ID NO. 16)) is not acetylated. In a specific embodiment, the antigen-binding fragment comprises light chain CDRs 1-3 of SEQ ID NOs: 14-16 and heavy chain CDRs 1-3 of SEQ ID NOs: 20, 18, and 21, wherein: (1) the ninth amino acid residue of the heavy chain CDR1 (i.e., the M in GYDFTHYGMN (SEQ ID NO. 20)) carries one or more of the following chemical modifications: acetylation, deamidation, and pyroglutamation (pyro Glu), the third amino acid residue of the heavy chain CDR2 (i.e., the N in WINTYTGEPTYAADFKR (SEQ ID NO. 18) carries one or more of the following chemical modifications: acetylation, deamidation, and pyroglutamation (pyro Glu), and the last amino acid residue of the heavy chain CDR1 (i.e., the N in GYDFTHYGMN (SEQ ID NO. 20)) is not acetylated; and (2) the eighth and eleventh amino acid residues of the light chain CDR1 (i.e., the two Ns in SASQDISNYLN (SEQ ID NO. 14) each carries one or more of the following chemical modifications: oxidation, acetylation, deamidation, and pyroglutamation (pyro Glu), and the second amino acid residue of the light chain CDR3 (i.e., the second Q in QQYSTVPWTF (SEQ ID NO. 16)) is not acetylated. The anti-VEGF antigen-binding fragments provided herein can be used in any method according to the invention described herein. In a preferred embodiment, the chemical modification(s) or lack of chemical modification(s) (as the case may be) described herein is determined by mass spectrometry.

Another contemplated administration route is subretinal administration via the suprachoroidal space, using a subretinal drug delivery device that has a catheter inserted and tunneled through the suprachoroidal space to inject into the subretinal space toward the posterior pole, where a small needle injects into the subretinal space. This route of administration allows the vitreous to remain intact and thus, there are fewer complication risks (less risk of gene therapy egress, and complications such as retinal detachments and macular holes), and without a vitrectomy, the resulting bleb may spread more diffusely allowing more of the surface area of the retina to be transduced with a smaller volume. The risk of induced cataract following this procedure is minimized, which is desirable for younger patients. Moreover, this procedure can deliver bleb under the fovea more safely than the standard transvitreal approach, which is desirable for patients with inherited retinal diseases effecting central vision where the target cells for transduction are in the macula. This procedure is also favorable for patients that have neutralizing antibodies (Nabs) to AAVs present in the systemic circulation which may impact other routes of delivery. Additionally, this method has shown to create blebs with less egress out the retinotomy site than the standard transvitreal approach.

Juxtascleral administration provides an additional administration route which avoids the risk of intraocular infection and retinal detachment, side effects commonly associated with injecting therapeutic agents directly into the eye.

In certain aspects, provided herein is a method of treating a human subject diagnosed with diabetic retinopathy (DR), comprising administering to the subretinal space in the eye of said human subject an expression vector encoding an anti-human vascular endothelial growth factor (hVEGF) antibody, wherein the expression vector is administered via subretinal delivery in a single dose about 1.6×10¹¹ GC/eye at a concentration of 6.2×10¹¹ GC/mL or about 2.5×10¹¹ GC/eye at a concentration of 1.0×10¹² GC/mL, wherein the anti-hVEGF antibody comprises a heavy chain comprising the amino acid sequence of SEQ ID NO. 2 or SEQ ID NO. 4, and a light chain comprising the amino acid sequence of SEQ ID NO. 1, or SEQ ID NO. 3; and wherein the expression vector is an AAV8 vector.

In certain aspects, provided herein is a method of treating a human subject diagnosed with diabetic retinopathy (DR), comprising administering to the subretinal space in the eye of said human subject an expression vector encoding an anti-human vascular endothelial growth factor (hVEGF) antibody, wherein the expression vector is administered via subretinal delivery in a single dose about 1.55×10¹¹ GC/eye at a concentration of 6.2×10¹¹ GC/mL or about 2.5×10¹¹ GC/eye at a concentration of 1.0×10¹² GC/mL, wherein the anti-hVEGF antibody comprises a heavy chain comprising the amino acid sequence of SEQ ID NO. 2 or SEQ ID NO. 4, and a light chain comprising the amino acid sequence of SEQ ID NO. 1, or SEQ ID NO. 3; and wherein the expression vector is an AAV8 vector.

In certain aspects, provided herein is a method of treating a human subject diagnosed with diabetic retinopathy (DR), comprising administering to the subretinal space in the eye of said human subject an expression vector encoding an anti-human vascular endothelial growth factor (hVEGF) antibody, wherein the expression vector is administered via subretinal delivery in a single dose about 1.6×10¹¹ GC/eye at a concentration of 6.4×10¹¹ GC/mL or about 2.5×10¹¹ GC/eye at a concentration of 1.0×10¹² GC/mL, wherein the anti-hVEGF antibody comprises a heavy chain comprising the amino acid sequence of SEQ ID NO. 2 or SEQ ID NO. 4, and a light chain comprising the amino acid sequence of SEQ ID NO. 1, or SEQ ID NO. 3; and wherein the expression vector is an AAV8 vector.

In certain aspects, provided herein is a single dose composition comprising 1.6×10¹¹ GC ata concentration of 6.2×10¹¹ GC/mL or 2.5×10¹¹ GC ata concentration of 1.0×10¹² GC/mL of an expression vector encoding an anti-human vascular endothelial growth factor (hVEGF) antibody in a formulation buffer (pH=7.4), wherein the formulation buffer comprises Dulbecco's phosphate buffered saline and 0.0001% Pluronic F68, wherein the anti-hVEGF antibody comprises a heavy chain comprising the amino acid sequence of SEQ ID NO. 2 or SEQ ID NO. 4, and a light chain comprising the amino acid sequence of SEQ ID NO. 1, or SEQ ID NO. 3; and wherein the wherein the expression vector is an AAV8 vector.

In certain aspects, provided herein is a single dose composition comprising 1.55×10¹¹ GC at a concentration of 6.2×10¹¹ GC/mL or 2.5×10¹¹ GC at a concentration of 1.0×10¹² GC/mL of an expression vector encoding an anti-human vascular endothelial growth factor (hVEGF) antibody in a formulation buffer (pH=7.4), wherein the formulation buffer comprises Dulbecco's phosphate buffered saline and 0.0001% Pluronic F68, wherein the anti-hVEGF antibody comprises a heavy chain comprising the amino acid sequence of SEQ ID NO. 2 or SEQ ID NO. 4, and a light chain comprising the amino acid sequence of SEQ ID NO. 1, or SEQ ID NO. 3; and wherein the wherein the expression vector is an AAV8 vector.

In certain aspects, provided herein is a single dose composition comprising 1.6×10¹¹ GC at a concentration of 6.4×10¹¹ GC/mL or 2.5×10¹¹ GC ata concentration of 1.0×10¹² GC/mL of an expression vector encoding an anti-human vascular endothelial growth factor (hVEGF) antibody in a formulation buffer (pH=7.4), wherein the formulation buffer comprises Dulbecco's phosphate buffered saline and 0.0001% Pluronic F68, wherein the anti-hVEGF antibody comprises a heavy chain comprising the amino acid sequence of SEQ ID NO. 2 or SEQ ID NO. 4, and a light chain comprising the amino acid sequence of SEQ ID NO. 1, or SEQ ID NO. 3; and wherein the wherein the expression vector is an AAV8 vector.

In certain aspects, provided herein is a single dose composition comprising about 6.0×10¹⁰ genome copies per eye, 1.6×10¹¹ genome copies per eye, 2.5×10¹¹ genome copies per eye, 5.0×10¹¹ genome copies per eye, or 3.0×10¹² genome copies per eye of an expression vector encoding an anti-human vascular endothelial growth factor (hVEGF) antibody in a formulation buffer (pH=7.4), wherein the formulation buffer comprises Dulbecco's phosphate buffered saline and 0.0001% Pluronic F68, wherein the anti-hVEGF antibody comprises a heavy chain comprising the amino acid sequence of SEQ ID NO. 2 or SEQ ID NO. 4, and a light chain comprising the amino acid sequence of SEQ ID NO. 1, or SEQ ID NO. 3; and wherein the wherein the expression vector is an AAV8 vector.

In certain embodiments, provided herein is a method for treating a subject with diabetic retinopathy (DR), wherein the subject has at least one eye with DR, the method comprising the steps of:
(1) determining the subject's ETDRS-DR Severity Scale (DRSS) Level, and
(2) if the subject's ETDRS-DRSS is Level 47, 53, 61 or 65 then administering to the subretinal space or the suprachoroidal space in the eye of the human subject an expression vector encoding an anti-human vascular endothelial growth factor (hVEGF) antibody.

In some embodiments, the method further comprises obtaining or having obtained a biological sample from the subject, and determining that the subject has a serum level of hemoglobin A1c of less than or equal to 10%.

In some embodiments, the method prevents progression to proliferative stages of retinopathy in the subject.

In certain embodiments, provided herein is a method for treating a subject with diabetic retinopathy, wherein the subject has at least one eye with moderately-severe non-proliferative diabetic retinopathy (NPDR), the method comprising the steps of:
(1) determining the subject's ETDRS-DR Severity Scale (DRSS) Level, and
(2) if the subject's ETDRS-DRSS is Level 47, then administering to the subretinal space or the suprachoroidal space in the eye of the human subject an expression vector encoding an anti-human vascular endothelial growth factor (hVEGF) antibody.

In certain embodiments, provided herein is a method for treating a subject with diabetic retinopathy, wherein the subject has at least one eye with severe NPDR, the method comprising the steps of:
(1) determining the subject's ETDRS-DR Severity Scale (DRSS) Level, and
(2) if the subject's ETDRS-DRSS is Level 53, then administering to the subretinal space or the suprachoroidal space in the eye of the human subject an expression vector encoding an anti-human vascular endothelial growth factor (hVEGF) antibody.

In certain embodiments, provided herein is a method for treating a subject with diabetic retinopathy, wherein the subject has at least one eye with mild proliferative diabetic retinopathy (PDR), the method comprising the steps of:
(1) determining the subject's ETDRS-DR Severity Scale (DRSS) Level, and
(2) if the subject's ETDRS-DRSS is Level 61, then administering to the subretinal space or the suprachoroidal space in the eye of the human subject an expression vector encoding an anti-human vascular endothelial growth factor (hVEGF) antibody.

In certain embodiments, provided herein is a method for treating a subject with diabetic retinopathy, wherein the subject has at least one eye with moderate PDR, the method comprising the steps of:
(1) determining the subject's ETDRS-DR Severity Scale (DRSS) Level, and
(2) if the subject's ETDRS-DRSS is Level 65, then administering to the subretinal space or the suprachoroidal space in the eye of the human subject an expression vector encoding an anti-human vascular endothelial growth factor (hVEGF) antibody.

ETDRS-DR severity scale (DRSS) Levels are determined using standard 4-widefield digital stereoscopic fundus photographs or equivalent; they may also be measured by monoscopic or stereo photography in accordance with Li et al., 2010, Retina Invest Ophthalmol Vis Sci. 2010; 51:3184-3192, or an analogous method.

In certain embodiments of the methods described herein, the method further comprises, after the administering step, a step of monitoring temperature of the surface of the eye using an infrared thermal camera. In a specific embodiment, the infrared thermal camera is an FLIR T530 infrared thermal camera. In a specific embodiment, the infrared thermal camera is an FLIR T420 infrared thermal camera. In a specific embodiment, the infrared thermal camera is an FLIR T440 infrared thermal camera. In a specific embodiment, the infrared thermal camera is an Fluke Ti400 infrared thermal camera. In a specific embodiment, the infrared thermal camera is an FLIRE60 infrared thermal camera. In a specific embodiment, the infrared resolution of the infrared thermal camera is equal to or greater than 75,000 pixels. In a specific embodiment, the thermal sensitivity of the infrared thermal camera is equal to or smaller than 0.05° C. at 30° C. In a specific embodiment, the field of view (FOV) of the infrared thermal camera is equal to or lower than 25°×25°.

### 3.1 Illustrative Embodiments

1. A method of treating a human subject diagnosed with diabetic retinopathy (DR), comprising administering to the subretinal space in the eye of said human subject an expression vector encoding an anti-human vascular endothelial growth factor (hVEGF) antibody, wherein the expression vector is administered via subretinal delivery in a single dose about 1.6×10¹¹ GC/eye at a concentration of 6.2×10¹¹ GC/mL or about 2.5×10¹¹ GC/eye at a concentration of 1.0×10¹² GC/mL, wherein the anti-hVEGF antibody comprises a heavy chain comprising the amino acid sequence of SEQ ID NO. 2 or SEQ ID NO. 4, and a light chain comprising the amino acid sequence of SEQ ID NO. 1, or SEQ ID NO. 3; and wherein the expression vector is an AAV8 vector.

2. The method of paragraph 1, wherein the administering is by injecting the expression vector into the subretinal space using a subretinal drug delivery device.

3. The method of any one of paragraphs 1-2, wherein the administering delivers a therapeutically effective amount of the anti-hVEGF antibody to the retina of said human subject.

4. The method of paragraph 3, wherein the therapeutically effective amount of the anti-hVEGF antibody is produced by human retinal cells of said human subject.

5. The method of paragraph 4, wherein the therapeutically effective amount of the anti-hVEGF antibody is produced by human photoreceptor cells, horizontal cells, bipolar cells, amacrine cells, retina ganglion cells, and/or retinal pigment epithelial cells in the external limiting membrane of said human subject.

6. The method of paragraph 5, wherein the human photoreceptor cells are cone cells and/or rod cells.

7. The method of paragraph 6, wherein the retina ganglion cells are midget cells, parasol cells, bistratified cells, giant retina ganglion cells, photosensitive ganglion cells, and/or Múller glia.

8. The method of any one of paragraphs 1-7, wherein the expression vector comprises the CB7 promoter.

9. The method of paragraph 8, wherein the expression vector is Construct II.

10. A single dose composition comprising 1.6×10¹¹ GC at a concentration of 6.2×10¹¹ GC/mL or 2.5×10¹¹ GC at a concentration of 1.0×10¹² GC/mL of an expression vector encoding an anti-human vascular endothelial growth factor (hVEGF) antibody in a formulation buffer (pH=7.4), wherein the formulation buffer comprises Dulbecco's phosphate buffered saline and 0.001% Pluronic F68, wherein the anti-hVEGF antibody comprises a heavy chain comprising the amino acid sequence of SEQ ID NO. 2 or SEQ ID NO. 4, and a light chain comprising the amino acid sequence of SEQ ID NO. 1, or SEQ ID NO. 3; and wherein the wherein the expression vector is an AAV8 vector.

11. The composition of paragraph 10, wherein the expression vector is II.

12. The method of any one of paragraphs 1-9, which further comprises, after the administering step, a step of monitoring the post ocular injection thermal profile of the injected material in the eye using an infrared thermal camera.

13. The method of paragraph 12, wherein the infrared thermal camera is a FLIR T530 infrared thermal camera.

14. A method of treating a human subject diagnosed with DR, comprising administering to the subretinal space in the eye of said human subject an expression vector encoding an anti-human vascular endothelial growth factor (hVEGF) antibody, wherein about 2.5×10¹¹ genome copies per eye of the expression vector are administered by double suprachoroidal injections, wherein the anti-hVEGF antibody comprises a heavy chain comprising the amino acid sequence of SEQ ID NO. 2 or SEQ ID NO. 4, and a light chain comprising the amino acid sequence of SEQ ID NO. 1, or SEQ ID NO. 3; and wherein the expression vector is an AAV8 vector.

15. A method of treating a human subject diagnosed with DR, comprising administering to the subretinal space in the eye of said human subject an expression vector encoding an anti-human vascular endothelial growth factor (hVEGF) antibody, wherein about 5.0×10¹¹ genome copies per eye of the expression vector are administered by double suprachoroidal injections, wherein the anti-hVEGF antibody comprises a heavy chain comprising the amino acid sequence of SEQ ID NO. 2 or SEQ ID NO. 4, and a light chain comprising the amino acid sequence of SEQ ID NO. 1, or SEQ ID NO. 3; and wherein the expression vector is an AAV8 vector.

16. The method of any one of paragraphs 14-15, wherein the administering delivers a therapeutically effective amount of the anti-hVEGF antibody to the retina of said human subject.

17. The method of paragraph 16, wherein the therapeutically effective amount of the anti-hVEGF antibody is produced by human retinal cells of said human subject.

18. The method of paragraph 17, wherein the therapeutically effective amount of the anti-hVEGF antibody is produced by human photoreceptor cells, horizontal cells, bipolar cells, amacrine cells, retina ganglion cells, and/or retinal pigment epithelial cells in the external limiting membrane of said human subject.

19. The method of paragraph 18, wherein the human photoreceptor cells are cone cells and/or rod cells.

20. The method of paragraph 19, wherein the retina ganglion cells are midget cells, parasol cells, bistratified cells, giant retina ganglion cells, photosensitive ganglion cells, and/or Múller glia.

21. The method of any one of paragraphs 14-20, wherein the expression vector comprises the CB7 promoter.

22. The method of paragraph 21, wherein the expression vector is Construct II.

23. The method of any one of paragraphs 14-22, which further comprises, after the administering step, a step of monitoring the post ocular injection thermal profile of the injected material in the eye using an infrared thermal camera.

24. The method of paragraph 23, wherein the infrared thermal camera is a FLIR T530 infrared thermal camera.

25. A single dose composition comprising about 6.0×10¹⁰ genome copies per eye, 1.6×10¹¹ genome copies per eye, 2.5×10¹¹ genome copies per eye, 5.0×10¹¹ genome copies per eye, or 3.0×10¹² genome copies per eye of an expression vector encoding an anti-human vascular endothelial growth factor (hVEGF) antibody in a formulation buffer (pH=7.4), wherein the formulation buffer comprises Dulbecco's phosphate buffered saline and 0.0001% Pluronic F68, wherein the anti-hVEGF antibody comprises a heavy chain comprising the amino acid sequence of SEQ ID NO. 2 or SEQ ID NO. 4, and a light chain comprising the amino acid sequence of SEQ ID NO. 1, or SEQ ID NO. 3; and wherein the wherein the expression vector is an AAV8 vector.

26. The composition of paragraph 16, wherein the expression vector is Construct II.

27. The method of any one of paragraphs 1-9 and 12-24, wherein the method does not result in shedding of the expression vector.

28. The method of any one of paragraphs 1-9 and 12-24, wherein less than 1000, less than 500, less than 100, less than 50 or less than 10 expression vector gene copies/5 μL are detectable by quantitative polymerase chain reaction in a biological fluid at any point after administration.

29. The method of any one of paragraphs 1-9 and 12-24, wherein 210 expression vector gene copies/5 μL or less are detectable by quantitative polymerase chain reaction in a biological fluid at any point after administration.

30. The method of any one of paragraphs 1-9 and 12-24, wherein less than 1000, less than 500, less than 100, less than 50 or less than 10 vector gene copies/5 μL are detectable by quantitative polymerase chain reaction in a biological fluid by 1, 2, 3, 4, 5, 6, 7, 8, 9, 10, 11, 12, 13 or 14 weeks after administration.

31. The method of any one of paragraphs 1-9 and 12-24, wherein no vector gene copies are detectable in a biological fluid by week 14 after administration of the vector.

32. The method of any one of paragraphs 28-31, wherein the biological fluid is tears, serum or urine.

### 4. BRIEF DESCRIPTION OF THE DRAWINGS

FIG. 1. The amino acid sequence of ranibizumab (top) showing 5 different residues in bevacizumab Fab (below). The starts of the variable and constant heavy chains (V_{H} and C_{H}) and light chains (V_{L} and V_{C}) are indicated by arrows (→), and the CDRs are underscored. Non-consensus glycosylation sites (“Gsite”) tyrosine-O-sulfation sites (“Ysite”) are indicated.

FIG. 2. Glycans that can be attached to HuGlyFabVEGFi. (Adapted from Bondt et al., 2014, Mol & Cell Proteomics 13.1: 3029-3039).

FIG. 3. The amino acid sequence of hyperglycosylated variants of ranibizumab (above) and bevacizumab Fab (below). The starts of the variable and constant heavy chains (V_{H} and C_{H}) and light chains (V_{L} and V_{C}) are indicated by arrows (→), and the CDRs are underscored. Non-consensus glycosylation sites (“Gsite”) and tyrosine-O-sulfation sites (“Ysite”) are indicated. Four hyperglycoslated variants are indicated with an asterisk (*).

FIG. 4. Schematic of AAV8-antiVEGFfab genome

FIG. 5. A suprachoroidal drug delivery device manufactured by Clearside® Biomedical, Inc.

FIG. 6. A subretinal drug delivery device comprising a catheter that can be inserted and tunneled through the suprachoroidal space toward the posterior pole, where a small needle injects into the subretinal space, manufactured by Janssen Pharmaceuticals, Inc.

FIGS. 7A-7D. Illustration of the posterior juxtascleral depot procedure.

FIG. 8. Clustal Multiple Sequence Alignment of AAV capsids 1-9 (SEQ ID NOs: 41-51). Amino acid substitutions (shown in bold in the bottom rows) can be made to AAV9 and AAV8 capsids by “recruiting” amino acid residues from the corresponding position of other aligned AAV capsids. Sequence regions designated by “HVR”=hypervariable regions.

FIGS. 9A and 9B. A micro volume injector drug delivery device manufactured by Altaviz.

FIGS. 10A and 10B. A drug delivery device manufactured by Visionisti OY. Specifically, FIG. 10A depicts the injection adapter, which is able to convert 30 g short hypodermic needles into a suprachoroidal/subretinal needles. The device is able to control the length of the needle tip exposed from the distal tip of the adapter. Adjustments can be made at 10 μL. The device has the ability to adjust for suprachoroidal delivery and/or ab-externo subretinal delivery. FIG. 8B depicts a needle adaptor guide which is able to keep the lids open and hold the needle at the optimal angle and depth for delivery. The needle adapter is locked into the stabilizing device. The needle adapter is an all-in-one tool for standardized and optimized in-office suprachoroidal and/or subretinal injections.

### 5. DETAILED DESCRIPTION OF THE INVENTION

Compositions and methods are described for the delivery of a fully human post-translationally modified (HuPTM) antibody against VEGF to the retina/vitreal humour in the eye(s) of patients (human subjects) diagnosed with diabetic retinopathy (DR). Antibodies include, but are not limited to, monoclonal antibodies, polyclonal antibodies, recombinantly produced antibodies, human antibodies, humanized antibodies, chimeric antibodies, synthetic antibodies, tetrameric antibodies comprising two heavy chain and two light chain molecules, antibody light chain monomers, antibody heavy chain monomers, antibody light chain dimers, antibody heavy chain dimers, antibody light chain-heavy chain pairs, intrabodies, heteroconjugate antibodies, monovalent antibodies, and antigen-binding fragments of full-length antibodies, and fusion proteins of the above. Such antigen-binding fragments include, but are not limited to,single-domain antibodies (variable domain of heavy chain antibodies (VHHs) or nanobodies), Fabs, F(ab′)₂s, and scFvs (single-chain variable fragments) of full-length anti-VEGF antibodies (preferably, full-length anti-VEGF monoclonal antibodies (mAbs)) (collectively referred to herein as “antigen-binding fragments”). In a preferred embodiment, the fully human post-translationally modified antibody against VEGF is a fully human post-translationally modified antigen-binding fragment of a monoclonal antibody (mAb) against VEGF (“HuPTMFabVEGFi”). In a further preferred embodiment, the HuPTMFabVEGFi is a fully human glycosylated antigen-binding fragment of an anti-VEGF mAb (“HuGlyFabVEGFi”). See, also, International Patent Application Publication No. WO/2017/180936 (International Patent Application No. PCT/US2017/027529, filed Apr. 14, 2017), International Patent Application Publication No. WO/2017/181021 (International Patent Application No. PCT/US2017/027650, filed April 14, 2017), and International Patent Application Publication No. WO2019/067540 (International Patent Application No. PCT/US2018/052855, filed Sep. 26, 2018),each of which is incorporated by reference herein in its entirety, for compositions and methods that can be used according to the invention described herein. In an alternative embodiment, full-length mAbs can be used. Delivery may be accomplished via gene therapy—e.g., by administering a viral vector or other DNA expression construct encoding an anti-VEGF antigen-binding fragment or mAb (or a hyperglycosylated derivative) to the suprachoroidal space, subretinal space (from a transvitreal approach or with a catheter through the suprachoroidal space), intraretinal space, vitreous cavity, and/or outer surface of the sclera (i.e., juxtascleral administration) in the eye(s) of patients (human subjects) diagnosed with diabetic retinopathy (DR), to create a permanent depot in the eye that continuously supplies the human PTM, e.g., human-glycosylated, transgene product. See, e.g., administration modes described in Section 5.3.2.

In certain embodiments, the patients have been shown to be responsive to treatment with an anti-VEGF antigen-binding fragment injected intravitreally prior to treatment with gene therapy. In specific embodiments, the patients have previously been treated with LUCENTIS® (ranibizumab), EYLEA® (aflibercept), and/or AVASTIN® (bevacizumab), and have been found to be responsive to one or more of said LUCENTIS (ranibizumab), EYLEA® (aflibercept), and/or AVASTIN® (bevacizumab).

Subjects to whom such viral vector or other DNA expression construct is delivered should be responsive to the anti-VEGF antigen-binding fragment encoded by the transgene in the viral vector or expression construct. To determine responsiveness, the anti-hVEGF antigen-binding fragment transgene product (e.g., produced in cell culture, bioreactors, etc.) may be administered directly to the subject, such as by intravitreal injection.

The HuPTMFabVEGFi, e.g., HuGlyFabVEGFi, encoded by the transgene can include, but is not limited to an antigen-binding fragment of an antibody that binds to hVEGF, such as bevacizumab; an anti-hVEGF Fab moiety such as ranibizumab; or such bevacizumab or ranibizumab Fab moieties engineered to contain additional glycosylation sites on the Fab domain (e.g., see Courtois et al., 2016, mAbs 8: 99-112 which is incorporated by reference herein in its entirety for it description of derivatives of bevacizumab that are hyperglycosylated on the Fab domain of the full length antibody).

The recombinant vector used for delivering the transgene should have a tropism for human retinal cells or photoreceptor cells. Such vectors can include non-replicating recombinant adeno-associated virus vectors (“rAAV”), particularly those bearing an AAV8 capsid are preferred. However, other viral vectors may be used, including but not limited to lentiviral vectors, vaccinia viral vectors, or non-viral expression vectors referred to as “naked DNA” constructs. Preferably, the HuPTMFabVEGFi, e.g., HuGlyFabVEGFi, transgene should be controlled by appropriate expression control elements, for example, the CB7 promoter (a chicken β-actin promoter and CMV enhancer), the RPE65 promoter, or opsin promoter to name a few, and can include other expression control elements that enhance expression of the transgene driven by the vector (e.g., introns such as the chicken β-actin intron, minute virus of mice (MVM) intron, human factor IX intron (e.g., FIX truncated intron 1), β-globin splice donor/immunoglobulin heavy chain spice acceptor intron, adenovirus splice donor/immunoglobulin splice acceptor intron, SV40 late splice donor/splice acceptor (19S/16S) intron, and hybrid adenovirus splice donor/IgG splice acceptor intron and polyA signals such as the rabbit β-globin polyA signal, human growth hormone (hGH) polyA signal, SV40 late polyA signal, synthetic polyA (SPA) signal, and bovine growth hormone (bGH) polyA signal). See, e.g., Powell and Rivera-Soto, 2015, Discov. Med., 19(102):49-57.

In preferred embodiments, gene therapy constructs are designed such that both the heavy and light chains are expressed. More specifically, the heavy and light chains should be expressed at about equal amounts, in other words, the heavy and light chains are expressed at approximately a 1:1 ratio of heavy chains to light chains. The coding sequences for the heavy and light chains can be engineered in a single construct in which the heavy and light chains are separated by a cleavable linker or IRES so that separate heavy and light chain polypeptides are expressed. See, e.g., Section 5.2.4 for specific leader sequences and Section 5.2.5 for specific IRES, 2A, and other linker sequences that can be used with the methods and compositions provided herein.

In certain embodiments, gene therapy constructs are supplied as a frozen sterile, single use solution of the AAV vector active ingredient in a formulation buffer. In a specific embodiment, the pharmaceutical compositions suitable for subretinal administration comprise a suspension of the recombinant (e.g., rHuGlyFabVEGFi) vector in a formulation buffer comprising a physiologically compatible aqueous buffer, a surfactant and optional excipients. In a specific embodiment, the construct is formulated in Dulbecco's phosphate buffered saline and 0.001% Pluronic F68, pH=7.4.

Therapeutically effective doses of the recombinant vector should be administered subretinally and/or intraretinally (e.g., by subretinal injection via the transvitreal approach (a surgical procedure), or subretinal administration via the suprachoroidal space) in a volume ranging from 0.1 mL to 0.5 mL, preferably in 0.1 to 0.30 mL (100-300 μl), and most preferably, in a volume of 0.25 mL (250 μl). Therapeutically effective doses of the recombinant vector should be administered suprachoroidally (e.g., by suprachoroidal injection) in a volume of 100 μl or less, for example, in a volume of 50-100 μl. Therapeutically effective doses of the recombinant vector should be administered to the outer surface of the sclera in a volume of 500 μl or less, for example, in a volume of 500 μl or less, for example, in a volume of 10-20 μl, 20-50 μl, 50-100 μl, 100-200 μl, 200-300 μl, 300-400 μl, or 400-500 μl. Subretinal injection is a surgical procedure performed by trained retinal surgeons that involves a partial vitrectomy with the subject under local anesthesia, and injection of the gene therapy into the retina. (see, e.g., Campochiaro et al., 2017, Hum Gen Ther 28(1):99-111, which is incorporated by reference herein in its entirety). In a specific embodiment, the subretinal administration is performed via the suprachoroidal space using a subretinal drug delivery device that comprises a catheter which can be inserted and tunneled through the suprachoroidal space to the posterior pole, where a small needle injects into the subretinal space (see, e.g., Baldassarre et al., 2017, Subretinal Delivery of Cells via the Suprachoroidal Space: Janssen Trial. In: Schwartz et al. (eds) Cellular Therapies for Retinal Disease, Springer, Cham; International Patent Application Publication No. WO 2016/040635 A1; each of which is incorporated by reference herein in its entirety). Suprachoroidal administration procedures involve administration of a drug to the suprachoroidal space of the eye, and are normally performed using a suprachoroidal drug delivery device such as a microinjector with a microneedle (see, e.g., Hariprasad, 2016, Retinal Physician 13: 20-23; Goldstein, 2014, Retina Today 9(5): 82-87; each of which is incorporated by reference herein in its entirety). The suprachoroidal drug delivery devices that can be used to deposit the expression vector in the suprachoroidal space according to the invention described herein include, but are not limited to, suprachoroidal drug delivery devices manufactured by Clearside® Biomedical, Inc. (see, for example, Hariprasad, 2016, Retinal Physician 13: 20-23). The subretinal drug delivery devices that can be used to deposit the expression vector in the subretinal space via the suprachoroidal space according to the invention described herein include, but are not limited to, subretinal drug delivery devices manufactured by Janssen Pharmaceuticals, Inc. (see, for example, International Patent Application Publication No. WO 2016/040635 A1). In a specific embodiment, administration to the outer surface of the sclera is performed by a juxtascleral drug delivery device that comprises a cannula, whose tip can be inserted and kept in direct apposition to the scleral surface. See Section 5.3.2 for more details of the different modes of administration. Suprachoroidal, subretinal, juxtascleral, intravitreal, subconjunctival, and/or intraretinal administration should result in delivery of the soluble transgene product to the retina, the vitreous humor, and/or the aqueous humor. The expression of the transgene product (e.g., the encoded anti-VEGF antibody) by retinal cells, e.g., rod, cone, retinal pigment epithelial, horizontal, bipolar, amacrine, ganglion, and/or Múller cells, results in delivery and maintenance of the transgene product in the retina, the vitreous humor, and/or the aqueous humor. In a specific embodiment, doses that maintain a concentration of the transgene product at a Cₘᵢₙ of at least 0.330 μg/mL in the vitreous humour, or 0.110 μg/mL in the aqueous humour (the anterior chamber of the eye) for three months are desired; thereafter, vitreous Cₘᵢₙ concentrations of the transgene product ranging from 1.70 to 6.60 μg/mL, and/or aqueous Cₘᵢₙ concentrations ranging from 0.567 to 2.20 μg/mL should be maintained. However, because the transgene product is continuously produced, maintenance of lower concentrations can be effective. In a specific embodiment, the concentration of the transgene product can be measured in patient samples of the vitreous humour and/or aqueous from the anterior chamber of the treated eye. Alternatively, vitreous humour concentrations can be estimated and/or monitored by measuring the patient's serum concentrations of the transgene product—the ratio of systemic to vitreal exposure to the transgene product is about 1:90,000. (E.g., see, vitreous humor and serum concentrations of ranibizumab reported in Xu L, et al., 2013, Invest. Opthal. Vis. Sci. 54: 1616-1624, at p. 1621 and Table 5 at p. 1623, which is incorporated by reference herein in its entirety).

Vector transgenes have the potential to spread to unintended recipients from shedding (release of vectors that did not infect the target cells and were cleared from the body via feces or bodily fluids), mobilization (transgene replication and transfer out of the target cell), or germ line transmission (genetic transmission to offspring through semen). Vector shedding may be determined for example by measuring vector DNA in biological fluids such as tears, serum or urine using quantitative polymerase chain reaction. In some embodiments, no vector gene copies are detectable in a biological fluid (e.g., tears, serum or urine) at any time point after administration of the vector. In some embodiments, less than 1000, less than 500, less than 100, less than 50 or less than 10 vector gene copies/5 μL are detectable by quantitative polymerase chain reaction in a biological fluid (e.g., tears, serum or urine) at any point after administration. In specific embodiments, 210 vector gene copies/5 μL or less are detectable in serum. In some embodiments, less than 1000, less than 500, less than 100, less than 50 or less than 10 vector gene copies/5 μL are detectable by quantitative polymerase chain reaction in a biological fluid (e.g., tears, serum or urine) by 1, 2, 3, 4, 5, 6, 7, 8, 9, 10, 11, 12, 13 or 14 weeks after administration. In specific embodiments, no vector gene copies are detectable in serum by week 14 after administration of the vector.

The invention has several advantages over standard of care treatments that involve repeated ocular injections of high dose boluses of the VEGF inhibitor that dissipate over time resulting in peak and trough levels. Sustained expression of the transgene product antibody, as opposed to injecting an antibody repeatedly, allows for a more consistent levels of antibody to be present at the site of action, and is less risky and more convenient for patients, since fewer injections need to be made, resulting in fewer doctor visits. Consistent protein production may leads to better clinical outcomes as edema rebound in the retina is less likely to occur. Furthermore, antibodies expressed from transgenes are post-translationally modified in a different manner than those that are directly injected because of the different microenvironment present during and after translation. Without being bound by any particular theory, this results in antibodies that have different diffusion, bioactivity, distribution, affinity, pharmacokinetic, and immunogenicity characteristics, such that the antibodies delivered to the site of action are “biobetters” in comparison with directly injected antibodies.

In addition, antibodies expressed from transgenes in vivo are not likely to contain degradation products associated with antibodies produced by recombinant technologies, such as protein aggregation and protein oxidation. Aggregation is an issue associated with protein production and storage due to high protein concentration, surface interaction with manufacturing equipment and containers, and purification with certain buffer systems. These conditions, which promote aggregation, do not exist in transgene expression in gene therapy. Oxidation, such as methionine, tryptophan, and histidine oxidation, is also associated with protein production and storage, and is caused by stressed cell culture conditions, metal and air contact, and impurities in buffers and excipients. The proteins expressed from transgenes in vivo may also oxidize in a stressed condition. However, humans, and many other organisms, are equipped with an antioxidation defense system, which not only reduces the oxidation stress, but sometimes also repairs and/or reverses the oxidation. Thus, proteins produced in vivo are not likely to be in an oxidized form. Both aggregation and oxidation could affect the potency, pharmacokinetics (clearance), and immunogenicity.

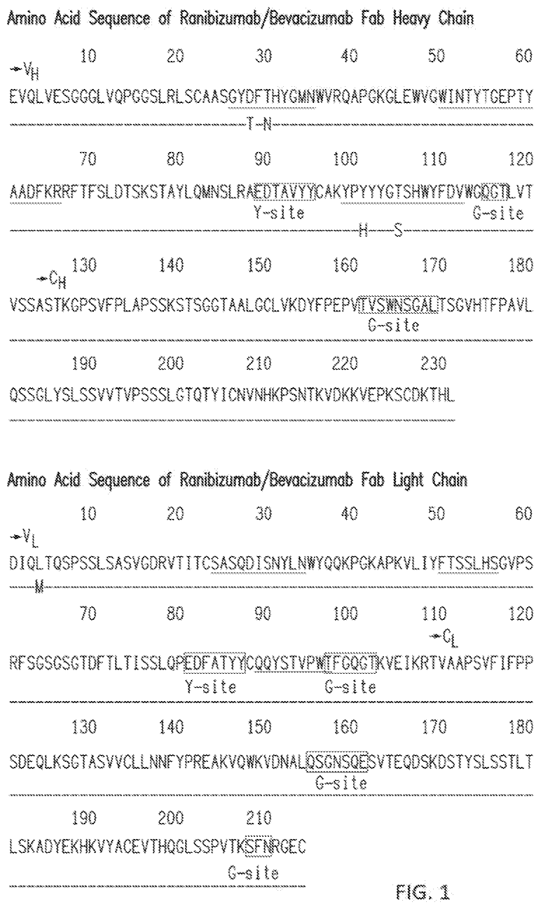

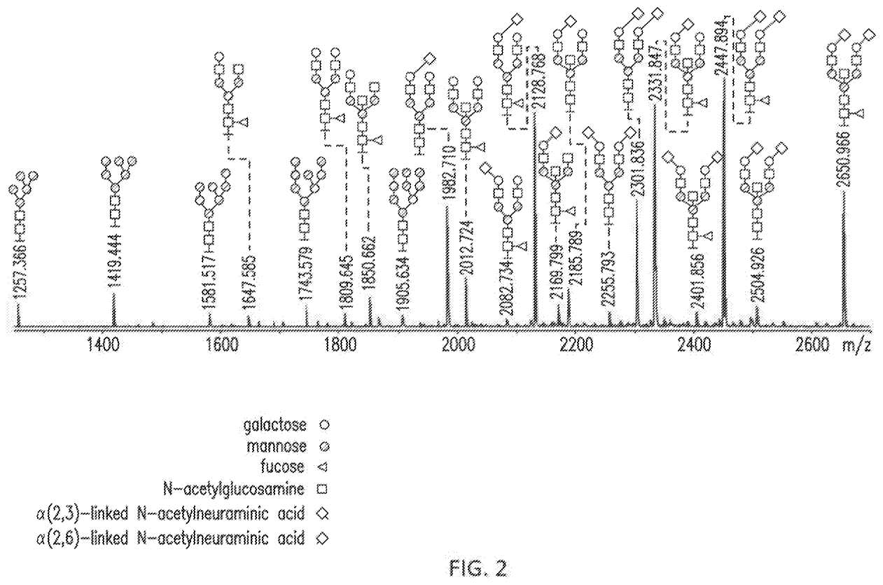

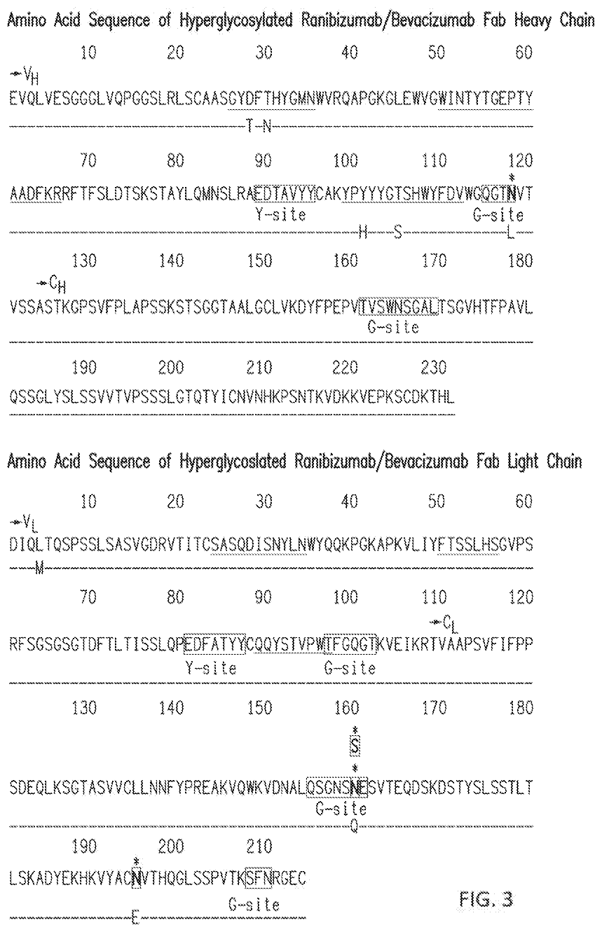

Without being bound by theory, the methods and compositions provided herein are based, in part, on the following principles:
(i) Human retinal cells are secretory cells that possess the cellular machinery for post-translational processing of secreted proteins—including glycosylation and tyrosine-O-sulfation, a robust process in retinal cells. (See, e.g., Wang et al., 2013, Analytical Biochem. 427: 20-28 and Adamis et al., 1993, BBRC 193: 631-638 reporting the production of glycoproteins by retinal cells; and Kanan et al., 2009, Exp. Eye Res. 89: 559-567 and Kanan & Al-Ubaidi, 2015, Exp. Eye Res. 133: 126-131 reporting the production of tyrosine-sulfated glycoproteins secreted by retinal cells, each of which is incorporated by reference in its entirety for post-translational modifications made by human retinal cells).
(ii) Contrary to the state of the art understanding, anti-VEGF antigen-binding fragments, such as ranibizumab (and the Fab domain of full length anti-VEGF mAbs such as bevacizumab) do indeed possess N-linked glycosylation sites. For example, see FIG. 1 which identifies non-consensus asparaginal (“N”) glycosylation sites in the C_{H} domain (TVSWN¹⁶⁵SGAL) and in the CL domain (QSGN¹⁵⁸SQE), as well as glutamine (“Q”) residues that are glycosylation sites in the VH domain (Q¹¹⁵GT) and V_{L} domain (TFQ¹⁰⁰GT) of ranibizumab (and corresponding sites in the Fab of bevacizumab). (See, e.g., Valliere-Douglass et al., 2009, J. Biol. Chem. 284: 32493-32506, and Valliere-Douglass et al., 2010, J. Biol. Chem. 285: 16012-16022, each of which is incorporated by reference in its entirety for the identification of N-linked glycosylation sites in antibodies).
(iii) While such non-canonical sites usually result in low level glycosylation (e.g., about 1-5%) of the antibody population, the functional benefits may be significant in immunoprivileged organs, such as the eye (See, e.g., van de Bovenkamp et al., 2016, J. Immunol. 196:1435-1441). For example, Fab glycosylation may affect the stability, half-life, and binding characteristics of an antibody. To determine the effects of Fab glycosylation on the affinity of the antibody for its target, any technique known to one of skill in the art may be used, for example, enzyme linked immunosorbent assay (ELISA), or surface plasmon resonance (SPR). To determine the effects of Fab glycosylation on the half-life of the antibody, any technique known to one of skill in the art may be used, for example, by measurement of the levels of radioactivity in the blood or organs (e.g., the eye) in a subject to whom a radiolabeled antibody has been administered. To determine the effects of Fab glycosylation on the stability, for example, levels of aggregation or protein unfolding, of the antibody, any technique known to one of skill in the art may be used, for example, differential scanning calorimetry (DSC), high performance liquid chromatography (HPLC), e.g., size exclusion high performance liquid chromatography (SEC-HPLC), capillary electrophoresis, mass spectrometry, or turbidity measurement. Provided herein, the HuPTMFabVEGFi, e.g., HuGlyFabVEGFi, transgene results in production of a Fab which is 0.5%, 1%, 2%, 3%, 4%, 5%, 6%, 7%, 8%, 9%, or 10% or more glycosylated at non-canonical sites. In certain embodiments, 0.5%, 1%, 2%, 3%, 4%, 5%, 6%, 7%, 8%, 9%, or 10% or more Fabs from a population of Fabs are glycosylated at non-canonical sites. In certain embodiments, 0.5%, 1%, 2%, 3%, 4%, 5%, 6%, 7%, 8%, 9%, or 10% or more non-canonical sites are glycosylated. In certain embodiments, the glycosylation of the Fab at these non-canonical sites is 25%, 50%, 100%, 200%, 300%, 400%, 500%, or more greater than the amount of glycosylation of these non-canonical sites in a Fab produced in HEK293 cells.
(iv) In addition to the glycosylation sites, anti-VEGF Fabs such as ranibizumab (and the Fab of bevacizumab) contain tyrosine (“Y”) sulfation sites in or near the CDRs; see FIG. 1 which identifies tyrosine-O-sulfation sites in the VH (EDTAVY⁹⁴Y⁹⁵) and VL (EDFATY⁸⁶) domains of ranibizumab (and corresponding sites in the Fab of bevacizumab). (See, e.g., Yang et al., 2015, Molecules 20:2138-2164, esp. at p. 2154 which is incorporated by reference in its entirety for the analysis of amino acids surrounding tyrosine residues subjected to protein tyrosine sulfation. The “rules” can be summarized as follows: Y residues with E or D within +5 to −5 position of Y, and where position −1 of Y is a neutral or acidic charged amino acid—but not a basic amino acid, e.g., R, K, or H that abolishes sulfation). Human IgG antibodies can manifest a number of other post-translational modifications, such as N-terminal modifications, C-terminal modifications, degradation or oxidation of amino acid residues, cysteine related variants, and glycation (See, e.g., Liu etal., 2014, mAbs 6(5):1145-1154).
(v) Glycosylation of anti-VEGF Fabs, such as ranibizumab or the Fab fragment of bevacizumab by human retinal cells will result in the addition of glycans that can improve stability, half-life and reduce unwanted aggregation and/or immunogenicity of the transgene product. (See, e.g., Bovenkamp etal., 2016, J. Immunol. 196: 1435-1441 for a review of the emerging importance of Fab glycosylation). Significantly, glycans that can be added to HuPTMFabVEGFi, e.g., HuGlyFabVEGFi, provided herein, are highly processed complex-type biantennary N-glycans that contain 2,6-sialic acid (e.g., see FIG. 2 depicting the glycans that may be incorporated into HuPTMFabVEGFi, e.g., HuGlyFabVEGFi) and bisecting GlcNAc, but not NGNA (N-Glycolylneuraminic acid, Neu5Gc). Such glycans are not present in ranibizumab (which is made in E. coli and is not glycosylated at all) or in bevacizumab (which is made in CHO cells that do not have the 2,6-sialyltransferase required to make this post-translational modification, nor do CHO cells product bisecting GlcNAc, although they do add Neu5Gc (NGNA) as sialic acid not typical (and potentially immunogenic) to humans instead of Neu5Ac (NANA)). See, e.g., Dumont et al., 2015, Crit. Rev. Biotechnol. (Early Online, published online Sep. 18, 2015, pp. 1-13 at p. 5). Moreover, CHO cells can also produce an immunogenic glycan, the α-Gal antigen, which reacts with anti-α-Gal antibodies present in most individuals, and at high concentrations can trigger anaphylaxis. See, e.g., Bosques, 2010, Nat Biotech 28: 1153-1156. The human glycosylation pattern of the HuPTMFabVEGFi, e.g., HuGlyFabVEGFi, provided herein, should reduce immunogenicity of the transgene product and improve efficacy.
(vi) Tyrosine-sulfation of anti-VEGF Fabs, such as ranibizumab or the Fab fragment of bevacizumab—a robust post-translational process in human retinal cells—could result in transgene products with increased avidity for VEGF. Indeed, tyrosine-sulfation of the Fab of therapeutic antibodies against other targets has been shown to dramatically increase avidity for antigen and activity. (See, e.g., Loos et al., 2015, PNAS 112: 12675-12680, and Choe et al., 2003, Cell 114: 161-170). Such post-translational modifications are not present on ranibizumab (which is made in E. coli a host that does not possess the enzymes required for tyrosine-sulfation), and at best is under-represented in bevacizumab—a CHO cell product. Unlike human retinal cells, CHO cells are not secretory cells and have a limited capacity for post-translational tyrosine-sulfation. (See, e.g., Mikkelsen & Ezban, 1991, Biochemistry 30: 1533-1537, esp. discussion at p. 1537).

For the foregoing reasons, the production of HuPTMFabVEGFi, e.g., HuGlyFabVEGFi, should result in a “biobetter” molecule for the treatment of diabetic retinopathy (DR) accomplished via gene therapy—e.g., by administering a viral vector or other DNA expression construct encoding HuPTMFabVEGFi, e.g., HuGlyFabVEGFi, to the suprachoroidal space, subretinal space, or outer surface of the sclera in the eye(s)of patients (human subjects) diagnosed with diabetic retinopathy (DR), (e.g., by suprachoroidal injection, subretinal injection via the transvitreal approach (a surgical procedure), subretinal administration via the suprachoroidal space, or a posterior juxtascleral depot procedure), to create a permanent depot in the eye that continuously supplies the fully-human post-translationally modified, e.g., human-glycosylated, sulfated transgene product produced by transduced retinal cells. The cDNA construct for the FabVEGFi should include a signal peptide that ensures proper co- and post-translational processing (glycosylation and protein sulfation) by the transduced retinal cells. Such signal sequences used by retinal cells may include but are not limited to:

(VEGF-A signal peptide)
(SEQ ID NO: 5)
MNFLLSWVHW SLALLLYLHH AKWSQA
(Fibulin-1 signal peptide)
(SEQ ID NO: 6)
MERAAPSRRV PLPLLLLGGL ALLAAGVDA
(Vitronectin signal peptide)
(SEQ ID NO: 7)
MAPLRPLLIL ALLAWVALA
(Complement Factor H signal peptide)
(SEQ ID NO: 8)
MRLLAKIICLMLWAICVA
(Opticin signal peptide)
(SEQ ID NO: 9)
MRLLAFLSLL ALVLQETGT
(Albumin signal peptide)
(SEQ ID NO: 22)
MKWVTFISLLFLFSSAYS
(Chymotrypsinogen signal peptide)
(SEQ ID NO: 23)
MAFLWLLSCWALLGTTFG
(Interleukin-2 signal peptide)
(SEQ ID NO: 24)
MYRMQLLSCIALILALVTNS
(Trypsinogen-2 signal peptide)
(SEQ ID NO: 25)
MNLLLILTFVAAAVA.

See, e.g., Stern et al., 2007, Trends Cell. Mol. Biol., 2:1-17 and Dalton & Barton, 2014, Protein Sci, 23: 517-525, each of which is incorporated by reference herein in its entirety for the signal peptides that can be used.

As an alternative, or an additional treatment to gene therapy, the HuPTMFabVEGFi product, e.g., HuGlyFabVEGFi glycoprotein, can be produced in human cell lines by recombinant DNA technology, and administered to patients diagnosed with diabetic retinopathy (DR) by by intravitreall injection. The HuPTMFabVEGFi product, e.g., glycoprotein, may also be administered to patients with diabetic retinopathy (DR). Human cell lines that can be used for such recombinant glycoprotein production include but are not limited to human embryonic kidney 293 cells (HEK293), fibrosarcoma HT-1080, HKB-11, CAP, HuH-7, and retinal cell lines, PER.C6, or RPE to name a few (e.g., see Dumont et al., 2015, Crit. Rev. Biotechnol. (Early Online, published online Sep. 18, 2015, pp. 1-13) “Human cell lines for biopharmaceutical manufacturing: history, status, and future perspectives” which is incorporated by reference in its entirety for a review of the human cell lines that could be used for the recombinant production of the HuPTMFabVEGFi product, e.g., HuGlyFabVEGFi glycoprotein). To ensure complete glycosylation, especially sialylation, and tyrosine-sulfation, the cell line used for production can be enhanced by engineering the host cells to co-express α-2,6-sialyltransferase (or both α-2,3- and α-2,6-sialyltransferases) and/or TPST-1 and TPST-2 enzymes responsible for tyrosine-O-sulfation in retinal cells.

Combinations of delivery of the HuPTMFabVEGFi, e.g., HuGlyFabVEGFi, to the eye/retina accompanied by delivery of other available treatments are encompassed by the methods provided herein. The additional treatments may be administered before, concurrently or subsequent to the gene therapy treatment. Available treatments for diabetic retinopathy (DR) that could be combined with the gene therapy provided herein include but are not limited to laser photocoagulation, photodynamic therapy with verteporfin, and intravitreal (IVT) injections with anti-VEGF agents, including but not limited to pegaptanib, ranibizumab, aflibercept, or bevacizumab. Additional treatments with anti-VEGF agents, such as biologics, may be referred to as “rescue” therapy.

Unlike small molecule drugs, biologics usually comprise a mixture of many variants with different modifications or forms that have a different potency, pharmacokinetics, and safety profile. It is not essential that every molecule produced either in the gene therapy or protein therapy approach be fully glycosylated and sulfated. Rather, the population of glycoproteins produced should have sufficient glycosylation (from about 1% to about 10% of the population), including 2,6-sialylation, and sulfation to demonstrate efficacy. The goal of gene therapy treatment provided herein is to slow or arrest the progression of retinal degeneration, and to slow or prevent loss of vision with minimal intervention/invasive procedures. Efficacy may be monitored by measuring BCVA (Best-Corrected Visual Acuity), intraocular pressure, slit lamp biomicroscopy, indirect ophthalmoscopy, SD-OCT (SD-Optical Coherence Tomography), electroretinography (ERG). Signs of vision loss, infection, inflammation and other safety events, including retinal detachment may also be monitored. Retinal thickness may be monitored to determine efficacy of the treatments provided herein. Without being bound by any particular theory, thickness of the retina may be used as a clinical readout, wherein the greater reduction in retinal thickness or the longer period of time before thickening of the retina, the more efficacious the treatment. Retinal thickness may be determined, for example, by SD-OCT. SD-OCT is a three-dimensional imaging technology which uses low-coherence interferometry to determine the echo time delay and magnitude of backscattered light reflected off an object of interest. OCT can be used to scan the layers of a tissue sample (e.g., the retina) with 3 to 15 μm axial resolution, and SD-OCT improves axial resolution and scan speed over previous forms of the technology (Schuman, 2008, Trans. Am. Opthamol. Soc. 106:426-458). Retinal function may be determined, for example, by ERG. ERG is a non-invasive electrophysiologic test of retinal function, approved by the FDA for use in humans, which examines the light sensitive cells of the eye (the rods and cones), and their connecting ganglion cells, in particular, their response to a flash stimulation.

### 5.1 N-Glycosylation, Tyrosine Sulfation, and O-Glycosylation

The amino acid sequence (primary sequence) of the anti-VEGF antigen-binding fragment of a HuPTMFabVEGFi, e.g., HuGlyFabVEGFi, used in the methods described herein comprises at least one site at which N-glycosylation or tyrosine sulfation takes place. In certain embodiments, the amino acid sequence of the anti-VEGF antigen-binding fragment comprises at least one N-glycosylation site and at least one tyrosine sulfation site. Such sites are described in detail below. In certain embodiments, the amino acid sequence of the anti-VEGF antigen-binding fragment comprises at least one O-glycosylation site, which can be in addition to one or more N-glycosylation sites and/or tyrosine sulfation sites present in said amino acid sequence.

### 5.1.1 N-Glycosylation

Reverse Glycosylation Sites

The canonical N-glycosylation sequence is known in the art to be Asn-X-Ser(or Thr), wherein X can be any amino acid except Pro. However, it recently has been demonstrated that asparagine (Asn) residues of human antibodies can be glycosylated in the context of a reverse consensus motif, Ser(or Thr)-X-Asn, wherein X can be any amino acid except Pro. See Valliere-Douglass et al., 2009, J. Biol. Chem. 284:32493-32506; and Valliere-Douglass et al., 2010, J. Biol. Chem. 285:16012-16022. As disclosed herein, and contrary to the state of the art understanding, anti-VEGF antigen-binding fragments for use in accordance with the methods described herein, e.g., ranibizumab, comprise several of such reverse consensus sequences. Accordingly, the methods described herein comprise use of anti-VEGF antigen-binding fragments that comprise at least one N-glycosylation site comprising the sequence Ser(or Thr)-X-Asn, wherein X can be any amino acid except Pro (also referred to herein as a “reverse N-glycosylation site”).

In certain embodiments, the methods described herein comprise use of an anti-VEGF antigen-binding fragment that comprises one, two, three, four, five, six, seven, eight, nine, ten, or more than ten N-glycosylation sites comprising the sequence Ser(or Thr)-X-Asn, wherein X can be any amino acid except Pro. In certain embodiments, the methods described herein comprise use of an anti-VEGF antigen-binding fragment that comprises one, two, three, four, five, six, seven, eight, nine, ten, or more than ten reverse N-glycosylation sites, as well as one, two, three, four, five, six, seven, eight, nine, ten, or more than ten non-consensus N-glycosylation sites (as defined herein, below).

In a specific embodiment, the anti-VEGF antigen-binding fragment comprising one or more reverse N-glycosylation sites used in the methods described herein is ranibizumab, comprising a light chain and a heavy chain of SEQ ID NOs. 1 and 2, respectively. In another specific embodiment, the anti-VEGF antigen-binding fragment comprising one or more reverse N-glycosylation sites used in the methods comprises the Fab of bevacizumab, comprising a light chain and a heavy chain of SEQ ID NOs. 3 and 4, respectively.

Non-Consensus Glycosylation Sites

In addition to reverse N-glycosylation sites, it recently has been demonstrated that glutamine (Gln) residues of human antibodies can be glycosylated in the context of a non-consensus motif, Gln-Gly-Thr. See Valliere-Douglass et al., 2010, J. Biol. Chem. 285:16012-16022. Surprisingly, anti-VEGF antigen-binding fragments for use in accordance with the methods described herein, e.g., ranibizumab, comprise several of such non-consensus sequences. Accordingly, the methods described herein comprise use of anti-VEGF antigen-binding fragments that comprise at least one N-glycosylation site comprising the sequence Gln-Gly-Thr (also referred to herein as a “non-consensus N-glycosylation site”).

In certain embodiments, the methods described herein comprise use of an anti-VEGF antigen-binding fragment that comprises one, two, three, four, five, six, seven, eight, nine, ten, or more than ten N-glycosylation sites comprising the sequence Gln-Gly-Thr.

In a specific embodiment, the anti-VEGF antigen-binding fragment comprising one or more non-consensus N-glycosylation sites used in the methods described herein is ranibizumab (comprising a light chain and a heavy chain of SEQ ID NOs. 1 and 2, respectively). In another specific embodiment, the anti-VEGF antigen-binding fragment comprising one or more non-consensus N-glycosylation sites used in the methods comprises the Fab of bevacizumab (comprising a light chain and a heavy chain of SEQ ID NOs. 3 and 4, respectively).

Engineered N-Glycosylation Sites

In certain embodiments, a nucleic acid encoding an anti-VEGF antigen-binding fragment is modified to include 1, 2, 3, 4, 5, 6, 7, 8, 9, 10, or more N-glycosylation sites (including the canonical N-glycosylation consensus sequence, reverse N-glycosylation site, and non-consensus N-glycosylation sites) than would normally be associated with the HuGlyFabVEGFi (e.g., relative to the number of N-glycosylation sites associated with the anti-VEGF antigen-binding fragment in its unmodified state). In specific embodiments, introduction of glycosylation sites is accomplished by insertion of N-glycosylation sites (including the canonical N-glycosylation consensus sequence, reverse N-glycosylation site, and non-consensus N-glycosylation sites) anywhere in the primary structure of the antigen-binding fragment, so long as said introduction does not impact binding of the antigen-binding fragment to its antigen, VEGF. Introduction of glycosylation sites can be accomplished by, e.g., adding new amino acids to the primary structure of the antigen-binding fragment, or the antibody from which the antigen-binding fragment is derived (i.e., the glycosylation sites are added, in full or in part), or by mutating existing amino acids in the antigen-binding fragment, or the antibody from which the antigen-binding fragment is derived, in order to generate the N-glycosylation sites (i.e., amino acids are not added to the antigen-binding fragment/antibody, but selected amino acids of the antigen-binding fragment/antibody are mutated so as to form N-glycosylation sites). Those of skill in the art will recognize that the amino acid sequence of a protein can be readily modified using approaches known in the art, e.g., recombinant approaches that include modification of the nucleic acid sequence encoding the protein.

In a specific embodiment, an anti-VEGF antigen-binding fragment used in the method described herein is modified such that, when expressed in retinal cells, it can be hyperglycosylated. See Courtois et al., 2016, mAbs 8:99-112 which is incorporated by reference herein in its entirety. In a specific embodiment, said anti-VEGF antigen-binding fragment is ranibizumab (comprising a light chain and a heavy chain of SEQ ID NOs. 1 and 2, respectively). In another specific embodiment, said anti-VEGF antigen-binding fragment comprises the Fab of bevacizumab (comprising a light chain and a heavy chain of SEQ ID NOs. 3 and 4, respectively).

N-Glycosylation of Anti-VEGF Antigen-Binding Fragments

Unlike small molecule drugs, biologics usually comprise a mixture of many variants with different modifications or forms that have a different potency, pharmacokinetics, and safety profile. It is not essential that every molecule produced either in the gene therapy or protein therapy approach be fully glycosylated and sulfated. Rather, the population of glycoproteins produced should have sufficient glycosylation (including 2,6-sialylation) and sulfation to demonstrate efficacy. The goal of gene therapy treatment provided herein is to slow or arrest the progression of retinal degeneration, and to slow or prevent loss of vision with minimal intervention/invasive procedures.

In a specific embodiment, an anti-VEGF antigen-binding fragment, e.g., ranibizumab, used in accordance with the methods described herein, when expressed in a retinal cell, could be glycosylated at 100% of its N-glycosylation sites. However, one of skill in the art will appreciate that not every N-glycosylation site of an anti-VEGF antigen-binding fragment need be N-glycosylated in order for benefits of glycosylation to be attained. Rather, benefits of glycosylation can be realized when only a percentage of N-glycosylation sites are glycosylated, and/or when only a percentage of expressed antigen-binding fragments are glycosylated. Accordingly, in certain embodiments, an anti-VEGF antigen-binding fragment used in accordance with the methods described herein, when expressed in a retinal cell, is glycosylated at 10%-20%, 20%-30%, 30%-40%, 40%-50%, 50%-60%, 60%-70%, 70%-80%, 80%-90%, or 90%-100% of it available N-glycosylation sites. In certain embodiments, when expressed in a retinal cell, 10%-20%, 20%-30%, 30%-40%, 40%-50%, 50%-60%, 60%-70%, 70%-80%, 80%-90%, or 90%-100% of the an anti-VEGF antigen-binding fragments used in accordance with the methods described herein are glycosylated at least one of their available N-glycosylation sites.

In a specific embodiment, at least 10%, 20% 30%, 40%, 50%, 60%, 70%, 75%, 80%, 85%, 90%, 95%, or 99% of the N-glycosylation sites present in an anti-VEGF antigen-binding fragment used in accordance with the methods described herein are glycosylated at an Asn residue (or other relevant residue) present in an N-glycosylation site, when the anti-VEGF antigen-binding fragment is expressed in a retinal cell. That is, at least 50%, 60%, 70%, 75%, 80%, 85%, 90%, 95%, or 99% of the N-glycosylation sites of the resultant HuGlyFabVEGFi are glycosylated.

In another specific embodiment, at least 10%, 20% 30%, 40%, 50%, 60%, 70%, 75%, 80%, 85%, 90%, 95%, or 99% of the N-glycosylation sites present in an anti-VEGF antigen-binding fragment used in accordance with the methods described herein are glycosylated with an identical attached glycan linked to the Asn residue (or other relevant residue) present in an N-glycosylation site, when the anti-VEGF antigen-binding fragment is expressed in a retinal cell. That is, at least 50%, 60%, 70%, 75%, 80%, 85%, 90%, 95%, or 99% of the N-glycosylation sites of the resultant HuGlyFabVEGFi an identical attached glycan.

When an anti-VEGF antigen-binding fragment, e.g., ranibizumab, used in accordance with the methods described herein is expressed in a retinal cell, the N-glycosylation sites of the of the antigen-binding fragment can be glycosylated with various different glycans. N-glycans of antigen-binding fragments have been characterized in the art. For example, Bondt et al., 2014, Mol. & Cell. Proteomics 13.11:3029-3039 (incorporated by reference herein in its entirety for it disclosure of Fab-associated N-glycans) characterizes glycans associated with Fabs, and demonstrates that Fab and Fc portions of antibodies comprise distinct glycosylation patterns, with Fab glycans being high in galactosylation, sialylation, and bisection (e.g., with bisecting GlcNAc) but low in fucosylation with respect to Fc glycans. Like Bondt, Huang et al., 2006, Anal. Biochem. 349:197-207 (incorporated by reference herein in its entirety for it disclosure of Fab-associated N-glycans) found that most glycans of Fabs are sialylated. However, in the Fab of the antibody examined by Huang (which was produced in a murine cell background), the identified sialic residues were N-Glycolylneuraminic acid (“Neu5Gc” or “NeuGc”) (which is not natural to humans) instead of N-acetylneuraminic acid (“Neu5Ac,” the predominant human sialic acid). In addition, Song et al., 2014, Anal. Chem. 86:5661-5666 (incorporated by reference herein in its entirety for it disclosure of Fab-associated N-glycans) describes a library of N-glycans associated with commercially available antibodies.

Importantly, when the anti-VEGF antigen-binding fragments, e.g., ranibizumab, used in accordance with the methods described herein are expressed in human retinal cells, the need for in vitro production in prokaryotic host cells (e.g., E. coli) or eukaryotic host cells (e.g., CHO cells) is circumvented. Instead, as a result of the methods described herein (e.g., use of retinal cells to express anti-hVEGF antigen-binding fragments), N-glycosylation sites of the anti-VEGF antigen-binding fragments are advantageously decorated with glycans relevant to and beneficial to treatment of humans. Such an advantage is unattainable when CHO cells or E. coli are utilized in antibody/antigen-binding fragment production, because e.g., CHO cells (1) do not express 2,6 sialyltransferase and thus cannot add 2,6 sialic acid during N-glycosylation and (2) can add Neu5Gc as sialic acid instead of Neu5Ac; and because E. coli does not naturally contain components needed for N-glycosylation. Accordingly, in one embodiment, an anti-VEGF antigen-binding fragment expressed in a retinal cell to give rise to a HuGlyFabVEGFi used in the methods of treatment described herein is glycosylated in the manner in which a protein is N-glycosylated in human retinal cells, e.g., retinal pigment cells, but is not glycosylated in the manner in which proteins are glycosylated in CHO cells. In another embodiment, an anti-VEGF antigen-binding fragment expressed in a retinal cell to give rise to a HuGlyFabVEGFi used in the methods of treatment described herein is glycosylated in the manner in which a protein is N-glycosylated in human retinal cells, e.g., retinal pigment cells, wherein such glycosylation is not naturally possible using a prokaryotic host cell, e.g., using E. coli.

In certain embodiments, a HuGlyFabVEGFi, e.g., ranibizumab, used in accordance with the methods described herein comprises one, two, three, four, five or more distinct N-glycans associated with Fabs of human antibodies. In a specific embodiment, said N-glycans associated with Fabs of human antibodies are those described in Bondt et al., 2014, Mol. & Cell. Proteomics 13.11:3029-3039, Huang et al., 2006, Anal. Biochem. 349:197-207, and/or Song et al., 2014, Anal. Chem. 86:5661-5666. In certain embodiments, a HuGlyFabVEGFi, e.g., ranibizumab, used in accordance with the methods described herein does not comprise detectable NeuGc and/or α-Gal antigen.

In a specific embodiment, the HuGlyFabVEGFi, e.g., ranibizumab, used in accordance with the methods described herein are predominantly glycosylated with a glycan comprising 2,6-linked sialic acid. In certain embodiments, HuGlyFabVEGFi comprising 2,6-linked sialic acid is polysialylated, i.e., contains more than one sialic acid. In certain embodiments, each N-glycosylation site of said HuGlyFabVEGFi comprises a glycan comprising 2,6-linked sialic acid, i.e., 100% of the N-glycosylation site of said HuGlyFabVEGFi comprise a glycan comprising 2,6-linked sialic acid. In another specific embodiment, at least 20%, 30%, 40%, 50%, 60%, 70%, 75%, 80%, 85%, 90%, 95%, or 99% of the N-glycosylation sites of a HuGlyFabVEGFi used in accordance with the methods described herein are glycosylated with a glycan comprising 2,6-linked sialic acid. In another specific embodiment, at least 10%-20%, 20%-30%, 30%-40%, 40%-50%, 50%-60%, 60%-70%, 70%-80%, 80% -90%, or 90%-99% of the N-glycosylation sites of a HuGlyFabVEGFi used in accordance with the methods described herein are glycosylated with a glycan comprising 2,6-linked sialic acid. In another specific embodiment, at least 20%, 30%, 40%, 50%, 60%, 70%, 75%, 80%, 85%, 90%, 95%, or 99% of the antigen-binding fragments expressed in a retinal cell in accordance with methods described herein (i.e., the antigen-binding fragments that give rise to HuGlyFabVEGFi, e.g., ranibizumab) are glycosylated with a glycan comprising 2,6-linked sialic acid. In another specific embodiment, at least 10%-20%, 20%-30%, 30%-40%, 40%-50%, 50%-60%, 60%-70%, 70%-80%, 80%-90%, or 90%-99% of the antigen-binding fragments expressed in a retinal cell in accordance with methods described herein (i.e., the Fabs that give rise to HuGlyFabVEGFi, e.g., ranibizumab) are glycosylated with a glycan comprising 2,6-linked sialic acid. In another specific embodiment, said sialic acid is Neu5Ac. In accordance with such embodiments, when only a percentage of the N-glycosylation sites of a HuGlyFabVEGFi are 2,6 sialylated or polysialylated, the remaining N-glycosylation can comprise a distinct N-glycan, or no N-glycan at all (i.e., remain non-glycosylated).

When a HuGlyFabVEGFi is 2,6 polysialylated, it comprises multiple sialic acid residues, e.g., 2, 3, 4, 5, 6, 7, 8, 9, 10 or more than 10 sialic acid residues. In certain embodiments, when a HuGlyFabVEGFi is polysialylated, it comprises 2-5, 5-10, 10-20, 20-30, 30-40, or 40-50 sialic acid residues. In certain embodiments, when a HuGlyFabVEGFi is polysialylated, it comprises 2,6-linked (sialic acid)ⁿ, wherein n can be any number from 1-100.

In a specific embodiment, the HuGlyFabVEGFi, e.g., ranibizumab, used in accordance with the methods described herein are predominantly glycosylated with a glycan comprising a bisecting GlcNAc. In certain embodiments, each N-glycosylation site of said HuGlyFabVEGFi comprises a glycan comprising a bisecting GlcNAc, i.e., 100% of the N-glycosylation site of said HuGlyFabVEGFi comprise a glycan comprising a bisecting GlcNAc. In another specific embodiment, at least 20%, 30%, 40%, 50%, 60%, 70%, 75%, 80%, 85%, 90%, 95%, or 99% of the N-glycosylation sites of a HuGlyFabVEGFi used in accordance with the methods described herein are glycosylated with a glycan comprising a bisecting GlcNAc. In another specific embodiment, at least 10%-20%, 20%-30%, 30%-40%, 40%-50%, 50%-60%, 60%-70%, 70%-80%, 80%-90%, or 90%-99% of the N-glycosylation sites of a HuGlyFabVEGFi used in accordance with the methods described herein are glycosylated with a glycan comprising a bisecting GlcNAc. In another specific embodiment, at least 20%, 30%, 40%, 50%, 60%, 70%, 75%, 80%, 85%, 90%, 95%, or 99% of the antigen-binding fragments expressed in a retinal cell in accordance with methods described herein (i.e., the antigen-binding fragments that give rise to HuGlyFabVEGFi, e.g., ranibizumab) are glycosylated with a glycan comprising a bisecting GlcNAc. In another specific embodiment, at least 10%-20%, 20%-30%, 30%-40%, 40%-50%, 50%-60%, 60%-70%, 70%-80%, 80%-90%, or 90%-99% of the antigen-binding fragments expressed in a retinal cell in accordance with methods described herein (i.e., the antigen-binding fragments that give rise to HuGlyFabVEGFi, e.g., ranibizumab) are glycosylated with a glycan comprising a bisecting GlcNAc.

In certain embodiments, the HuGlyFabVEGFi, e.g., ranibizumab, used in accordance with the methods described herein are hyperglycosylated, i.e., in addition to the N-glycosylation resultant from the naturally occurring N-glycosylation sites, said HuGlyFabVEGFi comprise glycans at N-glycosylation sites engineered to be present in the amino acid sequence of the antigen-binding fragment giving rise to HuGlyFabVEGFi. In certain embodiments, the HuGlyFabVEGFi, e.g., ranibizumab, used in accordance with the methods described herein is hyperglycosylated but does not comprise detectable NeuGc and/or α-Gal antigen.

Assays for determining the glycosylation pattern of antibodies, including antigen-binding fragments are known in the art. For example, hydrazinolysis can be used to analyze glycans. First, polysaccharides are released from their associated protein by incubation with hydrazine (the Ludger Liberate Hydrazinolysis Glycan Release Kit, Oxfordshire, UK can be used). The nucleophile hydrazine attacks the glycosidic bond between the polysaccharide and the carrier protein and allows release of the attached glycans. N-acetyl groups are lost during this treatment and have to be reconstituted by re-N-acetylation. Glycans may also be released using enzymes such as glycosidases or endoglycosidases, such as PNGase F and Endo H, which cleave cleanly and with fewer side reactions than hydrazines. The free glycans can be purified on carbon columns and subsequently labeled at the reducing end with the fluorophor 2-amino benzamide. The labeled polysaccharides can be separated on a GlycoSep-N column (GL Sciences) according to the HPLC protocol of Royle et al, Anal Biochem 2002, 304(1):70-90. The resulting fluorescence chromatogram indicates the polysaccharide length and number of repeating units. Structural information can be gathered by collecting individual peaks and subsequently performing MS/MS analysis. Thereby the monosaccharide composition and sequence of the repeating unit can be confirmed and additionally in homogeneity of the polysaccharide composition can be identified. Specific peaks of low or high molecular weight can be analyzed by MALDI-MS/MS and the result used to confirm the glycan sequence. Each peak in the chromatogram corresponds to a polymer, e.g., glycan, consisting of a certain number of repeat units and fragments, e.g., sugar residues, thereof. The chromatogram thus allows measurement of the polymer, e.g., glycan, length distribution. The elution time is an indication for polymer length, while fluorescence intensity correlates with molar abundance for the respective polymer, e.g., glycan. Other methods for assessing glycans associated with antigen-binding fragments include those described by Bondt et al., 2014, Mol. & Cell. Proteomics 13.11:3029-3039, Huang et al., 2006, Anal. Biochem. 349:197-207, and/or Song et al., 2014, Anal. Chem. 86:5661-5666.

Homogeneity or heterogeneity of the glycan patterns associated with antibodies (including antigen-binding fragments), as it relates to both glycan length or size and numbers glycans present across glycosylation sites, can be assessed using methods known in the art, e.g., methods that measure glycan length or size and hydrodynamic radius. HPLC, such as Size exclusion, normal phase, reversed phase, and anion exchange HPLC, as well as capillary electrophoresis, allows the measurement of the hydrodynamic radius. Higher numbers of glycosylation sites in a protein lead to higher variation in hydrodynamic radius compared to a carrier with less glycosylation sites. However, when single glycan chains are analyzed, they may be more homogenous due to the more controlled length. Glycan length can be measured by hydrazinolysis, SDS PAGE, and capillary gel electrophoresis. In addition, homogeneity can also mean that certain glycosylation site usage patterns change to a broader/narrower range. These factors can be measured by Glycopeptide LC-MS/MS.

Benefits of N-Glycosylation

N-glycosylation confers numerous benefits on the HuGlyFabVEGFi used in the methods described herein. Such benefits are unattainable by production of antigen-binding fragments in E. coli, because E. coli does not naturally possess components needed for N-glycosylation. Further, some benefits are unattainable through antibody production in, e.g., CHO cells, because CHO cells lack components needed for addition of certain glycans (e.g., 2,6 sialic acid and bisecting GlcNAc) and because CHO cells can add glycans, e.g., Neu5Gc not typical to humans. See, e.g., Song et al., 2014, Anal. Chem. 86:5661-5666. Accordingly, by virtue of the discovery set forth herein that anti-VEGF antigen-binding fragments, e.g., ranibizumab, comprise non-canonical N-glycosylation sites (including both reverse and non-consensus glycosylation sites), a method of expressing such anti-VEGF antigen-binding fragments in a manner that results in their glycosylation (and thus improved benefits associated with the antigen-binding fragments) has been realized. In particular, expression of anti-VEGF antigen-binding fragments in human retinal cells results in the production of HuGlyFabVEGFi (e.g., ranibizumab) comprising beneficial glycans that otherwise would not be associated with the antigen-binding fragments or their parent antibody.

While non-canonical glycosylation sites usually result in low level glycosylation (e.g., 1-5%) of the antibody population, the functional benefits may be significant in immunoprivileged organs, such as the eye (See, e.g., van de Bovenkamp et al., 2016, J. Immunol. 196:1435-1441). For example, Fab glycosylation may affect the stability, half-life, and binding characteristics of an antibody. To determine the effects of Fab glycosylation on the affinity of the antibody for its target, any technique known to one of skill in the art may be used, for example, enzyme linked immunosorbent assay (ELISA), or surface plasmon resonance (SPR). To determine the effects of Fab glycosylation on the half-life of the antibody, any technique known to one of skill in the art may be used, for example, by measurement of the levels of radioactivity in the blood or organs (e.g., the eye) in a subject to whom a radiolabeled antibody has been administered. To determine the effects of Fab glycosylation on the stability, for example, levels of aggregation or protein unfolding, of the antibody, any technique known to one of skill in the art may be used, for example, differential scanning calorimetry (DSC), high performance liquid chromatography (HPLC), e.g., size exclusion high performance liquid chromatography (SEC-HPLC), capillary electrophoresis, mass spectrometry, or turbidity measurement. Provided herein, the HuGlyFabVEGFi transgene results in production of an antigen-binding fragment which is 0.5%, 1%, 2%, 3%, 4%, 5%, 6%, 7%, 8%, 9%, or 10% or more glycosylated at non-canonical sites. In certain embodiments, 0.5%, 1%, 2%, 3%, 4%, 5%, 6%, 7%, 8%, 9%, or 10% or more antigen-binding fragments from a population of antigen-binding fragments are glycosylated at non-canonical sites. In certain embodiments, 0.5%, 1%, 2%, 3%, 4%, 5%, 6%, 7%, 8%, 9%, or 10% or more non-canonical sites are glycosylated. In certain embodiments, the glycosylation of the antigen-binding fragment at these non-canonical sites is 25%, 50%, 100%, 200%, 300%, 400%, 500%, or more greater than the amount of glycosylation of these non-canonical sites in an antigen-binding fragment produced in HEK293 cells.

The presence of sialic acid on HuGlyFabVEGFi used in the methods described herein can impact clearance rate of the HuGlyFabVEGFi, e.g., the rate of clearance from the vitreous humour. Accordingly, sialic acid patterns of a HuGlyFabVEGFi can be used to generate a therapeutic having an optimized clearance rate. Method of assessing antigen-binding fragment clearance rate are known in the art. See, e.g., Huang et al., 2006, Anal. Biochem. 349:197-207.

In another specific embodiment, a benefit conferred by N-glycosylation is reduced aggregation. Occupied N-glycosylation sites can mask aggregation prone amino acid residues, resulting in decreased aggregation. Such N-glycosylation sites can be native to an antigen-binding fragment used herein, or engineered into an antigen-binding fragment used herein, resulting in HuGlyFabVEGFi that is less prone to aggregation when expressed, e.g., expressed in retinal cells. Methods of assessing aggregation of antibodies are known in the art. See, e.g., Courtois et al., 2016, mAbs 8:99-112 which is incorporated by reference herein in its entirety.

In another specific embodiment, a benefit conferred by N-glycosylation is reduced immunogenicity. Such N-glycosylation sites can be native to an antigen-binding fragment used herein, or engineered into an antigen-binding fragment used herein, resulting in HuGlyFabVEGFi that is less prone to immunogenicity when expressed, e.g., expressed in retinal cells.

In another specific embodiment, a benefit conferred by N-glycosylation is protein stability. N-glycosylation of proteins is well-known to confer stability on them, and methods of assessing protein stability resulting from N-glycosylation are known in the art. See, e.g., Sola and Griebenow, 2009, J Pharm Sci., 98(4): 1223-1245.

In another specific embodiment, a benefit conferred by N-glycosylation is altered binding affinity. It is known in the art that the presence of N-glycosylation sites in the variable domains of an antibody can increase the affinity of the antibody for its antigen. See, e.g., Bovenkamp et al., 2016, J. Immunol. 196:1435-1441. Assays for measuring antibody binding affinity are known in the art. See, e.g., Wright et al., 1991, EMBO J. 10:2717-2723; and Leibiger et al., 1999, Biochem. J. 338:529-538.

### 5.1.2 Tyrosine Sulfation

Tyrosine sulfation occurs at tyrosine (Y) residues with glutamate (E) or aspartate (D) within +5 to −5 position of Y, and where position −1 of Y is a neutral or acidic charged amino acid, but not a basic amino acid, e.g., arginine (R), lysine (K), or histidine (H) that abolishes sulfation. Surprisingly, anti-VEGF antigen-binding fragments for use in accordance with the methods described herein, e.g., ranibizumab, comprise tyrosine sulfation sites (see FIG. 1). Accordingly, the methods described herein comprise use of anti-VEGF antigen-binding fragments, e.g., HuPTMFabVEGFi , that comprise at least one tyrosine sulfation site, such the anti-VEGF antigen-binding fragments, when expressed in retinal cells, can be tyrosine sulfated.

Importantly, tyrosine-sulfated antigen-binding fragments, e.g., ranibizumab, cannot be produced in E. coli, which naturally does not possess the enzymes required for tyrosine-sulfation. Further, CHO cells are deficient for tyrosine sulfation—they are not secretory cells and have a limited capacity for post-translational tyrosine-sulfation. See, e.g., Mikkelsen & Ezban, 1991, Biochemistry 30: 1533-1537. Advantageously, the methods provided herein call for expression of anti-VEGF antigen-binding fragments, e.g., HuPTMFabVEGFi , for example, ranibizumab, in retinal cells, which are secretory and do have capacity for tyrosine sulfation. See Kanan et al., 2009, Exp. Eye Res. 89: 559-567 and Kanan & Al-Ubaidi, 2015, Exp. Eye Res. 133: 126-131 reporting the production of tyrosine-sulfated glycoproteins secreted by retinal cells.

Tyrosine sulfation is advantageous for several reasons. For example, tyrosine-sulfation of the antigen-binding fragment of therapeutic antibodies against targets has been shown to dramatically increase avidity for antigen and activity. See, e.g., Loos et al., 2015, PNAS 112: 12675-12680, and Choe et al., 2003, Cell 114: 161-170. Assays for detection tyrosine sulfation are known in the art. See, e.g., Yang et al., 2015, Molecules 20:2138-2164.

### 5.1.3 O-Glycosylation

O-glycosylation comprises the addition of N-acetyl-galactosamine to serine or threonine residues by the enzyme. It has been demonstrated that amino acid residues present in the hinge region of antibodies can be O-glycosylated. In certain embodiments, the anti-VEGF antigen-binding fragments, e.g., ranibizumab, used in accordance with the methods described herein comprise all or a portion of their hinge region, and thus are capable of being O-glycosylated when expressed in human retinal cells. The possibility of O-glycosylation confers another advantage to the HuPTMFabVEGFi, e.g., HuGlyFabVEGFi, provided herein, as compared to, e.g., antigen-binding fragments produced in E. coli, again because the E. coli naturally does not contain machinery equivalent to that used in human O-glycosylation. (Instead, O-glycosylation in E. coli has been demonstrated only when the bacteria is modified to contain specific O-glycosylation machinery. See, e.g., Faridmoayer et al., 2007, J. Bacteriol. 189:8088-8098.) O-glycosylated HuPTMFabVEGFi, e.g., HuGlyFabVEGFi, by virtue of possessing glycans, shares advantageous characteristics with N-glycosylated HuGlyFabVEGFi (as discussed above).

### 5.2 Constructs and Formulations

For use in the methods provided herein are viral vectors or other DNA expression constructs encoding an anti-VEGF antigen-binding fragment or a hyperglycosylated derivative of an anti-VEGF antigen-binding fragment. The viral vectors and other DNA expression constructs provided herein include any suitable method for delivery of a transgene to a target cell (e.g., retinal pigment epithelial cells). The means of delivery of a transgene include viral vectors, liposomes, other lipid-containing complexes, other macromolecular complexes, synthetic modified mRNA, unmodified mRNA, small molecules, non-biologically active molecules (e.g., gold particles), polymerized molecules (e.g., dendrimers), naked DNA, plasmids, phages, transposons, cosmids, or episomes. In some embodiments, the vector is a targeted vector, e.g., a vector targeted to retinal pigment epithelial cells.

In some aspects, the disclosure provides for a nucleic acid for use, wherein the nucleic acid encodes a HuPTMFabVEGFi, e.g., HuGlyFabVEGFi operatively linked to a promoter selected from the group consisting of: the CB7 promoter (a chicken β-actin promoter and CMV enhancer), cytomegalovirus (CMV) promoter, Rous sarcoma virus (RSV) promoter, MMT promoter, EF-1 alpha promoter, UB6 promoter, chicken beta-actin promoter, CAG promoter, RPE65 promoter and opsin promoter. In a specific embodiment, HuPTMFabVEGFi is operatively linked to the CB7 promoter.

In certain embodiments, provided herein are recombinant vectors that comprise one or more nucleic acids (e.g. polynucleotides). The nucleic acids may comprise DNA, RNA, or a combination of DNA and RNA. In certain embodiments, the DNA comprises one or more of the sequences selected from the group consisting of promoter sequences, the sequence of the gene of interest (the transgene, e.g., an anti-VEGF antigen-binding fragment), untranslated regions, and termination sequences. In certain embodiments, viral vectors provided herein comprise a promoter operably linked to the gene of interest.

In certain embodiments, nucleic acids (e.g., polynucleotides) and nucleic acid sequences disclosed herein may be codon-optimized, for example, via any codon-optimization technique known to one of skill in the art (see, e.g., review by Quax et al., 2015, Mol Cell 59:149-161).

In a specific embodiment, the construct described herein is Construct I, wherein the Construct I comprises the following components: (1) AAV8 inverted terminal repeats that flank the expression cassette; (2) control elements, which include a) the CB7 promoter, comprising the CMV enhancer/chicken β-actin promoter, b) a chicken β-actin intron and c) a rabbit β-globin poly A signal; and (3) nucleic acid sequences coding for the heavy and light chains of anti-VEGF antigen-binding fragment, separated by a self-cleaving furin (F)/F2A linker, ensuring expression of equal amounts of the heavy and the light chain polypeptides.

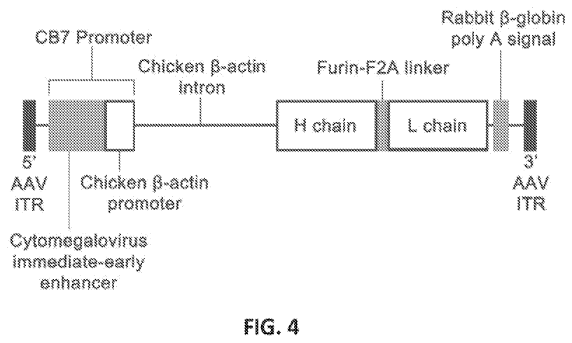

In another specific embodiment, the construct described herein is Construct II, wherein the Construct II comprises the following components: (1) AAV2 inverted terminal repeats that flank the expression cassette; (2) control elements, which include a) the CB7 promoter, comprising the CMV enhancer/chicken β-actin promoter, b) a chicken β-actin intron and c) a rabbit β-globin poly A signal; and (3) nucleic acid sequences coding for the heavy and light chains of anti-VEGF antigen-binding fragment, separated by a self-cleaving furin (F)/F2A linker, ensuring expression of equal amounts of the heavy and the light chain polypeptides. In a specific embodiment, the construct described herein is illustrated in FIG. 4.

5.2.1 mRNA

In certain embodiments, the vectors provided herein are modified mRNA encoding for the gene of interest (e.g., the transgene, for example, an anti-VEGF antigen-binding fragment moiety). The synthesis of modified and unmodified mRNA for delivery of a transgene to retinal pigment epithelial cells is taught, for example, in Hansson et al., J. Biol. Chem., 2015, 290(9):5661-5672, which is incorporated by reference herein in its entirety. In certain embodiments, provided herein is a modified mRNA encoding for an anti-VEGF antigen-binding fragment moiety.

### 5.2.2 Viral Vectors

Viral vectors include adenovirus, adeno-associated virus (AAV, e.g., AAV8), lentivirus, helper-dependent adenovirus, herpes simplex virus, poxvirus, hemagglutinin virus of Japan (HVJ), alphavirus, vaccinia virus, and retrovirus vectors. Retroviral vectors include murine leukemia virus (MLV)- and human immunodeficiency virus (HIV)-based vectors. Alphavirus vectors include semliki forest virus (SFV) and sindbis virus (SIN). In certain embodiments, the viral vectors provided herein are recombinant viral vectors. In certain embodiments, the viral vectors provided herein are altered such that they are replication-deficient in humans. In certain embodiments, the viral vectors are hybrid vectors, e.g., an AAV vector placed into a “helpless” adenoviral vector. In certain embodiments, provided herein are viral vectors comprising a viral capsid from a first virus and viral envelope proteins from a second virus. In specific embodiments, the second virus is vesicular stomatitus virus (VSV). In more specific embodiments, the envelope protein is VSV-G protein.

In certain embodiments, the viral vectors provided herein are HIV based viral vectors. In certain embodiments, HIV-based vectors provided herein comprise at least two polynucleotides, wherein the gag and pol genes are from an HIV genome and the env gene is from another virus.

In certain embodiments, the viral vectors provided herein are herpes simplex virus-based viral vectors. In certain embodiments, herpes simplex virus-based vectors provided herein are modified such that they do not comprise one or more immediately early (IE) genes, rendering them non-cytotoxic.

In certain embodiments, the viral vectors provided herein are MLV based viral vectors. In certain embodiments, MLV-based vectors provided herein comprise up to 8 kb of heterologous DNA in place of the viral genes.

In certain embodiments, the viral vectors provided herein are lentivirus-based viral vectors. In certain embodiments, lentiviral vectors provided herein are derived from human lentiviruses. In certain embodiments, lentiviral vectors provided herein are derived from non-human lentiviruses. In certain embodiments, lentiviral vectors provided herein are packaged into a lentiviral capsid. In certain embodiments, lentiviral vectors provided herein comprise one or more of the following elements: long terminal repeats, a primer binding site, a polypurine tract, att sites, and an encapsidation site.

In certain embodiments, the viral vectors provided herein are alphavirus-based viral vectors. In certain embodiments, alphavirus vectors provided herein are recombinant, replication-defective alphaviruses. In certain embodiments, alphavirus replicons in the alphavirus vectors provided herein are targeted to specific cell types by displaying a functional heterologous ligand on their virion surface.

In certain embodiments, the viral vectors provided herein are AAV based viral vectors. In preferred embodiments, the viral vectors provided herein are AAV8 based viral vectors. In certain embodiments, the AAV8 based viral vectors provided herein retain tropism for retinal cells. In certain embodiments, the AAV-based vectors provided herein encode the AAV rep gene (required for replication) and/or the AAV cap gene (required for synthesis of the capsid proteins). Multiple AAV serotypes have been identified. In certain embodiments, AAV-based vectors provided herein comprise components from one or more serotypes of AAV. In certain embodiments, AAV based vectors provided herein comprise capsid components from one or more of AAV1, AAV2, AAV3, AAV4, AAV5, AAV6, AAV7, AAV8, AAV9, AAV10, AAV11, or AAVrh10. In preferred embodiments, AAV based vectors provided herein comprise components from one or more of AAV8, AAV9, AAV10, AAV11, or AAVrh10 serotypes.

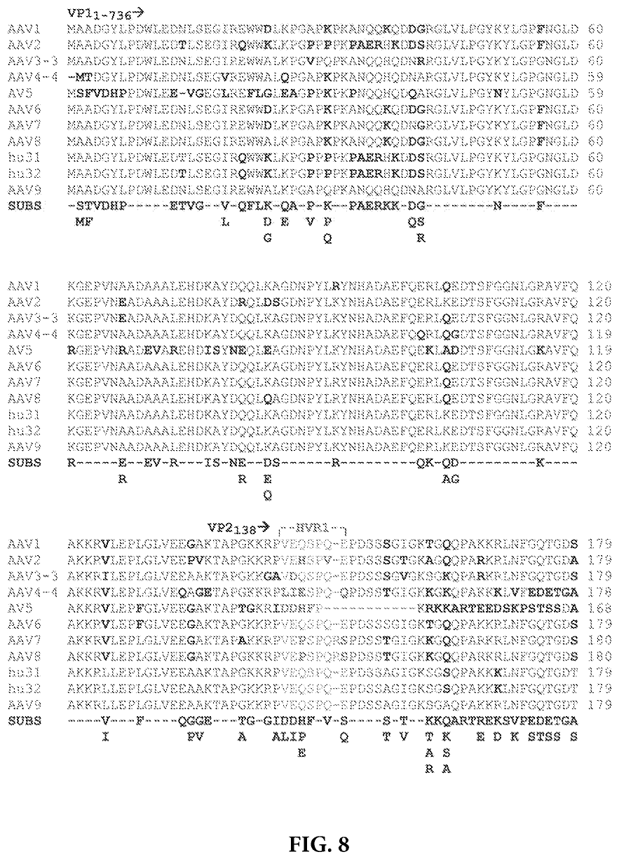

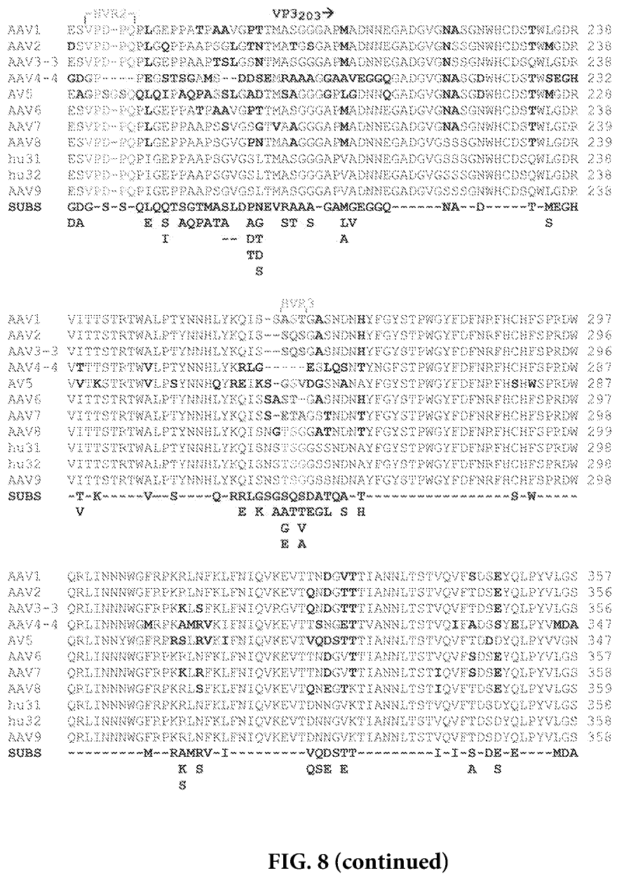

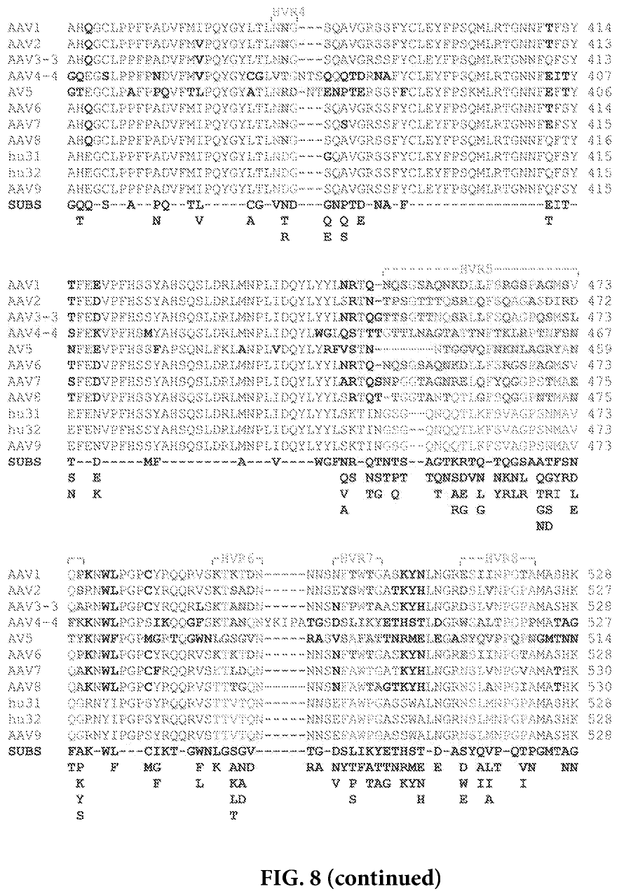

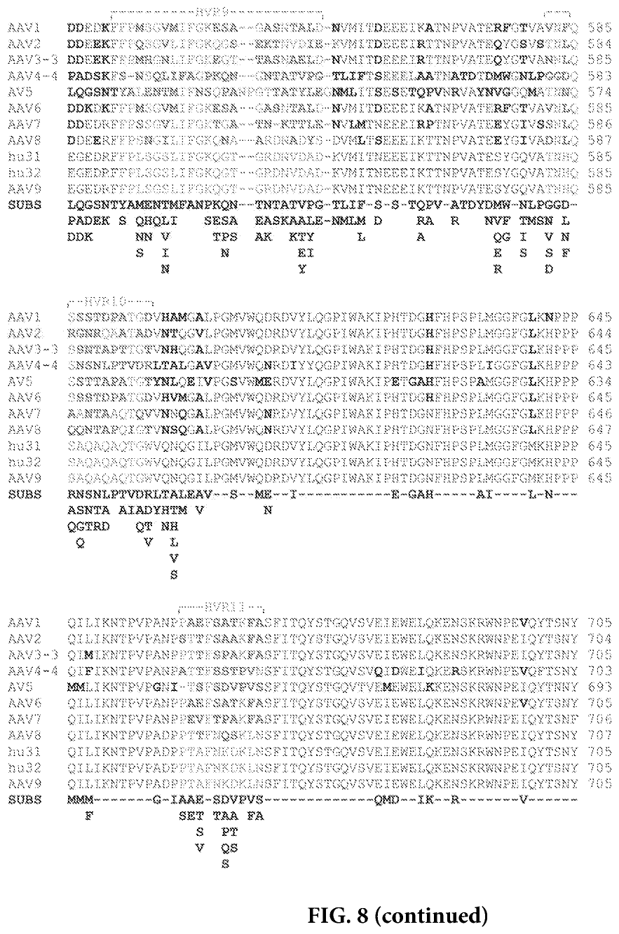

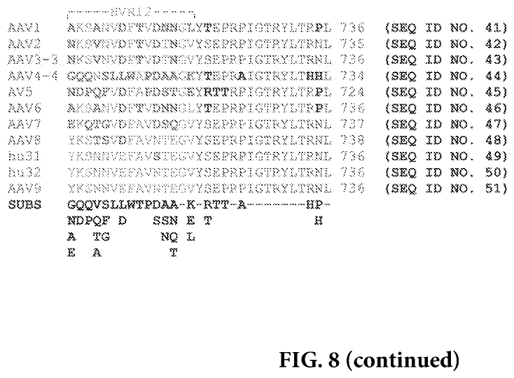

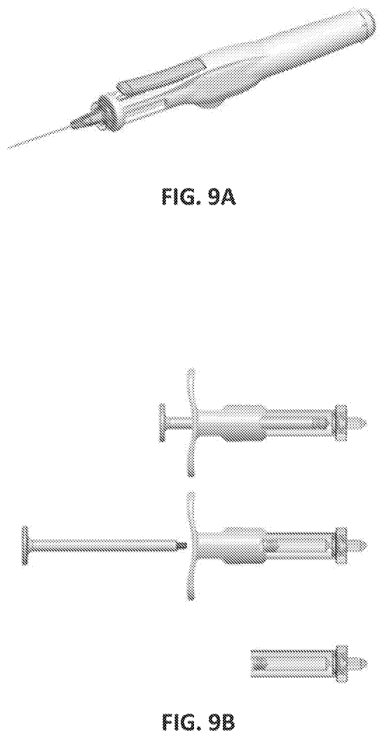

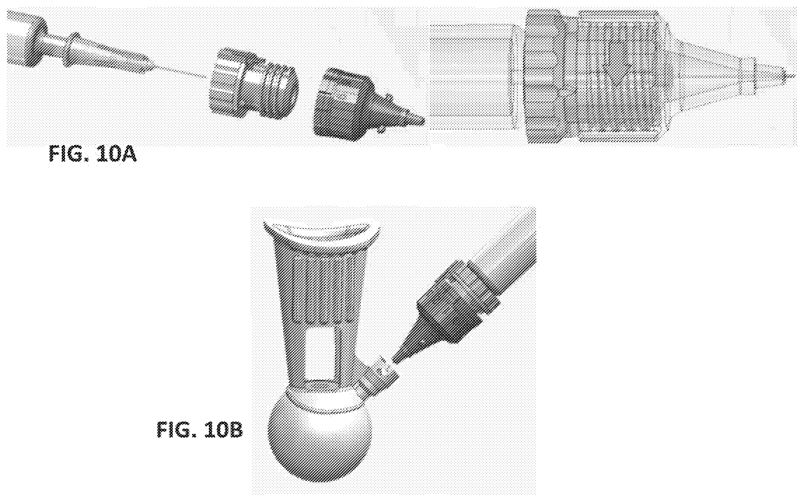

Provided in particular embodiments are AAV8 vectors comprising a viral genome comprising an expression cassette for expression of the transgene, under the control of regulatory elements and flanked by ITRs and a viral capsid that has the amino acid sequence of the AAV8 capsid protein or is at least 95%, 96%, 97%, 98%, 99% or 99.9% identical to the amino acid sequence of the AAV8 capsid protein (SEQ ID NO: 48) while retaining the biological function of the AAV8 capsid. In certain embodiments, the encoded AAV8 capsid has the sequence of SEQ ID NO: 48 with 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 amino acid substitutions and retaining the biological function of the AAV8 capsid. FIG. 8 provides a comparative alignment of the amino acid sequences of the capsid proteins of different AAV serotypes with potential amino acids that may be substituted at certain positions in the aligned sequences based upon the comparison in the row labeled SUBS. Accordingly, in specific embodiments, the AAV8 vector comprises an AAV8 capsid variant that has 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 amino acid substitutions identified in the SUBS row of FIG. 8 that are not present at that position in the native AAV8 sequence.

In certain embodiments, the AAV that is used in the methods described herein is Anc80 or Anc80L65, as described in Zinn et al., 2015, Cell Rep. 12(6): 1056-1068, which is incorporated by reference in its entirety. In certain embodiments, the AAV that is used in the methods described herein comprises one of the following amino acid insertions: LGETTRP or LALGETTRP, as described in U.S. Pat. Nos. 9,193,956; 9458517; and 9,587,282 and US patent application publication no. 2016/0376323, each of which is incorporated herein by reference in its entirety. In certain embodiments, the AAV that is used in the methods described herein is AAV.7m8, as described in U.S. Pat. Nos. 9,193,956; 9,458,517; and 9,587,282 and US patent application publication no. 2016/0376323, each of which is incorporated herein by reference in its entirety. In certain embodiments, the AAV that is used in the methods described herein is any AAV disclosed in U.S. Pat. No. 9,585,971, such as AAV-PHP.B. In certain embodiments, the AAV that is used in the methods described herein is an AAV disclosed in any of the following patents and patent applications, each of which is incorporated herein by reference in its entirety: U.S. Pat. Nos. 7,906,111; 8,524,446; 8,999,678; 8,628,966; 8,927,514; 8,734,809; US 9,284,357; 9,409,953; 9,169,299; 9,193,956; 9458517; and 9,587,282 US patent application publication nos. 2015/0374803; 2015/0126588; 2017/0067908; 2013/0224836; 2016/0215024; 2017/0051257; and International Patent Application Nos. PCT/US2015/034799; PCT/EP2015/053335.

AAV8-based viral vectors are used in certain of the methods described herein. Nucleic acid sequences of AAV based viral vectors and methods of making recombinant AAV and AAV capsids are taught, for example, in U.S. Pat. Nos. 7,282,199 B2, 7,790,449 B2, 8,318,480 B2, 8,962,332 B2 and International Patent Application No. PCT/EP2014/076466, each of which is incorporated herein by reference in its entirety. In one aspect, provided herein are AAV (e.g., AAV8)-based viral vectors encoding a transgene (e.g., an anti-VEGF antigen-binding fragment). In specific embodiments, provided herein are AAV8-based viral vectors encoding an anti-VEGF antigen-binding fragment. In more specific embodiments, provided herein are AAV8-based viral vectors encoding ranibizumab.

In certain embodiments, a single-stranded AAV (ssAAV) may be used supra. In certain embodiments, a self-complementary vector, e.g., scAAV, may be used (see, e.g., Wu, 2007, Human Gene Therapy, 18(2):171-82, McCarty et al, 2001, Gene Therapy, Vol 8, Number 16, Pages 1248-1254; and U.S. Pat. Nos. 6,596,535; 7,125,717; and 7,456,683, each of which is incorporated herein by reference in its entirety).

In certain embodiments, the viral vectors used in the methods described herein are adenovirus based viral vectors. A recombinant adenovirus vector may be used to transfer in the anti-VEGF antigen-binding fragment. The recombinant adenovirus can be a first generation vector, with an E1 deletion, with or without an E3 deletion, and with the expression cassette inserted into either deleted region. The recombinant adenovirus can be a second generation vector, which contains full or partial deletions of the E2 and E4 regions. A helper-dependent adenovirus retains only the adenovirus inverted terminal repeats and the packaging signal (phi). The transgene is inserted between the packaging signal and the 3′ITR, with or without stuffer sequences to keep the genome close to wild-type size of approx. 36 kb. An exemplary protocol for production of adenoviral vectors may be found in Alba et al., 2005, “Gutless adenovirus: last generation adenovirus for gene therapy,” Gene Therapy 12:S18-S27, which is incorporated by reference herein in its entirety.

In certain embodiments, the viral vectors used in the methods described herein are lentivirus based viral vectors. A recombinant lentivirus vector may be used to transfer in the anti-VEGF antigen-binding fragment. Four plasmids are used to make the construct: Gag/pol sequence containing plasmid, Rev sequence containing plasmids, Envelope protein containing plasmid (i.e. VSV-G), and Cis plasmid with the packaging elements and the anti-VEGF antigen-binding fragment gene.

For lentiviral vector production, the four plasmids are co-transfected into cells (i.e., HEK293 based cells), whereby polyethylenimine or calcium phosphate can be used as transfection agents, among others. The lentivirus is then harvested in the supernatant (lentiviruses need to bud from the cells to be active, so no cell harvest needs/should be done). The supernatant is filtered (0.45 μm) and then magnesium chloride and benzonase added. Further downstream processes can vary widely, with using TFF and column chromatography being the most GMP compatible ones. Others use ultracentrifugation with/without column chromatography. Exemplary protocols for production of lentiviral vectors may be found in Lesch et al., 2011, “Production and purification of lentiviral vector generated in 293T suspension cells with baculoviral vectors,” Gene Therapy 18:531-538, and Ausubel et al., 2012, “Production of CGMP-Grade Lentiviral Vectors,” Bioprocess Int. 10(2):32-43, both of which are incorporated by reference herein in their entireties.

In a specific embodiment, a vector for use in the methods described herein is one that encodes an anti-VEGF antigen-binding fragment (e.g., ranibizumab) such that, upon introduction of the vector into a relevant cell (e.g., a retinal cell in vivo or in vitro), a glycosylated and or tyrosine sulfated variant of the anti-VEGF antigen-binding fragment is expressed by the cell. In a specific embodiment, the expressed anti-VEGF antigen-binding fragment comprises a glycosylation and/or tyrosine sulfation pattern as described in Section 5.1, above.

### 5.2.3 Promoters and Modifiers of Gene Expression

In certain embodiments, the vectors provided herein comprise components that modulate gene delivery or gene expression (e.g., “expression control elements”). In certain embodiments, the vectors provided herein comprise components that modulate gene expression. In certain embodiments, the vectors provided herein comprise components that influence binding or targeting to cells. In certain embodiments, the vectors provided herein comprise components that influence the localization of the polynucleotide (e.g., the transgene) within the cell after uptake. In certain embodiments, the vectors provided herein comprise components that can be used as detectable or selectable markers, e.g., to detect or select for cells that have taken up the polynucleotide.

In certain embodiments, the viral vectors provided herein comprise one or more promoters. In certain embodiments, the promoter is a constitutive promoter. In certain embodiments, the promoter is an inducible promoter. Inducible promoters may be preferred so that transgene expression may be turned on and off as desired for therapeutic efficacy. Such promoters include, for example, hypoxia-induced promoters and drug inducible promoters, such as promoters induced by rapamycin and related agents. Hypoxia-inducible promoters include promoters with HIF binding sites, see, for example, Schödel, et al., 2011, Blood 117(23):e207-e217 and Kenneth and Rocha, 2008, Biochem J. 414:19-29, each of which is incorporated by reference for teachings of hypoxia-inducible promoters. In addition, hypoxia-inducible promoters that may be used in the constructs include the erythropoietin promoter and N-WASP promoter (see, Tsuchiya, 1993, J. Biochem. 113:395 for disclosure of the erythropoietin promoter and Salvi, 2017, Biochemistry and Biophysics Reports 9:13-21 for disclosure of N-WASP promoter, both of which are incorporated by reference for the teachings of hypoxia-induced promoters). Alternatively, the constructs may contain drug inducible promoters, for example promoters inducible by administration of rapamycin and related analogs (see, for example, International Patent Application Publication Nos. W094/18317, WO 96/20951, WO 96/41865, WO 99/10508, WO 99/10510, WO 99/36553, and WO 99/41258, and U.S. Pat. No. US 7,067,526 (disclosing rapamycin analogs), which are incorporated by reference herein for their disclosure of drug inducible promoters). In certain embodiments the promoter is a hypoxia-inducible promoter. In certain embodiments, the promoter comprises a hypoxia-inducible factor (HIF) binding site. In certain embodiments, the promoter comprises a HIF-1α binding site. In certain embodiments, the promoter comprises a HIF-2α binding site. In certain embodiments, the HIF binding site comprises an RCGTG motif. For details regarding the location and sequence of HIF binding sites, see, e.g., Schodel, et al., Blood, 2011, 117(23):e207-e217, which is incorporated by reference herein in its entirety. In certain embodiments, the promoter comprises a binding site for a hypoxia induced transcription factor other than a HIF transcription factor. In certain embodiments, the viral vectors provided herein comprise one or more IRES sites that is preferentially translated in hypoxia. For teachings regarding hypoxia-inducible gene expression and the factors involved therein, see, e.g., Kenneth and Rocha, Biochem J., 2008, 414:19-29, which is incorporated by reference herein in its entirety.

In certain embodiments, the promoter is a CB7 promoter (see Dinculescu et al., 2005, Hum Gene Ther 16: 649-663, incorporated by reference herein in its entirety). In some embodiments, the CB7 promoter includes other expression control elements that enhance expression of the transgene driven by the vector. In certain embodiments, the other expression control elements include chicken β-actin intron and/or rabbit β-globin polA signal. In certain embodiments, the promoter comprises a TATA box. In certain embodiments, the promoter comprises one or more elements. In certain embodiments, the one or more promoter elements may be inverted or moved relative to one another. In certain embodiments, the elements of the promoter are positioned to function cooperatively. In certain embodiments, the elements of the promoter are positioned to function independently. In certain embodiments, the viral vectors provided herein comprise one or more promoters selected from the group consisting of the human CMV immediate early gene promoter, the SV40 early promoter, the Rous sarcoma virus (RS) long terminal repeat, and rat insulin promoter. In certain embodiments, the vectors provided herein comprise one or more long terminal repeat (LTR) promoters selected from the group consisting of AAV, MLV, MMTV, SV40, RSV, HIV-1, and HIV-2 LTRs. In certain embodiments, the vectors provided herein comprise one or more tissue specific promoters (e.g., a retinal pigment epithelial cell-specific promoter). In certain embodiments, the viral vectors provided herein comprise a RPE65 promoter. In certain embodiments, the vectors provided herein comprise a VMD2 promoter.

In certain embodiments, the viral vectors provided herein comprise one or more regulatory elements other than a promoter. In certain embodiments, the viral vectors provided herein comprise an enhancer. In certain embodiments, the viral vectors provided herein comprise a repressor. In certain embodiments, the viral vectors provided herein comprise an intron or a chimeric intron. In certain embodiments, the viral vectors provided herein comprise a polyadenylation sequence.

### 5.2.4 Signal Peptides

In certain embodiments, the vectors provided herein comprise components that modulate protein delivery. In certain embodiments, the viral vectors provided herein comprise one or more signal peptides. Signal peptides may also be referred to herein as “leader sequences” or “leader peptides”. In certain embodiments, the signal peptides allow for the transgene product (e.g., the anti-VEGF antigen-binding fragment moiety) to achieve the proper packaging (e.g. glycosylation) in the cell. In certain embodiments, the signal peptides allow for the transgene product (e.g., the anti-VEGF antigen-binding fragment moiety) to achieve the proper localization in the cell. In certain embodiments, the signal peptides allow for the transgene product (e.g., the anti-VEGF antigen-binding fragment moiety) to achieve secretion from the cell. Examples of signal peptides to be used in connection with the vectors and transgenes provided herein may be found in Table 1.

TABLE 1
Signal peptides for use with the vectors provided herein.

| SEQ | | |
|---|---|---|
| ID NO. | Signal Peptide | Sequence |
| 5 | VEGF-A signal peptide | MNFLLSWVHW SLALLLYLHH |
| | | AKWSQA |
| 6 | Fibulin-1 signal peptide | MERAAPSRRV PLPLLLLGGL |
| | | ALLAAGVDA |
| 7 | Vitronectin signal peptide | MAPLRPLLIL ALLAWVALA |
| 8 | Complement Factor H signal peptide | MRLLAKIICLMLWAICVA |
| 9 | Opticin signal peptide | MRLLAFLSLL ALVLQETGT |
| 22 | Albumin signal peptide | MKWVTFISLLFLFSSAYS |
| 23 | Chymotrypsinogen signal peptide | MAFLWLLSCWALLGTTFG |
| 24 | Interleukin-2 signal peptide | MYRMQLLSCIALILALVTNS |
| 25 | Trypsinogen-2 signal peptide | MNLLLILTFVAAAVA |

### 5.2.5 Polycistronic Messages—IRES and F2A Linkers

Internal ribosome entry sites. A single construct can be engineered to encode both the heavy and light chains separated by a cleavable linker or IRES so that separate heavy and light chain polypeptides are expressed by the transduced cells. In certain embodiments, the viral vectors provided herein provide polycistronic (e.g., bicistronic) messages. For example, the viral construct can encode the heavy and light chains separated by an internal ribosome entry site (IRES) elements (for examples of the use of IRES elements to create bicistronic vectors see, e.g., Gurtu et al., 1996, Biochem. Biophys. Res. Comm. 229(1):295-8, which is herein incorporated by reference in its entirety). IRES elements bypass the ribosome scanning model and begin translation at internal sites. The use of IRES in AAV is described, for example, in Furling et al., 2001, Gene Ther 8(11): 854-73, which is herein incorporated by reference in its entirety. In certain embodiments, the bicistronic message is contained within a viral vector with a restraint on the size of the polynucleotide(s) therein. In certain embodiments, the bicistronic message is contained within an AAV virus-based vector (e.g., an AAV8-based vector).

Furin-F2A linkers. In other embodiments, the viral vectors provided herein encode the heavy and light chains separated by a cleavable linker such as the self-cleaving furin/F2A (F/F2A) linkers (Fang et al., 2005, Nature Biotechnology 23: 584-590, and Fang, 2007, Mol Ther 15: 1153-9, each of which is incorporated by reference herein in its entirety).

For example, a furin-F2A linker may be incorporated into an expression cassette to separate the heavy and light chain coding sequences, resulting in a construct with the structure:

Leader-Heavy chain-Furin site-F2A site-Leader-Light chain-PolyA.

The F2A site, with the amino acid sequence LLNFDLLKLAGDVESNPGP (SEQ ID NO: 26) is self-processing, resulting in “cleavage” between the final G and P amino acid residues. Additional linkers that could be used include but are not limited to:

T2A:
(SEQ ID NO: 27)
(GSG) E G R G S L L T C G D V E E N P G P;
P2A:
(SEQ ID NO: 28)
(GSG) A T N F S L L K Q A G D V E E N P G P;
E2A:
(SEQ ID NO: 29)
(GSG) Q C T N Y A L L K L A G D V E S N P G P;
F2A:
(SEQ ID NO: 30)
(GSG) V K Q T L N F D L L K L A G D V E S N P G P.

A peptide bond is skipped when the ribosome encounters the F2A sequence in the open reading frame, resulting in the termination of translation, or continued translation of the downstream sequence (the light chain). This self-processing sequence results in a string of additional amino acids at the end of the C-terminus of the heavy chain. However, such additional amino acids are then cleaved by host cell Furin at the furin sites, located immediately prior to the F2A site and after the heavy chain sequence, and further cleaved by carboxypeptidases. The resultant heavy chain may have one, two, three, or more additional amino acids included at the C-terminus, or it may not have such additional amino acids, depending on the sequence of the Furin linker used and the carboxypeptidase that cleaves the linker in vivo (See, e.g., Fang et al., 17 April 2005, Nature Biotechnol. Advance Online Publication; Fang et al., 2007, Molecular Therapy 15(6):1153-1159; Luke, 2012, Innovations in Biotechnology, Ch. 8, 161-186). Furin linkers that may be used comprise a series of four basic amino acids, for example, RKRR, RRRR, RRKR, or RKKR. Once this linker is cleaved by a carboxypeptidase, additional amino acids may remain, such that an additional zero, one, two, three or four amino acids may remain on the C-terminus of the heavy chain, for example, R, RR, RK, RKR, RRR, RRK, RKK, RKRR, RRRR, RRKR, or RKKR. In certain embodiments, one the linker is cleaved by an carboxypeptidase, no additional amino acids remain. In certain embodiments, 0.5%, 1%, 2%, 3%, 4%, 5%, 10%, 15%, or 20%, or less but more than 0% of the antibody, e.g., antigen-binding fragment, population produced by the constructs for use in the methods described herein has one, two, three, or four amino acids remaining on the C-terminus of the heavy chain after cleavage. In certain embodiments, 0.5-1%, 0.5%-2%, 0.5%-3%, 0.5%-4%, 0.5%-5%, 0.5%-10%, 0.5%-20%, 1%-2%, 1%-3%, 1%-4%, 1%-5%, 1%-10%, 1%-20%, 2%-3%, 2%-4%, 2%-5%, 2%-10%, 2%-20%, 3%-4%, 3%-5%, 3%-10%, 3%-20%, 4%-5%, 4%-10%, 4%-20%, 5%-10%, 5%-20%, or 10%-20% of the antibody, e.g., antigen-binding fragment, population produced by the constructs for use in the methods described herein has one, two, three, or four amino acids remaining on the C-terminus of the heavy chain after cleavage. In certain embodiments, the furin linker has the sequence R-X-K/R-R, such that the additional amino acids on the C-terminus of the heavy chain are R, RX, RXK, RXR, RXKR, or RXRR, where X is any amino acid, for example, alanine (A). In certain embodiments, no additional amino acids may remain on the C-terminus of the heavy chain.

In certain embodiments, an expression cassette described herein is contained within a viral vector with a restraint on the size of the polynucleotide(s) therein. In certain embodiments, the expression cassette is contained within an AAV virus-based vector (e.g., an AAV8-based vector).

### 5.2.6 Untranslated Regions

In certain embodiments, the viral vectors provided herein comprise one or more untranslated regions (UTRs), e.g., 3′ and/or 5′ UTRs. In certain embodiments, the UTRs are optimized for the desired level of protein expression. In certain embodiments, the UTRs are optimized for the mRNA half life of the transgene. In certain embodiments, the UTRs are optimized for the stability of the mRNA of the transgene. In certain embodiments, the UTRs are optimized for the secondary structure of the mRNA of the transgene.

### 5.2.7 Inverted Terminal Repeats

In certain embodiments, the viral vectors provided herein comprise one or more inverted terminal repeat (ITR) sequences. ITR sequences may be used for packaging the recombinant gene expression cassette into the virion of the viral vector. In certain embodiments, the ITR is from an AAV, e.g., AAV8 or AAV2 (see, e.g., Yan et al., 2005, J. Virol., 79(1):364-379; U.S. Pat. Nos. 7,282,199 B2, 7,790,449 B2, 8,318,480 B2, 8,962,332 B2 and International Patent Application No. PCT/EP2014/076466, each of which is incorporated herein by reference in its entirety).

### 5.2.8 Transgenes

The HuPTMFabVEGFi, e.g., HuGlyFabVEGFi encoded by the transgene can include, but is not limited to an antigen-binding fragment of an antibody that binds to VEGF, such as bevacizumab; an anti-VEGF Fab moiety such as ranibizumab; or such bevacizumab or ranibizumab Fab moieties engineered to contain additional glycosylation sites on the Fab domain (e.g., see Courtois et al., 2016, mAbs 8: 99-112 which is incorporated by reference herein in its entirety for it description of derivatives of bevacizumab that are hyperglycosylated on the Fab domain of the full length antibody).

In certain embodiments, the vectors provided herein encode an anti-VEGF antigen-binding fragment transgene. In specific embodiments, the anti-VEGF antigen-binding fragment transgene is controlled by appropriate expression control elements for expression in retinal cells: In certain embodiments, the anti-VEGF antigen-binding fragment transgene comprises bevacizumab Fab portion of the light and heavy chain cDNA sequences (SEQ ID NOs. 10 and 11, respectively). In certain embodiments, the anti-VEGF antigen-binding fragment transgene comprises ranibizumab light and heavy chain cDNA sequences (SEQ ID NOs. 12 and 13, respectively). In certain embodiments, the anti-VEGF antigen-binding fragment transgene encodes a bevacizumab Fab, comprising a light chain and a heavy chain of SEQ ID NOs: 3 and 4, respectively. In certain embodiments, the anti-VEGF antigen-binding fragment transgene encodes an antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 3. In certain embodiments, the anti-VEGF antigen-binding fragment transgene encodes an antigen-binding fragment comprising a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 4. In certain embodiments, the anti-VEGF antigen-binding fragment transgene encodes an antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 3 and a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 4. In certain embodiments, the anti-VEGF antigen-binding fragment transgene encodes a hyperglycosylated ranibizumab, comprising a light chain and a heavy chain of SEQ ID NOs: 1 and 2, respectively. In certain embodiments, the anti-VEGF antigen-binding fragment transgene encodes an antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 1. In certain embodiments, the anti-VEGF antigen-binding fragment transgene encodes an antigen-binding fragment comprising a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 2. In certain embodiments, the anti-VEGF antigen-binding fragment transgene encodes an antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 1 and a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 2.

In certain embodiments, the anti-VEGF antigen-binding fragment transgene encodes a hyperglycosylated bevacizumab Fab, comprising a light chain and a heavy chain of SEQ ID NOs: 3 and 4, with one or more of the following mutations: L118N (heavy chain), E195N (light chain), or Q160N or Q1605 (light chain). In certain embodiments, the anti-VEGF antigen-binding fragment transgene encodes a hyperglycosylated ranibizumab, comprising a light chain and a heavy chain of SEQ ID NOs: 1 and 2, with one or more of the following mutations: L118N (heavy chain), E195N (light chain), or Q160N or Q1605 (light chain). The sequences of the antigen-binding fragment transgene cDNAs may be found, for example, in Table 2. In certain embodiments, the sequence of the antigen-binding fragment transgene cDNAs is obtained by replacing the signal sequence of SEQ ID NOs: 10 and 11 or SEQ ID NOs: 12 and 13 with one or more signal sequences listed in Table 1.

In certain embodiments, the anti-VEGF antigen-binding fragment transgene encodes an antigen-binding fragment and comprises the nucleotide sequences of the six bevacizumab CDRs. In certain embodiments, the anti-VEGF antigen-binding fragment transgene encodes an antigen-binding fragment and comprises the nucleotide sequences of the six ranibizumab CDRs. In certain embodiments, the anti-VEGF antigen-binding fragment transgene encodes an antigen-binding fragment comprising a heavy chain variable region comprising heavy chain CDRs 1-3 of ranibizumab (SEQ ID NOs: 20, 18, and 21). In certain embodiments, the anti-VEGF antigen-binding fragment transgene encodes an antigen-binding fragment comprising a light chain variable region comprising light chain CDRs 1-3 of ranibizumab (SEQ ID NOs: 14-16). In certain embodiments, the anti-VEGF antigen-binding fragment transgene encodes an antigen-binding fragment comprising a heavy chain variable region comprising heavy chain CDRs 1-3 of bevacizumab (SEQ ID NOs: 17-19). In certain embodiments, the anti-VEGF antigen-binding fragment transgene encodes an antigen-binding fragment comprising a light chain variable region comprising light chain CDRs 1-3 of bevacizumab (SEQ ID NOs: 14-16). In certain embodiments, the anti-VEGF antigen-binding fragment transgene encodes an antigen-binding fragment comprising a heavy chain variable region comprising heavy chain CDRs 1-3 of ranibizumab (SEQ ID NOs: 20, 18, and 21) and a light chain variable region comprising light chain CDRs 1-3 of ranibizumab (SEQ ID NOs: 14-16). In certain embodiments, the anti-VEGF antigen-binding fragment transgene encodes an antigen-binding fragment comprising a heavy chain variable region comprising heavy chain CDRs 1-3 of bevacizumab (SEQ ID NOs: 17-19) and a light chain variable region comprising light chain CDRs 1-3 of bevacizumab (SEQ ID NOs: 14-16).

In certain embodiments, the anti-VEGF antigen-binding fragment transgene encodes an antigen-binding fragment comprising a light chain variable region comprising light chain CDRs 1-3 of SEQ ID NOs: 14-16, wherein the second amino acid residue of the light chain CDR3 (i.e., the second Q in QQYSTVPWTF (SEQ ID NO. 16)) does not carry one or more of the following chemical modifications: oxidation, acetylation, deamidation, and pyroglutamation (pyro Glu). In a specific embodiment, the anti-VEGF antigen-binding fragment transgene encodes an antigen-binding fragment comprising a light chain variable region comprising light chain CDRs 1-3 of SEQ ID NOs: 14-16, wherein the eighth and eleventh amino acid residues of the light chain CDR1 (i.e., the two Ns in SASQDISNYLN (SEQ ID NO. 14) each carries one or more of the following chemical modifications: oxidation, acetylation, deamidation, and pyroglutamation (pyro Glu), and the second amino acid residue of the light chain CDR3 (i.e., the second Q in QQYSTVPWTF (SEQ ID NO. 16)) does not carry one or more of the following chemical modifications: oxidation, acetylation, deamidation, and pyroglutamation (pyro Glu). In a specific embodiment, the anti-VEGF antigen-binding fragment transgene encodes an antigen-binding fragment comprising a light chain variable region comprising light chain CDRs 1-3 of SEQ ID NOs: 14-16, wherein the second amino acid residue of the light chain CDR3 (i.e., the second Q in QQYSTVPWTF (SEQ ID NO. 16)) is not acetylated. In a specific embodiment, the anti-VEGF antigen-binding fragment transgene encodes an antigen-binding fragment comprising a light chain variable region comprising light chain CDRs 1-3 of SEQ ID NOs: 14-16, wherein the eighth and eleventh amino acid residues of the light chain CDR1 (i.e., the two Ns in SASQDISNYLN (SEQ ID NO. 14) each carries one or more of the following chemical modifications: oxidation, acetylation, deamidation, and pyroglutamation (pyro Glu), and the second amino acid residue of the light chain CDR3 (i.e., the second Q in QQYSTVPWTF (SEQ ID NO. 16)) is not acetylated. In a preferred embodiment, the chemical modification(s) or lack of chemical modification(s) (as the case may be) described herein is determined by mass spectrometry.

In certain embodiments, the anti-VEGF antigen-binding fragment transgene encodes an antigen-binding fragment comprising a heavy chain variable region comprising heavy chain CDRs 1-3 of SEQ ID NOs: 20, 18, and 21, wherein the last amino acid residue of the heavy chain CDR1 (i.e., the N in GYDFTHYGMN (SEQ ID NO. 20)) does not carry one or more of the following chemical modifications: oxidation, acetylation, deamidation, and pyroglutamation (pyro Glu). In a specific embodiment, the anti-VEGF antigen-binding fragment transgene encodes an antigen-binding fragment comprising a heavy chain variable region comprising heavy chain CDRs 1-3 of SEQ ID NOs: 20, 18, and 21, wherein the ninth amino acid residue of the heavy chain CDR1 (i.e., the M in GYDFTHYGMN (SEQ ID NO. 20)) carries one or more of the following chemical modifications: acetylation, deamidation, and pyroglutamation (pyro Glu), the third amino acid residue of the heavy chain CDR2 (i.e., the N in WINTYTGEPTYAADFKR (SEQ ID NO. 18) carries one or more of the following chemical modifications: acetylation, deamidation, and pyroglutamation (pyro Glu), and the last amino acid residue of the heavy chain CDR1 (i.e., the N in GYDFTHYGMN (SEQ ID NO. 20)) does not carry one or more of the following chemical modifications: oxidation, acetylation, deamidation, and pyroglutamation (pyro Glu). In a specific embodiment, the anti-VEGF antigen-binding fragment transgene encodes an antigen-binding fragment comprising a heavy chain variable region comprising heavy chain CDRs 1-3 of SEQ ID NOs: 20, 18, and 21, wherein the last amino acid residue of the heavy chain CDR1 (i.e., the N in GYDFTHYGMN (SEQ ID NO. 20)) is not acetylated. In a specific embodiment, the anti-VEGF antigen-binding fragment transgene encodes an antigen-binding fragment comprising a heavy chain variable region comprising heavy chain CDRs 1-3 of SEQ ID NOs: 20, 18, and 21, wherein the ninth amino acid residue of the heavy chain CDR1 (i.e., the M in GYDFTHYGMN (SEQ ID NO. 20)) carries one or more of the following chemical modifications: acetylation, deamidation, and pyroglutamation (pyro Glu), the third amino acid residue of the heavy chain CDR2 (i.e., the N in WINTYTGEPTYAADFKR (SEQ ID NO. 18) carries one or more of the following chemical modifications: acetylation, deamidation, and pyroglutamation (pyro Glu), and the last amino acid residue of the heavy chain CDR1 (i.e., the N in GYDFTHYGMN (SEQ ID NO. 20)) is not acetylated. In a preferred embodiment, the chemical modification(s) or lack of chemical modification(s) (as the case may be) described herein is determined by mass spectrometry.

In certain embodiments, the anti-VEGF antigen-binding fragment transgene encodes an antigen-binding fragment comprising a light chain variable region comprising light chain CDRs 1-3 of SEQ ID NOs: 14-16 and a heavy chain variable region comprising heavy chain CDRs 1-3 of SEQ ID NOs: 20, 18, and 21, wherein the second amino acid residue of the light chain CDR3 (i.e., the second Q in QQYSTVPWTF (SEQ ID NO. 16)) does not carry one or more of the following chemical modifications: oxidation, acetylation, deamidation, and pyroglutamation (pyro Glu), and wherein the last amino acid residue of the heavy chain CDR1 (i.e., the N in GYDFTHYGMN (SEQ ID NO. 20)) does not carry one or more of the following chemical modifications: oxidation, acetylation, deamidation, and pyroglutamation (pyro Glu). In a specific embodiment, the anti-VEGF antigen-binding fragment transgene encodes an antigen-binding fragment comprising a light chain variable region comprising light chain CDRs 1-3 of SEQ ID NOs: 14-16 and a heavy chain variable region comprising heavy chain CDRs 1-3 of SEQ ID NOs: 20, 18, and 21, wherein: (1) the ninth amino acid residue of the heavy chain CDR1 (i.e., the M in GYDFTHYGMN (SEQ ID NO. 20)) carries one or more of the following chemical modifications: acetylation, deamidation, and pyroglutamation (pyro Glu), the third amino acid residue of the heavy chain CDR2 (i.e., the N in WINTYTGEPTYAADFKR (SEQ ID NO. 18) carries one or more of the following chemical modifications: acetylation, deamidation, and pyroglutamation (pyro Glu), and the last amino acid residue of the heavy chain CDR1 (i.e., the N in GYDFTHYGMN (SEQ ID NO. 20)) does not carry one or more of the following chemical modifications: oxidation, acetylation, deamidation, and pyroglutamation (pyro Glu); and (2) the eighth and eleventh amino acid residues of the light chain CDR1 (i.e., the two Ns in SASQDISNYLN (SEQ ID NO. 14) each carries one or more of the following chemical modifications: oxidation, acetylation, deamidation, and pyroglutamation (pyro Glu), and the second amino acid residue of the light chain CDR3 (i.e., the second Q in QQYSTVPWTF (SEQ ID NO. 16)) does not carry one or more of the following chemical modifications: oxidation, acetylation, deamidation, and pyroglutamation (pyro Glu). In a specific embodiment, the anti-VEGF antigen-binding fragment transgene encodes an antigen-binding fragment comprising a light chain variable region comprising light chain CDRs 1-3 of SEQ ID NOs: 14-16 and a heavy chain variable region comprising heavy chain CDRs 1-3 of SEQ ID NOs: 20, 18, and 21, wherein the second amino acid residue of the light chain CDR3 (i.e., the second Q in QQYSTVPWTF (SEQ ID NO. 16)) is not acetylated, and wherein the last amino acid residue of the heavy chain CDR1 (i.e., the N in GYDFTHYGMN (SEQ ID NO. 20)) is not acetylated. In a specific embodiment, the antigen-binding fragment comprises a heavy chain CDR1 of SEQ ID NO. 20, wherein: (1) the ninth amino acid residue of the heavy chain CDR1 (i.e., the M in GYDFTHYGMN (SEQ ID NO. 20)) carries one or more of the following chemical modifications: acetylation, deamidation, and pyroglutamation (pyro Glu), the third amino acid residue of the heavy chain CDR2 (i.e., the N in WINTYTGEPTYAADFKR (SEQ ID NO. 18) carries one or more of the following chemical modifications: acetylation, deamidation, and pyroglutamation (pyro Glu), and the last amino acid residue of the heavy chain CDR1 (i.e., the N in GYDFTHYGMN (SEQ ID NO. 20)) is not acetylated; and (2) the eighth and eleventh amino acid residues of the light chain CDR1 (i.e., the two Ns in SASQDISNYLN (SEQ ID NO. 14) each carries one or more of the following chemical modifications: oxidation, acetylation, deamidation, and pyroglutamation (pyro Glu), and the second amino acid residue of the light chain CDR3 (i.e., the second Q in QQYSTVPWTF (SEQ ID NO. 16)) is not acetylated. In a preferred embodiment, the chemical modification(s) or lack of chemical modification(s) (as the case may be) described herein is determined by mass spectrometry.

In certain aspects, also provided herein are anti-VEGF antigen-binding fragments comprising light chain CDRs 1-3 of SEQ ID NOs: 14-16 and heavy chain CDRs 1-3 of SEQ ID NOs: 20, 18, and 21, and transgenes encoding such antigen-VEGF antigen-binding fragments, wherein the second amino acid residue of the light chain CDR3 (i.e., the second Q in QQYSTVPWTF (SEQ ID NO. 16)) does not carry one or more of the following chemical modifications: oxidation, acetylation, deamidation, and pyroglutamation (pyro Glu). In a specific embodiment, the antigen-binding fragment comprises light chain CDRs 1-3 of SEQ ID NOs: 14-16 and heavy chain CDRs 1-3 of SEQ ID NOs: 20, 18, and 21, wherein the eighth and eleventh amino acid residues of the light chain CDR1 (i.e., the two Ns in SASQDISNYLN (SEQ ID NO. 14) each carries one or more of the following chemical modifications: oxidation, acetylation, deamidation, and pyroglutamation (pyro Glu), and the second amino acid residue of the light chain CDR3 (i.e., the second Q in QQYSTVPWTF (SEQ ID NO. 16)) does not carry one or more of the following chemical modifications: oxidation, acetylation, deamidation, and pyroglutamation (pyro Glu). In a specific embodiment, the antigen-binding fragment comprises light chain CDRs 1-3 of SEQ ID NOs: 14-16 and heavy chain CDRs 1-3 of SEQ ID NOs: 20, 18, and 21, wherein the second amino acid residue of the light chain CDR3 (i.e., the second Q in QQYSTVPWTF (SEQ ID NO. 16)) is not acetylated. In a specific embodiment, the antigen-binding fragment comprises light chain CDRs 1-3 of SEQ ID NOs: 14-16 and heavy chain CDRs 1-3 of SEQ ID NOs: 20, 18, and 21, wherein the eighth and eleventh amino acid residues of the light chain CDR1 (i.e., the two Ns in SASQDISNYLN (SEQ ID NO. 14) each carries one or more of the following chemical modifications: oxidation, acetylation, deamidation, and pyroglutamation (pyro Glu), and the second amino acid residue of the light chain CDR3 (i.e., the second Q in QQYSTVPWTF (SEQ ID NO. 16)) is not acetylated. The anti-VEGF antigen-binding fragments and transgenes provided herein can be used in any method according to the invention described herein. In a preferred embodiment, the chemical modification(s) or lack of chemical modification(s) (as the case may be) described herein is determined by mass spectrometry.

In certain aspects, also provided herein are anti-VEGF antigen-binding fragments comprising light chain CDRs 1-3 of SEQ ID NOs: 14-16 and heavy chain CDRs 1-3 of SEQ ID NOs: 20, 18, and 21, and transgenes encoding such antigen-VEGF antigen-binding fragments, wherein the last amino acid residue of the heavy chain CDR1 (i.e., the N in GYDFTHYGMN (SEQ ID NO. 20)) does not carry one or more of the following chemical modifications: oxidation, acetylation, deamidation, and pyroglutamation (pyro Glu). In a specific embodiment, the antigen-binding fragment comprises light chain CDRs 1-3 of SEQ ID NOs: 14-16 and heavy chain CDRs 1-3 of SEQ ID NOs: 20, 18, and 21, wherein the ninth amino acid residue of the heavy chain CDR1 (i.e., the M in GYDFTHYGMN (SEQ ID NO. 20)) carries one or more of the following chemical modifications: acetylation, deamidation, and pyroglutamation (pyro Glu), the third amino acid residue of the heavy chain CDR2 (i.e., the N in WINTYTGEPTYAADFKR (SEQ ID NO. 18) carries one or more of the following chemical modifications: acetylation, deamidation, and pyroglutamation (pyro Glu), and the last amino acid residue of the heavy chain CDR1 (i.e., the N in GYDFTHYGMN (SEQ ID NO. 20)) does not carry one or more of the following chemical modifications: oxidation, acetylation, deamidation, and pyroglutamation (pyro Glu). In a specific embodiment, the antigen-binding fragment comprises light chain CDRs 1-3 of SEQ ID NOs: 14-16 and heavy chain CDRs 1-3 of SEQ ID NOs: 20, 18, and 21, wherein the last amino acid residue of the heavy chain CDR1 (i.e., the N in GYDFTHYGMN (SEQ ID NO. 20)) is not acetylated. In a specific embodiment, the antigen-binding fragment comprises light chain CDRs 1-3 of SEQ ID NOs: 14-16 and heavy chain CDRs 1-3 of SEQ ID NOs: 20, 18, and 21, wherein the ninth amino acid residue of the heavy chain CDR1 (i.e., the M in GYDFTHYGMN (SEQ ID NO. 20)) carries one or more of the following chemical modifications: acetylation, deamidation, and pyroglutamation (pyro Glu), the third amino acid residue of the heavy chain CDR2 (i.e., the N in WINTYTGEPTYAADFKR (SEQ ID NO. 18) carries one or more of the following chemical modifications: acetylation, deamidation, and pyroglutamation (pyro Glu), and the last amino acid residue of the heavy chain CDR1 (i.e., the N in GYDFTHYGMN (SEQ ID NO. 20)) is not acetylated. The anti-VEGF antigen-binding fragments and transgenes provided herein can be used in any method according to the invention described herein. In a preferred embodiment, the chemical modification(s) or lack of chemical modification(s) (as the case may be) described herein is determined by mass spectrometry.

In certain aspects, also provided herein are anti-VEGF antigen-binding fragments comprising light chain CDRs 1-3 of SEQ ID NOs: 14-16 and heavy chain CDRs 1-3 of SEQ ID NOs: 20, 18, and 21, and transgenes encoding such antigen-VEGF antigen-binding fragments, wherein the last amino acid residue of the heavy chain CDR1 (i.e., the N in GYDFTHYGMN (SEQ ID NO. 20)) does not carry one or more of the following chemical modifications: oxidation, acetylation, deamidation, and pyroglutamation (pyro Glu), and the second amino acid residue of the light chain CDR3 (i.e., the second Q in QQYSTVPWTF (SEQ ID NO. 16)) does not carry one or more of the following chemical modifications: oxidation, acetylation, deamidation, and pyroglutamation (pyro Glu). In a specific embodiment, the antigen-binding fragment comprises light chain CDRs 1-3 of SEQ ID NOs: 14-16 and heavy chain CDRs 1-3 of SEQ ID NOs: 20, 18, and 21, wherein: (1) the ninth amino acid residue of the heavy chain CDR1 (i.e., the M in GYDFTHYGMN (SEQ ID NO. 20)) carries one or more of the following chemical modifications: acetylation, deamidation, and pyroglutamation (pyro Glu), the third amino acid residue of the heavy chain CDR2 (i.e., the N in WINTYTGEPTYAADFKR (SEQ ID NO. 18) carries one or more of the following chemical modifications: acetylation, deamidation, and pyroglutamation (pyro Glu), and the last amino acid residue of the heavy chain CDR1 (i.e., the N in GYDFTHYGMN (SEQ ID NO. 20)) does not carry one or more of the following chemical modifications: oxidation, acetylation, deamidation, and pyroglutamation (pyro Glu); and (2) the eighth and eleventh amino acid residues of the light chain CDR1 (i.e., the two Ns in SASQDISNYLN (SEQ ID NO. 14) each carries one or more of the following chemical modifications: oxidation, acetylation, deamidation, and pyroglutamation (pyro Glu), and the second amino acid residue of the light chain CDR3 (i.e., the second Q in QQYSTVPWTF (SEQ ID NO. 16)) does not carry one or more of the following chemical modifications: oxidation, acetylation, deamidation, and pyroglutamation (pyro Glu). In a specific embodiment, the antigen-binding fragment comprises light chain CDRs 1-3 of SEQ ID NOs: 14-16 and heavy chain CDRs 1-3 of SEQ ID NOs: 20, 18, and 21, wherein the last amino acid residue of the heavy chain CDR1 (i.e., the N in GYDFTHYGMN (SEQ ID NO. 20)) is not acetylated, and the second amino acid residue of the light chain CDR3 (i.e., the second Q in QQYSTVPWTF (SEQ ID NO. 16)) is not acetylated. In a specific embodiment, the antigen-binding fragment comprises light chain CDRs 1-3 of SEQ ID NOs: 14-16 and heavy chain CDRs 1-3 of SEQ ID NOs: 20, 18, and 21, wherein: (1) the ninth amino acid residue of the heavy chain CDR1 (i.e., the M in GYDFTHYGMN (SEQ ID NO. 20)) carries one or more of the following chemical modifications: acetylation, deamidation, and pyroglutamation (pyro Glu), the third amino acid residue of the heavy chain CDR2 (i.e., the N in WINTYTGEPTYAADFKR (SEQ ID NO. 18) carries one or more of the following chemical modifications: acetylation, deamidation, and pyroglutamation (pyro Glu), and the last amino acid residue of the heavy chain CDR1 (i.e., the N in GYDFTHYGMN (SEQ ID NO. 20)) is not acetylated; and (2) the eighth and eleventh amino acid residues of the light chain CDR1 (i.e., the two Ns in SASQDISNYLN (SEQ ID NO. 14) each carries one or more of the following chemical modifications: oxidation, acetylation, deamidation, and pyroglutamation (pyro Glu), and the second amino acid residue of the light chain CDR3 (i.e., the second Q in QQYSTVPWTF (SEQ ID NO. 16)) is not acetylated. The anti-VEGF antigen-binding fragments and transgenes provided herein can be used in any method according to the invention described herein. In a preferred embodiment, the chemical modification(s) or lack of chemical modification(s) (as the case may be) described herein is determined by mass spectrometry.

TABLE 2
Exemplary transgene sequences

| | VEGF | |
|---|---|---|
| SEQ | antigen- | |
| ID | binding | |
| NO. | fragment | Sequence |
| 1 | ranibizumab | DIQLTQSPSSLSASVGDRVTITCSASQDISNYLNWYQQKPGKAPKVLIYFTSS |
| | Fab | LHSGVPSRFSGSGSGTDFTLTISSLQPEDFATYYCQQYSTVPWTFGQGTKVEI |
| | Amino | KRTVAAPSVFIFPPSDEQLKSGTASVVCLLNNFYPREAKVQWKVDNALQSGNS |
| | Acid | QESVTEQDSKDSTYSLSSTLTLSKADYEKHKVYACEVTHQGLSSPVTKSFNRG |
| | Sequence | EC |
| | (Light | |
| | chain) | |
| 2 | ranibizumab | EVQLVESGGGLVQPGGSLRLSCAASGYDFTHYGMNWVRQAPGKGLEWVGWINT |
| | Fab | YTGEPTYAADFKRRFTFSLDTSKSTAYLQMNSLRAEDTAVYYCAKYPYYYGTS |
| | Amino | HWYFDVWGQGTLVTVSSASTKGPSVFPLAPSSKSTSGGTAALGCLVKDYFPEP |
| | Acid | VTVSWNSGALTSGVHTFPAVLQSSGLYSLSSVVTVPSSSLGTQTYICNVNHKP |
| | Sequence | SNTKVDKKVEPKSCDKTHL |
| | (Heavy | |
| | chain) | |
| 3 | bevacizumab | DIQMIQSPSSLSASVGDRVTITCSASQDISNYLNWYQQKPGKAPKVLIYFTSS |
| | Fab | LHSGVPSRFSGSGSGTDFTLTISSLQPEDFATYYCQQYSTVPWTFGQGTKVEI |
| | Amino | KRTVAAPSVFIFPPSDEQLKSGTASVVCLLNNFYPREAKVQWKVDNALQSGNS |
| | Acid | QESVTEQDSKDSTYSLSSTLTLSKADYEKHKVYACEVTHQGLSSPVTKSFNRG |
| | Sequence | EC |
| | (Light | |
| | chain) | |
| 4 | bevacizumab | EVQLVESGGGLVQPGGSLRLSCAASGYTFTNYGMNWVRQAPGKGLEWVGWINT |
| | Fab | YTGEPTYAADFKRRFIFSLDISKSTAYLQMNSLRAEDTAVYYCAKYPHYYGSS |
| | Amino | HWYFDVWGQGTLVTVSSASTKGPSVFPLAPSSKSTSGGTAALGCLVKDYFPEP |
| | Acid | VTVSWNSGALTSGVHTFPAVLQSSGLYSLSSVVTVPSSSLGTQTYICNVNHKP |
| | Sequence | SNTKVDKKVEPKSCDKTHL |
| | (Heavy | |
| | chain) | |
| 10 | bevacizumab | gctagcgcca ccatgggctg gtcctgcatc atcctgttcc |
| | cDNA | tggtggccac cgccaccggc gtgcactccg acatccagat |
| | (Light | gacccagtcc ccctcctccc tgtccgcctc cgtgggcgac |
| | chain) | cgggtgacca tcacctgctc cgcctcccag gacatctcca |
| | | actacctgaa ctggtaccag cagaagcccg gcaaggcccc |
| | | caaggtgctg atctacttca cctcctccct gcactccggc |
| | | gtgccctccc ggttctccgg ctccggctcc ggcaccgact |
| | | tcaccctgac catctcctcc ctgcagcccg aggacttcgc |
| | | cacctactac tgccagcagt actccaccgt gccctggacc |
| | | ttcggccagg gcaccaaggt ggagatcaag cggaccgtgg |
| | | ccgccccctc cgtgttcatc ttccccccct ccgacgagca |
| | | gctgaagtcc ggcaccgcct ccgtggtgtg cctgctgaac |
| | | aacttctacc cccgggaggc caaggtgcag tggaaggtgg |
| | | acaacgccct gcagtccggc aactcccagg agtccgtgac |
| | | cgagcaggac tccaaggact ccacctactc cctgtcctcc |
| | | accctgaccc tgtccaaggc cgactacgag aagcacaagg |
| | | tgtacgcctg cgaggtgacc caccagggcc tgtcctcccc |
| | | cgtgaccaag tccttcaacc ggggcgagtg ctgagcggcc gcctcgag |
| 11 | bevacizumab | gctagcgcca ccatgggctg gtcctgcatc atcctgttcc |
| | cDNA | tggtggccac cgccaccggc gtgcactccg aggtgcagct |
| | (Heavy | ggtggagtcc ggcggcggcc tggtgcagcc cggcggctcc |
| | chain) | ctgcggctgt cctgcgccgc ctccggctac accttcacca |
| | | actacggcat gaactgggtg cggcaggccc ccggcaaggg |
| | | cctggagtgg gtgggctgga tcaacaccta caccggcgag |
| | | cccacctacg ccgccgactt caagcggcgg ttcaccttct |
| | | ccctggacac ctccaagtcc accgcctacc tgcagatgaa |
| | | ctccctgcgg gccgaggaca ccgccgtgta ctactgcgcc |
| | | aagtaccccc actactacgg ctcctcccac tggtacttcg |
| | | acgtgtgggg ccagggcacc ctggtgaccg tgtcctccgc |
| | | ctccaccaag ggcccctccg tgttccccct ggccccctcc |
| | | tccaagtcca cctccggcgg caccgccgcc ctgggctgcc |
| | | tggtgaagga ctacttcccc gagcccgtga ccgtgtcctg |
| | | gaactccggc gccctgacct ccggcgtgca caccttcccc |
| | | gccgtgctgc agtcctccgg cctgtactcc ctgtcctccg |
| | | tggtgaccgt gccctcctcc tccctgggca cccagaccta |
| | | catctgcaac gtgaaccaca agccctccaa caccaaggtg |
| | | gacaagaagg tggagcccaa gtcctgcgac aagacccaca |
| | | cctgcccccc ctgccccgcc cccgagctgc tgggcggccc |
| | | ctccgtgttc ctgttccccc ccaagcccaa ggacaccctg |
| | | atgatctccc ggacccccga ggtgacctgc gtggtggtgg |
| | | acgtgtccca cgaggacccc gaggtgaagt tcaactggta |
| | | cgtggacggc gtggaggtgc acaacgccaa gaccaagccc |
| | | cgggaggagc agtacaactc cacctaccgg gtggtgtccg |
| | | tgctgaccgt gctgcaccag gactggctga acggcaagga |
| | | gtacaagtgc aaggtgtcca acaaggccct gcccgccccc |
| | | atcgagaaga ccatctccaa ggccaagggc cagccccggg |
| | | agccccaggt gtacaccctg cccccctccc gggaggagat |
| | | gaccaagaac caggtgtccc tgacctgcct ggtgaagggc |
| | | ttctacccct ccgacatcgc cgtggagtgg gagtccaacg |
| | | gccagcccga gaacaactac aagaccaccc cccccgtgct |
| | | ggactccgac ggctccttct tcctgtactc caagctgaccgtggacaagt |
| | | cccggtggca gcagggcaac gtgttctcct gctccgtgat |
| | | gcacgaggcc ctgcacaacc actacaccca gaagtccctg |
| | | tccctgtccc ccggcaagtg agcggccgcc |
| 12 | ranibizumab | gagctccatg gagtttttca aaaagacggc acttgccgca |
| | cDNA | ctggttatgg gttttagtgg tgcagcattg gccgatatcc |
| | (Light | agctgaccca gagcccgagc agcctgagcg caagcgttgg |
| | chain | tgatcgtgtt accattacct gtagcgcaag ccaggatatt |
| | comprising | agcaattatc tgaattggta tcagcagaaa ccgggtaaag |
| | a | caccgaaagt tctgatttat tttaccagca gcctgcatag |
| | signal | cggtgttccg agccgtttta gcggtagcgg tagtggcacc |
| | sequence) | gattttaccc tgaccattag cagcctgcag ccggaagatt |
| | | ttgcaaccta ttattgtcag cagtatagca ccgttccgtg |
| | | gacctttggt cagggcacca aagttgaaat taaacgtacc |
| | | gttgcagcac cgagcgtttt tatttttccg cctagtgatg |
| | | aacagctgaa aagcggcacc gcaagcgttg tttgtctgct |
| | | gaataatttt tatccgcgtg aagcaaaagt gcagtggaaa |
| | | gttgataatg cactgcagag cggtaatagc caagaaagcg |
| | | ttaccgaaca ggatagcaaa gatagcacct atagcctgag |
| | | cagcaccctg accctgagca aagcagatta tgaaaaacac |
| | | aaagtgtatg cctgcgaagt tacccatcag ggtctgagca |
| | | gtccggttac caaaagtttt aatcgtggcg aatgctaata |
| | | gaagcttggt acc |
| 13 | ranibizumab | gagctcatat gaaatacctg ctgccgaccg ctgctgctgg |
| | cDNA | tctgctgctc ctcgctgccc agccggcgat ggccgaagtt |
| | (Heavy | cagctggttg aaagcggtgg tggtctggtt cagcctggtg |
| | chain | gtagcctgcg tctgagctgt gcagcaagcg gttatgattt |
| | comprising | tacccattat ggtatgaatt gggttcgtca ggcaccgggt |
| | a | aaaggtctgg aatgggttgg ttggattaat acctataccg |
| | signal | gtgaaccgac ctatgcagca gattttaaac gtcgttttac |
| | sequence) | ctttagcctg gataccagca aaagcaccgc atatctgcag |
| | | atgaatagcc tgcgtgcaga agataccgca gtttattatt |
| | | gtgccaaata tccgtattac tatggcacca gccactggta |
| | | tttcgatgtt tggggtcagg gcaccctggt taccgttagc |
| | | agcgcaagca ccaaaggtcc gagcgttttt ccgctggcac |
| | | cgagcagcaa aagtaccagc ggtggcacag cagcactggg |
| | | ttgtctggtt aaagattatt ttccggaacc ggttaccgtg |
| | | agctggaata gcggtgcact gaccagcggt gttcatacct |
| | | ttccggcagt tctgcagagc agcggtctgt atagcctgag |
| | | cagcgttgtt accgttccga gcagcagcct gggcacccag |
| | | acctatattt gtaatgttaa tcataaaccg agcaatacca |
| | | aagtggataa aaaagttgag ccgaaaagct gcgataaaac |
| | | ccatctgtaa tagggtacc |
| | bevacizumab | SASQDISNYLN |
| | Light | FTSSLHS |
| | Chain | QQYSTVPWT |
| | CDRs | |
| | (14, 15, | |
| | and 16) | |
| | bevacizumab | GYTFTNYGMN |
| | Heavy | WINTYTGEPTYAADFKR |
| | Chain | YPHYYGSSHWYFDV |
| | CDRs | |
| | (17, 18, | |
| | and 19) | |
| | ranibizumab | SASQDISNYLN |
| | Light | FTSSLHS |
| | Chain | QQYSTVPWT |
| | CDRs | |
| | (14, 15, | |
| | and 16) | |
| | ranibizumab | GYDFTHYGMN |
| | Heavy | WINTYTGEPTYAADFKR |
| | Chain | YPYYYGTSHWYFDV |
| | CDRs | |
| | (20, 18, | |
| | and 21) | |

### 5.2.9 Constructs

In certain embodiments, the viral vectors provided herein comprise the following elements in the following order: a) a constitutive or a hypoxia-inducible promoter sequence, and b) a sequence encoding the transgene (e.g., an anti-VEGF antigen-binding fragment moiety). In certain embodiments, the sequence encoding the transgene comprises multiple ORFs separated by IRES elements. In certain embodiments, the ORFs encode the heavy and light chain domains of the anti-VEGF antigen-binding fragment. In certain embodiments, the sequence encoding the transgene comprises multiple subunits in one ORF separated by F/F2A sequences. In certain embodiments, the sequence comprising the transgene encodes the heavy and light chain domains of the anti-VEGF antigen-binding fragment separated by an F/F2A sequence. In certain embodiments, the viral vectors provided herein comprise the following elements in the following order: a) a constitutive or a hypoxia-inducible promoter sequence, and b) a sequence encoding the transgene (e.g., an anti-VEGF antigen-binding fragment moiety), wherein the transgene comprises the signal peptide of VEGF (SEQ ID NO: 5), and wherein the transgene encodes a light chain and a heavy chain sequence separated by an IRES element. In certain embodiments, the viral vectors provided herein comprise the following elements in the following order: a) a constitutive or a hypoxia-inducible promoter sequence, and b) a sequence encoding the transgene (e.g., an anti-VEGF antigen-binding fragment moiety), wherein the transgene comprises the signal peptide of VEGF (SEQ ID NO: 5), and wherein the transgene encodes a light chain and a heavy chain sequence separated by a cleavable F/F2A sequence.

In certain embodiments, the viral vectors provided herein comprise the following elements in the following order: a) a first ITR sequence, b) a first linker sequence, c) a constitutive or a hypoxia-inducible promoter sequence, d) a second linker sequence, e) an intron sequence, f) a third linker sequence, g) a first UTR sequence, h) a sequence encoding the transgene (e.g., an anti-VEGF antigen-binding fragment moiety), i) a second UTR sequence, j) a fourth linker sequence, k) a poly A sequence, 1) a fifth linker sequence, and m) a second ITR sequence.

In certain embodiments, the viral vectors provided herein comprise the following elements in the following order: a) a first ITR sequence, b) a first linker sequence, c) a constitutive or a hypoxia-inducible promoter sequence, d) a second linker sequence, e) an intron sequence, f) a third linker sequence, g) a first UTR sequence, h) a sequence encoding the transgene (e.g., an anti-VEGF antigen-binding fragment moiety), i) a second UTR sequence, j) a fourth linker sequence, k) a poly A sequence, 1) a fifth linker sequence, and m) a second ITR sequence, wherein the transgene comprises the signal peptide of VEGF (SEQ ID NO: 5), and wherein the transgene encodes a light chain and a heavy chain sequence separated by a cleavable F/F2A sequence.

In a specific embodiment, the construct described herein is Construct I, wherein the Construct I comprises the following components: (1) AAV8 inverted terminal repeats that flank the expression cassette; (2) control elements, which include a) the CB7 promoter, comprising the CMV enhancer/chicken β-actin promoter, b) a chicken β-actin intron and c) a rabbit β-globin poly A signal; and (3) nucleic acid sequences coding for the heavy and light chains of anti-VEGF antigen-binding fragment, separated by a self-cleaving furin (F)/F2A linker, ensuring expression of equal amounts of the heavy and the light chain polypeptides.

In another specific embodiment, the construct described herein is Construct II, wherein the Construct II comprises the following components: (1) AAV2 inverted terminal repeats that flank the expression cassette; (2) control elements, which include a) the CB7 promoter, comprising the CMV enhancer/chicken β-actin promoter, b) a chicken β-actin intron and c) a rabbit β-globin poly A signal; and (3) nucleic acid sequences coding for the heavy and light chains of anti-VEGF antigen-binding fragment, separated by a self-cleaving furin (F)/F2A linker, ensuring expression of equal amounts of the heavy and the light chain polypeptides.

### 5.2.10 Manufacture and Testing of Vectors

The viral vectors provided herein may be manufactured using host cells. The viral vectors provided herein may be manufactured using mammalian host cells, for example, A549 , WEHI, 10T1/2, BHK, MDCK, COS1, COS7, BSC 1, BSC 40, BMT 10, VERO, W138, HeLa, 293, Saos, C2C12, L, HT1080, HepG2, primary fibroblast, hepatocyte, and myoblast cells. The viral vectors provided herein may be manufactured using host cells from human, monkey, mouse, rat, rabbit, or hamster.

The host cells are stably transformed with the sequences encoding the transgene and associated elements (i.e., the vector genome), and the means of producing viruses in the host cells, for example, the replication and capsid genes (e.g., the rep and cap genes of AAV). For a method of producing recombinant AAV vectors with AAV8 capsids, see Section IV of the Detailed Description of U.S. Pat. No. 7,282,199 B2, which is incorporated herein by reference in its entirety. Genome copy titers of said vectors may be determined, for example, by TAQMAN® analysis. Virions may be recovered, for example, by CsCl2 sedimentation.

In vitro assays, e.g., cell culture assays, can be used to measure transgene expression from a vector described herein, thus indicating, e.g., potency of the vector. For example, the PER.C6® Cell Line (Lonza), a cell line derived from human embryonic retinal cells, or retinal pigment epithelial cells, e.g., the retinal pigment epithelial cell line hTERT RPE-1 (available from ATCC®), can be used to assess transgene expression. Once expressed, characteristics of the expressed product (i.e., HuGlyFabVEGFi) can be determined, including determination of the glycosylation and tyrosine sulfation patterns associated with the HuGlyFabVEGFi. Glycosylation patterns and methods of determining the same are discussed in Section 5.1.1, while tyrosine sulfation patterns and methods of determining the same are discussed in Section 5.1.2. In addition, benefits resulting from glycosylation/sulfation of the cell-expressed HuGlyFabVEGFi can be determined using assays known in the art, e.g., the methods described in Sections 5.1.1 and 5.1.2.

### 5.2.11 Compositions

Compositions are described comprising a vector encoding a transgene described herein and a suitable carrier. A suitable carrier (e.g., for suprachoroidal, subretinal, juxtascleral, intravitreal, subconjunctival, and/or intraretinal administration) would be readily selected by one of skill in the art.

In certain embodiments, gene therapy constructs are supplied as a frozen sterile, single use solution of the AAV vector active ingredient in a formulation buffer. In a specific embodiment, the pharmaceutical compositions suitable for subretinal administration comprise a suspension of the recombinant (e.g., rHuGlyFabVEGFi) vector in a formulation buffer comprising a physiologically compatible aqueous buffer, a surfactant and optional excipients. In a specific embodiment, the gene therapy construct is formulated in Dulbecco's phosphate buffered saline and 0.001% Pluronic F68, pH=7.4.

### 5.3 GENE THERAPY

Methods are described for the administration of a therapeutically effective amount of a transgene construct to human subjects having an ocular disease, in particular an ocular disease caused by increased neovascularization. More particularly, methods for administration of a therapeutically effective amount of a transgene construct to patients having diabetic retinopathy (DR), in particular, for suprachoroidal, subretinal, juxtascleral, intravitreal, subconjunctival, and/or intraretinal administration (e.g., by suprachoroidal injection, subretinal injection via the transvitreal approach (a surgical procedure), subretinal administration via the suprachoroidal space, or a posterior juxtascleral depot procedure), are described.

Methods are described for suprachoroidal, subretinal, juxtascleral, intravitreal, subconjunctival, and/or intraretinal administration of a therapeutically effective amount of a transgene construct to patients diagnosed with diabetic retinopathy (e.g., by suprachoroidal injection, subretinal injection via the transvitreal approach (a surgical procedure), or subretinal administration via the suprachoroidal space).

Also provided herein are methods for suprachoroidal, subretinal, juxtascleral, intravitreal, subconjunctival, and/or intraretinal of a therapeutically effective amount of a transgene construct (e.g., by suprachoroidal injection, subretinal injection via the transvitreal approach (a surgical procedure), subretinal administration via the suprachoroidal space, or a posterior juxtascleral depot procedure) and methods of administration of a therapeutically effective amount of a transgene construct to the retinal pigment epithelium.

### 5.3.1 Target Patient Populations

The subjects treated in accordance with the methods described herein can be any mammals such as rodents, domestic animals such as dogs or cats, or primates, e.g. non-human primates. In a preferred embodiment, the subject is a human. In certain embodiments, the methods provided herein are for the administration to patients diagnosed with an ocular disease, in particular an ocular disease caused by increased neovascularization. In certain embodiments, the methods provided herein are for the administration to patients diagnosed with diabetic retinopathy (DR).

In certain embodiments, the methods provided herein are for the administration to patients diagnosed with severe diabetic retinopathy. In certain embodiments, the methods provided herein are for the administration to patients diagnosed with attenuated diabetic retinopathy.

In certain embodiments, the methods provided herein are for the administration to patients diagnosed with moderately-severe NPDR. In certain embodiments, the methods provided herein are for the administration to patients diagnosed with severe NPDR. In certain embodiments, the methods provided herein are for the administration to patients diagnosed with mild PDR. In certain embodiments, the methods provided herein are for the administration to patients diagnosed with moderate PDR.

In certain embodiments, the methods provided herein are for the administration to patients whose ETDRS-DRSS Levels are 47, 53, 61 or 65. In certain embodiments, the methods provided herein are for the administration to patients whose ETDRS-DRSS Levels are Level 47. In certain embodiments, the methods provided herein are for the administration to patients whose ETDRS-DRSS Levels are Level 53. In certain embodiments, the methods provided herein are for the administration to patients whose ETDRS-DRSS Levels are Level 61. In certain embodiments, the methods provided herein are for the administration to patients whose ETDRS-DRSS Levels are Level 65.

In certain embodiments, the subject treated in accordance with the methods described herein is female. In certain embodiments, the subject treated in accordance with the methods described herein is male. In certain embodiments, the subject treated in accordance with the methods described herein can be of any age. In certain embodiments, the subject treated in accordance with the methods described herein is 18 years old or older. In certain embodiments, the subject treated in accordance with the methods described herein is between 18-89 years of age. In certain embodiments, the subject treated in accordance with the methods described herein has DR secondary to diabetes mellitus Type 1. In certain embodiments, the subject treated in accordance with the methods described herein has DR secondary to diabetes mellitus Type 2. In certain embodiments, the subject treated in accordance with the methods described herein is 18 years old or older with DR secondary to diabetes mellitus Type 1 or Type 2. In certain embodiments, the subject treated in accordance with the methods described herein is between 18-89 years of age with DR secondary to diabetes mellitus Type 1 or Type 2.

In a specific embodiment, the subject treated in accordance with the methods described herein is a woman without childbearing potential.

In specific embodiments, the subject treated in accordance with the methods described herein is phakic. In other specific embodiments, the subject treated in accordance with the methods described herein is pseudophakic.

In certain embodiments, the subject treated in accordance with the methods described herein has a hemoglobin A1c≤10% (as confirmed by laboratory assessments).

In certain embodiments, the subject treated in accordance with the methods described herein has best-corrected visual acuity (BCVA) in the eye to be treated of >69 ETDRS letters (approximate Snellen equivalent 20/40 or better).

In certain embodiments, provided herein is a method for treating a subject with diabetic retinopathy (DR), wherein the subject has at least one eye with DR, the method comprising the steps of:
(1) determining the subject's ETDRS-DR Severity Scale (DRSS) Level, and
(2) if the subject's ETDRS-DRSS is Level 47, 53, 61 or 65 then administering to the subretinal space or the suprachoroidal space in the eye of the human subject an expression vector encoding an anti-human vascular endothelial growth factor (hVEGF) antibody.

In some embodiments, the method further comprises obtaining or having obtained a biological sample from the subject, and determining that the subject has a serum level of hemoglobin A1c of less than or equal to 10%.

In some embodiments, the method prevents progression to proliferative stages of retinopathy in the subject.

In certain embodiments, provided herein is a method for treating a subject with diabetic retinopathy, wherein the subject has at least one eye with moderately-severe non-proliferative diabetic retinopathy (NPDR), the method comprising the steps of:
(1) determining the subject's ETDRS-DR Severity Scale (DRSS) Level, and
(2) if the subject's ETDRS-DRSS is Level 47, then administering to the subretinal space or the suprachoroidal space in the eye of the human subject an expression vector encoding an anti-human vascular endothelial growth factor (hVEGF) antibody.

In certain embodiments, provided herein is a method for treating a subject with diabetic retinopathy, wherein the subject has at least one eye with severe NPDR, the method comprising the steps of:
(1) determining the subject's ETDRS-DR Severity Scale (DRSS) Level, and
(2) if the subject's ETDRS-DRSS is Level 53, then administering to the subretinal space or the suprachoroidal space in the eye of the human subject an expression vector encoding an anti-human vascular endothelial growth factor (hVEGF) antibody.

In certain embodiments, provided herein is a method for treating a subject with diabetic retinopathy, wherein the subject has at least one eye with mild proliferative diabetic retinopathy (PDR), the method comprising the steps of:
(1) determining the subject's ETDRS-DR Severity Scale (DRSS) Level, and
(2) if the subject's ETDRS-DRSS is Level 61, then administering to the subretinal space or the suprachoroidal space in the eye of the human subject an expression vector encoding an anti-human vascular endothelial growth factor (hVEGF) antibody.

In certain embodiments, provided herein is a method for treating a subject with diabetic retinopathy, wherein the subject has at least one eye with moderate PDR, the method comprising the steps of:
(1) determining the subject's ETDRS-DR Severity Scale (DRSS) Level, and
(2) if the subject's ETDRS-DRSS is Level 65, then administering to the subretinal space or the suprachoroidal space in the eye of the human subject an expression vector encoding an anti-human vascular endothelial growth factor (hVEGF) antibody.

ETDRS-DR severity scale (DRSS) Levels are determined using standard 4-widefield digital stereoscopic fundus photographs or equivalent; they may also be measured by monoscopic or stereo photography in accordance with Li et al., 2010, Retina Invest Ophthalmol Vis Sci. 2010;51:3184-3192, or an analogous method.

### 5.3.2 Dosage and Mode of Administration

Therapeutically effective doses of the recombinant vector should be administered subretinally and/or intraretinally (e.g., by subretinal injection via the transvitreal approach (a surgical procedure), or via the suprachoroidal space) in a volume ranging from 0.1 mL to 0.5 mL, preferably in 0.1 to 0.30 mL (100—300 μ1), and most preferably, in a volume of 0.25 mL (250 μl). Therapeutically effective doses of the recombinant vector should be administered suprachoroidally (e.g., by suprachoroidal injection) in a volume of 100 μl or less, for example, in a volume of 50-100 μl. Therapeutically effective doses of the recombinant vector should be administered to the ourter surface of the sclera in a volume of 500 μl or less, for example, in a volume of 500 μl or less, for example, in a volume of 10-20 μl, 20-50 μl, 50-100 μl, 100-200 μl, 200-300 μl, 300-400 μl, or 400-500 μl. Therapeutically effective doses of the recombinant vector may also be administered to the outer surface of the sclera in two or more injections of a volume of 500 μl or less, for example, a volume of 10-20 μl, 20-50 μl, 50-100 μl, 100-200 μl, 200-300 μl, 300-400 ˜l, or 400-500 μl. The two or more injections may be administered during the same visit.

In certain embodiments, the recombinant vector is administered suprachoroidally (e.g., by suprachoroidal injection). In a specific embodiment, suprachorodial administration (e.g., an injection into the suprachoroidal space) is performed using a suprachoroidal drug delivery device. Suprachoroidal drug delivery devices are often used in suprachoroidal administration procedures, which involve administration of a drug to the suprachoroidal space of the eye (see, e.g., Hariprasad, 2016, Retinal Physician 13: 20-23; Goldstein, 2014, Retina Today 9(5): 82-87; Baldassarre et al., 2017; each of which is incorporated by reference herein in its entirety). The suprachoroidal drug delivery devices that can be used to deposit the expression vector in the subretinal space according to the invention described herein include, but are not limited to, suprachoroidal drug delivery devices manufactured by Clearside® Biomedical, Inc. (see, for example, Hariprasad, 2016, Retinal Physician 13: 20-23) and MedOne suprachoroidal catheters.

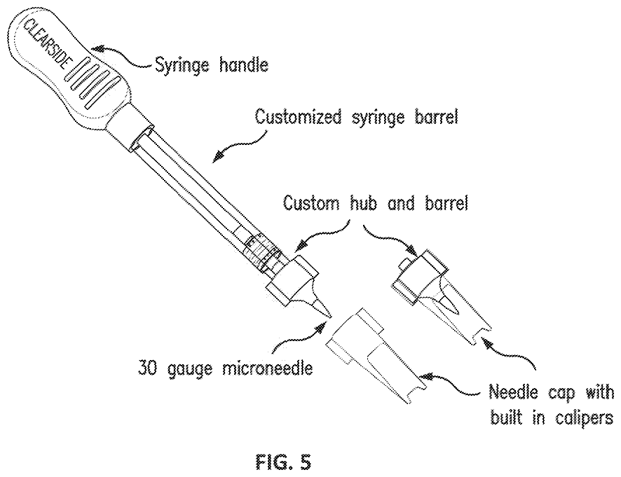

In a specific embodiment, the suprachoroidal drug delivery device is a syringe with a 1 millimeter 30 gauge needle (see FIG. 5). During an injection using this device, the needle pierces to the base of the sclera and fluid containing drug enters the suprachoroidal space, leading to expansion of the suprachoroidal space. As a result, there is tactile and visual feedback during the injection. Following the injection, the fluid flows posteriorly and absorbs dominantly in the choroid and retina. This results in the production of transgene protein from all retinal cell layers and choroidal cells. Using this type of device and procedure allows for a quick and easy in-office procedure with low risk of complications. A max volume of 100 μl can be injected into the suprachoroidal space.

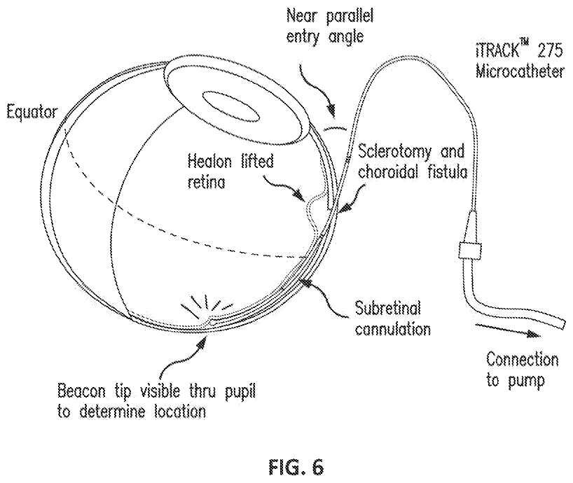

In certain embodiments, the recombinant vector is administered subretinally via the suprachoroidal space by use of a subretinal drug delivery device. In certain embodiments, the subretinal drug delivery device is a catheter which is inserted and tunneled through the suprachoroidal space around to the back of the eye during a surgical procedure to deliver drug to the subretinal space(see FIG. 6). This procedure allows the vitreous to remain intact and thus, there are fewer complication risks (less risk of gene therapy egress, and complications such as retinal detachments and macular holes), and without a vitrectomy, the resulting bleb may spread more diffusely allowing more of the surface area of the retina to be transduced with a smaller volume. The risk of induced cataract following this procedure is minimized, which is desirable for younger patients. Moreover, this procedure can deliver bleb under the fovea more safely than the standard transvitreal approach, which is desirable for patients with inherited retinal diseases effecting central vision where the target cells for transduction are in the macula. This procedure is also favorable for patients that have neutralizing antibodies (Nabs) to AAVs present in the systemic circulation which may impact other routes of delivery (such as suprachoroidal and intravitreal). Additionally, this method has shown to create blebs with less egress out the retinotomy site than the standard transvitreal approach. The subretinal drug delivery device originally manufactured by Janssen Pharmaceuticals, Inc. now by Orbit Biomedical Inc. (see, for example, Subretinal Delivery of Cells via the Suprachoroidal Space: Janssen Trial. In: Schwartz et al. (eds) Cellular Therapies for Retinal Disease, Springer, Cham; International Patent Application Publication No. WO 2016/040635 A1) can be used for such purpose.

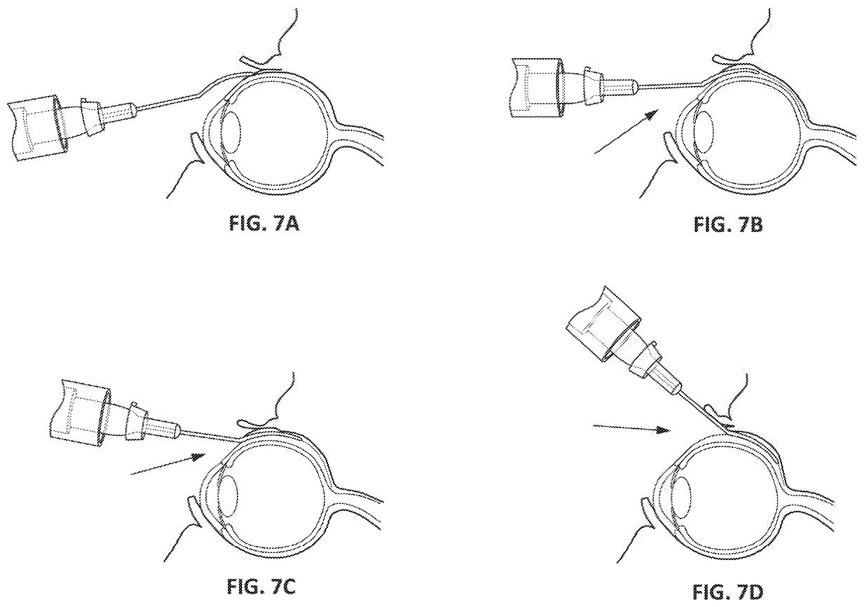

In certain embodiments, the recombinant vector is administered to the outer surface of the sclera (for example, by the use of a juxtascleral drug delivery device that comprises a cannula, whose tip can be inserted and kept in direct apposition to the scleral surface). In a specific embodiment, administration to the outer surface of the sclera is performed using a posterior juxtascleral depot procedure, which involves drug being drawn into a blunt-tipped curved cannula and then delivered in direct contact with the outer surface of the sclera without puncturing the eyeball. In particular, following the creation of a small incision to bare sclera, the cannula tip is inserted (see FIG. 7A). The curved portion of the cannula shaft is inserted, keeping the cannula tip in direct apposition to the scleral surface (see FIG. 7B-7D). After complete insertion of the cannula (FIG. 7D), the drug is slowly injected while gentle pressure is maintained along the top and sides of the cannula shaft with sterile cotton swabs. This method of delivery avoids the risk of intraocular infection and retinal detachment, side effects commonly associated with injecting therapeutic agents directly into the eye.

Doses that maintain a concentration of the transgene product at a Cmin of at least 0.330 μg/mL in the Vitreous humour, or 0.110 μg/mL in the Aqueous humour (the anterior chamber of the eye) for three months are desired; thereafter, Vitreous Cmin concentrations of the transgene product ranging from 1.70 to 6.60 μg/mL, and/or Aqueous Cmin concentrations ranging from 0.567 to 2.20 μg/mL should be maintained. However, because the transgene product is continuously produced (under the control of a constitutive promoter or induced by hypoxic conditions when using an hypoxia-inducible promoter), maintenance of lower concentrations can be effective. Vitreous humour concentrations can be measured directly in patient samples of fluid collected from the vitreous humour or the anterior chamber, or estimated and/or monitored by measuring the patient's serum concentrations of the transgene product—the ratio of systemic to vitreal exposure to the transgene product is about 1:90,000. (E.g., see, vitreous humor and serum concentrations of ranibizumab reported in Xu L, et al., 2013, Invest. Opthal. Vis. Sci. 54: 1616-1624, at p. 1621 and Table 5 at p. 1623, which is incorporated by reference herein in its entirety).

In certain embodiment, described herein is an micro volume injector delivery system, which is manufactured by Altaviz (see FIGS. 7A and 7B) (see, e.g. International Patent Application Publication No. WO 2013/177215, United States Patent Application Publication No. 2019/0175825, and United States Patent Application Publication No. 2019/0167906) that can be used for any administration route described herein for eye administration. The micro volume injector delivery system may include a gas-powered module providing high force delivery and improved precision, as described in United States Patent Application Publication No. 2019/0175825 and United States Patent Application Publication No. 2019/0167906. In addition, the micro volume injector delivery system may include a hydraulic drive for providing a consistent dose rate, and a low-force activation lever for controlling the gas-powered module and, in turn, the fluid delivery. In certain embodiment, the micro volume injector delivery system can be used for micro volume injector is a micro volume injector with dose guidance and can be used with, for example, a suprachoroidal needle (for example, the Clearside® needle), a subretinal needle, an intravitreal needle, a juxtascleral needle, a subconjunctival needle, and/or intraretinal needle. The benefits of using micro volume injector include: (a) more controlled delivery (for example, due to having precision injection flow rate control and dose guidance), (b) single surgeon, single hand, one finger operation; (c) pneumatic drive with 10 μL increment dosage; (d) divorced from the vitrectomy machine; (e) 400 μL syringe dose; (f) digitally guided delivery; (g) digitally recorded delivery; and (h) agnostic tip (for example, the MedOne 38 g needle and the Dorc 41 g needle can be used for subretinal delivery, while the Clearside® needle and the Visionisti OY adaptor can be used for subretinal delivery).

In certain embodiments of the methods described herein, the recombinant vector is administered suprachoroidally (e.g., by suprachoroidal injection). In a specific embodiment, suprachoroidal administration (e.g., an injection into the suprachoroidal space) is performed using a suprachoroidal drug delivery device. Suprachoroidal drug delivery devices are often used in suprachoroidal administration procedures, which involve administration of a drug to the suprachoroidal space of the eye (see, e.g., Hariprasad, 2016, Retinal Physician 13: 20-23; Goldstein, 2014, Retina Today 9(5): 82-87; Baldassarre et al., 2017; each of which is incorporated by reference herein in its entirety). The suprachoroidal drug delivery devices that can be used to deposit the recombinant vector in the suprachoroidal space according to the invention described herein include, but are not limited to, suprachoroidal drug delivery devices manufactured by Clearside® Biomedical, Inc. (see, for example, Hariprasad, 2016, Retinal Physician 13: 20-23) and MedOne suprachoroidal catheters. In another embodiment, the suprachoroidal drug delivery device that can be used in accordance with the methods described herein comprises the micro volume injector delivery system, which is manufactured by Altaviz (see FIGS. 7A and 7B) (see, e.g. International Patent Application Publication No. WO 2013/177215, United States Patent Application Publication No. 2019/0175825, and United States Patent Application Publication No. 2019/0167906) that can be used for any administration route described herein for eye administration. The micro volume injector delivery system may include a gas-powered module providing high force delivery and improved precision, as described in United States Patent Application Publication No. 2019/0175825 and United States Patent Application Publication No. 2019/0167906. In addition, the micro volume injector delivery system may include a hydraulic drive for providing a consistent dose rate, and a low-force activation lever for controlling the gas-powered module and, in turn, the fluid delivery. The micro volume injector is a micro volume injector with dose guidance and can be used with, for example, a suprachoroidal needle (for example, the Clearside® needle) or a subretinal needle. The benefits of using micro volume injector include: (a) more controlled delivery (for example, due to having precision injection flow rate control and dose guidance), (b) single surgeon, single hand, one finger operation; (c) pneumatic drive with 10 μL increment dosage; (d) divorced from the vitrectomy machine; (e) 400 μL syringe dose; (f) digitally guided delivery; (g) digitally recorded delivery; and (h) agnostic tip (for example, the MedOne 38 g needle and the Dorc 41 g needle can be used for subretinal delivery, while the Clearside® needle and the Visionisti OY adaptor can be used for suprachoroidal delivery). In another embodiment, the suprachoroidal drug delivery device that can be used in accordance with the methods described herein is a tool that comprises a normal length hypodermic needle with an adaptor (and preferably also a needle guide) manufactured by Visionisti OY, which adaptor turns the normal length hypodermic needle into a suprachoroidal needle by controlling the length of the needle tip exposing from the adapter (see FIG. 8) (see, for example, U.S. Design Pat. No. D878,575; and International Patent Application. Publication No. WO/2016/083669) In a specific embodiment, the suprachoroidal drug delivery device is a syringe with a 1 millimeter 30 gauge needle (see FIG. 1). During an injection using this device, the needle pierces to the base of the sclera and fluid containing drug enters the suprachoroidal space, leading to expansion of the suprachoroidal space. As a result, there is tactile and visual feedback during the injection. Following the injection, the fluid flows posteriorly and absorbs dominantly in the choroid and retina. This results in the production of therapeutic product from all retinal cell layers and choroidal cells. Using this type of device and procedure allows for a quick and easy in-office procedure with low risk of complications. A max volume of 100 μl can be injected into the suprachoroidal space.

In a specific embodiment, the intravitreal administration is performed with a intravitreal drug delivery device that comprises the micro volume injector delivery system, which is manufactured by Altaviz (see FIGS. 7A and 7B) (see, e.g. International Patent Application Publication No. WO 2013/177215) , United States Patent Application Publication No. 2019/0175825, and United States Patent Application Publication No. 2019/0167906) that can be used for any administration route described herein for eye administration. The micro volume injector delivery system may include a gas-powered module providing high force delivery and improved precision, as described in United States Patent Application Publication No. 2019/0175825 and United States Patent Application Publication No. 2019/0167906. In addition, the micro volume injector delivery system may include a hydraulic drive for providing a consistent dose rate, and a low-force activation lever for controlling the gas-powered module and, in turn, the fluid delivery. The micro volume injector is a micro volume injector with dose guidance and can be used with, for example, a intravitreal needle. The benefits of using micro volume injector include: (a) more controlled delivery (for example, due to having precision injection flow rate control and dose guidance), (b) single surgeon, single hand, one finger operation; (c) pneumatic drive with 10 μL increment dosage; (d) divorced from the vitrectomy machine; (e) 400 μL syringe dose; (f) digitally guided delivery; (g) digitally recorded delivery; and (h) agnostic tip. In a specific embodiment, the subretinal administration is performed with a subretinal drug delivery device that comprises the micro volume injector delivery system, which is manufactured by Altaviz (see FIGS. 7A and 7B) (see, e.g. International Patent Application Publication No. WO 2013/177215, United States Patent Application Publication No. 2019/0175825, and United States Patent Application Publication No. 2019/0167906) that can be used for any administration route described herein for eye administration. The micro volume injector delivery system may include a gas-powered module providing high force delivery and improved precision, as described in United States Patent Application Publication No. 2019/0175825 and United States Patent Application Publication No. 2019/0167906. In addition, the micro volume injector delivery system may include a hydraulic drive for providing a consistent dose rate, and a low-force activation lever for controlling the gas-powered module and, in turn, the fluid delivery. Micro volume injector is a micro volume injector with dose guidance and can be used with, for example, a subretinal needle. The benefits of using micro volume injector include: (a) more controlled delivery (for example, due to having precision injection flow rate control and dose guidance), (b) single surgeon, single hand, one finger operation; (c) pneumatic drive with 10 μL it increment dosage; (d) divorced from the vitrectomy machine; (e) 400 μL syringe dose; (f) digitally guided delivery; (g) digitally recorded delivery; and (h) agnostic tip (for example, the MedOne 38 g needle and the Dorc 41 g needle can be used for subretinal delivery, while the Clearside® needle and the Visionisti OY adaptor can be used for suprachoroidal delivery).

In certain embodiments, the recombinant vector is administered to the outer surface of the sclera (for example, by the use of a juxtascleral drug delivery device that comprises a cannula, whose tip can be inserted and kept in direct apposition to the scleral surface). In a specific embodiment, administration to the outer surface of the sclera is performed using a posterior juxtascleral depot procedure, which involves drug being drawn into a blunt-tipped curved cannula and then delivered in direct contact with the outer surface of the sclera without puncturing the eyeball. In particular, following the creation of a small incision to bare sclera, the cannula tip is inserted (see FIG. 7A). The curved portion of the cannula shaft is inserted, keeping the cannula tip in direct apposition to the scleral surface (see FIGS. 7B-7D). After complete insertion of the cannula (FIG. 7D), the drug is slowly injected while gentle pressure is maintained along the top and sides of the cannula shaft with sterile cotton swabs. This method of delivery avoids the risk of intraocular infection and retinal detachment, side effects commonly associated with injecting therapeutic agents directly into the eye. In a specific embodiment, the juxtascleral administration is performed with a juxtascleral drug delivery device that comprises the micro volume injector delivery system, which is manufactured by Altaviz (see FIGS. 7A and 7B) (see, e.g. International Patent Application Publication No. WO 2013/177215, United States Patent Application Publication No. 2019/0175825, and United States Patent Application Publication No. 2019/0167906) that can be used for any administration route described herein for eye administration. The micro volume injector delivery system may include a gas-powered module providing high force delivery and improved precision, as described in United States Patent Application Publication No. 2019/0175825 and United States Patent Application Publication No. 2019/0167906. In addition, the micro volume injector delivery system may include a hydraulic drive for providing a consistent dose rate, and a low-force activation lever for controlling the gas-powered module and, in turn, the fluid delivery. Micro Volume Injector is a micro volume injector with dose guidance and can be used with, for example, a juxtascleral needle. The benefits of using micro volume injector include: (a) more controlled delivery (for example, due to having precision injection flow rate control and dose guidance), (b) single surgeon, single hand, one finger operation; (c) pneumatic drive with 10 μL increment dosage; (d) divorced from the vitrectomy machine; (e) 400 μL syringe dose; (f) digitally guided delivery; (g) digitally recorded delivery; and (h) agnostic tip.

In certain embodiments, dosages are measured by genome copies per ml or the number of genome copies administered to the eye of the patient (e.g., administered suprachoroidally, subretinally, intravitreally, juxtasclerally, subconjunctivally, and/or intraretinally (e.g., by suprachoroidal injection, subretinal injection via the transvitreal approach (a surgical procedure), subretinal administration via the suprachoroidal space, or a posterior juxtascleral depot procedure). In certain embodiments, 2.4×10¹¹ genome copies per ml to 1×10¹³ genome copies per ml are administered. In a specific embodiment, 2.4×10¹¹ genome copies per ml to 5×10¹¹ genome copies per ml are administered. In another specific embodiment, 5×10¹¹ genome copies per ml to 1×10¹² genome copies per ml are administered. In another specific embodiment, 1×10¹² genome copies per ml to 5×10¹² genome copies per ml are administered. In another specific embodiment, 5×10¹² genome copies per ml to 1×10¹³ genome copies per ml are administered. In another specific embodiment, about 2.4×10¹¹ genome copies per ml are administered. In another specific embodiment, about 5×10¹¹ genome copies per ml are administered. In another specific embodiment, about 1×10¹² genome copies per ml are administered. In another specific embodiment, about 5×10¹² genome copies per ml are administered. In another specific embodiment, about 1×10¹³ genome copies per ml are administered. In certain embodiments, 1×10⁹ to 1×10¹² genome copies are administered. In specific embodiments, 3×10⁹ to 2.5×10¹¹ genome copies are administered. In specific embodiments, 1×10⁹ to 2.5×10¹¹ genome copies are administered. In specific embodiments, 1×10⁹ to 1×10¹¹ genome copies are administered. In specific embodiments, 1×10⁹ to 5×10⁹ genome copies are administered. In specific embodiments, 6×10⁹ to 3×10¹⁰ genome copies are administered. In specific embodiments, 4×10¹⁰ to 1×10¹¹ genome copies are administered. In specific embodiments, 2×10¹¹ to 1×10¹² genome copies are administered. In a specific embodiment, about 3×10⁹ genome copies are administered (which corresponds to about 1.2×10¹⁰ genome copies per ml in a volume of 250 μl). In another specific embodiment, about 1×10¹⁰ genome copies are administered (which corresponds to about 4×10¹⁰ genome copies per ml in a volume of 250 μl). In another specific embodiment, about 6×10¹⁰ genome copies are administered (which corresponds to about 2.4×10¹¹ genome copies per ml in a volume of 250 μl). In another specific embodiment, about 1.6×10¹¹ genome copies are administered (which corresponds to about 6.2×10¹¹ genome copies per ml in a volume of 250 μl). In another specific embodiment, about 1.55×10¹¹ genome copies are administered (which corresponds to about 6.2×10¹¹ genome copies per ml in a volume of 250 μl). In another specific embodiment, about 1.6×10¹¹ genome copies are administered (which corresponds to about 6.4×10¹¹ genome copies per ml in a volume of 250 μl). In another specific embodiment, about 2.5×10¹¹ genome copies (which corresponds to about 1.0×10¹² in a volume of 250 μl) are administered.

In certain embodiments, about 3.0×10¹³ genome copies per eye are administered. In certain embodiments, up to 3.0×10¹³ genome copies per eye are administered.

In certain embodiments, about 6.0×10¹⁰ genome copies per eye are administered. In certain embodiments, about 1.6×10¹¹ genome copies per eye are administered. In certain embodiments, about 2.5×10¹¹ genome copies per eye are administered. In certain embodiments, about 5.0×10¹¹ genome copies per eye are administered. In certain embodiments, about 3×10¹² genome copies per eye are administered. In certain embodiments, about 1.0×10¹² genome copies per ml per eye are administered. In certain embodiments, about 2.5×10¹² genome copies per ml per eye are administered.

In certain embodiments, about 6.0×10¹⁰ genome copies per eye are administered by subretinal injection. In certain embodiments, about 1.6×10¹¹ genome copies per eye are administered by subretinal injection. In certain embodiments, about 2.5×10¹¹ genome copies per eye are administered by subretinal injection. In certain embodiments, about 3.0×10¹³ genome copies per eye are administered by subretinal injection. In certain embodiments, up to 3.0×10¹³ genome copies per eye are administered by subretinal injection.

In certain embodiments, about 2.5×10¹¹ genome copies per eye are administered by suprachoroidal injection. In certain embodiments, about 5.0×10¹¹ genome copies per eye are administered by suprachoroidal injection. In certain embodiments, about 3×10¹² genome copies per eye are administered by suprachoroidal injection. In certain embodiments, about 2.5×10¹¹ genome copies per eye are administered by a single suprachoroidal injection. In certain embodiments, about 5.0×10¹¹ genome copies per eye are administered by double suprachoroidal injections. In certain embodiments, about 3.0×10¹³ genome copies per eye are administered by suprachoroidal injection. In certain embodiments, up to 3.0×10¹³ genome copies per eye are administered by suprachoroidal injection. In certain embodiments, about 2.5×10¹² genome copies per ml per eye are administered by a single suprachoroidal injection in a volume of 100 μl. In certain embodiments, about 2.5×10¹² genome copies per ml per eye are administered by double suprachoroidal injections, wherein each injection is in a volume of 100 μl.

As used herein and unless otherwise specified, the term “about” means within plus or minus 10% of a given value or range.

In certain embodiments, the term “about” encompasses the exact number recited.

In certain embodiments, an infrared thermal camera can be used to detect changes in the thermal profile of the ocular surface after the administering of a solution which is cooler than body temperature to detect changes in the thermal profile of the ocular surface that allows for visualization of the spread of the solution, e.g., within the SCS, and can potentially determine whether the administration was successfully completed. This is because in certain embodiments the formulation containing the recombinant vector to be administered is initially frozen, brought to room temperature (68-72° F.), and thawed for a short period of time (e.g., at least 30 minutes) before administration, and thus the formulation is colder than the human eye (about 92° F.) (and sometimes even colder than room temperature) at the time of injection. The drug product is typically used within 4 hours of thaw and the warmest the solution would be is room temperature. In a preferred embodiment, the procedure is videoed with infrared video.

Infrared thermal cameras can detect small changes in temperature. They capture infrared energy through a lens and convert the energy into an electronic signal. The infrared light is focused onto an infrared sensor array which converts the energy into a thermal image. The infrared thermal camera can be used for any method of administration to the eye, including any administration route described herein, for example, suprachoroidal administration, subretinal administration, subconjunctival administration, intravitreal administration, or administration with the use of a slow infusion catheter in to the suprachoroidal space. In a specific embodiment, the infrared thermal camera is an FLIR T530 infrared thermal camera. The FLIR T530 infrared thermal camera can capture slight temperature differences with an accuracy of ±3.6° F. The camera has an infrared resolution of 76,800 pixels. The camera also utilizes a 24° lens capturing a smaller field of view. A smaller field of view in combination with a high infrared resolution contributes to more detailed thermal profiles of what the operator is imaging. However, other infrared camera can be used that have different abilities and accuracy for capturing slight temperature changes, with different infrared resolutions, and/or with different degrees of lens.

In a specific embodiment, the infrared thermal camera is an FLIR T420 infrared thermal camera. In a specific embodiment, the infrared thermal camera is an FLIR T440 infrared thermal camera. In a specific embodiment, the infrared thermal camera is an Fluke Ti400 infrared thermal camera. In a specific embodiment, the infrared thermal camera is an FLIRE60 infrared thermal camera. In a specific embodiment, the infrared resolution of the infrared thermal camera is equal to or greater than 75,000 pixels. In a specific embodiment, the thermal sensitivity of the infrared thermal camera is equal to or smaller than 0.05° C. at 30° C. In a specific embodiment, the field of view (FOV) of the infrared thermal camera is equal to or lower than 25°×25°.

In certain embodiments, an iron filer is used with the infrared thermal camera to detect changes in the thermal profile of the ocular surface. In a preferred embodiment, the use of an iron filter is able to a generate pseudo-color image, wherein the warmest or high temperature parts are colored white, intermediate temperatures are reds and yellows, and the coolest or low temperature parts are black. In certain embodiments, other types of filters can also be used to generate pseudo-color images of the thermal profile.

The thermal profile for each administration method can be different. For example, in one embodiment, a successful suprachoroidal injection can be characterized by: (a) a slow, wide radial spread of the dark color, (b) very dark color at the beginning, and (c) a gradual change of injectate to lighter color, i.e., a temperature gradient noted by a lighter color. In one embodiment, an unsuccessful suprachoroidal injection can be characterized by: (a) no spread of the dark color, and (b) a minor change in color localized to the injection site without any distribution. In certain embodiments, the small localized temperature drop is result from cannula (low temperature) touching the ocular tissues (high temperature). In one embodiment, a successful intravitreal injection can be characterized by: (a) no spread of the dark color, (b) an initial change to very dark color localized to the injection site, and (c) a gradual and uniform change of the entire eye to darker color. In one embodiment, an extraocular efflux can be characterized by: (a) quick flowing streams on outside on the exterior surface of the eye, (b) very dark color at the beginning, and (c) a quick change to lighter color.

### 5.3.3 Sampling and Monitoring of Efficacy

Effects of the methods of treatment provided herein on visual deficits may be measured by BCVA (Best-Corrected Visual Acuity), intraocular pressure, slit lamp biomicroscopy, and/or indirect ophthalmoscopy. Extraocular movement may also be assessed. The intraocular pressure measurements may be conducted using Tonopen or Goldmann applanation tonometry. The slit lamp examination may include an evaluation of the lids/lashes, conjunctiva/sclera, cornea, anterior chamber, iris, lens, and/or vitreous body.

In specific embodiments, effects of the methods provided herein on visual deficits may be measured by whether the human patient's eye that is treated by a method described herein achieves BCVA of greater than 43 letters post-treatment (e.g., 46-50 weeks or 98-102 weeks post-treatment). A BCVA of 43 letters corresponds to 20/160 approximate Snellen equivalent. In a specific embodiment, the human patient's eye that is treated by a method described herein achieves BCVA of greater than 43 letters post-treatment (e.g., 46-50 weeks or 98-102 weeks post-treatment).

In specific embodiments, effects of the methods provided herein on visual deficits may be measured by whether the human patient's eye that is treated by a method described herein achieves BCVA of greater than 84 letters post-treatment (e.g., 46-50 weeks or 98-102 weeks post-treatment). A BCVA of 84 letters corresponds to 20/20 approximate Snellen equivalent. In a specific embodiment, the human patient's eye that is treated by a method described herein achieves BCVA of greater than 84 letters post-treatment (e.g., 46-50 weeks or 98-102 weeks post-treatment). The BCVA testing may be conducted at a distance of 4 meters using ETDRS charts. For participants with reduced vision (inability to read ≥20 letters correctly at 4 meters), the BCVA testing may be conducted at a distance of 1 meter.

Effects of the methods of treatment provided herein on physical changes to eye/retina may be measured by SD-OCT (SD-Optical Coherence Tomography).

Efficacy may be monitored as measured by electroretinography (ERG).

Effects of the methods of treatment provided herein may be monitored by measuring signs of vision loss, infection, inflammation and other safety events, including retinal detachment.

Retinal thickness may be monitored to determine efficacy of the treatments provided herein. Without being bound by any particular theory, thickness of the retina may be used as a clinical readout, wherein the greater reduction in retinal thickness or the longer period of time before thickening of the retina, the more efficacious the treatment. Retinal function may be determined, for example, by ERG. ERG is a non-invasive electrophysiologic test of retinal function, approved by the FDA for use in humans, which examines the light sensitive cells of the eye (the rods and cones), and their connecting ganglion cells, in particular, their response to a flash stimulation. Retinal thickness may be determined, for example, by SD-OCT. SD-OCT is a three-dimensional imaging technology which uses low-coherence interferometry to determine the echo time delay and magnitude of backscattered light reflected off an object of interest. OCT can be used to scan the layers of a tissue sample (e.g., the retina) with 3 to 15 μm axial resolution, and SD-OCT improves axial resolution and scan speed over previous forms of the technology (Schuman, 2008, Trans. Am. Opthamol. Soc. 106:426-458).

Effects of the methods provided herein may also be measured by a change from baseline in National Eye Institute Visual Functioning Questionnaire, the Rasch-scored version (NEI-VFQ-28-R) (composite score; activity limitation domain score; and socio-emotional functioning domain score). Effects of the methods provided herein may also be measured by a change from baseline in National Eye Institute Visual Functioning Questionnaire 25-item version (NEI-VFQ-25) (composite score and mental health subscale score). Effects of the methods provided herein may also be measured by a change from baseline in Macular Disease Treatment Satisfaction Questionnaire (MacTSQ) (composite score; safety, efficacy, and discomfort domain score; and information provision and convenience domain score).

In specific embodiments, the efficacy of a method described herein is reflected by an improvement in vision at about 4 weeks, 12 weeks, 6 months, 12 months, 24 months, 36 months, or at other desired timepoints. In a specific embodiment, the improvement in vision is characterized by an increase in BCVA, for example, an increase by 1 letter, 2 letters, 3 letters, 4 letters, 5 letters, 6 letters, 7 letters, 8 letters, 9 letters, 10 letters, 11 letters, or 12 letters, or more. In a specific embodiment, the improvement in vision is characterized by a 5%, 10%, 15%, 20%, 30%, 40%, 50% or more increase in visual acuity from baseline.

In specific embodiments, the efficacy of a method described herein is reflected by an reduction in central retinal thickness (CRT) at about 4 weeks, 12 weeks, 6 months, 12 months, 24 months, 36 months, or at other desired timepoint, for example, a 5%, 10%, 15%, 20%, 30%, 40%, 50% or more decrease in central retinal thickness from baseline.

In s specific embodiments, there is no inflammation in the eye after treatment or little inflammation in the eye after treatment (for example, an increase in the level of inflammation byl0%, 5%, 2%, 1% or less from baseline). Effects of the methods provided herein on visual deficits may be measured by OptoKinetic Nystagmus (OKN).

Without being bound by theory, this visual acuity screening uses the principles of the OKN involuntary reflex to objectively assess whether a patient's eyes can follow a moving target. By using OKN, no verbal communication is needed between the tester and the patient. As such, OKN can be used to measure visual acuity in pre-verbal and/or non-verbal patients. In certain embodiments, OKN is used to measure visual acuity in patients that are 1 month old, 2 months old, 3 months old, 4 months old, 5 months old, 6 months old, 7 months old, 8 months old, 9 months old, 10 months old, 11 months old, 1 year old, 1.5 years old, 2 years old, 2.5 years old, 3 years old, 3.5 years old, 4 years old, 4.5 years old, or 5 years old. In certain embodiments, an iPad is used to measure visual acuity through detection of the OKN reflex when a patient is looking at movement on the iPad.

Without being bound by theory, this visual acuity screening uses the principles of the OKN involuntary reflex to objectively assess whether a patient's eyes can follow a moving target. By using OKN, no verbal communication is needed between the tester and the patient. As such, OKN can be used to measure visual acuity in pre-verbal and/or non-verbal patients. In certain embodiments, OKN is used to measure visual acuity in patients that are less than 1.5 months old, 2 months old, 3 months old, 4 months old, 5 months old, 6 months old, 7 months old, 8 months old, 9 months old, 10 months old, 11 months old, 1 year old, 1.5 years old, 2 years old, 2.5 years old, 3 years old, 3.5 years old, 4 years old, 4.5 years old, or 5 years old. In another specific embodiment, OKN is used to measure visual acuity in patients that are 1-2 months old, 2-3 months old, 3-4 months old, 4-5 months old, 5-6 months old, 6-7 months old, 7-8 months old, 8-9 months old, 9-10 months old, 10-11 months old, 11 months to 1 year old, 1-1.5 years old, 1.5-2 years old, 2-2.5 years old, 2.5-3 years old, 3-3.5 years old, 3.5-4 years old, 4-4.5 years old, or 4.5-5 years old. In another specific embodiment, OKN is used to measure visual acuity in patients that are 6 months to 5 years old. In certain embodiments, an iPad is used to measure visual acuity through detection of the OKN reflex when a patient is looking at movement on the iPad.

If the human patient is a child, visual function can be assessed using an optokinetic nystagmus (OKN)-based approach or a modified OKN-based approach.

Vector shedding may be determined for example by measuring vector DNA in biological fluids such as tears, serum or urine using quantitative polymerase chain reaction. In some embodiments, no vector gene copies are detectable in urine at any time point after administration of the vector. In some embodiments, less than 1000, less than 500, less than 100, less than 50 or less than 10 vector gene copies/5 μL are detectable by quantitative polymerase chain reaction in a biological fluid (e.g., tears, serum or urine) at any point after administration. In specific embodiments, 210 vector gene copies/5 μL or less are detectable in serum. In some embodiments, less than 1000, less than 500, less than 100, less than 50 or less than 10 vector gene copies/5 μL are detectable by quantitative polymerase chain reaction in a biological fluid (e.g., tears, serum or urine) by 1, 2, 3, 4, 5, 6, 7, 8, 9, 10, 11, 12, 13 or 14 weeks after administration. In specific embodiments, no vector gene copies are detectable in a biological fluid (e.g., tears, serum or urine) by Week 14 after administration of the vector. In some embodiments, no vector gene copies are detectable in a biological fluid (e.g., tears, serum or urine) at any time point after administration of the vector.

In some embodiments, patients treated in accordance with a method provided herein are monitored for the development of Center Involved-Diabetic Macular Edema (CI-DME), cataracts, neovascularization, retinal detachment, diabetes complications, vessel regression, area of leakage, and/or area of retinal nonperfusion. Development of CI-DME, cataracts, neovascularization, retinal detachment, diabetes complications, vessel regression, area of leakage, and area of retinal nonperfusion may be assessed by any method known in the art or provided herein. Diabetic complications developed in a subject may require panretinal photocoagulation (PRP), anti-VEGF therapy and/or surgical intervention). Diabetic complications may be sight-threatening. Cataracts developed in a subject may require surgery. In some embodiments, the vital signs (e.g., heart rate, blood pressure) of a patient treated in accordance with the methods provided herein may be monitored.

The safety of a method of treatment described herein may be assessed by assays known in the art. In certain embodiments, the safety of a method of treatment described herein is assessed by serum chemistry measurements of, e.g., levels of glucose, blood urea nitrogen, creatinine, sodium, potassium, chloride, carbon dioxide, calcium, total protein albumin total bilirubin, direct bilirubin, alkaline phosphatase, alanine aminotransferase, aspartate aminotransferase, and/or creatine kinase. In certain embodiments, the safety of a method of treatment described herein is assessed by hematological measurements of, e.g., platelets, hematocrit, hemoglobin, red blood cells, white blood cells, neutrophils, lymphocytes, monocytes, eosinophils, basophils, mean corpuscular volume, mean corpuscular hemoglobin and/or mean corpuscular hemoglobin concentration. In certain embodiments, the safety of a method of treatment described herein is assessed by urinalysis, e.g., a dipstick test for levels of glucose, ketones, protein, and/or blood (if warranted, a microscopic evaluation may be completed). In certain embodiments, the safety of a method of treatment described herein is assessed by measurements of coagulation (e.g., prothrombin time and/or partial thromboplastin time) or by measurements of hemoglobin A1c.

In certain embodiments, the effects of a method provided herein are determined by statistical analysis. Statistical inference may be done at a significance level of 2-sided α=0.2. Statistical endpoints may be summarized with a corresponding 80% confidence interval.

The effects of a method provided herein may be determined by Fisher's Exact test, wherein a treated population is tested against a historical rate of response (e.g., 5%) in an untreated population.

### 5.4 COMBINATION THERAPIES

The methods of treatment provided herein may be combined with one or more additional therapies. In one aspect, the methods of treatment provided herein are administered with laser photocoagulation. In one aspect, the methods of treatment provided herein are administered with photodynamic therapy with verteporfin.

In one aspect, the methods of treatment provided herein are administered with intravitreal (IVT) injections with anti-VEGF agents, including but not limited to HuPTMFabVEGFi, e.g., HuGlyFabVEGFi produced in human cell lines (Dumont et al., 2015, supra), or other anti-VEGF agents such as pegaptanib, ranibizumab, aflibercept, or bevacizumab.

The additional therapies may be administered before, concurrently or subsequent to the gene therapy treatment.

The efficacy of the gene therapy treatment may be indicated by the elimination of or reduction in the number of rescue treatments using standard of care, for example, intravitreal injections with anti-VEGF agents, including but not limited to HuPTMFabVEGFi, e.g., HuGlyFabVEGFi produced in human cell lines, or other anti-VEGF agents such as pegaptanib, ranibizumab, aflibercept, or bevacizumab.

TABLE 3
TABLE OF SEQUENCES

| SEQ | | |
|---|---|---|
| ID | | |
| NO: | Description | Sequence |
| 1 | Ranibizumab | DIQLTQSPSSLSASVGDRVTITCSASQDISNYLNWYQQKPGKAPKVLIYFTSSLH |
| | Fab Amino | SGVPSRFSGSGSGTDFTLTISSLQPEDFATYYCQQYSTVPWTFGQGTKVEIKRTV |
| | Acid Sequence | AAPSVFIFPPSDEQLKSGTASVVCLLNNFYPREAKVQWKVDNALQSGNSQESVTE |
| | (Light chain) | QDSKDSTYSLSSTLTLSKADYEKHKVYACEVTHQGLSSPVTKSFNRGEC |
| 2 | Ranibizumab | EVQLVESGGGLVQPGGSLRLSCAASGYDFTHYGMNWVRQAPGKGLEWVGWINTYT |
| | Fab Amino | GEPTYAADFKRRFTFSLDTSKSTAYLQMNSLRAEDTAVYYCAKYPYYYGTSHWYF |
| | Acid Sequence | DVWGQGTLVTVSSASTKGPSVFPLAPSSKSTSGGTAALGCLVKDYFPEPVTVSWN |
| | (Heavy chain) | SGALTSGVHTFPAVLQSSGLYSLSSVVTVPSSSLGTQTYICNVNHKPSNTKVDKK |
| | | VEPKSCDKTHL |
| 3 | Bevacizumab | DIQMTQSPSSLSASVGDRVTITCSASQDISNYLNWYQQKPGKAPKVLIYFTSSLH |
| | Fab Amino | SGVPSRFSGSGSGTDFTLTISSLQPEDFATYYCQQYSTVPWTFGQGTKVEIKRTV |
| | Acid Sequence | AAPSVFIFPPSDEQLKSGTASVVCLLNNFYPREAKVQWKVDNALQSGNSQESVTE |
| | (Light chain) | QDSKDSTYSLSSTLTLSKADYEKHKVYACEVTHQGLSSPVTKSFNRGEC |
| 4 | Bevacizumab | EVQLVESGGGLVQPGGSLRLSCAASGYTFTNYGMNWVRQAPGKGLEWVGWINTYT |
| | Fab Amino | GEPTYAADFKRRFTFSLDTSKSTAYLQMNSLRAEDTAVYYCAKYPHYYGSSHWYF |
| | Acid Sequence | DVWGQGTLVTVSSASTKGPSVFPLAPSSKSTSGGTAALGCLVKDYFPEPVTVSWN |
| | (Heavy chain) | SGALTSGVHTFPAVLQSSGLYSLSSVVTVPSSSLGTQTYICNVNHKPSNTKVDKK |
| | | VEPKSCDKTHL |
| 5 | VEGF-A signal | MNFLLSWVHW SLALLLYLHH AKWSQA |
| | peptide | |
| 6 | Fibulin-1 | MERAAPSRRV PLPLLLLGGL ALLAAGVDA |
| | signal peptide | |
| 7 | Vitronectin | MAPLRPLLIL ALLAWVALA |
| | signal peptide | |
| 8 | Complement | MRLLAKIICLMLWAICVA |
| | Factor H signal | |
| | peptide | |
| 9 | Opticin signal | MRLLAFLSLL ALVLQETGT |
| | peptide | |
| 10 | Bevacimmab | gctagcgcca ccatgggctg gtcctgcatc atcctgttcc tggtggccac |
| | cDNA | cgccaccggc gtgcactccg acatccagat gacccagtcc ccctcctccc |
| | (Light chain) | tgtccgcctc cgtgggcgac cgggtgacca tcacctgctc cgcctcccag |
| | | gacatctcca actacctgaa ctggtaccag cagaagcccg gcaaggcccc |
| | | caaggtgctg atctacttca cctcctccct gcactccggc gtgccctccc |
| | | ggttctccgg ctccggctcc ggcaccgact tcaccctgac catctcctcc |
| | | ctgcagcccg aggacttcgc cacctactac tgccagcagt actccaccgt |
| | | gccctggacc ttcggccagg gcaccaaggt ggagatcaag cggaccgtgg |
| | | ccgccccctc cgtgttcatc ttccccccct ccgacgagca gctgaagtcc |
| | | ggcaccgcct ccgtggtgtg cctgctgaac aacttctacc cccgggaggc |
| | | caaggtgcag tggaaggtgg acaacgccct gcagtccggc aactcccagg |
| | | agtccgtgac cgagcaggac tccaaggact ccacctactc cctgtcctcc |
| | | accctgaccc tgtccaaggc cgactacgag aagcacaagg tgtacgcctg |
| | | cgaggtgacc caccagggcc tgtcctcccc cgtgaccaag tccttcaacc |
| | | ggggcgagtg ctgagcggcc gcctcgag |
| 11 | Bevacizumab | gctagcgcca ccatgggctg gtcctgcatc atcctgttcc tggtggccac |
| | cDNA (Heavy | cgccaccggc gtgcactccg aggtgcagct ggtggagtcc ggcggcggcc |
| | chain) | tggtgcagcc cggcggctcc ctgcggctgt cctgcgccgc ctccggctac |
| | | accttcacca actacggcat gaactgggtg cggcaggccc ccggcaaggg |
| | | cctggagtgg gtgggctgga tcaacaccta caccggcgag cccacctacg |
| | | ccgccgactt caagcggcgg ttcaccttct ccctggacac ctccaagtcc |
| | | accgcctacc tgcagatgaa ctccctgcgg gccgaggaca ccgccgtgta |
| | | ctactgcgcc aagtaccccc actactacgg ctcctcccac tggtacttcg |
| | | acgtgtgggg ccagggcacc ctggtgaccg tgtcctccgc ctccaccaag |
| | | ggcccctccg tgttccccct ggccccctcc tccaagtcca cctccggcgg |
| | | caccgccgcc ctgggctgcc tggtgaagga ctacttcccc gagcccgtga |
| | | ccgtgtcctg gaactccggc gccctgacct ccggcgtgca caccttcccc |
| | | gccgtgctgc agtcctccgg cctgtactcc ctgtcctccg tggtgaccgt |
| | | gccctcctcc tccctgggca cccagaccta catctgcaac gtgaaccaca |
| | | agccctccaa caccaaggtg gacaagaagg tggagcccaa gtcctgcgac |
| | | aagacccaca cctgcccccc ctgccccgcc cccgagctgc tgggcggccc |
| | | ctccgtgttc ctgttccccc ccaagcccaa ggacaccctg atgatctccc |
| | | ggacccccga ggtgacctgc gtggtggtgg acgtgtccca cgaggacccc |
| | | gaggtgaagt tcaactggta cgtggacggc gtggaggtgc acaacgccaa |
| | | gaccaagccc cgggaggagc agtacaactc cacctaccgg gtggtgtccg |
| | | tgctgaccgt gctgcaccag gactggctga acggcaagga gtacaagtgc |
| | | aaggtgtcca acaaggccct gcccgccccc atcgagaaga ccatctccaa |
| | | ggccaagggc cagccccggg agccccaggt gtacaccctg cccccctccc |
| | | gggaggagat gaccaagaac caggtgtccc tgacctgcct ggtgaagggc |
| | | ttctacccct ccgacatcgc cgtggagtgg gagtccaacg gccagcccga |
| | | gaacaactac aagaccaccc cccccgtgct ggactccgac ggctccttct |
| | | tcctgtactc caagctgacc gtggacaagt cccggtggca gcagggcaac |
| | | gtgttctcct gctccgtgat gcacgaggcc ctgcacaacc actacaccca |
| | | gaagtccctg tccctgtccc ccggcaagtg agcggccgcc |
| 12 | Ranibizumab | gagctccatg gagtttttca aaaagacggc acttgccgca ctggttatgg |
| | cDNA (Light | gttttagtgg tgcagcattg gccgatatcc agctgaccca gagcccgagc |
| | chain | agcctgagcg caagcgttgg tgatcgtgtt accattacct gtagcgcaag |
| | comprising a | ccaggatatt agcaattatc tgaattggta tcagcagaaa ccgggtaaag |
| | signal | caccgaaagt tctgatttat tttaccagca gcctgcatag cggtgttccg |
| | sequence) | agccgtttta gcggtagcgg tagtggcacc gattttaccc tgaccattag |
| | | cagcctgcag ccggaagatt ttgcaaccta ttattgtcag cagtatagca |
| | | ccgttccgtg gacctttggt cagggcacca aagttgaaat taaacgtacc |
| | | gttgcagcac cgagcgtttt tatttttccg cctagtgatg aacagctgaa |
| | | aagcggcacc gcaagcgttg tttgtctgct gaataatttt tatccgcgtg |
| | | aagcaaaagt gcagtggaaa gttgataatg cactgcagag cggtaatagc |
| | | caagaaagcg ttaccgaaca ggatagcaaa gatagcacct atagcctgag |
| | | cagcaccctg accctgagca aagcagatta tgaaaaacac aaagtgtatg |
| | | cctgcgaagt tacccatcag ggtctgagca gtccggttac caaaagtttt |
| | | aatcgtggcg aatgctaata gaagcttggt acc |
| 13 | Ranibizumab | gagctcatat gaaatacctg ctgccgaccg ctgctgctgg tctgctgctc |
| | cDNA (Heavy | ctcgctgccc agccggcgat ggccgaagtt cagctggttg aaagcggtgg |
| | chain | tggtctggtt cagcctggtg gtagcctgcg tctgagctgt gcagcaagcg |
| | comprising a | gttatgattt tacccattat ggtatgaatt gggttcgtca ggcaccgggt |
| | signal | aaaggtctgg aatgggttgg ttggattaat acctataccg gtgaaccgac |
| | sequence) | ctatgcagca gattttaaac gtcgttttac ctttagcctg gataccagca |
| | | aaagcaccgc atatctgcag atgaatagcc tgcgtgcaga agataccgca |
| | | gtttattatt gtgccaaata tccgtattac tatggcacca gccactggta |
| | | tttcgatgtt tggggtcagg gcaccctggt taccgttagc agcgcaagca |
| | | ccaaaggtcc gagcgttttt ccgctggcac cgagcagcaa aagtaccagc |
| | | ggtggcacag cagcactggg ttgtctggtt aaagattatt ttccggaacc |
| | | ggttaccgtg agctggaata gcggtgcact gaccagcggt gttcatacct |
| | | ttccggcagt tctgcagagc agcggtctgt atagcctgag cagcgttgtt |
| | | accgttccga gcagcagcct gggcacccag acctatattt gtaatgttaa |
| | | tcataaaccg agcaatacca aagtggataa aaaagttgag ccgaaaagct |
| | | gcgataaaac ccatctgtaa tagggtacc |
| 14 | Bevacizumab | SASQDISNYLN |
| | and | |
| | Ranibizumab | |
| | Light Chain | |
| | CDR1 | |
| 15 | Bevacizumab | FTSSLHS |
| | and | |
| | Ranibizumab | |
| | Light Chain | |
| | CDR2 | |
| 16 | Bevacizumab | QQYSTVPWT |
| | and | |
| | Ranibizumab | |
| | Light Chain | |
| | CDR3 | |
| 17 | Bevacizumab | GYTFTNYGMN |
| | Heavy Chain | |
| | CDR1 | |
| 18 | Bevacizumab | WINTYTGEPTYAADFKR |
| | and | |
| | Ranibizumab | |
| | Heavy Chain | |
| | CDR2 | |
| 19 | Bevacizumab | YPHYYGSSHWYFDV |
| | Heavy Chain | |
| | CDR3 | |
| 20 | Ranibizumab | GYDFTHYGMN |
| | Heavy Chain | |
| | CDR1 | |
| 21 | Ranibizumab | YPYYYGTSHWYFDV |
| | Heavy Chain | |
| | CDR3 | |
| 22 | Albumin signal | MKWVTFISLLFLFSSAYS |
| | peptide | |
| 23 | Chymotrypsino | MAFLWLLSCWALLGTTFG |
| | gen signal | |
| | peptide | |
| 24 | Interleukin-2 | MYRMQLLSCIALILALVTNS |
| | signal peptide | |
| 25 | Trypsinogen-2 | MNLLL I LT FVAAAVA |
| | signal peptide | |
| 26 | F2A site | LLNFDLLKLAGDVESNPGP |
| 27 | T2A site | (GSG) EGRGSLLTCGDVEENPGP |
| 28 | P2A site | (GSG) ATNFSLLKQAGDVEENPGP |
| 29 | E2A site | (GSG) QCTNYALLKLAGDVESNPGP |
| 30 | F2A site | (GSG) VKQTLNFDLLKLAGDVESNPGP |
| 31 | Furin linker | RKRR |
| 32 | Furin linker | RRRR |
| 33 | Furin linker | RRKR |
| 34 | Furin linker | RKKR |
| 35 | Furin linker | R-X-K/R-R |
| 36 | Furin linker | RXKR |
| 37 | Furin linker | RXRR |
| 38 | Ranibizumab | MDIQLTQSPSSLSASVGDRVTITCSASQDISNYLNWYQQKPGKAPKVLIYFTSSL |
| | Fab amino acid | HSGVPSRFSGSGSGTDFTLTISSLQPEDFATYYCQQYSTVPWTFGQGTKVEIKRT |
| | sequence (Light | VAAPSVFIFPPSDEQLKSGTASVVCLLNNFYPREAKVQWKVDNALQSGNSQESVT |
| | chain) | EQDSKDSTYSLSSTLTLSKADYEKHKVYACEVTHQGLSSPVTKSFNRGEC |
| 39 | Ranibizumab | MEVQLVESGGGLVQPGGSLRLSCAASGYDFTHYGMNWVRQAPGKGLEWVGWINTY |
| | Fab amino acid | TGEPTYAADFKRRFTFSLDTSKSTAYLQMNSLRAEDTAVYYCAKYPYYYGTSHWY |
| | sequence | FDVWGQGTLVTVSSASTKGPSVFPLAPSSKSTSGGTAALGCLVKDYFPEPVTVSW |
| | (Heavy chain) | NSGALTSGVHTFPAVLQSSGLYSLSSVVTVPSSSLGTQTYICNVNHKPSNTKVDK |
| | | KVEPKSCDKTHLRKRR |
| 40 | Ranibizumab | MEVQLVESGGGLVQPGGSLRLSCAASGYDFTHYGMNWVRQAPGKGLEWVGWINTY |
| | Fab amino acid | TGEPTYAADFKRRFTFSLDTSKSTAYLQMNSLRAEDTAVYYCAKYPYYYGTSHWY |
| | sequence | FDVWGQGTLVTVSSASTKGPSVFPLAPSSKSTSGGTAALGCLVKDYFPEPVTVSW |
| | (Heavy chain) | NSGALTSGVHTFPAVLQSSGLYSLSSVVTVPSSSLGTQTYICNVNHKPSNTKVDK |
| | | KVEPKSCDKTHL |
| 41 | AAV1 | MAADGYLPDWLEDNLSEGIREWWDLKPGAPKPKANQQKQDDGRGLVLPGYKYLGP |
| | | FNGLDKGEPVNAADAAALEHDKAYDQQLKAGDNPYLRYNHADAEFQERLQEDTSF |
| | | GGNLGRAVFQAKKRVLEPLGLVEEGAKTAPGKKRPVEQSPQEPDSSSGIGKTGQQ |
| | | PAKKRLNFGQTGDSESVPDPQPLGEPPATPAAVGPTTMASGGGAPMADNNEGADG |
| | | VGNASGNWHCDSTWLGDRVITTSTRTWALPTYNNHLYKQISSASTGASNDNHYFG |
| | | YSTPWGYFDFNRFHCHFSPRDWQRLINNNWGFRPKRLNFKLFNIQVKEVTTNDGV |
| | | TTIANNLTSTVQVFSDSEYQLPYVLGSAHQGCLPPFPADVFMIPQYGYLTLNNGS |
| | | QAVGRSSFYCLEYFPSQMLRTGNNFTFSYTFEEVPFHSSYAHSQSLDRLMNPLID |
| | | QYLYYLNRTQNQSGSAQNKDLLFSRGSPAGMSVQPKNWLPGPCYRQQRVSKTKTD |
| | | NNNSNFTWTGASKYNLNGRESIINPGTAMASHKDDEDKFFPMSGVMIFGKESAGA |
| | | SNTALDNVMITDEEEIKATNPVATERFGTVAVNFQSSSTDPATGDVHAMGALPGM |
| | | VWQDRDVYLQGPIWAKIPHTDGHFHPSPLMGGFGLKNPPPQILIKNTPVPANPPA |
| | | EFSATKFASFITQYSTGQVSVEIEWELQKENSKRWNPEVQYTSNYAKSANVDFTV |
| | | DNNGLYTEPRPIGTRYLTRPL |
| 42 | AAV2 | MAADGYLPDWLEDTLSEGIRQWWKLKPGPPPPKPAERHKDDSRGLVLPGYKYLGP |
| | | FNGLDKGEPVNEADAAALEHDKAYDRQLDSGDNPYLKYNHADAEFQERLKEDTSF |
| | | GGNLGRAVFQAKKRVLEPLGLVEEPVKTAPGKKRPVEHSPVEPDSSSGTGKAGQQ |
| | | PARKRLNFGQTGDADSVPDPQPLGQPPAAPSGLGTNTMATGSGAPMADNNEGADG |
| | | VGNSSGNWHCDSTWMGDRVITTSTRTWALPTYNNHLYKQISSQSGASNDNHYFGY |
| | | STPWGYFDFNRFHCHFSPRDWQRLINNNWGFRPKRLNFKLFNIQVKEVTQNDGTT |
| | | TIANNLTSTVQVFTDSEYQLPYVLGSAHQGCLPPFPADVFMVPQYGYLTLNNGSQ |
| | | AVGRSSFYCLEYFPSQMLRTGNNFTFSYTFEDVPFHSSYAHSQSLDRLMNPLIDQ |
| | | YLYYLSRTNTPSGTTTQSRLQFSQAGASDIRDQSRNWLPGPCYRQQRVSKTSADN |
| | | NNSEYSWTGATKYHLNGRDSLVNPGPAMASHKDDEEKFFPQSGVLIFGKQGSEKT |
| | | NVDIEKVMITDEEEIRTTNPVATEQYGSVSTNLQRGNRQAATADVNTQGVLPGMV |
| | | WQDRDVYLQGPIWAKIPHTDGHFHPSPLMGGFGLKHPPPQILIKNTPVPANPSTT |
| | | FSAAKFASFITQYSTGQVSVEIEWELQKENSKRWNPEIQYTSNYNKSVNVDFTVD |
| | | TNGVYSEPRPIGTRYLTRNL |
| 43 | AAV3-3 | MAADGYLPDWLEDNLSEGIREWWALKPGVPQPKANQQHQDNRRGLVLPGYKYLGP |
| | | GNGLDKGEPVNEADAAALEHDKAYDQQLKAGDNPYLKYNHADAEFQERLQEDTSF |
| | | GGNLGRAVFQAKKRILEPLGLVEEAAKTAPGKKGAVDQSPQEPDSSSGVGKSGKQ |
| | | PARKRLNFGQTGDSESVPDPQPLGEPPAAPTSLGSNTMASGGGAPMADNNEGADG |
| | | VGNSSGNWHCDSQWLGDRVITTSTRTWALPTYNNHLYKQISSQSGASNDNHYFGY |
| | | STPWGYFDFNRFHCHFSPRDWQRLINNNWGFRPKKLSFKLFNIQVRGVTQNDGTT |
| | | TIANNLTSTVQVFTDSEYQLPYVLGSAHQGCLPPFPADVFMVPQYGYLTLNNGSQ |
| | | AVGRSSFYCLEYFPSQMLRTGNNFQFSYTFEDVPFHSSYAHSQSLDRLMNPLIDQ |
| | | YLYYLNRTQGTTSGTTNQSRLLFSQAGPQSMSLQARNWLPGPCYRQQRLSKTAND |
| | | NNNSNFPWTAASKYHLNGRDSLVNPGPAMASHKDDEEKFFPMHGNLIFGKEGTTA |
| | | SNAELDNVMITDEEEIRTTNPVATEQYGTVANNLQSSNTAPTTGTVNHQGALPGM |
| | | VWQDRDVYLQGPIWAKIPHTDGHFHPSPLMGGFGLKHPPPQIMIKNTPVPANPPT |
| | | TESPAKFASFITQYSTGQVSVEIEWELQKENSKRWNPEIQYTSNYNKSVNVDFTV |
| | | DTNGVYSEPRPIGTRYLTRNL |
| 44 | AAV4-4 | MTDGYLPDWLEDNLSEGVREWWALQPGAPKPKANQQHQDNARGLVLPGYKYLGPG |
| | | NGLDKGEPVNAADAAALEHDKAYDQQLKAGDNPYLKYNHADAEFQQRLQGDTSFG |
| | | GNLGRAVFQAKKRVLEPLGLVEQAGETAPGKKRPLIESPQQPDSSTGIGKKGKQP |
| | | AKKKLVFEDETGAGDGPPEGSTSGAMSDDSEMRAAAGGAAVEGGQGADGVGNASG |
| | | DWHCDSTWSEGHVTTTSTRTWVLPTYNNHLYKRLGESLQSNTYNGFSTPWGYFDF |
| | | NRFHCHFSPRDWQRLINNNWGMRPKAMRVKIFNIQVKEVTTSNGETTVANNLTST |
| | | VQIFADSSYELPYVMDAGQEGSLPPFPNDVFMVPQYGYCGLVTGNTSQQQTDRNA |
| | | FYCLEYFPSQMLRTGNNFEITYSFEKVPFHSMYAHSQSLDRLMNPLIDQYLWGLQ |
| | | STTTGTTLNAGTATTNETKLRPTNESNEKKNWLPGPSIKQQGFSKTANQNYKIPA |
| | | TGSDSLIKYETHSTLDGRWSALTPGPPMATAGPADSKFSNSQLIFAGPKQNGNTA |
| | | TVPGTLIFTSEEELAATNATDTDMWGNLPGGDQSNSNLPTVDRLTALGAVPGMVW |
| | | QNRDIYYQGPIWAKIPHTDGHFHPSPLIGGFGLKHPPPQIFIKNTPVPANPATTF |
| | | SSTPVNSFITQYSTGQVSVQIDWEIQKERSKRWNPEVQFTSNYGQQNSLLWAPDA |
| | | AGKYTEPRAIGTRYLTHHL |
| 45 | AAV5 | MSFVDHPPDWLEEVGEGLREFLGLEAGPPKPKPNQQHQDQARGLVLPGYNYLGPG |
| | | NGLDRGEPVNRADEVAREHDISYNEQLEAGDNPYLKYNHADAEFQEKLADDTSFG |
| | | GNLGKAVFQAKKRVLEPFGLVEEGAKTAPTGKRIDDHFPKRKKARTEEDSKPSTS |
| | | SDAEAGPSGSQQLQIPAQPASSLGADTMSAGGGGPLGDNNQGADGVGNASGDWHC |
| | | DSTWMGDRVVTKSTRTWVLPSYNNHQYREIKSGSVDGSNANAYFGYSTPWGYFDF |
| | | NRFHSHWSPRDWQRLINNYWGFRPRSLRVKIFNIQVKEVTVQDSTTTIANNLTST |
| | | VQVFTDDDYQLPYVVGNGTEGCLPAFPPQVFTLPQYGYATLNRDNTENPTERSSF |
| | | FCLEYFPSKMLRTGNNFEFTYNFEEVPFHSSFAPSQNLFKLANPLVDQYLYRFVS |
| | | TNNTGGVQFNKNLAGRYANTYKNWFPGPMGRTQGWNLGSGVNRASVSAFATTNRM |
| | | ELEGASYQVPPQPNGMTNNLQGSNTYALENTMIFNSQPANPGTTATYLEGNMLIT |
| | | SESETQPVNRVAYNVGGQMATNNQSSTTAPATGTYNLQEIVPGSVWMERDVYLQG |
| | | PIWAKIPETGAHFHPSPAMGGFGLKHPPPMMLIKNTPVPGNITSFSDVPVSSFIT |
| | | QYSTGQVTVEMEWELKKENSKRWNPEIQYTNNYNDPQFVDFAPDSTGEYRTTRPI |
| | | GTRYLTRPL |
| 46 | AAV6 | MAADGYLPDWLEDNLSEGIREWWDLKPGAPKPKANQQKQDDGRGLVLPGYKYLGP |
| | | FNGLDKGEPVNAADAAALEHDKAYDQQLKAGDNPYLRYNHADAEFQERLQEDTSF |
| | | GGNLGRAVFQAKKRVLEPFGLVEEGAKTAPGKKRPVEQSPQEPDSSSGIGKTGQQ |
| | | PAKKRLNFGQTGDSESVPDPQPLGEPPATPAAVGPTTMASGGGAPMADNNEGADG |
| | | VGNASGNWHCDSTWLGDRVITTSTRTWALPTYNNHLYKQISSASTGASNDNHYFG |
| | | YSTPWGYFDENREHCHFSPRDWQRLINNNWGFRPKRLNEKLENIQVKEVTTNDGV |
| | | TTIANNLTSTVQVFSDSEYQLPYVLGSAHQGCLPPFPADVFMIPQYGYLTLNNGS |
| | | QAVGRSSFYCLEYFPSQMLRTGNNFTFSYTFEDVPFHSSYAHSQSLDRLMNPLID |
| | | QYLYYLNRTQNQSGSAQNKDLLFSRGSPAGMSVQPKNWLPGPCYRQQRVSKTKTD |
| | | NNNSNFTWTGASKYNLNGRESIINPGTAMASHKDDKDKFFPMSGVMIFGKESAGA |
| | | SNTALDNVMITDEEEIKATNPVATERFGTVAVNLQSSSTDPATGDVHVMGALPGM |
| | | VWQDRDVYLQGPIWAKIPHTDGHFHPSPLMGGFGLKHPPPQILIKNTPVPANPPA |
| | | EFSATKFASFITQYSTGQVSVEIEWELQKENSKRWNPEVQYTSNYAKSANVDFTV |
| | | DNNGLYTEPRPIGTRYLTRPL |
| 47 | AAV7 | MAADGYLPDWLEDNLSEGIREWWDLKPGAPKPKANQQKQDNGRGLVLPGYKYLGP |
| | | FNGLDKGEPVNAADAAALEHDKAYDQQLKAGDNPYLRYNHADAEFQERLQEDTSF |
| | | GGNLGRAVFQAKKRVLEPLGLVEEGAKTAPAKKRPVEPSPQRSPDSSTGIGKKGQ |
| | | QPARKRLNFGQTGDSESVPDPQPLGEPPAAPSSVGSGTVAAGGGAPMADNNEGAD |
| | | GVGNASGNWHCDSTWLGDRVITTSTRTWALPTYNNHLYKQISSETAGSTNDNTYF |
| | | GYSTPWGYFDENREHCHFSPRDWQRLINNNWGFRPKKLRFKLFNIQVKEVTTNDG |
| | | VTTIANNLTSTIQVFSDSEYQLPYVLGSAHQGCLPPFPADVFMIPQYGYLTLNNG |
| | | SQSVGRSSFYCLEYFPSQMLRTGNNFEFSYSFEDVPFHSSYAHSQSLDRLMNPLI |
| | | DQYLYYLARTQSNPGGTAGNRELQFYQGGPSTMAEQAKNWLPGPCFRQQRVSKTL |
| | | DQNNNSNFAWTGATKYHLNGRNSLVNPGVAMATHKDDEDRFFPSSGVLIFGKTGA |
| | | TNKTTLENVLMTNEEEIRPTNPVATEEYGIVSSNLQAANTAAQTQVVNNQGALPG |
| | | MVWQNRDVYLQGPIWAKIPHTDGNFHPSPLMGGFGLKHPPPQILIKNTPVPANPP |
| | | EVFTPAKFASFITQYSTGQVSVEIEWELQKENSKRWNPEIQYTSNFEKQTGVDFA |
| | | VDSQGVYSEPRPIGTRYLTRNL |
| 48 | AAV8 | MAADGYLPDWLEDNLSEGIREWWALKPGAPKPKANQQKQDDGRGLVLPGYKYLGP |
| | | FNGLDKGEPVNAADAAALEHDKAYDQQLQAGDNPYLRYNHADAEFQERLQEDTSF |
| | | GGNLGRAVFQAKKRVLEPLGLVEEGAKTAPGKKRPVEPSPQRSPDSSTGIGKKGQ |
| | | QPARKRLNFGQTGDSESVPDPQPLGEPPAAPSGVGPNTMAAGGGAPMADNNEGAD |
| | | GVGSSSGNWHCDSTWLGDRVITTSTRTWALPTYNNHLYKQISNGTSGGATNDNTY |
| | | EGYSTPWGYFDENREHCHFSPRDWQRLINNNWGFRPKRLSFKLFNIQVKEVTQNE |
| | | GTKTIANNLTSTIQVFTDSEYQLPYVLGSAHQGCLPPFPADVFMIPQYGYLTLNN |
| | | GSQAVGRSSFYCLEYFPSQMLRTGNNFQFTYTFEDVPFHSSYAHSQSLDRLMNPL |
| | | IDQYLYYLSRTQTTGGTANTQTLGFSQGGPNTMANQAKNWLPGPCYRQQRVSTTT |
| | | GQNNNSNFAWTAGTKYHLNGRNSLANPGIAMATHKDDEERFFPSNGILIFGKQNA |
| | | ARDNADYSDVMLTSEEEIKTTNPVATEEYGIVADNLQQQNTAPQIGTVNSQGALP |
| | | GMVWQNRDVYLQGPIWAKIPHTDGNFHPSPLMGGFGLKHPPPQILIKNTPVPADP |
| | | PTTFNQSKLNSFITQYSTGQVSVEIEWELQKENSKRWNPEIQYTSNYYKSTSVDF |
| | | AVNTEGVYSEPRPIGTRYLTRNL |
| 49 | hu31 | MAADGYLPDWLEDTLSEGIRQWWKLKPGPPPPKPAERHKDDSRGLVLPGYKYLGP |
| | | GNGLDKGEPVNAADAAALEHDKAYDQQLKAGDNPYLKYNHADAEFQERLKEDTSF |
| | | GGNLGRAVFQAKKRLLEPLGLVEEAAKTAPGKKRPVEQSPQEPDSSAGIGKSGSQ |
| | | PAKKKLNFGQTGDTESVPDPQPIGEPPAAPSGVGSLTMASGGGAPVADNNEGADG |
| | | VGSSSGNWHCDSQWLGDRVITTSTRTWALPTYNNHLYKQISNSTSGGSSNDNAYF |
| | | GYSTPWGYFDENREHCHFSPRDWQRLINNNWGFRPKRLNEKLENIQVKEVTDNNG |
| | | VKTIANNLTSTVQVFTDSDYQLPYVLGSAHEGCLPPFPADVFMIPQYGYLTLNDG |
| | | GQAVGRSSFYCLEYFPSQMLRTGNNFQFSYEFENVPFHSSYAHSQSLDRLMNPLI |
| | | DQYLYYLSKTINGSGQNQQTLKFSVAGPSNMAVQGRNYIPGPSYRQQRVSTTVTQ |
| | | NNNSEFAWPGASSWALNGRNSLMNPGPAMASHKEGEDRFFPLSGSLIFGKQGTGR |
| | | DNVDADKVMITNEEEIKTTNPVATESYGQVATNHQSAQAQAQTGWVQNQGILPGM |
| | | VWQDRDVYLQGPIWAKIPHTDGNFHPSPLMGGFGMKHPPPQILIKNTPVPADPPT |
| | | AFNKDKLNSFITQYSTGQVSVEIEWELQKENSKRWNPEIQYTSNYYKSNNVEFAV |
| | | STEGVYSEPRPIGTRYLTRNL |
| 50 | hu32 | MAADGYLPDWLEDTLSEGIRQWWKLKPGPPPPKPAERHKDDSRGLVLPGYKYLGP |
| | | GNGLDKGEPVNAADAAALEHDKAYDQQLKAGDNPYLKYNHADAEFQERLKEDTSF |
| | | GGNLGRAVFQAKKRLLEPLGLVEEAAKTAPGKKRPVEQSPQEPDSSAGIGKSGSQ |
| | | PAKKKLNFGQTGDTESVPDPQPIGEPPAAPSGVGSLTMASGGGAPVADNNEGADG |
| | | VGSSSGNWHCDSQWLGDRVITTSTRTWALPTYNNHLYKQISNSTSGGSSNDNAYF |
| | | GYSTPWGYFDENREHCHFSPRDWQRLINNNWGFRPKRLNEKLENIQVKEVTDNNG |
| | | VKTIANNLTSTVQVFTDSDYQLPYVLGSAHEGCLPPFPADVFMIPQYGYLTLNDG |
| | | SQAVGRSSFYCLEYFPSQMLRTGNNFQFSYEFENVPFHSSYAHSQSLDRLMNPLI |
| | | DQYLYYLSKTINGSGQNQQTLKFSVAGPSNMAVQGRNYIPGPSYRQQRVSTTVTQ |
| | | NNNSEFAWPGASSWALNGRNSLMNPGPAMASHKEGEDRFFPLSGSLIFGKQGTGR |
| | | DNVDADKVMITNEEEIKTTNPVATESYGQVATNHQSAQAQAQTGWVQNQGILPGM |
| | | VWQDRDVYLQGPIWAKIPHTDGNFHPSPLMGGFGMKHPPPQILIKNTPVPADPPT |
| | | AFNKDKLNSFITQYSTGQVSVEIEWELQKENSKRWNPEIQYTSNYYKSNNVEFAV |
| | | NTEGVYSEPRPIGTRYLTRNL |
| 51 | AAV9 | MAADGYLPDWLEDNLSEGIREWWALKPGAPQPKANQQHQDNARGLVLPGYKYLGP |
| | | GNGLDKGEPVNAADAAALEHDKAYDQQLKAGDNPYLKYNHADAEFQERLKEDTSF |
| | | GGNLGRAVFQAKKRLLEPLGLVEEAAKTAPGKKRPVEQSPQEPDSSAGIGKSGAQ |
| | | PAKKRLNFGQTGDTESVPDPQPIGEPPAAPSGVGSLTMASGGGAPVADNNEGADG |
| | | VGSSSGNWHCDSQWLGDRVITTSTRTWALPTYNNHLYKQISNSTSGGSSNDNAYF |
| | | GYSTPWGYFDENREHCHFSPRDWQRLINNNWGFRPKRLNEKLENIQVKEVTDNNG |
| | | VKTIANNLTSTVQVFTDSDYQLPYVLGSAHEGCLPPFPADVFMIPQYGYLTLNDG |
| | | SQAVGRSSFYCLEYFPSQMLRTGNNFQFSYEFENVPFHSSYAHSQSLDRLMNPLI |
| | | DQYLYYLSKTINGSGQNQQTLKFSVAGPSNMAVQGRNYIPGPSYRQQRVSTTVTQ |
| | | NNNSEFAWPGASSWALNGRNSLMNPGPAMASHKEGEDRFFPLSGSLIFGKQGTGR |
| | | DNVDADKVMITNEEEIKTTNPVATESYGQVATNHQSAQAQAQTGWVQNQGILPGM |
| | | VWQDRDVYLQGPIWAKIPHTDGNFHPSPLMGGFGMKHPPPQILIKNTPVPADPPT |
| | | AFNKDKLNSFITQYSTGQVSVEIEWELQKENSKRWNPEIQYTSNYYKSNNVEFAV |
| | | NTEGVYSEPRPIGTRYLTRNL |

### 6. EXAMPLES

### 6.1 Example 1: Bevacizumab Fab cDNA-Based Vector

A bevacizumab Fab cDNA-based vector is constructed comprising a transgene comprising bevacizumab Fab portion of the light and heavy chain cDNA sequences (SEQ ID NOs. 10 and 11, respectively). The transgene also comprises nucleic acids comprising a signal peptide chosen from the group listed in Table 1. The nucleotide sequences encoding the light chain and heavy chain are separated by IRES elements or 2A cleavage sites to create a bicistronic vector. Optionally, the vector additionally comprises a hypoxia-inducible promoter.

### 6.2 Example 2: Ranibizumab cDNA-Based Vector

A ranibizumab Fab cDNA-based vector is constructed comprising a transgene comprising ranibizumab Fab light and heavy chain cDNAs (the portions of SEQ ID NOs.12 and 13, respectively not encoding the signal peptide). The transgene also comprises nucleic acids comprising a signal peptide chosen from the group listed in Table 1. The nucleotide sequences encoding the light chain and heavy chain are separated by IRES elements or 2A cleavage sites to create a bicistronic vector. Optionally, the vector additionally comprises a hypoxia-inducible promoter.

### 6.3 Example 3: Hyperglycosylated Bevacizumab Fab cDNA-Based Vector

A hyperglycosylated bevacizumab Fab cDNA-based vector is constructed comprising a transgene comprising bevacizumab Fab portion of the light and heavy chain cDNA sequences (SEQ ID NOs. 10 and 11, respectively) with mutations to the sequence encoding one or more of the following mutations: L118N (heavy chain), E195N (light chain), or Q160N or Q1605 (light chain). The transgene also comprises nucleic acids comprising a signal peptide chosen from the group listed in Table 1. The nucleotide sequences encoding the light chain and heavy chain are separated by IRES elements or 2A cleavage sites to create a bicistronic vector. Optionally, the vector additionally comprises a hypoxia-inducible promoter.

### 6.4 Example 4: Hyperglycosylated Ranibizumab cDNA-Based Vector

A hyperglycosylated ranibizumab Fab cDNA-based vector is constructed comprising a transgene comprising ranibizumab Fab light and heavy chain cDNAs (the portions of SEQ ID NOs.12 and 13, respectively not encoding the signal peptide), with mutations to the sequence encoding one or more of the following mutations: L118N (heavy chain), E195N (light chain), or Q160N or Q1605 (light chain). The transgene also comprises nucleic acids comprising a signal peptide chosen from the group listed in Table 1. The nucleotide sequences encoding the light chain and heavy chain are separated by IRES elements or 2A cleavage sites to create a bicistronic vector. Optionally, the vector additionally comprises a hypoxia-inducible promoter.

### 6.5 Example 5: Ranibizumab Based HuGlyFabVEGFi

A ranibizumab Fab cDNA-based vector (see Example 2) is expressed in the PER.C6® Cell Line (Lonza) in the AAV8 background. The resultant product, ranibizumab-based HuGlyFabVEGFi is determined to be stably produced. N-glycosylation of the HuGlyFabVEGFi is confirmed by hydrazinolysis and MS/MS analysis. See, e.g., Bondt et al., Mol. & Cell. Proteomics 13.11:3029-3039. Based on glycan analysis, HuGlyFabVEGFi is confirmed to be N-glycosylated, with 2,6 sialic acid a predominant modification. Advantageous properties of the N-glycosylated HuGlyFabVEGFi are determined using methods known in the art. The HuGlyFabVEGFi can be found to have increased stability and increased affinity for its antigen (VEGF). See Sola and Griebenow, 2009, J Pharm Sci., 98(4): 1223-1245 for methods of assessing stability and Wright et al., 1991, EMBO J. 10:2717-2723 and Leibiger et al., 1999, Biochem. J. 338:529-538 for methods of assessing affinity.

### 6.6 Example 6: An Open-label Phase 2a Dose Assessment of Construct II Gene Therapy in Participants with Diabetic Retinopathy

This example provides an overview of a phase 2a, dose assessment of Construct II gene therapy in participants with diabetic retinopathy (DR). The sustained, stable expression of the Construct II transgene product following a 1-time gene therapy treatment for DR could potentially reduce the treatment burden of currently available therapies while maintaining vision with a favorable benefit:risk profile. The current proof of concept study is intended to evaluate the safety and efficacy of Construct II gene therapy at 2 different dose levels in participants with DR.

### 6.6.1 Objectives and Endpoints

TABLE 4
Primary and Secondary Objectives and Endpoints

| | |
|---|---|
| Objectives | Endpoints |

Primary

| | | |
|---|---|---|
| Efficacy | To evaluate the effect of | Proportion of participants achieving a 2-step |
| | Construct II on the | or greater improvement in ETDRS-DRSS on |
| | ETDRS-DRSS at | 4-widefield digital stereoscopic fundus |
| | Week 24 | photography at Week 24 |

Secondary

| | | |
|---|---|---|
| Efficacy | To evaluate the effect of | Proportion of participants achieving a 2-step |
| | Construct II on the | or greater improvement in ETDRS-DRSS on |
| | ETDRS-DRSS at | 4-widefield digital stereoscopic fundus |
| | additional time points | photography at Week 12 |
| | | Proportion of participants achieving a 0-step |
| | | (n change), 1-step, 2-step, or 3-step |
| | | improvement in ETDRS-DRSS on |
| | | 4-widefield digital stereoscopic fundus |
| | | photography at Week 12 and Week 24 |
| | | Proportion of participants achieving a 1-step |
| | | or greater, or a 3-step or greater improvement |
| | | in ETDRS-DRSS on 4-widefield digital |
| | | stereoscopic fundus photography at Week 12 |
| | | and Week 24 |
| | | Proportion of participants with a 1-step or |
| | | greater, a 2-step or greater, or a 3-step or |
| | | greater worsening in ETDRS-DRSS on |
| | | 4-widefield digital stereoscopic fundus |
| | | photography at Week 12 and Week 24 |
| | | Proportion of participants graded as Level 61 |
| | | or 65 (PDR) at baseline achieving regression |
| | | to Level 47 or 53 (NPDR) |
| Safety/Immunogenicity | To assess the safety, | Proportion of participants with cataracts |
| | tolerability, and | meeting the protocol-specified criteria for |
| | immunogenicity of | removal at either Week 18 or Week 24, or at |
| | Construct II | an unscheduled visit prior to Week 18 |
| | | Incidences of ocular and systemic AEs |
| | | Immunogenicity measurements (serum |
| | | neutralizing antibodies to AAV8 and serum |
| | | antibodies to Construct II TP) over 24 |
| | | weeks |
| Safety/Efficacy | To evaluate the | Proportion of participants requiring any |
| | need for additional | additional intervention for diabetic |
| | SOC intervention | complications to Week 24 |
| | due to diabetic | Proportion of participants with any sight- |
| | complications | threatening diabetes complications to Week 24 |
| | | Proportion of participants developing diabetic |
| | | complications (eg, CI-DME or |
| | | neovascularization) requiring anti-VEGF |
| | | treatment per SOC through Week 24; for this |
| | | population, the following endpoints will be |
| | | evaluated: |
| | | Number of anti-VEGF injections received |
| | | up to Week 24 |
| | | Duration of time from study intervention |
| | | (Day 1) to first anti-VEGF administration |
| | | per SOC |
| | | Proportion of participants developing diabetic |
| | | complications (eg, neovascularization due to |
| | | DR) requiring PRP per SOC through |
| | | Week 24; for this population, the following |
| | | endpoints will be evaluated: |
| | | Duration of time from study intervention |
| | | (Day 1) to first PRP |
| | | Proportion of participants requiring more |
| | | than 1 PRP |
| | | Proportion of participants developing diabetic |
| | | complications (eg, retinal detachment) |
| | | requiring surgical intervention (pneumatic |
| | | retinopexy, cryopexy, or scleral buckle) per |
| | | SOC; for this population, the following |
| | | endpoint will be evaluated: |
| | | Duration of time from study intervention |
| | | (Day 1) to surgical intervention |
| Pharmacodynamics | To measure | Aqueous Construct II TP concentrations at |
| | aqueous Construct | Week 4, Week 12, and Week 24 |
| | II TP concentrations | |

Exploratory

| | | |
|---|---|---|
| Efficacy/Safety | To evaluate the | Proportion of participants with visual stability |
| | effect of Construct | (within 5 ETDRS letters or ±5 ETDRS |
| | II on vision | letters) from baseline to Week 24 |
| | outcomes in all | Proportion of participants with vision gain |
| | evaluable | or vision loss >5 ETDRS letters from |
| | participants | baseline to Week 24 |
| | To evaluate the | Mean change in CST on SD-OCT at Week 12 |
| | effect of Construct | and Week 24 |
| | II on anatomic | Proportion of participants achieving ≤250 μm |
| | outcomes evaluated | CST on SD-OCT at Week 12 and Week 24 |
| | using SD-OCT in | Proportion of participants with clinically |
| | all evaluable | significant macular thickening in CST ≥30 |
| | participants | μm from baseline at Week 12 and Week 24 |
| | | Mean change in macular volume and |
| | | percent reduction in macular volume from |
| | | baseline based on SD-OCT, as determined |
| | | by the CRC |
| | To assess evidence | Proportion of participants graded as Level |
| | of vessel regression | 61 or 65 at baseline with evidence of vessel |
| | for participants with | regression at Week 24 based on FA |
| | baseline PDR | |
| | (Level 61 or 65) | |
| | To assess changes | Proportion of participants graded as Level |
| | in the area of | 61 or 65 at baseline with change in area of |
| | leakage for | leakage from baseline to Week 24 based on |
| | participants with | FA |
| | baseline PDR | |
| | (Level 61 or 65) | |
| | To assess changes | Mean change from baseline in the area of |
| | from baseline in the | retinal nonperfusion at Week 24 based on |
| | area of retinal | FA in all evaluable participants |
| | nonperfusion in all | |
| | evaluable | |
| | participants | |
| Biomarkers | To measure | VEGF-A concentration in aqueous fluid at |
| | aqueous VEGF-A | assessed time points |
| | concentration | |

AAV8 = adeno-associated virus serotype 8;
AE = adverse event;
CI-DME = center involved-diabetic macular edema;
CRC = central reading center;
CST = central subfield thickness;
DR = diabetic retinopathy;
DRSS = Diabetic Retinopathy Severity Scale;
ETDRS = Early Treatment Diabetic Retinopathy Study;
FA = fluorescein angiography;
PDR = proliferative diabetic retinopathy;
PRP = panretinal photocoagulation;
SD-OCT = spectral domain-optical coherence tomography;
SOC = standard of care;
TP = transgene product;
VEGF = vascular endothelial growth factor

### 6.6.2 Inclusion Criteria

Participants must meet all the following criteria in order to be eligible for this study. All ocular criteria refer to the study eye: (1) men or women ≥18 years of age with DR secondary to diabetes mellitus Type 1 or 2. Participants must have a hemoglobin A1c≤10% (as confirmed by laboratory assessments obtained at Screening or by a documented laboratory report dated within 60 days prior to Screening); (2) participant deemed to be an appropriate surgical candidate, per the investigator; (3) study eye with moderately-severe NPDR, severe NPDR, mild PDR, or moderate PDR (ETDRS-DRSS Levels 47, 53, 61, or 65 using standard 4-widefield digital stereoscopic fundus photographs, as determined by the CRC) for which PRP or anti-VEGF injections can be safely deferred, in the opinion of the investigator, for at least 6 months after Screening; (4) no evidence in the study eye of high-risk characteristics typically associated with vision loss, per the investigator, including the following: (i) new vessels within 1-disc area of the optic nerve, or vitreous or preretinal hemorrhage associated with less extensive new vessels at the optic disc, or with new vessels elsewhere that are half a disc area or more in size, and (ii) no evidence in the study eye of anterior segment (eg, iris or angle) neovascularization on clinical examination; (5) best-corrected visual acuity (BCVA) in the study eye of >69 ETDRS letters (approximate Snellen equivalent 20/40 or better); note: if both eyes are eligible, the study eye must be the participant's worse-seeing eye, as determined by the investigator prior to enrollment; (6) prior history of CI-DME in the study eye is acceptable if no intravitreal anti-VEGF or short-acting steroid injections have been given within the last 6 months, AND no more than 10 documented injections have been given in the 3 years prior to Screening; (7) women must be postmenopausal ≥1 year or surgically sterilized. If not, women must have a negative serum pregnancy test at Screening, have negative confirmatory urine; pregnancy test results at Day 1 (Construct II surgery day), and be willing to have additional pregnancy tests during the study; (8) women of childbearing potential, their male partners, and sexually active male participants with female partners of childbearing potential must be willing to use a highly effective method of contraception from Screening until 24 weeks after vector administration. Cessation of birth control after this point must be discussed with a responsible physician; (9) must be willing and able to comply with all study procedures and be available for the duration of the study; (10) must be willing and able to provide written, signed informed consent.

### 6.6.3 Exclusion Criteria

Participants are excluded from the study if any of the following criteria apply: (1) presence of any active CI-DME, as determined by the investigator, on clinical examination or within the center subfield of the study eye using the following threshold: Heidelberg Spectralis: 320 μm; (2) neovascularization in the study eye from a cause other than DR, per investigator; (3) evidence in the study eye, as determined by the investigator, of ischemia in the study eye involving >50% of the peripheral retina, or the fovea or papillomacular area on baseline FA; (4) evidence in the study eye of optic nerve pallor on clinical exam or optic disc neovascularization on baseline FA, as determined by investigator; (5) any evidence of or documented history of PRP in the study eye, or any evidence of focal or grid laser outside the posterior pole in the study eye; (6) ocular or periocular infection in the study eye that may interfere with the surgical procedure; (7) any ocular condition in the study eye that could require surgical intervention within the 6 months after Screening (vitreous hemorrhage, cataract that does not meet the inclusion criteria, retinal traction, epiretinal membrane, etc) or any condition in the study eye that may, in the opinion of the investigator, increase the risk to the participant, require either medical or surgical intervention during the study to prevent or treat vision loss, or interfere with the study procedures or assessments; (8) active or history of retinal detachment in the study eye; (9) presence of an implant in the study eye at Screening (excluding intraocular lens [IOL]); (10) pentacam Nuclear Staging score >1 as scanned by the Pentacam device and verified by the CRC, or not meeting other baseline cataract criteria as outlined in Section 6.6.5(c); (11) documented existing cortical or posterior subcapsular cataract on either clinical examination by investigator, or lens imaging as determined by the CRC, and/or having a nuclear lens image grade above AREDS level 2 (mild nuclear opacities), as determined by the CRC; (12) advanced glaucoma in the study eye (ie, uncontrolled, despite 2 or more drop treatments or an intervention such as a tube or shunt), as assessed through consultation with the participant's glaucoma specialist or documented history of glaucoma surgery; (13) history of intraocular surgery in the study eye within 12 weeks prior to Screening; yttrium aluminum garnet capsulotomy is permitted if performed >10 weeks prior to Screening; (14) history of intravitreal therapy in the study eye, including anti-VEGF therapy, within 6 months prior to Screening, and documentation of more than 10 prior anti-VEGF or short-acting steroid intravitreal injections in the study eye for DME within 3 years of Screening; (15) any prior intravitreal steroid injection in the study eye within 6 months prior to Screening, administration in the study eye of Ozurdex® within 12 months prior to Screening, or administration in the study eye of Iluvien® within 36 months prior to Screening; (16) any prior systemic anti-VEGF treatment within the 6 months prior to or plans to use systemic anti-VEGF therapy during the next 6 months after Screening; (17) history of therapy known to have caused retinal toxicity, or concomitant therapy with any drug that may affect VA or with known retinal toxicity, e.g., chloroquine or hydroxychloroquine; (18) myocardial infarction, cerebrovascular accident, or transient ischemic attacks within the 6 months prior to Screening; (19) uncontrolled hypertension (systolic blood pressure [BP] >180 mmHg, diastolic BP >100 mmHg) despite maximal medical treatment; note that if BP is brought below 180/100 mmHg and stabilized by antihypertensive treatment as determined by the investigator and/or primary care physician, the participant can be rescreened for eligibility; (20) a systemic condition that, in the opinion of the investigator, would preclude participation in the study (poor glycemic control, uncontrolled hypertension, etc); (21) any concomitant treatment that, in the opinion of the investigator, may interfere with the ocular surgical procedure or the healing process; (22) history of malignancy or hematologic malignancy that may compromise the immune system requiring chemotherapy and/or radiation in the 5 years prior to Screening. Localized basal cell carcinoma will be permitted; (23) has a serious, chronic, or unstable medical or psychological condition that, in the opinion of the investigator, may compromise the participant's safety or ability to complete all assessments and follow-up in the study; (24) any participant with the following laboratory values at Screening will be withdrawn from the study: (i) aspartate aminotransferase (AST)/alanine aminotransferase (ALT) >2.5×upper limit of normal (ULN), (ii) total bilirubin >1.5×ULN, unless the participant has a previously known history of Gilbert's syndrome and a fractionated bilirubin that shows conjugated bilirubin <35% of total bilirubin, (iii) prothrombin time >1.5×ULN, unless the participant is anticoagulated. Participants who are anticoagulated will be monitored by local labs and managed per local practice to hold or bridge anticoagulant therapy for the study procedure; consultation with the Medical Monitor is required if the participant is anticoagulated, (iv) hemoglobin <10 g/dL for male participants and <9 g/dL for female participants, (v) Platelets <100×103/μL, (vi) estimated glomerular filtration rate <30 mL/min/1.73 m²; (25) history of chronic renal failure requiring dialysis or kidney transplant; (26) initiation of intensive insulin treatment (pump or multiple daily injections) within the 6 months prior to Screening or plans to do so within 6 months of Screening; (27) currently taking anticoagulation therapy for which holding anticoagulation therapy for Construct II administration is not indicated or considered to be unsafe in the opinion of the treating investigator (ie, retinal surgeon), as well as the physician prescribing anticoagulation for the participant, as verified by the Medical Monitor; (28) participation in any other gene therapy study, including Construct II, or receipt of any investigational product within 30 days prior to enrollment or 5 half-lives of the investigational product, whichever is longer, or any plans to use an investigational product within 6 months following enrollment; (29) known hypersensitivity to ranibizumab or any of its components.

### 6.6.4 Study Intervention

Study intervention is defined as any investigational intervention(s), marketed product(s), placebo, or medical device(s) intended to be administered to a study participant according to the study protocol.

Eligible participants will be assigned to receive a single dose of either Construct II (Dose 1) or a single dose of Construct II (Dose 2). All participants will receive study intervention on Day 1 via subretinal delivery in an operating room.

TABLE 5
Summary of Study Intervention(s)

| Arm Name | Construct II Dose 1 | Construct II Dose 2 |
|---|---|---|
| Type | Gene therapy (AAV8.CB7.CI.amd42.rBG) | |
| Dose Formulation | Solution | |
| Unit Dose | 6.2 × 10¹¹ GC/mL | 1.0 × 10¹² GC/mL |
| Strength | | |
| Dosage Level(s) | 250 μL | 250 μL |
| | (1.6 × 10¹¹ GC/eye) | (2.5 × 10¹¹ GC/eye) |
| | one-time dose | one-time dose |

| | |
|---|---|
| Route of | Subretinal delivery |
| Administration | |
| Physical | Construct II investigational product is supplied as a frozen, sterile, |
| Description | single- use solution of the AAV vector active ingredient |
| | (AAV8.CB7.CI.amd42.rBG) in a formulation buffer. The vector is |
| | formulated in Dulbecco's phosphate buffered saline and 0.001% |
| | Pluronic F68, pH = 7.4. The solution appears clear to opalescent, |
| | colorless, and free of visible particulates at room temperature. |
| Packaging and | Study intervention will be supplied as a sterile, single-use solution in |
| Labeling | 2-mL Crystal Zenith ® vials sealed with latex free rubber stoppers and |
| | aluminum flip-off seals. Each vial will be labeled as required per |
| | applicable regulatory requirements. |

Participants in this study will be randomized (1:1) at Screening using an interactive response technology system to receive Construct II (Dose 1) or Construct II (Dose 2).

### 6.6.5 Prior and Concomitant Therapy

(a) Medications and Therapies

The following medications are prohibited prior to entry into the study:

Any prior systemic or ocular anti-VEGF treatment in the study eye within the 6 months prior to Screening.

More than 10 prior, documented, anti-VEGF or short-acting steroid intravitreal injections in the study eye for DME within 3 years of Screening.

Any prior intravitreal short-acting steroid injection in the study eye within 6 months prior to Screening, administration in the study eye of Ozurdex within 12 months prior to Screening, or administration in the study eye of Iluvien within 36 months prior to Screening.

Initiation of intensive insulin treatment (pump or multiple daily injections) within the 6 months prior to Screening; for participants meeting this criterion, modification of the regimen is permitted during the study, as recommended and documented by their primary care provider or other treatment provider.

Participants must not have used any concomitant treatment that, in the opinion of the investigator, could interfere with Construct II administration or the healing process.

Participants are prohibited from taking anticoagulation therapy for which holding anticoagulation therapy for Construct II administration is not indicated or considered to be unsafe in the opinion of the treating investigator (ie, retinal surgeon), as well as the physician prescribing anticoagulation for the participant.

Participants must not have used any investigational product within 30 days prior to enrollment or within 5 half-lives of the investigational product, whichever is longer.

The following concomitant medications are prohibited during the study:

Anti-VEGF therapy in the study eye during the 6 months after Screening, except in the situations described in Section 6.6.5(b) for treatment of ocular diabetes complications.

Initiation of intensive insulin treatment (pump or multiple daily injections) is not allowed during the study; as indicated previously, modification of the treatment regimen is allowed during the study if initiation of treatment occurred at least 6 months prior to Screening.

Postoperative care for participants receiving Construct II is described in the Procedures Manual. There are no other restrictions on prior or concomitant therapy in this study.

(b) Treatment of Ocular Diabetes Complications

All complications of ocular diabetes will be managed in accordance with each study centers SOC.

During the study, participants who develop diabetic complications requiring anti-VEGF treatment per SOC may be administered therapy as required. If needed, the study centers will provide their own supply of FDA-approved anti-VEGF therapy. Development of CI-DME must be recorded as an AE and the number of anti-VEGF injections received, and the timing of all administrations, must also be recorded in the source documents and eCRF.

Participants who develop diabetic complications requiring PRP SOC must have the time of PRP recorded in the source documents and eCRF.

Participants who develop diabetic complications requiring surgical intervention SOC (either pneumatic retinopexy, cryopexy, or scleral buckle) must have the type of intervention and the time of intervention recorded in the source documents and eCRF.

(c) Intervention for Cataract Formation

Screening

During the Screening visit, a series of assessments will be completed to determine eligibility and establish the participant's baseline cataract status. These assessments include the following: (1) assessing the participant's symptoms per SOC; (2) performing a clinical examination to determine whether any signs of cortical cataract or posterior subcapsular cataract are present; (3) imaging with the Oculus Pentacam Nuclear Staging system; and (4) imaging the participant's lens with standardized anterior segment photographs, which will be submitted to the CRC for grading and confirmation of study eligibility. Participants with cataracts at the Screening visit who meet Exclusion Criterion #11 must not be enrolled.

On-study Cataract Evaluation and Intervention

During the study, the cataract surgeon will continue to assess participants for the presence of cataracts meeting the criteria for removal specified below.

The criterion for medically indicated cataract extraction, which is to be reported as an AE, is as follows: the retina investigator is unable to adequately view and/or image the retina in order to safely monitor and manage diabetic eye disease and/or general retinal status.

If the criterion for medically indicated cataract extraction is met at any postbaseline visit, an unscheduled visit for cataract extraction surgery will be scheduled within 5 business days by the study coordinator with the cataract surgeon.

If the criterion for medically indicated cataract extraction is not met, but the participant meets 2 or more of the following secondary criteria at any postbaseline visit, the study coordinator will schedule the participant for an unscheduled visit for cataract extraction surgery to be performed within 10 business days by the cataract surgeon. The secondary criteria, which are also to be reported as AEs, are as follows:

Vision change: A decrease in BCVA of ≥5 ETDRS letters, relative to the best value recorded during the study (baseline or postbaseline), that is also associated, per the cataract surgeon, with changes from baseline in the lens.

Refractive shift: A change in refractive error ≥1 diopter during BCVA recorded at any study visit (relative to the refractive error at baseline) that is also associated, per the cataract surgeon, with changes from baseline in the lens.

Structural: A change of >1 grade from baseline on the Pentacam Nuclear Staging score, reflecting increased opacification within the lens from baseline.

Participant-reported: Visual function change from baseline as reported by the participant.

CRC Imaging: A change of >1 grade/subfield from baseline on the nuclear, cortical, or posterior subcapsular scales (AREDS cataract scale [see the Procedures Manual for details]), as determined by the CRC.

A monofocal, 1-piece acrylic IOL is the lens of choice for use in this study. In some instances, a toric (astigmatism-correcting) IOL could be considered, but any difference in cost between a monofocal IOL and a toric lens is the responsibility of the participant unless otherwise approved by the Sponsor and the Medical Monitor. Multifocal or other premium IOLs are excluded during the study, as they may diminish the ability to accurately track any changes in retinal pathology. Silicone optic IOLs will not be used because of their potential to complicate any subsequent retinal procedures. The cataract surgeon may provide the participant with a recommendation that is most likely to provide optimal postoperative VA and visual function.

A postoperative, SOC protocol intended to limit complications will be followed. The preferred SOC protocol includes: fluroquinolone drops 4-times daily for 1 week, Ilevro (nepafenac) 2-times daily for 1 month, and a steroid taper with prednisolone acetate starting with 4-times daily for 1 week, tapering down 1 week at a time to 3-times daily, 2-times daily, and, finally, 1-time daily. For participant safety, alternative postoperative protocols may be used where appropriate, and with approval by the Medical Monitor.

### 6.7 Example 7: An Open-Label Phase 2a Dose Assessment of Construct II Gene Therapy in Participants with Diabetic Retinopathy

This example is an updated version of Example 6 and provides an overview of a phase 2a, dose assessment of Construct II gene therapy in participants with diabetic retinopathy (DR). The sustained, stable expression of the Construct II transgene product following a one-time gene therapy treatment for DR could potentially reduce the treatment burden of currently available therapies while maintaining vision with a favorable benefit:risk profile. The current proof of concept study is intended to evaluate the safety and efficacy of Construct II gene therapy at 2 different dose levels in participants with DR.

### 6.7.1 Objectives and Endpoints

TABLE 6
Primary and Secondary Objectives and Endpoints

| | |
|---|---|
| Objectives | Endpoints |

Primary

| | | |
|---|---|---|
| Efficacy | To evaluate the effect of | Proportion of participants achieving a 2-step or |
| | Construct II on the | greater improvement in ETDRS-DRSS on 4- |
| | ETDRS-DRSS at | widefield digital stereoscopic fundus |
| | Week 24 | photography at Week 24 |

Secondary

| | | |
|---|---|---|
| Efficacy | To evaluate the effect of | Proportion of participants achieving a 2-step or |
| | Construct II on the | greater improvement in ETDRS-DRSS on 4- |
| | ETDRS-DRSS at | widefield digital stereoscopic fundus |
| | additional time points | photography at Week 12 |
| | | Proportion of participants achieving a 0-step |
| | | (no change), 1-step, 2-step, or 3-step |
| | | improvement in ETDRS-DRSS on |
| | | 4-widefield digital stereoscopic fundus |
| | | photography at Week 12 and Week 24 |
| | | Proportion of participants achieving a 1-step or |
| | | greater, or a 3-step or greater improvement in |
| | | ETDRS-DRSS on 4-widefield digital |
| | | stereoscopic fundus photography at Week 12 |
| | | and Week 24 |
| | | Proportion of participants with a 1-step or |
| | | greater, a 2-step or greater, or a 3-step or |
| | | greater worsening in ETDRS-DRSS on |
| | | 4-widefield digital stereoscopic fundus |
| | | photography at Week 12 and Week 24 |
| | | Proportion of participants graded as Level 61 or |
| | | 65 (PDR) at baseline achieving regression to |
| | | Level 47 or 53 (NPDR) |
| Safety/Immunogenicity | To assess the safety, | Proportion of phakic participants with |
| | tolerability, and | cataracts meeting the protocol-specified |
| | immunogenicity of | criteria for removal at either Week 18 or Week |
| | Construct II | 24, or at an unscheduled visit prior to Week 18 |
| | | Incidences of ocular and systemic AEs |
| | | Immunogenicity measurements (serum |
| | | neutralizing antibodies to AAV8 and serum |
| | | antibodies to Construct II TP) over 24 weeks |
| Safety/Efficacy | To evaluate the need | Proportion of participants requiring any |
| | for additional SOC | additional intervention for diabetic |
| | intervention due to | complications to Week 24 |
| | diabetic complications | Proportion of participants with any sight- |
| | | threatening diabetes complications to Week 24 |
| | | Proportion of participants developing diabetic |
| | | complications (eg, CI-DME or |
| | | neovascularization) requiring anti-VEGF |
| | | treatment per SOC through Week 24; for this |
| | | population, the following endpoints will be |
| | | evaluated: |
| | | Number of anti-VEGF injections received |
| | | up to Week 24 |
| | | Duration of time from study intervention |
| | | (Day 1) to first anti-VEGF administration |
| | | per SOC |
| | | Proportion of participants developing diabetic |
| | | complications (eg, neovascularization due to |
| | | DR) requiring PRP per SOC through |
| | | Week 24; for this population, the following |
| | | endpoints will be evaluated: |
| | | Duration of time from study intervention |
| | | (Day 1) to first PRP |
| | | Proportion of participants requiring more |
| | | than 1 PRP |
| | | Proportion of participants developing diabetic |
| | | complications (eg, retinal detachment) requiring |
| | | surgical intervention (pneumatic retinopexy, |
| | | cryopexy, or scleral buckle) per SOC; for this |
| | | population, the following endpoint will be |
| | | evaluated: |
| | | Duration of time from study intervention (Day |
| | | 1) to surgical intervention |
| Pharmacodynamics | To measure aqueous | Aqueous Construct II TP concentrations at |
| | and serum Construct II | assessed time points |
| | TP concentrations | Serum Construct II TP concentrations at |
| | | assessed time points |

Exploratory

| | | |
|---|---|---|
| Efficacy/Safety | To evaluate the effect | Proportion of participants with visual stability |
| | of Construct II on | (within 5 ETDRS letters or ±5 ETDRS letters) |
| | vision outcomes in all | from baseline to Week 24 |
| | evaluable participants | Proportion of participants with vision gain or |
| | | vision loss >5 ETDRS letters from baseline to |
| | | Week 24 |
| | To evaluate the effect | Mean change in CST on SD-OCT at Week 12 |
| | of Construct II on | and Week 24 |
| | anatomic outcomes | Proportion of participants achieving ≤290 μm |
| | evaluated using SD- | CST on SD-OCT at Week 12 and Week 24 |
| | OCT in all evaluable | Proportion of participants with clinically |
| | participants | significant macular thickening in CST ≥30 |
| | | μm from baseline at Week 12 and Week 24 |
| | | Mean change in macular volume and percent |
| | | reduction in macular volume from baseline |
| | | based on SD-OCT, as determined by the CRC |
| | To assess evidence of | Proportion of participants graded as Level 61 |
| | vessel regression for | or 65 at baseline with evidence of vessel |
| | participants with | regression at Week 24 based on FA |
| | baseline PDR (Level | |
| | 61 or 65) | |
| | To assess changes in | Proportion of participants graded as Level 61 |
| | the area of leakage for | or 65 at baseline with change in area of |
| | participants with | leakage from baseline to Week 24 based on |
| | baseline PDR (Level | FA |
| | 61 or 65) | |
| | To assess changes | Mean change from baseline in the area of |
| | from baseline in the | retinal nonperfusion at Week 24 based on FA |
| | area of retinal | in all evaluable participants |
| | nonperfusion in all | |
| | evaluable participants | |
| Biomarkers | To measure aqueous | VEGF-A concentration in aqueous fluid at |
| | VEGF-A concentration | assessed time points |

AAV8 = adeno-associated virus serotype 8;
AE = adverse event;
CI-DME = center involved-diabetic macular edema;
CRC = central reading center;
CST = central subfield thickness;
DR = diabetic retinopathy;
DRSS = Diabetic Retinopathy Severity Scale;
ETDRS = Early Treatment Diabetic Retinopathy Study;
FA = fluorescein angiography;
PDR = proliferative diabetic retinopathy;
PRP = panretinal photocoagulation;
SD-OCT = spectral domain-optical coherence tomography;
SOC = standard of care;
TP = transgene product;
VEGF = vascular endothelial growth factor

### 6.7.2 Inclusion Criteria

Participants must meet all the following criteria in order to be eligible for this study. All ocular criteria refer to the study eye: (1) men or women between 18-89 years of age with DR secondary to diabetes mellitus Type 1 or 2. Participants must have a hemoglobin A1c≤10% (as confirmed by laboratory assessments obtained at Screening or by a documented laboratory report dated within 60 days prior to Screening); (2) participant deemed to be an appropriate surgical candidate, per the investigator; (3) study eye with moderately-severe NPDR, severe NPDR, mild PDR, or moderate PDR (ETDRS-DRSS Levels 47, 53, 61, or 65 using standard 4-widefield digital stereoscopic fundus photographs, as determined by the CRC) for which PRP or anti-VEGF injections can be safely deferred, in the opinion of the investigator, for at least 6 months after Screening; (4) no evidence in the study eye of high-risk characteristics typically associated with vision loss, per the investigator, including the following: (i) new vessels within 1-disc area of the optic nerve, or vitreous or preretinal hemorrhage associated with less extensive new vessels at the optic disc, or with new vessels elsewhere that are half a disc area or more in size, and (ii) no evidence in the study eye of anterior segment (eg, iris or angle) neovascularization on clinical examination; (5) best-corrected visual acuity (BCVA) in the study eye of >69 ETDRS letters (approximate Snellen equivalent 20/40 or better); note: if both eyes are eligible, the study eye must be the participant's worse-seeing eye, as determined by the investigator prior to enrollment; (6) prior history of CI-DME in the study eye is acceptable if no intravitreal anti-VEGF or short-acting steroid injections have been given within the last 6 months, AND no more than 10 documented injections have been given in the 3 years prior to Screening; (7) sexually active male participants with female partners of childbearing potential must be willing to use condoms plus a medically accepted form of partner contraception from Screening until 24 weeks after vector administration; (9) must be willing and able to comply with all study procedures and be available for the duration of the study; (10) must be willing and able to provide written, signed informed consent.

### 6.7.3 Exclusion Criteria

Participants are excluded from the study if any of the following criteria apply: (1) women of childbearing potential, defined as neither postmenopausal nor surgically sterile. Postmenopausal is defined to be documented 12 consecutive months without menses. Surgically sterile is defined as having bilateral tubal ligation/bilateral salpingectomy, bilateral tubal occlusive procedure, hysterectomy, or bilateral oophorectomy; (2) presence of any active CI-DME, as determined by the investigator, on clinical examination or within the center subfield of the study eye using the following threshold: Heidelberg Spectralis: 320 μm; (3) neovascularization in the study eye from a cause other than DR, per investigator; (4) evidence in the study eye, as determined by the investigator, of ischemia in the study eye involving >50% of the peripheral retina, or the fovea or papillomacular area on baseline FA; (5) evidence in the study eye of optic nerve pallor on clinical exam, as determined by investigator; (6) any evidence of or documented history of PRP or retinal laser in the study eye; (7) ocular or periocular infection in the study eye that may interfere with the surgical procedure; (8) any ocular condition in the study eye that could require surgical intervention within the 6 months after Screening (vitreous hemorrhage, cataract that does not meet the inclusion criteria, retinal traction, epiretinal membrane, etc) or any condition in the study eye that may, in the opinion of the investigator, increase the risk to the participant, require either medical or surgical intervention during the study to prevent or treat vision loss, or interfere with the study procedures or assessments; (9) active or history of retinal detachment in the study eye; (10) presence of an implant in the study eye at Screening (excluding intraocular lens [IOL]); (11) for phakic participants, Pentacam Nuclear Staging score ≥1 as scanned by the Pentacam device and verified by the CRC, or not meeting other baseline cataract criteria as outlined in Section 6.7.5(c); (12) advanced glaucoma in the study eye (ie, uncontrolled, despite 2 or more drop treatments or an intervention such as a tube or shunt), as assessed through consultation with the participant's glaucoma specialist or documented history of glaucoma surgery; (13) history of intraocular surgery in the study eye within 12 weeks prior to Screening; yttrium aluminum garnet (YAG) capsulotomy is permitted if performed >10 weeks prior to Screening; (14) history of intravitreal therapy in the study eye, including anti-VEGF therapy, within 6 months prior to Screening, and documentation of more than 10 prior anti-VEGF or short-acting steroid intravitreal injections in the study eye for DME within 3 years of Screening; (15) any prior intravitreal steroid injection in the study eye within 6 months prior to Screening, administration in the study eye of Ozurdex® within 12 months prior to Screening, or administration in the study eye of Iluvien® within 36 months prior to Screening; (16) any prior systemic anti-VEGF treatment within the 6 months prior to or plans to use systemic anti-VEGF therapy during the next 6 months after Screening; (17) history of therapy known to have caused retinal toxicity, or concomitant therapy with any drug that may affect VA or with known retinal toxicity, e.g., chloroquine or hydroxychloroquine; (18) myocardial infarction, cerebrovascular accident, or transient ischemic attacks within the 6 months prior to Screening; (19) uncontrolled hypertension (systolic blood pressure [BP] >180 mmHg, diastolic BP >100 mmHg) despite maximal medical treatment; note that if BP is brought below 180/100 mmHg and stabilized by antihypertensive treatment as determined by the investigator and/or primary care physician, the participant can be rescreened for eligibility; (20) a systemic condition that, in the opinion of the investigator, would preclude participation in the study (poor glycemic control, uncontrolled hypertension, etc); (21) any concomitant treatment that, in the opinion of the investigator, may interfere with the ocular surgical procedure or the healing process; (22) history of malignancy or hematologic malignancy that may compromise the immune system requiring chemotherapy and/or radiation in the 5 years prior to Screening. Localized basal cell carcinoma will be permitted; (23) has a serious, chronic, or unstable medical or psychological condition that, in the opinion of the investigator, may compromise the participant's safety or ability to complete all assessments and follow-up in the study; (24) any participant with the following laboratory values at Screening will be withdrawn from the study: (i) aspartate aminotransferase (AST)/alanine aminotransferase (ALT) >2.5×upper limit of normal (ULN), (ii) total bilirubin >1.5×ULN, unless the participant has a previously known history of Gilbert's syndrome and a fractionated bilirubin that shows conjugated bilirubin <35% of total bilirubin, (iii) prothrombin time >1.5×ULN, unless the participant is anticoagulated. Participants who are anticoagulated will be monitored by local labs and managed per local practice to hold or bridge anticoagulant therapy for the study procedure; consultation with the Medical Monitor is required if the participant is anticoagulated, (iv) hemoglobin <10 g/dL for male participants and <9 g/dL for female participants, (v) Platelets <100×103/μL, (vi) estimated glomerular filtration rate <30 mL/min/1.73 m²; (25) history of chronic renal failure requiring dialysis or kidney transplant; (26) initiation of intensive insulin treatment (pump or multiple daily injections) within the 6 months prior to Screening or plans to do so within 6 months of Screening; (27) currently taking anticoagulation therapy for which holding anticoagulation therapy for Construct II administration is not indicated or considered to be unsafe in the opinion of the treating investigator (ie, retinal surgeon), as well as the physician prescribing anticoagulation for the participant, as verified by the Medical Monitor; (28) participation in any other gene therapy study, including Construct II, or receipt of any investigational product within 30 days prior to enrollment or 5 half-lives of the investigational product, whichever is longer, or any plans to use an investigational product within 6 months following enrollment; (29) known hypersensitivity to ranibizumab or any of its components.

### 6.7.4 Study Intervention

Study intervention is defined as any investigational intervention(s), marketed product(s), placebo, or medical device(s) intended to be administered to a study participant according to the study protocol.

Eligible participants will be assigned to receive a single dose of either Construct II (Dose 1) or a single dose of Construct II (Dose 2). All participants will receive study intervention on Day 1 via subretinal delivery in an operating room.

TABLE 7
Summary of Study Intervention(s)

| Arm Name | Construct II Dose 1 | Construct II Dose 2 |
|---|---|---|
| Type | Gene therapy (AAV8.CB7.CI.amd42.rBG) | |
| Dose Formulation | Solution | |
| Unit Dose | 6.2 × 10¹¹ GC/mL | 1.0 × 10¹² GC/mL |
| Strength | | |
| Dosage Level(s) | 250 μL | 250 μL |
| | (1.6 × 10¹¹ GC/eye) | (2.5 × 10¹¹ GC/eye) |
| | one-time dose | one-time dose |

| | |
|---|---|
| Route of | Subretinal delivery |
| Administration | |
| Physical | Construct II investigational product is supplied as a frozen, sterile, |
| Description | single- use solution of the AAV vector active ingredient |
| | (AAV8.CB7.CI.amd42.rBG) in a formulation buffer. The solution |
| | appears clear to opalescent, colorless, and free of visible particulates at |
| | room temperature. |
| Packaging and | Study intervention will be supplied as a sterile, single-use solution in |
| Labeling | 2-mL Crystal Zenith ® vials sealed with latex free rubber stoppers and |
| | aluminum flip-off seals. Each vial will be labeled as required per |
| | applicable regulatory requirements. |

Participants in this study will be randomized (1:1) at Screening using an interactive response technology system to receive Construct II (Dose 1) or Construct II (Dose 2).

### 6.7.5 Prior and Concomitant Therapy

(a) Medications and Therapies

The following medications are prohibited prior to entry into the study:

Any prior systemic or ocular anti-VEGF treatment in the study eye within the 6 months prior to Screening.

More than 10 prior, documented, anti-VEGF or short-acting steroid intravitreal injections in the study eye for DME within 3 years of Screening.

Any prior intravitreal short-acting steroid injection in the study eye within 6 months prior to Screening, administration in the study eye of Ozurdex within 12 months prior to Screening, or administration in the study eye of Iluvien within 36 months prior to Screening.

Initiation of intensive insulin treatment (pump or multiple daily injections) within the 6 months prior to Screening; for participants meeting this criterion, modification of the regimen is permitted during the study, as recommended and documented by their primary care provider or other treatment provider.

Participants must not have used any concomitant treatment that, in the opinion of the investigator, could interfere with Construct II administration or the healing process.

Participants are prohibited from taking anticoagulation therapy for which holding anticoagulation therapy for Construct II administration is not indicated or considered to be unsafe in the opinion of the treating investigator (ie, retinal surgeon), as well as the physician prescribing anticoagulation for the participant.

Participants must not have used any investigational product within 30 days prior to enrollment or within 5 half-lives of the investigational product, whichever is longer.

The following concomitant medications are prohibited during the study:

Anti-VEGF therapy in the study eye during the 6 months after Screening, except in the situations described in Section 6.7.5(b) for treatment of ocular diabetes complications.

Initiation of intensive insulin treatment (pump or multiple daily injections) is not allowed during the study; as indicated previously, modification of the treatment regimen is allowed during the study if initiation of treatment occurred at least 6 months prior to Screening.

Postoperative care for participants receiving Construct II is described in the Procedures Manual. There are no other restrictions on prior or concomitant therapy in this study.

(b) Treatment of Ocular Diabetes Complications

All complications of ocular diabetes will be managed in accordance with each study centers SOC and must be documented as an AE.

During the study, participants who develop diabetic complications requiring anti-VEGF treatment per SOC may be administered therapy as required. If needed, the study centers will provide their own supply of FDA-approved anti-VEGF therapy. The number of anti-VEGF injections received, and the timing of all administrations, must also be recorded in the source documents and eCRF.

Participants who develop diabetic complications requiring PRP SOC must have the time of PRP recorded in the source documents and eCRF.

Participants who develop diabetic complications requiring surgical intervention SOC (either pneumatic retinopexy, cryopexy, or scleral buckle) must have the type of intervention and the time of intervention recorded in the source documents and eCRF.

(c) Intervention for Cataract Formation

Baseline Screening for Phakic Participants

During the Screening visit, a series of assessments will be completed to determine eligibility and establish the participant's baseline cataract status for phakic participants only. These assessments include the following: (1) assessing the participant's symptoms per SOC; (2) performing a clinical examination to determine whether any clinically significant cataract, per cataract investigator, is present; (3) imaging the lens nucleus with the Oculus Pentacam Nuclear Staging (PNS) system. Pentacam grade ≤1 is acceptable for inclusion into the study. Pentacam eligibility should be determined at the site, and Pentacam scan should be submitted to the CRC for verification; and (4) imaging the participant's cortex and posterior capsule of the lens with standardized red reflex anterior segment photographs, which will be submitted to the CRC for grading and confirmation of study eligibility. Any subject with either cortical or posterior subcapsular lens image grade ≥Level 2 AREDS (mild opacities) will not be eligible.

On-Study Cataract Evaluation and Intervention for Phakic Participants

During the study, the retina investigator and cataract investigator will continue to assess participants for the presence of cataracts meeting the criteria for removal specified below.

The criterion for medically indicated cataract extraction, which is to be reported as an AE, is as follows: the retina investigator is unable to adequately view and/or image the retina in order to safely monitor and manage diabetic eye disease and/or general retinal status.

If the criterion for medically indicated cataract extraction is met at any postbaseline visit, an unscheduled visit for cataract extraction surgery will be scheduled as soon as possible by the study coordinator with the cataract investigator.

If the criterion for medically indicated cataract extraction is not met, but the participant meets either of the following two secondary criteria at any postbaseline visit, (BCVA decrease or participant-reported, described below), the study coordinator should schedule an unscheduled visit as soon as possible to obtain confirmatory Pentacam and CRC-graded lens photos (if not already available at that visit):
1. BCVA decrease: a decrease in BCVA of >5 ETDRS letters, relative to the best value recorded during the study (baseline or postbaseline) believed to be the result of worsening of cataract.
2. Participant-reported: visual symptoms resulting in lifestyle impairment as reported by the participant believed to be the result of worsening of cataract.

If during the unscheduled visit, a change in nuclear sclerosis from baseline on Pentacam Nuclear Staging of ≥1 grade or CRC-graded cortical or posterior subcapsular red reflex lens imaging of moderate cataract (ie, 5% involvement of central 5 mm) is confirmed, the secondary criteria for cataract extraction gas been met. This should be reported as an AE, and the study coordinator should schedule the unscheduled visit for cataract extraction as soon as possible by the cataract investigator.

A monofocal, 1-piece acrylic IOL is the lens of choice for use in this study. In some instances, a toric (astigmatism-correcting) IOL could be considered, but any difference in cost between a monofocal IOL and a toric lens is the responsibility of the participant unless otherwise approved by the Sponsor and the Medical Monitor. Multifocal or other premium IOLs are excluded during the study, as they may diminish the ability to accurately track any changes in retinal pathology. Silicone optic IOLs will not be used because of their potential to complicate any subsequent retinal procedures. The cataract surgeon may provide the participant with a recommendation that is most likely to provide optimal postoperative VA and visual function.

A postoperative, SOC protocol intended to limit complications will be followed. The preferred SOC protocol includes: fluroquinolone drops 4-times daily for 1 week, Ilevro (nepafenac) 2-times daily for 1 month, and a steroid taper with prednisolone acetate starting with 4-times daily for 1 week, tapering down 1 week at a time to 3-times daily, 2-times daily, and, finally, 1-time daily. For participant safety, alternative postoperative protocols may be used where appropriate, and with approval by the Medical Monitor.

### 6.8 Example 8: A Phase 2, Randomized, Dose-Escalation, Observation-Controlled Study to Evaluate the Efficacy, Safety, and Tolerability of Construct II Gene Therapy Delivered via One or Two Suprachoroidal Space (SCS) Injections in Participants with Diabetic Retinopathy (DR) without Center Involved-Diabetic Macular Edema (CI-DME)

### 6.8.1 Objectives and Endpoints

TABLE 8
Objectives and Endpoints

| | |
|---|---|
| Objectives | Endpoints |

Primary

| | | |
|---|---|---|
| Efficacy | To evaluate the effect of | Proportion of participants achieving a 2-step |
| | Construct II on DR by | or greater improvement in DR by |
| | the ETDRS-DRSS at | ETDRS-DRSS on 4-widefield digital |
| | Week 48 | stereoscopic fundus photography at Week 48 |

Secondary

| | | |
|---|---|---|
| Efficacy | To evaluate the effect of | Proportion of participants achieving a 2-step |
| | Construct II on DR | or greater improvement in DR per |
| | (ETDRS-DRSS) over | ETDRS-DRSS on 4-widefield digital |
| | time | stereoscopic fundus photography at Week 4, |
| | | Week 12, and Week 24 |
| | | Proportion of participants achieving a 0-step |
| | | (no change), a 1-step or greater, or a 3-step or |
| | | greater improvement in DR per |
| | | ETDRS-DRSS on 4-widefield digital |
| | | stereoscopic fundus photography at Week 4, |
| | | Week 12, Week 24, and Week 48 |
| | | Proportion of participants with a 1-step or |
| | | greater, a 2-step or greater, or a 3-step or |
| | | greater worsening in DR per ETDRS-DRSS |
| | | on 4-widefield digital stereoscopic fundus |
| | | photography at Week 4, Week 12, Week 24, |
| | | and Week 48 |
| | | Proportion of participants graded as Level 61 |
| | | or 65 (PDR) at baseline achieving regression |
| | | to Level 47 or 53 (NPDR) at Week 24 and |
| | | Week 48 |
| Safety/Immunogenicity | To assess the safety, | Incidences of overall and ocular AEs |
| | tolerability, and | Immunogenicity measurements (AAV8: |
| | immunogenicity of | NAbs, TAbs, and ELISpot; Construct II |
| | Construct II | protein: TAbs and ELISpot) over 24 weeks |
| Safety/Efficacy | To evaluate the need for | Proportion of participants requiring any |
| | additional SOC | additional intervention for ocular diabetic |
| | intervention due to | complications to Week 48 |
| | ocular diabetic | Proportion of participants with any sight- |
| | complications | threatening ocular diabetic complications to |
| | | Week 48 |
| | | Proportion of participants developing ocular |
| | | diabetic complications (eg, CI-DME or |
| | | neovascularization) requiring anti-VEGF |
| | | treatment per SOC through Week 48; for this |
| | | population, the following endpoints will be |
| | | evaluated: |
| | | Number of anti-VEGF injections received |
| | | Duration of time from study intervention |
| | | (Day 1) to first anti-VEGF administration |
| | | per SOC |
| | | Proportion of participants developing ocular |
| | | diabetic complications (eg, neovascularization |
| | | due to DR) requiring PRP per SOC through |
| | | Week 48; for this population, the following |
| | | endpoints will be evaluated: |
| | | Duration of time from study intervention |
| | | (Day 1) to first PRP |
| | | Proportion of participants requiring more |
| | | than 1 PRP |
| | | Proportion of participants developing ocular |
| | | diabetic complications (eg, retinal |
| | | detachment) requiring surgical intervention |
| | | (pneumatic retinopexy, cryopexy, or scleral |
| | | buckle) per SOC; for this population, the |
| | | following endpoint will be evaluated: |
| | | Duration of time from study intervention |
| | | (Day 1) to surgical intervention |
| Pharmacodynamics | To measure aqueous | Aqueous Construct II TP concentrations at |
| | and serum Construct II | assessed time points |
| | TP concentrations | Serum Construct II TP concentrations at |
| | | assessed time points |

Exploratory

| | | |
|---|---|---|
| Efficacy/Safety | To evaluate the effect of | Proportion of participants with visual stability |
| | Construct II on vision | (within 5 ETDRS letters or ±5 ETDRS |
| | outcomes (BCVA in all | letters) from baseline to Week 48 |
| | Construct II treated | Proportion of participants with vision gain or |
| | participants) | vision loss >5 ETDRS letters from baseline to |
| | | Week 48 |
| | To evaluate the effect of | Proportion of participants with clinically |
| | Construct II on visual | significant changes in visual field from |
| | field in all Construct II | baseline to Week 48, as determined by the |
| | treated participants | investigator |
| | To evaluate the effect of | Mean change from baseline in CST on |
| | Construct II on | SD-OCT at Week 24 and Week 48 |
| | anatomic outcomes | Proportion of participants achieving ≤290 μm |
| | assessed using SD-OCT | in CST on SD-OCT at Week 24 and Week 48 |
| | in all Construct II | Proportion of participants with clinically |
| | treated participants | significant macular thickening in CST ≥30 |
| | | μm from baseline at Week 24 and Week 48, |
| | | as determined by the CRC |
| | | Mean change in macular volume and percent |
| | | reduction in macular volume at Week 48 |
| | | relative to baseline on SD-OCT, as determined |
| | | by the CRC |
| | To assess evidence of | Proportion of participants graded as Level 61 |
| | vessel regression on FA | or 65 at baseline with evidence of vessel |
| | for participants with | regression at Week 24 and Week 48 based on |
| | baseline PDR (Level 61 | FA, as determined by the CRC |
| | or 65) | |
| | To assess changes in the | Proportion of participants graded as Level 61 |
| | area of leakage on FA | or 65 at baseline with change in the area of |
| | for participants with | leakage from baseline to Week 24 and |
| | baseline PDR (Level 61 | Week 48 based on FA, as determined by the |
| | or 65) | CRC |
| | To assess changes from | Mean change from baseline in the area of |
| | baseline in the area of | retinal nonperfusion at Week 24 and Week 48 |
| | retinal nonperfusion on | based on FA in all evaluable participants, as |
| | Optos widefield FA in | determined by the CRC |
| | all evaluable | |
| | participants | |
| Biomarkers | To measure aqueous | VEGF-A concentration in aqueous humor at |
| | VEGF-A concentration | assessed time points |

AAV8 = adeno-associated virus serotype 8;
AE = adverse event;
BCVA = best-corrected visual acuity;
CI-DME = center involved-diabetic macular edema;
CRC = central reading center;
CST = central subfield thickness;
DR = diabetic retinopathy;
DRSS = Diabetic Retinopathy Severity Scale;
ELISpot = enzyme-linked ImmunoSpot;
ETDRS = Early Treatment Diabetic Retinopathy Study;
FA = fluorescein angiography;
NAb = neutralizing antibody;
PDR = proliferative diabetic retinopathy;
PRP = panretinal photocoagulation;
SD-OCT = spectral domain-optical coherence tomography;
SOC = standard of care;
TAb = total binding antibody;
TP = transgene product;
VEGF = vascular endothelial growth factor

### 6.8.2 Inclusion Criteria

All Participants Entering the Study

Construct II TP concentrations (ng/mL) in aqueous and serum at assessed time points will be summarized descriptively by treatment arm and by the study overall. Participants must meet all the following criteria in order to be eligible for this study. All ocular criteria refer to the study eye:
1. Men or women 25-89 years of age with DR secondary to diabetes mellitus Type 1 or
2. Participants must have a hemoglobin A1c ≤10% (as confirmed by laboratory assessments obtained at Screening Visit 2 or by a documented laboratory report dated within 60 days prior to Screening Visit 2).
2. Must have a negative or low (≤300) serum titer result for AAV8 NAbs within 180 days prior to Screening Visit 2.
3. Study eye with moderately-severe NPDR, severe NPDR, or mild PDR (ETDRS-DRSS levels 47, 53, or 61 using standard 4-widefield digital stereoscopic fundus photographs, as determined by the CRC) for which PRP or anti-VEGF injections can be safely deferred, in the opinion of the investigator, for at least 6 months after Screening Visit 2.
4. No evidence in the study eye of high-risk characteristics typically associated with vision loss, per the investigator, including the following:
   New vessels within 1-disc area of the optic nerve
   Vitreous or preretinal hemorrhage associated with less extensive new vessels at the optic disc, or with new vessels elsewhere that are half a disc area or more in size.
   No evidence in the study eye of anterior segment (eg, iris or angle) neovascularization on clinical examination.
5. Best-corrected visual acuity in the study eye of ≥69 ETDRS letters (approximate Snellen equivalent 20/40 or better); note: if both eyes are eligible, the study eye must be the participant's worse-seeing eye, as determined by the investigator prior to enrollment.
6. Prior history of CI-DME in the study eye is acceptable if no intravitreal anti-VEGF or short-acting steroid injections have been given within the last 6 months, AND no more than 10 documented injections have been given in the 3 years prior to Screening Visit 2.
7. Sexually active male participants with female partners of childbearing potential must be willing to use condoms plus a medically accepted form of partner contraception from Screening Visit 2 until 24 weeks after vector administration.
8. Must be willing and able to comply with all study procedures and be available for the duration of the study.
9. Must be willing and able to provide written, signed informed consent.

Observation Control Arm Participants Following Week 48 who Switch to Construct II

Participants in the ranibizumab control arm who choose, following Week 48, to switch to treatment with Construct II must meet all of the following criteria at the Week 49 visit:
1. Study eye must be the eye that qualified at randomization.
2. Must qualify for NAb titer for the cohort requirements they will switch into.
3. Participants must, in the opinion of the investigator, have achieved adequate response to ranibizumab at Week 49 and the investigator must recommend switching to Construct II after consultation with the Sponsor.
4. Study eye with moderately-severe NPDR, severe NPDR, or mild PDR (ETDRS-DRSS levels 47, 53, or 61 using standard 4-widefield digital stereoscopic fundus photographs, as determined by the CRC).
5. No evidence in the study eye of high-risk characteristics typically associated with vision loss, per the investigator, including the following:
   New vessels within 1-disc area of the optic nerve, or vitreous or preretinal hemorrhage associated with less extensive new vessels at the optic disc, or with new vessels elsewhere that are half a disc area or more in size.
6. No evidence in the study eye of anterior segment (e.g., iris or angle) neovascularization on clinical examination.
7. BCVA in the study eye of >69 ETDRS letters (approximate Snellen equivalent 20/40 or better).
8. Women must be postmenopausal (defined as being at least 12 consecutive months without menses) or surgically sterilized (i.e., having a bilateral tubal ligation/bilateral salpingectomy, bilateral tubal occlusive procedure, hysterectomy, or bilateral oophorectomy). If not, women must have negative serum and urine pregnancy tests at Day 1 and be willing to undergo additional pregnancy testing during the study
9. All WOCBP (and their male partners) must be willing to use a highly effective method of contraception and male participants engaged in a sexual relationship with a WOCBP must be willing to use condoms from Week 54 until 24 weeks after Construct II administration.

### 6.8.3 Exclusion Criteria

All Participants Entering the Study

Participants are excluded from the study if any of the following criteria apply:
1. Women of childbearing potential (ie, women who are not postmenopausal or surgically sterile) are excluded from this clinical study.
   Postmenopausal is defined to be documented 12 consecutive months without menses.
   Surgically sterile is defined as having bilateral tubal ligation/bilateral salpingectomy, bilateral tubal occlusive procedure, hysterectomy, or bilateral oophorectomy.
2. Presence of any active CI-DME, as determined by the investigator, on clinical examination or within the center subfield of the study eye, as determined by SD-OCT evaluated by CRC, using the following threshold:
   Heidelberg Spectralis: ≥320 μm
3. Neovascularization in the study eye from a cause other than DR, per investigator.
4. Evidence in the study eye of optic nerve pallor on clinical examination, as determined by the investigator.
5. Any evidence or documented history of PRP or retinal laser in the study eye.
6. Ocular or periocular infection in the study eye that may interfere with the SCS procedure.
7. Any ocular condition in the study eye that could require surgical intervention within the 6 months after Screening Visit 2 (vitreous hemorrhage, cataract, retinal traction, epiretinal membrane, etc) or any condition in the study eye that may, in the opinion of the investigator, increase the risk to the participant, require either medical or surgical intervention during the study to prevent or treat vision loss, or interfere with the study procedures or assessments.
8. Active or history of retinal detachment in the study eye.
9. Presence of an implant in the study eye at Screening Visit 2 (excluding intraocular lens).
10. Participants who had a prior vitrectomy. 11. Advanced glaucoma in the study eye, as defined by an TOP >23 mmHg, not controlled by 2 IOP-lowering medications, any invasive procedure to treat glaucoma (eg, shunt, tube, or MIGS devices; however, selective laser trabeculectomy and argon laser trabeculoplasty are permitted), or visual field loss encroaching on central fixation.
12. History of intraocular surgery in the study eye within 12 weeks prior to Screening Visit 2; yttrium aluminum garnet (YAG) capsulotomy is permitted if performed >10 weeks prior to Screening Visit 2.
13. History of intravitreal therapy in the study eye, including anti-VEGF therapy, within 6 months prior to Screening Visit 2, and documentation of more than 10 prior anti-VEGF or short-acting steroid intravitreal injections in the study eye within 3 years of Screening Visit 2.
14. Any prior intravitreal steroid injection in the study eye within 6 months prior to Screening Visit 2, administration in the study eye of Ozurdex® within 12 months prior to Screening Visit 2, or administration in the study eye of Iluvien® within 36 months prior to Screening Visit 2.
15. Any prior systemic anti-VEGF treatment within the 6 months prior to or plans to use systemic anti-VEGF therapy during the next 48 weeks after Screening Visit 2.
16. History of therapy known to have caused retinal toxicity, or concomitant therapy with any drug that may affect VA or with known retinal toxicity, eg, chloroquine or hydroxychloroquine.
17. Myocardial infarction, cerebrovascular accident, or transient ischemic attacks within the 6 months prior to Screening Visit 2.
18. Uncontrolled hypertension (systolic blood pressure [BP] >180 mmHg, diastolic BP >100 mmHg) despite maximal medical treatment; note that if BP is brought below 180/100 mmHg and stabilized by antihypertensive treatment, as determined by the investigator and/or primary care physician, the participant can be rescreened for eligibility.
19. A systemic condition that, in the opinion of the investigator, would preclude participation in the study (poor glycemic control, uncontrolled hypertension, etc).
20. Any concomitant treatment that, in the opinion of the investigator, may interfere with the ocular surgical procedure or the healing process.
21. History of malignancy with or without therapy or hematologic malignancy that may compromise the immune system requiring chemotherapy and/or radiation in the 5 years prior to Screening Visit 2. Localized basal cell carcinoma will be permitted.
22. Has a serious, chronic, or unstable medical or psychological condition that, in the opinion of the investigator, may compromise the participant's safety or ability to complete all assessments and follow-up in the study.
23. Any participant with the following laboratory values at Screening Visit 2 will be withdrawn from the study:
   Aspartate aminotransferase (AST)/alanine aminotransferase (ALT) >2.5×upper limit of normal (ULN).
   Total bilirubin >1.5×ULN, unless the participant has a previously known history of Gilbert's syndrome and a fractionated bilirubin that shows conjugated bilirubin <35% of total bilirubin.
   Prothrombin time >1.5×ULN, unless the participant is anticoagulated.
   Hemoglobin <10 g/dL for male participants and <9 g/dL for female participants.
   Platelets <100×10³/μL.
   Estimated glomerular filtration rate <30 mL/min/1.73 m².
24. History of chronic renal failure requiring dialysis or kidney transplant.
25. Initiation of intensive insulin treatment (pump or multiple daily injections) within the 6 months prior to Screening Visit 2 or plans to do so within 48 weeks of Day 1.
26. Participation in any other gene therapy study, including Construct II, or receipt of any investigational product within 30 days prior to enrollment or 5 half-lives of the investigational product, whichever is longer, or any plans to use an investigational product within 6 months following enrollment.
27. Known hypersensitivity to ranibizumab or any of its components.

Observation Control Arm Participants Following Week 48 who Switch to Construct II

Participants in the observation control arm who choose, following Week 48, to switch to treatment with Construct II will be ineligible to do so if they meet any of the exclusion criteria specified for screening with the exceptions of treatments in the study eye administered as SOC for diabetic complications (ie, receiving SOC in the study eye is not exclusionary for rolling into Construct II at Week 49).

### 6.8.4 Study Intervention(s) Administered

Eligible participants will be assigned either to receive a single dose of Construct II (Dose 1 or Dose 2) in the study eye or be followed for observation only.

TABLE 9
Information regarding Construct II

| Arm Name | Construct II Dose 1 | Construct II Dose 2 |
|---|---|---|
| Type | Gene therapy (AAV8.CB7.CI.amd42.RBG) | |
| Dose Formulation | Solution | |
| Unit Dose | 1.0 × 10¹² GC/mL | 2.5 × 10¹² GC/mL |
| Strength | | |
| Dosage Level(s) | 100 μL | 100 μL |
| | (2.5 × 10¹¹ GC/eye) | (5.0 × 10¹¹ GC/eye) |
| | delivered via a | delivered via 2 SCS |
| | single SCS injection | injections at the same visit |

| | |
|---|---|
| Route of | Suprachoroidal space injection in the study eye using a microinjector. |
| Administration | |
| Physical | Construct II investigational product is supplied as a frozen, sterile, single-use |
| Description | solution of the AAV vector active ingredient (AAV8.CB7.CI.amd42.RBG) in a |
| | formulation buffer. The solution appears clear to opalescent, colorless, and free |
| | of visible particulates at room temperature. |
| Packaging and | Construct II will be supplied as a sterile, single-use solution in 2-mL Crystal |
| Labeling | Zenith ® vials sealed with latex-free rubber stoppers and aluminum flip-off seals. |
| | Each vial will be labeled as required per country regulatory requirements. |

### 6.9 Example 9: Use of an Infrared Thermal Camera to Monitor Injection in Pigs

The FLIR T530 infrared thermal camera was used to characterize post ocular injection thermal profiles in live pigs. Alternatively, an FLIR T420, FLIR T440, Fluke Ti400, or FLIRE60 infrared thermal camera is used. Suprachoroidal (FIG. 6), unsuccessful suprachoroidal, intravitreal, and extraocular efflux injections of room temperature saline (68-72° F). were assessed in the study. Dose volume was 100 μL for every injection with the solution from the refrigerator to room temperature for injection.

Infrared camera lens to ocular surface distance was established at approximately 1 ft. The manual temperature range on the camera for viewing was set to ˜80-90° F. Imaging operator held the camera and set the center screen cursor aimed at the injection site during video recordings. Pigs received a retrobulbar injection of saline to proptose the eye for better visibility, and eye lids were cut and retracted back to expose the sclera at the site of injection. The iron filter was used during thermal video recordings.

A successful suprachoroidal injection was characterized by: (a) a slow, wide radial spread of the dark color, (b) very dark color at the beginning, and (c) a gradual change of injectate to lighter color, i.e., a temperature gradient noted by a lighter color. An unsuccessful suprachoroidal injection was characterized by: (a) no spread of the dark color, and (b) a minor change in color localized to the injection site. A successful intravitreal injection was characterized by: (a) no spread of the dark color, (b) an initial change to very dark color localized to the injection site, and (c) a gradual and uniform change of the entire eye to darker color occurring after the injection developing with time. Extraocular efflux was characterized by: (a) quick flowing streams on outside exterior of the eye, (b) very dark color at the beginning, and (c) a quick change to lighter color.

### 6.10 Example 10: Use of an Infrared Thermal Camera to Monitor Injection in Human Patients

A subject presenting with diabetic retinopathy (DR) is administered AAV8 that encodes ranibizumab Fab (e.g., by subretinal administration, suprachoroidal administration, or intravitreal administration) at a dose sufficient to produce a concentration of the transgene product at a Cmin of at least 0.330 μg/mL in the Vitreous humour for three months. The FLIR T530 infrared thermal camera is used to evaluate the injection during the procedure and is available to evaluate after the injection to confirm either that the administration is successfully completed or misdose of the administration. Alternatively, an FLIR T420, FLIR T440, Fluke Ti400, or FLIRE60 infrared thermal camera is used. Following treatment, the subject is evaluated clinically for signs of clinical effect and improvement in signs and symptoms of DR.

### 6.11 Example 11: A Phase 2, Randomized, Dose-Escalation, Observation-Controlled Study to Evaluate the Efficacy, Safety, and Tolerability of Construct II Gene Therapy Delivered via One or Two Suprachoroidal Space (SCS) Injections in Participants with Diabetic Retinopathy (DR) Without Center Involved-Diabetic Macular Edema (CI-DME)

This example is an updated version of Example 8 and provides an overview of a phase 2a, dose assessment of Construct II gene therapy in participants with diabetic retinopathy (DR).

### 6.11.1 Objectives and Endpoints

TABLE 10
Objectives and Endpoints

| | |
|---|---|
| Objectives | Endpoints |

Primary

| | | |
|---|---|---|
| Efficacy | To evaluate the effect of | Proportion of participants achieving a 2-step |
| | Construct II on DR by | or greater improvement in DR by |
| | the ETDRS-DRSS at | ETDRS-DRSS on 4-widefield digital |
| | Week 48 | stereoscopic fundus photography at Week 48 |

Secondary

| | | |
|---|---|---|
| Efficacy | To evaluate the effect of | Proportion of participants achieving a 2-step |
| | Construct II on DR | or greater improvement in DR per |
| | (ETDRS-DRSS) over | ETDRS-DRSS on 4-widefield digital |
| | time | stereoscopic fundus photography at Week 4, |
| | | Week 12, and Week 24 |
| | | Proportion of participants achieving a 0-step |
| | | (no change), a 1-step or greater, or a 3-step or |
| | | greater improvement in DR per |
| | | ETDRS-DRSS on 4-widefield digital |
| | | stereoscopic fundus photography at Week 4, |
| | | Week 12, Week 24, and Week 48 |
| | | Proportion of participants with a 1-step or |
| | | greater, a 2-step or greater, or a 3-step or |
| | | greater worsening in DR per ETDRS-DRSS |
| | | on 4-widefield digital stereoscopic fundus |
| | | photography at Week 4, Week 12, Week 24, |
| | | and Week 48 |
| | | Proportion of participants graded as Level 61 |
| | | (PDR) at baseline achieving regression to |
| | | Level 47 or 53 (NPDR) at Week 24 and Week |
| | | 48 |
| Safety/Immunogenicity | To assess the safety, | Incidences of overall and ocular AEs |
| | tolerability, and | Immunogenicity measurements (AAV8: |
| | immunogenicity of | NAbs, TAbs, and ELISpot; Construct II TP: |
| | Construct II | anti-Construct II TP antibodies and ELISpot) |
| | | over 48 weeks |
| Safety/Efficacy | To evaluate the need for | Proportion of participants requiring any |
| | additional SOC | additional intervention for ocular diabetic |
| | intervention due to | complications to Week 48 |
| | ocular diabetic | Proportion of participants with any sight- |
| | complications | threatening ocular diabetic complications to |
| | | Week 48 |
| | | Proportion of participants developing ocular |
| | | diabetic complications (eg, CI-DME or |
| | | neovascularization) requiring anti-VEGF |
| | | treatment per SOC through Week 48; for this |
| | | population, the following endpoints will be |
| | | evaluated: |
| | | Number of anti-VEGF injections received |
| | | Duration of time from study intervention |
| | | (Day 1) to first anti-VEGF administration |
| | | per SOC |
| | | Proportion of participants developing ocular |
| | | diabetic complications (eg, neovascularization |
| | | due to DR) requiring PRP per SOC through |
| | | Week 48; for this population, the following |
| | | endpoints will be evaluated: |
| | | Duration of time from study intervention |
| | | (Day 1) to first PRP |
| | | Proportion of participants requiring more |
| | | than 1 PRP |
| | | Proportion of participants developing ocular |
| | | diabetic complications (eg, retinal |
| | | detachment) requiring surgical intervention |
| | | (pneumatic retinopexy, cryopexy, or scleral |
| | | buckle) per SOC; for this population, the |
| | | following endpoint will be evaluated: |
| | | Duration of time from study intervention |
| | | (Day 1) to surgical intervention |
| Pharmacodynamics | To measure aqueous | Aqueous Construct II TP concentration at |
| | and serum Construct II | assessed time points |
| | TP concentrations | Serum Construct II TP concentration at |
| | | assessed time points |

Exploratory

| | | |
|---|---|---|
| Efficacy/Safety | To evaluate the effect of | Proportion of participants with visual stability |
| | Construct II on vision | (within 5 ETDRS letters or ±5 ETDRS |
| | outcomes (BCVA in all | letters) from baseline to Week 48 |
| | Construct II treated | Proportion of participants with vision gain or |
| | participants) | vision loss >5 ETDRS letters from baseline to |
| | | Week 48 |
| | To evaluate the effect of | Proportion of participants with clinically |
| | Construct II on visual | significant changes in visual field from |
| | field in all Construct II | baseline to Week 48, as determined by the |
| | treated participants | investigator |
| | To evaluate the effect of | Mean change from baseline in CST on |
| | Construct II on | SD-OCT at Week 24 and Week 48 |
| | anatomic outcomes | Proportion of participants achieving ≤290 μm |
| | assessed using SD-OCT | in CST on SD-OCT at Week 24 and Week 48 |
| | in all Construct II | Proportion of participants with clinically |
| | treated participants | significant macular thickening in CST ≥30 |
| | | μm from baseline at Week 24 and Week 48, |
| | | as determined by the CRC |
| | | Mean change in macular volume and percent |
| | | reduction in macular volume at Week 48 |
| | | relative to baseline on SD-OCT, as determined |
| | | by the CRC |
| | To assess evidence of | Proportion of participants graded as Level 61 |
| | vessel regression on FA | at baseline with evidence of vessel regression |
| | for participants with | at Week 24 and Week 48 based on FA, as |
| | baseline PDR | determined by the CRC |
| | (Level 61) | |
| | To assess changes in the | Proportion of participants graded as Level 61 |
| | area of leakage on FA | at baseline with change in the area of leakage |
| | for participants with | from baseline to Week 24 and Week 48 based |
| | baseline PDR | on FA, as determined by the CRC |
| | (Level 61) | |
| | To assess changes from | Mean change from baseline in the area of |
| | baseline in the area of | retinal nonperfusion at Week 24 and Week 48 |
| | retinal nonperfusion on | based on FA in all evaluable participants, as |
| | Optos widefield FA in | determined by the CRC |
| | all evaluable | |
| | participants | |

AAV8 = adeno-associated virus serotype 8;
AE = adverse event;
BCVA = best-corrected visual acuity;
CI-DME = center involved-diabetic macular edema;
CRC = central reading center;
CST = central subfield thickness;
DR = diabetic retinopathy;
DRSS = Diabetic Retinopathy Severity Scale;
ELISpot = enzyme-linked ImmunoSpot;
ETDRS = Early Treatment Diabetic Retinopathy Study;
FA = fluorescein angiography;
NAb = neutralizing antibody;
PDR = proliferative diabetic retinopathy;
PRP = panretinal photocoagulation;
SD-OCT = spectral domain-optical coherence tomography;
SOC = standard of care;
TAb = total binding antibody;
TP = transgene product;
VEGF = vascular endothelial growth factor

### 6.11.2 Inclusion Criteria

Participants must meet all the following criteria in order to be eligible for this study. All ocular criteria refer to the study eye:
1. Men or women 25-89 years of age with DR secondary to diabetes mellitus Type 1 or 2. Participants must have a hemoglobin A1c ≤10% (as confirmed by laboratory assessments obtained at Screening Visit 2 or by a documented laboratory report dated within 60 days prior to Screening Visit 2).
2. Study eye with moderately-severe NPDR, severe NPDR, or mild PDR (ETDRS-DRSS levels 47, 53, or 61 using standard 4-widefield digital stereoscopic fundus photographs, as determined by the CRC) for which PRP or anti-VEGF injections can be safely deferred, in the opinion of the investigator, for at least 6 months after Screening Visit 2.
3. No evidence in the study eye of high-risk characteristics typically associated with vision loss, per the investigator, including the following:
   New vessels within 1-disc area of the optic nerve
   Vitreous or preretinal hemorrhage associated with less extensive new vessels at the optic disc, or with new vessels elsewhere that are half a disc area or more in size.
   No evidence in the study eye of anterior segment (eg, iris or angle) neovascularization on clinical examination.
4. Must have a negative or low (≤300) serum titer result for AAV8 NAbs.
5. Best-corrected visual acuity in the study eye of ≥69 ETDRS letters (approximate Snellen equivalent 20/40 or better); note: if both eyes are eligible, the study eye must be the participant's worse-seeing eye, as determined by the investigator, prior to enrollment.
6. Prior history of CI-DME in the study eye is acceptable if no intravitreal anti-VEGF or short-acting steroid injections have been given within the last 6 months, AND no more than 10 documented injections have been given in the 3 years prior to Screening Visit 2.
7. Sexually active male participants with female partners of childbearing potential must be willing to use condoms plus a medically accepted form of partner contraception from Screening Visit 2 until 24 weeks after vector administration.
8. Must be willing and able to comply with all study procedures and be available for the duration of the study.
9. Must be willing and able to provide written, signed informed consent.

### 6.11.3 Exclusion Criteria

Participants are excluded from the study if any of the following criteria apply:
1. Women of childbearing potential (ie, women who are not postmenopausal or surgically sterile) are excluded from this clinical study.
   Postmenopausal is defined to be documented 12 consecutive months without menses.
   Surgically sterile is defined as having bilateral tubal ligation/bilateral salpingectomy, bilateral tubal occlusive procedure, hysterectomy, or bilateral oophorectomy.
2. Presence of any active CI-DME, as determined by the investigator, on clinical examination or within the central subfield thickness (CST) of the study eye, as determined by SD-OCT evaluated by CRC, using the following threshold:
   Heidelberg Spectralis: CST greater than 320 μm
3. Neovascularization in the study eye from a cause other than DR, per investigator.
4. Evidence in the study eye of optic nerve pallor on clinical examination, as determined by the investigator.
5. Any evidence or documented history of PRP or retinal laser in the study eye.
6. Ocular or periocular infection in the study eye that may interfere with the SCS procedure.
7. Any ocular condition in the study eye that could require surgical intervention within the 6 months after Screening Visit 2 (vitreous hemorrhage, cataract, retinal traction, epiretinal membrane, etc) or any condition in the study eye that may, in the opinion of the investigator, increase the risk to the participant, require either medical or surgical intervention during the study to prevent or treat vision loss, or interfere with the study procedures or assessments.
8. Active or history of retinal detachment in the study eye.
9. Presence of an implant in the study eye at Screening Visit 2 (excluding intraocular lens).
10. Participants who had a prior vitrectomy surgery.
11. Advanced glaucoma in the study eye, as defined by an TOP >23 mmHg, not controlled by 2 TOP-lowering medications, any invasive procedure to treat glaucoma (eg, shunt, tube, or MIGS devices; however, selective laser trabeculectomy and argon laser trabeculoplasty are permitted), or visual field loss encroaching on central fixation.
12. History of intraocular surgery in the study eye within 12 weeks prior to Screening Visit 2; yttrium aluminum garnet (YAG) capsulotomy is permitted if performed >10 weeks prior to Screening Visit 2.
13. History of intravitreal therapy in the study eye, including anti-VEGF therapy, within 6 months prior to Screening Visit 2, and documentation of more than 10 prior anti-VEGF or short-acting steroid intravitreal injections in the study eye within 36 months of Screening Visit 2.
14. Any prior intravitreal steroid injection in the study eye within 6 months prior to Screening Visit 2, administration in the study eye of Ozurdex° within 12 months prior to Screening Visit 2, or administration in the study eye of Iluvien® within 36 months prior to Screening Visit 2.
15. Any prior systemic anti-VEGF treatment within the 6 months prior to or plans to use systemic anti-VEGF therapy during the next 48 weeks after Screening Visit 2.
16. History of therapy known to have caused retinal toxicity, or concomitant therapy with any drug that may affect VA or with known retinal toxicity, eg, chloroquine or hydroxychloroquine.
17. Myocardial infarction, cerebrovascular accident, or transient ischemic attacks within the 6 months prior to Screening Visit 2.
18. Uncontrolled hypertension (systolic blood pressure [BP] >180 mmHg, diastolic BP >100 mmHg) despite maximal medical treatment; note that if BP is brought below 180/100 mmHg and stabilized by antihypertensive treatment, as determined by the investigator and/or primary care physician, the participant can be rescreened for eligibility.
19. A systemic condition that, in the opinion of the investigator, would preclude participation in the study (poor glycemic control, uncontrolled hypertension, etc).
20. Any concomitant treatment that, in the opinion of the investigator, may interfere with the ocular procedure or the healing process.
21. History of malignancy with or without therapy or hematologic malignancy that may compromise the immune system requiring chemotherapy and/or radiation in the 5 years prior to Screening Visit 2. Localized basal cell carcinoma will be permitted.
22. Has a serious, chronic, or unstable medical or psychological condition that, in the opinion of the investigator, may compromise the participant's safety or ability to complete all assessments and follow-up in the study.
23. Meets any one of the following exclusionary laboratory values at Screening Visit 2:
   Aspartate aminotransferase (AST) and/or alanine aminotransferase (ALT) >2.5×upper limit of normal (ULN).
   Total bilirubin >1.5×ULN, unless the participant has a previously known history of Gilbert's syndrome and a fractionated bilirubin that shows conjugated bilirubin <35% of total bilirubin.
   Prothrombin time >1.5×ULN, unless the participant is anticoagulated.
   Hemoglobin <10 g/dL for male participants and <9 g/dL for female participants. Platelets <100×10³/μL.
   Estimated glomerular filtration rate <30 mL/min/1.73 m².
24. History of chronic renal failure requiring dialysis or kidney transplant.
25. Initiation of intensive insulin treatment (pump or multiple daily injections) within the 6 months prior to Screening Visit 2 or plans to do so within 48 weeks of Day 1.
26. Participation in any other gene therapy study, including Construct II, or receipt of any investigational product within 30 days prior to enrollment or 5 half-lives of the investigational product, whichever is longer, or any plans to use an investigational product within 6 months following enrollment.
27. Known hypersensitivity to ranibizumab or any of its components.

### 6.11.4 Study Intervention(s) Administered

Eligible participants will be assigned either to receive a single dose of Construct II (Dose 1 or Dose 2) in the study eye or be followed for observation only. Information regarding Construct II follows.

TABLE 11
Information regarding Construct II

| Arm Name | Construct II Dose 1 | Construct II Dose 2 |
|---|---|---|
| Type | Gene therapy (AAV8.CB7.CI.amd42.RBG) | |
| Dose Formulation | Solution | |
| Unit Dose | 2.5 × 10¹² GC/mL | 2.5 × 10¹² GC/mL |
| Strength | | |
| Dosage Level(s) | 2.5 × 10¹¹ GC/eye | 5.0 × 10¹¹ GC/eye |
| | delivered via a single 100 μL | delivered via two 100 μL SCS injections |
| | SCS injection (100 μL total volume) | at the same visit (200 μL total volume) |

| | |
|---|---|
| Route of | Suprachoroidal space injection in the study eye using a microinjector. |
| Administration | |
| Physical | Construct II investigational product is supplied as a frozen, sterile, single-use solution of |
| Description | the AAV vector active ingredient (AAV8.CB7.CI.amd42.RBG) in a formulation buffer. |
| | The solution appears clear to opalescent, colorless, and free of visible particulates at |
| | room temperature. |
| Packaging and | Construct II will be supplied as a sterile, single-use solution in 2-mL Crystal Zenith ® |
| Labeling | vials sealed with latex-free rubber stoppers and aluminum flip-off seals. Each vial will |
| | be labeled as required per country regulatory requirements. |

### 6.11.5 Vector Shedding

Sampling of blood (serum), urine, and tears will be performed for Construct II participants for measurement of vector concentrations. Refer to the Investigator Laboratory Manual for additional information regarding the processing, handling, and shipping of the samples.

Shedding data collected in these biological fluids provide a shedding profile of Construct II in the target patient population and is used to estimate the potential of transmission to untreated individuals. Shedding will be measured using quantitative polymerase chain reaction.

### 6.12 Example 12: Toxicity Study of Construct II in Cynomolgous Monkeys

In cynomolgus monkeys, Construct II was administered suprachoroidally at doses up to 3×10¹² GC/eye using a microinjector device. Animals were evaluated after 3 months.

In this study, the microinjector successfully administered Construct II into the SCS space, and there were no observed adverse findings associated with the use of the device or Construct II. There was widespread biodistribution determined by transduction in the retina and RPE/choroid, and detectable TP (anti-VEGF Fab) in both the aqueous and vitreous humor. The no observed adverse effect level (NOAEL) in this study was the highest dose tested, 3×10¹² GC/eye. At all doses in the 3-month non-human primate (NHP) toxicity study, vector DNA was detected in the liver, indicating that the vector may enter systemic circulation through the choriocapillaries following suprachoroidal injection. At the highest dose tested (3×10¹² GC/eye), low levels of vector DNA were also detected in additional peripheral tissues (occipital lobe, hippocampus, thalamus, heart, lung, kidney, and ovaries). However, there was no increase in serum concentrations of anti-VEGF Fab or any evidence of systemic toxicity. Furthermore, the presence of vector DNA in whole blood at the end of the study, an observation commonly seen in gene therapy, may have influenced some of the peripheral biodistribution observed.

In summary, for suprachoroidal dosing in NHPs, the NOAEL was the highest dose tested, 3×10¹² GC/eye. The presence of vector DNA in the liver is of unknown significance as there were no increases in serum anti-VEGF Fab. At the highest dose only, low levels of vector DNA were also detected in additional peripheral tissues and are of unknown significance as vector DNA was detected in the blood at the same timepoint. Therefore, for peripheral tissue biodistribution, a weight-based safety margin has been used. At the highest dose, 3×10¹² GC/eye or 1.5×10¹² GC/kg, there was no evidence of an increase in systemic concentrations of TP that correlated to vector DNA in the liver, or evidence of any liver changes observed. Therefore, in humans, doses up to 1.5×10¹¹ GC/kg are considered acceptable, as it is equivalent to a dose 10-fold lower than the highest dose administered in the 3-month toxicity study.

Within the SCS microinjector, a single injection volume of 100 μL can be easily administered in humans. Each microneedle is graduated to a total of 100 μL per needle.

### 7. EQUIVALENTS

Although the invention is described in detail with reference to specific embodiments thereof, it will be understood that variations which are functionally equivalent are within the scope of this invention. Indeed, various modifications of the invention in addition to those shown and described herein will become apparent to those skilled in the art from the foregoing description and accompanying drawings. Such modifications are intended to fall within the scope of the appended claims. Those skilled in the art will recognize, or be able to ascertain using no more than routine experimentation, many equivalents to the specific embodiments of the invention described herein. Such equivalents are intended to be encompassed by the following claims.

All publications, patents and patent applications mentioned in this specification are herein incorporated by reference into the specification to the same extent as if each individual publication, patent or patent application was specifically and individually indicated to be incorporated herein by reference in their entireties.

What is claimed:

1. A method of treating a human subject diagnosed with diabetic retinopathy (DR), comprising administering to the subretinal space in the eye of said human subject an expression vector encoding an anti-human vascular endothelial growth factor (hVEGF) antibody, wherein the expression vector is administered via subretinal delivery in a single dose about 1.6×10¹¹ GC/eye at a concentration of 6.2×10¹¹ GC/mL or about 2.5×10¹¹ GC/eye at a concentration of 1.0×10¹² GC/mL, wherein the anti-hVEGF antibody comprises a heavy chain comprising the amino acid sequence of SEQ ID NO. 2 or SEQ ID NO. 4, and a light chain comprising the amino acid sequence of SEQ ID NO. 1, or SEQ ID NO. 3; and wherein the expression vector is an AAV8 vector.

2. The method of claim 1, wherein the administering is by injecting the expression vector into the subretinal space using a subretinal drug delivery device.

3. The method of any one of claims 1-2, wherein the administering delivers a therapeutically effective amount of the anti-hVEGF antibody to the retina of said human subject.

4. The method of claim 3, wherein the therapeutically effective amount of the anti-hVEGF antibody is produced by human retinal cells of said human subject.

5. The method of claim 4, wherein the therapeutically effective amount of the anti-hVEGF antibody is produced by human photoreceptor cells, horizontal cells, bipolar cells, amacrine cells, retina ganglion cells, and/or retinal pigment epithelial cells in the external limiting membrane of said human subject.

6. The method of claim 5, wherein the human photoreceptor cells are cone cells and/or rod cells.

7. The method of claim 6, wherein the retina ganglion cells are midget cells, parasol cells, bistratified cells, giant retina ganglion cells, photosensitive ganglion cells, and/or Müllner glia.

8. The method of any one of claims 1-7, wherein the expression vector comprises the CB7 promoter.

9. The method of claim 8, wherein the expression vector is Construct II.

10. A single dose composition comprising 1.6×10¹¹ GC at a concentration of 6.2×10¹¹ GC/mL or 2.5×10¹¹ GC at a concentration of 1.0×10¹² GC/mL of an expression vector encoding an anti-human vascular endothelial growth factor (hVEGF) antibody in a formulation buffer (pH=7.4), wherein the formulation buffer comprises Dulbecco's phosphate buffered saline and 0.001% Pluronic F68, wherein the anti-hVEGF antibody comprises a heavy chain comprising the amino acid sequence of SEQ ID NO. 2 or SEQ ID NO. 4, and a light chain comprising the amino acid sequence of SEQ ID NO. 1, or SEQ ID NO. 3; and wherein the wherein the expression vector is an AAV8 vector.

11. The composition of claim 10, wherein the expression vector is Construct II.

12. The method of any one of claims 1-9, which further comprises, after the administering step, a step of monitoring the post ocular injection thermal profile of the injected material in the eye using an infrared thermal camera.

13. The method of claim 12, wherein the infrared thermal camera is a FLIR T530 infrared thermal camera.

14. A method of treating a human subject diagnosed with DR, comprising administering to the subretinal space in the eye of said human subject an expression vector encoding an anti-human vascular endothelial growth factor (hVEGF) antibody, wherein about 2.5×10¹¹ genome copies per eye of the expression vector are administered by double suprachoroidal injections, wherein the anti-hVEGF antibody comprises a heavy chain comprising the amino acid sequence of SEQ ID NO. 2 or SEQ ID NO. 4, and a light chain comprising the amino acid sequence of SEQ ID NO. 1, or SEQ ID NO. 3; and wherein the expression vector is an AAV8 vector.

15. A method of treating a human subject diagnosed with DR, comprising administering to the subretinal space in the eye of said human subject an expression vector encoding an anti-human vascular endothelial growth factor (hVEGF) antibody, wherein about 5.0×10¹¹ genome copies per eye of the expression vector are administered by double suprachoroidal injections, wherein the anti-hVEGF antibody comprises a heavy chain comprising the amino acid sequence of SEQ ID NO. 2 or SEQ ID NO. 4, and a light chain comprising the amino acid sequence of SEQ ID NO. 1, or SEQ ID NO. 3; and wherein the expression vector is an AAV8 vector.

16. The method of any one of claims 14-15, wherein the administering delivers a therapeutically effective amount of the anti-hVEGF antibody to the retina of said human subject.

17. The method of claim 16, wherein the therapeutically effective amount of the anti-hVEGF antibody is produced by human retinal cells of said human subject.

18. The method of claim 17, wherein the therapeutically effective amount of the anti-hVEGF antibody is produced by human photoreceptor cells, horizontal cells, bipolar cells, amacrine cells, retina ganglion cells, and/or retinal pigment epithelial cells in the external limiting membrane of said human subject.

19. The method of claim 18, wherein the human photoreceptor cells are cone cells and/or rod cells.

20. The method of claim 19, wherein the retina ganglion cells are midget cells, parasol cells, bistratified cells, giant retina ganglion cells, photosensitive ganglion cells, and/or Múller glia.

21. The method of any one of claims 14-20, wherein the expression vector comprises the CB7 promoter.

22. The method of claim 21, wherein the expression vector is Construct II.

23. The method of any one of claims 14-22, which further comprises, after the administering step, a step of monitoring the post ocular injection thermal profile of the injected material in the eye using an infrared thermal camera.

24. The method of claim 23, wherein the infrared thermal camera is a FLIR T530 infrared thermal camera.

25. A single dose composition comprising about 6.0×10¹⁰ genome copies per eye, 1.6×10¹¹ genome copies per eye, 2.5×10¹¹ genome copies per eye, 5.0×10¹¹ genome copies per eye, or 3.0×10¹² genome copies per eye of an expression vector encoding an anti-human vascular endothelial growth factor (hVEGF) antibody in a formulation buffer (pH=7.4), wherein the formulation buffer comprises Dulbecco's phosphate buffered saline and 0.0001% Pluronic F68, wherein the anti-hVEGF antibody comprises a heavy chain comprising the amino acid sequence of SEQ ID NO. 2 or SEQ ID NO. 4, and a light chain comprising the amino acid sequence of SEQ ID NO. 1, or SEQ ID NO. 3; and wherein the wherein the expression vector is an AAV8 vector.

26. The composition of claim 25, wherein the expression vector is Construct II.

27. The method of any one of claims 1-9 and 12-24, wherein the method does not result in shedding of the expression vector.

28. The method of any one of claims 1-9 and 12-24, wherein less than 1000, less than 500, less than 100, less than 50 or less than 10 expression vector gene copies/5 μL are detectable by quantitative polymerase chain reaction in a biological fluid at any point after administration.

29. The method of any one of claims 1-9 and 12-24, wherein 210 expression vector gene copies/5 μL or less are detectable by quantitative polymerase chain reaction in a biological fluid at any point after administration.

30. The method of any one of claims 1-9 and 12-24, wherein less than 1000, less than 500, less than 100, less than 50 or less than 10 vector gene copies/5 μL are detectable by quantitative polymerase chain reaction in a biological fluid by 1, 2, 3, 4, 5, 6, 7, 8, 9, 10, 11, 12, 13 or 14 weeks after administration.

31. The method of any one of claims 1-9 and 12-24, wherein no vector gene copies are detectable in a biological fluid by week 14 after administration of the vector.

32. The method of any one of claims 28-31, wherein the biological fluid is tears, serum or urine.


SEQUENCE LISTING (51 sequences)

SEQ ID NO: 1 - protein, 214 aa, Artificial Sequence
  Ranibizumab Fab Amino Acid Sequence - light chain
DIQLTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 2 - protein, 231 aa, Artificial Sequence
  Ranibizumab Fab Amino Acid Sequence - heavy chain
EVQLVESGGGLVQPGGSLRLSCAASGYDFTHYGMNWVRQAPGKGLEWVGWINTYTGEPTYAADFKRRFTFSLDTSKSTAYLQMNSLRAEDTAVYYCAKYPYYYGTSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHL

SEQ ID NO: 3 - protein, 214 aa, Artificial Sequence
  Bevacizumab Fab Amino Acid Sequence - Light chain
DIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 4 - protein, 231 aa, Artificial Sequence
  Bevacizumab Fab Amino Acid Sequence - Heavy chain
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHL

SEQ ID NO: 5 - protein, 26 aa, Artificial Sequence
  VEGF-A signal peptide
MNFLLSWVHWSLALLLYLHHAKWSQA

SEQ ID NO: 6 - protein, 29 aa, Artificial Sequence
  Fibulin-1 signal peptide
MERAAPSRRVPLPLLLLGGLALLAAGVDA

SEQ ID NO: 7 - protein, 19 aa, Artificial Sequence
  Vitronectin signal peptide
MAPLRPLLILALLAWVALA

SEQ ID NO: 8 - protein, 18 aa, Artificial Sequence
  Complement Factor H signal peptide
MRLLAKIICLMLWAICVA

SEQ ID NO: 9 - protein, 19 aa, Artificial Sequence
  Opticin signal peptide
MRLLAFLSLLALVLQETGT

SEQ ID NO: 10 - DNA, 728 bp, Artificial Sequence
  Bevacizumab cDNA - Light chain
gctagcgccaccatgggctggtcctgcatcatcctgttcctggtggccaccgccaccggcgtgcactccgacatccagatgacccagtccccctcctccctgtccgcctccgtgggcgaccgggtgaccatcacctgctccgcctcccaggacatctccaactacctgaactggtaccagcagaagcccggcaaggcccccaaggtgctgatctacttcacctcctccctgcactccggcgtgccctcccggttctccggctccggctccggcaccgacttcaccctgaccatctcctccctgcagcccgaggacttcgccacctactactgccagcagtactccaccgtgccctggaccttcggccagggcaccaaggtggagatcaagcggaccgtggccgccccctccgtgttcatcttccccccctccgacgagcagctgaagtccggcaccgcctccgtggtgtgcctgctgaacaacttctacccccgggaggccaaggtgcagtggaaggtggacaacgccctgcagtccggcaactcccaggagtccgtgaccgagcaggactccaaggactccacctactccctgtcctccaccctgaccctgtccaaggccgactacgagaagcacaaggtgtacgcctgcgaggtgacccaccagggcctgtcctcccccgtgaccaagtccttcaaccggggcgagtgctgagcggccgcctcgag

SEQ ID NO: 11 - DNA, 1440 bp, Artificial Sequence
  Bevacizumab cDNA - heavy chain
gctagcgccaccatgggctggtcctgcatcatcctgttcctggtggccaccgccaccggcgtgcactccgaggtgcagctggtggagtccggcggcggcctggtgcagcccggcggctccctgcggctgtcctgcgccgcctccggctacaccttcaccaactacggcatgaactgggtgcggcaggcccccggcaagggcctggagtgggtgggctggatcaacacctacaccggcgagcccacctacgccgccgacttcaagcggcggttcaccttctccctggacacctccaagtccaccgcctacctgcagatgaactccctgcgggccgaggacaccgccgtgtactactgcgccaagtacccccactactacggctcctcccactggtacttcgacgtgtggggccagggcaccctggtgaccgtgtcctccgcctccaccaagggcccctccgtgttccccctggccccctcctccaagtccacctccggcggcaccgccgccctgggctgcctggtgaaggactacttccccgagcccgtgaccgtgtcctggaactccggcgccctgacctccggcgtgcacaccttccccgccgtgctgcagtcctccggcctgtactccctgtcctccgtggtgaccgtgccctcctcctccctgggcacccagacctacatctgcaacgtgaaccacaagccctccaacaccaaggtggacaagaaggtggagcccaagtcctgcgacaagacccacacctgccccccctgccccgcccccgagctgctgggcggcccctccgtgttcctgttcccccccaagcccaaggacaccctgatgatctcccggacccccgaggtgacctgcgtggtggtggacgtgtcccacgaggaccccgaggtgaagttcaactggtacgtggacggcgtggaggtgcacaacgccaagaccaagccccgggaggagcagtacaactccacctaccgggtggtgtccgtgctgaccgtgctgcaccaggactggctgaacggcaaggagtacaagtgcaaggtgtccaacaaggccctgcccgcccccatcgagaagaccatctccaaggccaagggccagccccgggagccccaggtgtacaccctgcccccctcccgggaggagatgaccaagaaccaggtgtccctgacctgcctggtgaagggcttctacccctccgacatcgccgtggagtgggagtccaacggccagcccgagaacaactacaagaccaccccccccgtgctggactccgacggctccttcttcctgtactccaagctgaccgtggacaagtcccggtggcagcagggcaacgtgttctcctgctccgtgatgcacgaggccctgcacaaccactacacccagaagtccctgtccctgtcccccggcaagtgagcggccgcc

SEQ ID NO: 12 - DNA, 733 bp, Artificial Sequence
  Ranibizumab cDNA (Light chain comprising a signal sequence)
gagctccatggagtttttcaaaaagacggcacttgccgcactggttatgggttttagtggtgcagcattggccgatatccagctgacccagagcccgagcagcctgagcgcaagcgttggtgatcgtgttaccattacctgtagcgcaagccaggatattagcaattatctgaattggtatcagcagaaaccgggtaaagcaccgaaagttctgatttattttaccagcagcctgcatagcggtgttccgagccgttttagcggtagcggtagtggcaccgattttaccctgaccattagcagcctgcagccggaagattttgcaacctattattgtcagcagtatagcaccgttccgtggacctttggtcagggcaccaaagttgaaattaaacgtaccgttgcagcaccgagcgtttttatttttccgcctagtgatgaacagctgaaaagcggcaccgcaagcgttgtttgtctgctgaataatttttatccgcgtgaagcaaaagtgcagtggaaagttgataatgcactgcagagcggtaatagccaagaaagcgttaccgaacaggatagcaaagatagcacctatagcctgagcagcaccctgaccctgagcaaagcagattatgaaaaacacaaagtgtatgcctgcgaagttacccatcagggtctgagcagtccggttaccaaaagttttaatcgtggcgaatgctaatagaagcttggtacc

SEQ ID NO: 13 - DNA, 779 bp, Artificial Sequence
  Ranibizumab cDNA (Heavy chain comprising a signal sequence)
gagctcatatgaaatacctgctgccgaccgctgctgctggtctgctgctcctcgctgcccagccggcgatggccgaagttcagctggttgaaagcggtggtggtctggttcagcctggtggtagcctgcgtctgagctgtgcagcaagcggttatgattttacccattatggtatgaattgggttcgtcaggcaccgggtaaaggtctggaatgggttggttggattaatacctataccggtgaaccgacctatgcagcagattttaaacgtcgttttacctttagcctggataccagcaaaagcaccgcatatctgcagatgaatagcctgcgtgcagaagataccgcagtttattattgtgccaaatatccgtattactatggcaccagccactggtatttcgatgtttggggtcagggcaccctggttaccgttagcagcgcaagcaccaaaggtccgagcgtttttccgctggcaccgagcagcaaaagtaccagcggtggcacagcagcactgggttgtctggttaaagattattttccggaaccggttaccgtgagctggaatagcggtgcactgaccagcggtgttcatacctttccggcagttctgcagagcagcggtctgtatagcctgagcagcgttgttaccgttccgagcagcagcctgggcacccagacctatatttgtaatgttaatcataaaccgagcaataccaaagtggataaaaaagttgagccgaaaagctgcgataaaacccatctgtaatagggtacc

SEQ ID NO: 14 - protein, 11 aa, Artificial Sequence
  Bevacizumab and Ranibizumab Light Chain CDR1
SASQDISNYLN

SEQ ID NO: 15 - protein, 7 aa, Artificial Sequence
  Bevacizumab and Ranibizumab Light Chain CDR2
FTSSLHS

SEQ ID NO: 16 - protein, 9 aa, Artificial Sequence
  Bevacizumab and Ranibizumab Light Chain CDR3
QQYSTVPWT

SEQ ID NO: 17 - protein, 10 aa, Artificial Sequence
  Bevacizumab Heavy Chain CDR1
GYTFTNYGMN

SEQ ID NO: 18 - protein, 17 aa, Artificial Sequence
  Bevacizumab Heavy Chain CDR2
WINTYTGEPTYAADFKR

SEQ ID NO: 19 - protein, 14 aa, Artificial Sequence
  Bevacizumab Heavy Chain CDR3
YPHYYGSSHWYFDV

SEQ ID NO: 20 - protein, 10 aa, Artificial Sequence
  Ranibizumab Heavy Chain CDR1
GYDFTHYGMN

SEQ ID NO: 21 - protein, 14 aa, Artificial Sequence
  Ranibizumab Heavy Chain CDR3
YPYYYGTSHWYFDV

SEQ ID NO: 22 - protein, 18 aa, Artificial Sequence
  Albumin signal peptide
MKWVTFISLLFLFSSAYS

SEQ ID NO: 23 - protein, 18 aa, Artificial Sequence
  Chymotrypsinogen signal peptide
MAFLWLLSCWALLGTTFG

SEQ ID NO: 24 - protein, 20 aa, Artificial Sequence
  Interleukin-2 signal peptide
MYRMQLLSCIALILALVTNS

SEQ ID NO: 25 - protein, 15 aa, Artificial Sequence
  Trypsinogen-2 signal peptide
MNLLLILTFVAAAVA

SEQ ID NO: 26 - protein, 19 aa, Artificial Sequence
  F2A site
LLNFDLLKLAGDVESNPGP

SEQ ID NO: 27 - protein, 21 aa, Artificial Sequence
  Linker T2A
GSGEGRGSLLTCGDVEENPGP

SEQ ID NO: 28 - protein, 22 aa, Artificial Sequence
  Linker P2A
GSGATNFSLLKQAGDVEENPGP

SEQ ID NO: 29 - protein, 23 aa, Artificial Sequence
  Linker E2A
GSGQCTNYALLKLAGDVESNPGP

SEQ ID NO: 30 - protein, 25 aa, Artificial Sequence
  Linker F2A
GSGVKQTLNFDLLKLAGDVESNPGP

SEQ ID NO: 31 - protein, 4 aa, Artificial Sequence
  Furin linker
RKRR

SEQ ID NO: 32 - protein, 4 aa, Artificial Sequence
  Furin linker
RRRR

SEQ ID NO: 33 - protein, 4 aa, Artificial Sequence
  Furin linker
RRKR

SEQ ID NO: 34 - protein, 4 aa, Artificial Sequence
  Furin linker
RKKR

SEQ ID NO: 35 - protein, 4 aa, Artificial Sequence
  Furin linker
  MISC_FEATURE (2)..(2): X = any amino acid
  MISC_FEATURE (3)..(3): X = Lys or Arg
RXXR

SEQ ID NO: 36 - protein, 4 aa, Artificial Sequence
  Furin linker
  MISC_FEATURE (2)..(2): X = any amino acid
RXKR

SEQ ID NO: 37 - protein, 4 aa, Artificial Sequence
  Furin linker
  MISC_FEATURE (2)..(2): X = any amino acid
RXRR

SEQ ID NO: 38 - protein, 215 aa, Artificial Sequence
  Ranibizumab Fab Amino Acid Sequence - light chain
MDIQLTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 39 - protein, 236 aa, Artificial Sequence
  Ranibizumab Fab Amino Acid Sequence - heavy chain
MEVQLVESGGGLVQPGGSLRLSCAASGYDFTHYGMNWVRQAPGKGLEWVGWINTYTGEPTYAADFKRRFTFSLDTSKSTAYLQMNSLRAEDTAVYYCAKYPYYYGTSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHLRKRR

SEQ ID NO: 40 - protein, 232 aa, Artificial Sequence
  Ranibizumab Fab Amino Acid Sequence - heavy chain
MEVQLVESGGGLVQPGGSLRLSCAASGYDFTHYGMNWVRQAPGKGLEWVGWINTYTGEPTYAADFKRRFTFSLDTSKSTAYLQMNSLRAEDTAVYYCAKYPYYYGTSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHL

SEQ ID NO: 41 - protein, 736 aa, Artificial Sequence
  AAV1
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEEVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDEDKFFPMSGVMIFGKESAGASNTALDNVMITDEEEIKATNPVATERFGTVAVNFQSSSTDPATGDVHAMGALPGMVWQDRDVYLQGPIWAKIPHTDGHFHPSPLMGGFGLKNPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 42 - protein, 735 aa, Artificial Sequence [sequence omitted]

SEQ ID NO: 43 - protein, 736 aa, Artificial Sequence
  AAV3-3
MAADGYLPDWLEDNLSEGIREWWALKPGVPQPKANQQHQDNRRGLVLPGYKYLGPGNGLDKGEPVNEADAAALEHDKAYDQQLKAGDNPYLKYNHADAEFQERLQEDTSFGGNLGRAVFQAKKRILEPLGLVEEAAKTAPGKKGAVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVRGVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTGTVNHQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRNL

SEQ ID NO: 44 - protein, 734 aa, Artificial Sequence
  AAV4-4
MTDGYLPDWLEDNLSEGVREWWALQPGAPKPKANQQHQDNARGLVLPGYKYLGPGNGLDKGEPVNAADAAALEHDKAYDQQLKAGDNPYLKYNHADAEFQQRLQGDTSFGGNLGRAVFQAKKRVLEPLGLVEQAGETAPGKKRPLIESPQQPDSSTGIGKKGKQPAKKKLVFEDETGAGDGPPEGSTSGAMSDDSEMRAAAGGAAVEGGQGADGVGNASGDWHCDSTWSEGHVTTTSTRTWVLPTYNNHLYKRLGESLQSNTYNGFSTPWGYFDFNRFHCHFSPRDWQRLINNNWGMRPKAMRVKIFNIQVKEVTTSNGETTVANNLTSTVQIFADSSYELPYVMDAGQEGSLPPFPNDVFMVPQYGYCGLVTGNTSQQQTDRNAFYCLEYFPSQMLRTGNNFEITYSFEKVPFHSMYAHSQSLDRLMNPLIDQYLWGLQSTTTGTTLNAGTATTNFTKLRPTNFSNFKKNWLPGPSIKQQGFSKTANQNYKIPATGSDSLIKYETHSTLDGRWSALTPGPPMATAGPADSKFSNSQLIFAGPKQNGNTATVPGTLIFTSEEELAATNATDTDMWGNLPGGDQSNSNLPTVDRLTALGAVPGMVWQNRDIYYQGPIWAKIPHTDGHFHPSPLIGGFGLKHPPPQIFIKNTPVPANPATTFSSTPVNSFITQYSTGQVSVQIDWEIQKERSKRWNPEVQFTSNYGQQNSLLWAPDAAGKYTEPRAIGTRYLTHHL

SEQ ID NO: 45 - protein, 724 aa, Artificial Sequence
  AAV5
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 46 - protein, 736 aa, Artificial Sequence
  AAV6
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQSSSTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 47 - protein, 737 aa, Artificial Sequence
  AAV7
MAADGYLPDWLEDNLSEGIREWWDLKPGAPKPKANQQKQDNGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPAKKRPVEPSPQRSPDSSTGIGKKGQQPARKRLNFGQTGDSESVPDPQPLGEPPAAPSSVGSGTVAAGGGAPMADNNEGADGVGNASGNWHCDSTWLGDRVITTSTRTWALPTYNNHLYKQISSETAGSTNDNTYFGYSTPWGYFDFNRFHCHFSPRDWQRLINNNWGFRPKKLRFKLFNIQVKEVTTNDGVTTIANNLTSTIQVFSDSEYQLPYVLGSAHQGCLPPFPADVFMIPQYGYLTLNNGSQSVGRSSFYCLEYFPSQMLRTGNNFEFSYSFEDVPFHSSYAHSQSLDRLMNPLIDQYLYYLARTQSNPGGTAGNRELQFYQGGPSTMAEQAKNWLPGPCFRQQRVSKTLDQNNNSNFAWTGATKYHLNGRNSLVNPGVAMATHKDDEDRFFPSSGVLIFGKTGATNKTTLENVLMTNEEEIRPTNPVATEEYGIVSSNLQAANTAAQTQVVNNQGALPGMVWQNRDVYLQGPIWAKIPHTDGNFHPSPLMGGFGLKHPPPQILIKNTPVPANPPEVFTPAKFASFITQYSTGQVSVEIEWELQKENSKRWNPEIQYTSNFEKQTGVDFAVDSQGVYSEPRPIGTRYLTRNL

SEQ ID NO: 48 - protein, 738 aa, Artificial Sequence
  AAV8
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 49 - protein, 736 aa, Artificial Sequence
  hu31
MAADGYLPDWLEDTLSEGIRQWWKLKPGPPPPKPAERHKDDSRGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGSQPAKKKLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGGQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVSTEGVYSEPRPIGTRYLTRNL

SEQ ID NO: 50 - protein, 736 aa, Artificial Sequence
  hu32
MAADGYLPDWLEDTLSEGIRQWWKLKPGPPPPKPAERHKDDSRGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGSQPAKKKLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 51 - protein, 736 aa, Artificial Sequence
  AAV9
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL